(19) United States
(12) Patent Application Publication
(10) Pub. No.: US 2026/0290502 A1
(43) Pub. Date: Sep. 24, 2026

(54) METHODS AND COMPOSITIONS FOR IMPROVED MULTIPLEX GENOTYPING AND SEQUENCING

(71) Applicant: COVARIANCE BIOSCIENCES, LLC, Cleveland, OH (US)

(72) Inventor: Alexander MIRON, Pepper Pike, OH (US)

(73) Assignee: COVARIANCE BIOSCIENCES, LLC, Cleveland, OH (US)

(21) Appl. No.: 19/570,731

(22) Filed: Mar. 18, 2026

Related U.S. Application Data
(63) Continuation of application No. 17/255,722, filed on Jun. 28, 2019, now Pat. No. 12,609,184.
(60) Provisional application No. 62/692,293, filed on Jun. 29, 2018.

(51) Int. Cl. G16B 25/20; C12Q 1/686; G16B 40/00

(52) U.S. Cl. CPC G16B 25/20; C12Q 1/686; G16B 40/00

(57) ABSTRACT

The technology described herein is directed to methods of designing primers for multiplex PCR amplification. Also described herein are methods for equalization of reads in these approaches. A variation is described herein that permits single base multiplexed sequencing on an NGS platform. Also described herein are methods to rapidly analyze NGS sequencing data to automatically provide genotype or sequencing results and methods to identify and quantify low abundance rare variants in clinically relevant genes in a minority of tumor cells from a complex mixture of cells.

### CROSS-REFERENCE TO RELATED APPLICATIONS

This application is a continuation under 35 U.S.C. § 120 of U.S. application Ser. No. 17/255,722, filed Dec. 23, 2020, which is a 35 U.S.C. § 371 National Phase Entry Application of International Application No. PCT/US2019/039921, filed Jun. 28, 2019, which designates the U.S., and which claims benefit under 35 U.S.C. § 119(e) of U.S. Provisional Application No. 62/692,293 filed Jun. 29, 2018, the contents of each of which are incorporated herein by reference in their entireties.

### SEQUENCE LISTING

The instant application contains a Sequence Listing which has been submitted electronically in XML format via Patent Center and is hereby incorporated by reference in its entirety. Said XML copy, created on Mar. 17, 2026, is named 079753-093010USC1_SL.xml and is 1,828,479 bytes in size.

### TECHNICAL FIELD

The technology described herein relates to methods and compositions for multiplexed genotyping and sequencing.

### BACKGROUND

So-called “Next Generation Sequencing” (NGS) or “NextGen” nucleic acid sequencing has revolutionized nucleic acid analysis and bioinformatics. These sequencing approaches rely on the concept of massively parallel processing, in which millions of short sequence reads are generated in parallel, followed by in silico assembly of the resulting data into corresponding genomic, expressed gene or other relevant sequences.

While the details vary, most NGS approaches rely upon at least one multiplex amplification step. This is particularly true when NGS approaches are applied to the genotyping of known genomic sequence variations, including, but not limited to single nucleotide polymorphisms (SNPs). For such approaches, when hundreds to thousands of target variable sequences are genotyped in a single reaction, interactions between primers can result in products in which primers in the reaction serve as both template and primer to generate so-called “primer dimer” extension products.

### SUMMARY

Described herein are improved methods of designing primers for multiplex PCR amplification. By minimizing primer:primer interactions, the primer-design methods described herein can dramatically improve throughput in NGS-based multiplex genotyping methods. Thus, provided herein are methods for primer design that permit large amplicon number multiplexing and genotype calling.

It has also been recognized that uneven amplification of genotyping targets reduces the efficiency, and therefore the throughput of NGS-based multiplex genotyping approaches. Accordingly, also described herein are methods for equalization of reads in these approaches. Embodiments of the methods use anti-sense oligos in various concentrations to inhibit high-performing amplicons from forming, and thereby allow poorer performing amplicons to produce more data. Some embodiments use anti-sense oligos with additional sequences on their 5′-end that permanently inactivate primers in solution once annealed and extended (inactivator oligos). Other embodiments use anti-sense oligos with reversible binding characteristics having only complementary sequences (attenuator oligos).

The methods described herein permit efficient sequencing and genotype calling in high multiplex. Thus, described herein are methods for performing multiplex PCR that results in even reads produced by NGS instruments for each amplicon (for 2-10,000 amplicons) and each sample (for 1-100,000 DNA samples). In one embodiment, a variation is described herein that permits single base multiplexed sequencing on an NGS platform, in order to avoid sequencing additional regions where this is beneficial.

Also described herein are methods to rapidly analyze NGS sequencing data using personalized software to automatically provide genotype or sequencing results, and methods to identify and quantify low abundance rare variants in clinically relevant genes in a minority of tumor cells from a complex mixture of cells.

In one aspect, described herein is a method for primer design that allows for large amplicon number multiplexing. In one embodiment, the method comprises the synthesis and use of a computer algorithm to perform steps as described herein to identify a primer set that permits large amplicon number multiplexing, which can be followed by synthesis of the designed primer set, e.g., for multiplex genotyping, among other uses. In one embodiment, the primer design algorithm is incorporated into the PlexForm™ software package.

In one aspect, described herein is a method of preparing a primer set for multiplex amplification or genotyping, the method comprising: A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of: 1) identifying all possible primers of 17 to 35 nucleotides within 100 base pairs of each genomic target sequence variation in set N of variable genomic target sequences; 2) for each member of set N, selecting a subset of primer pairs from the set of step (1) that satisfies the conditions for a primer selection algorithm; 3) evaluating specificity of primer pairs chosen in step (2) in the genome, keeping only those pairs predicted to be specific for their respective targets; 4) selecting a set of optimized primers for the amplification of target gene set N, where the optimal primers are selected to minimize primer-primer interactions with other primers in the set by iterative calculation of predicted ΔG (or delta G, Gibbs free energy) for interactions between primers to generate a fitness score and use of a fitness score optimization method selected from one or a combination of the group consisting of: a) a Monte Carlo random or pseudo-random selection method; b) a golden section search; c) gradient descent; d) minima hopping; e) genetic algorithm; f) neural networks; g) cluster analysis, in which substitution is picked to minimize score; and h) cluster analysis to create bins; and B) synthesizing the optimized primer set selected in step (4).

In another aspect, described herein is a method of preparing a primer set for multiplex amplification or genotyping, the method comprising: A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of: 1) identifying all possible primers of 17 to 35 nucleotides within 100 base pairs of each genomic target sequence variation in set N of variable genomic target sequences; 2) for the primers identified in step (1), randomly selecting a primer pair for each target in set N that satisfies the conditions for a primer selection algorithm; 3) evaluating specificity of primer pairs chosen in step (2) in the genome, keeping only those pairs predicted to be specific for their respective targets; 4) repeat step (2) on the primer pairs kept from step (3) to generate set P, a population of randomly selected primer sets for each target in set N; 5) generating a Fitness Score for each member of population P based upon ΔG for all possible interactions between candidate primers in each member of the population; 6) picking acceptable member(s) of the population P based on Fitness Score; 7) repeating steps (4)-(6) iteratively until a set of primer pairs for target genes identified in step (6) has a Fitness Score at a predetermined threshold; and B) synthesizing the primer set identified in step (7).

In one embodiment of this method, the Fitness Score is generated according to the rule: a) G=the set of ΔG's for all possible interactions determined for a given member of set P; and b) Fitness Score is calculated by: i) for each member of set P, calculating the sum, S, of |ΔG|^{Q} for each ΔG value in that member, wherein Q is a weighting factor constant exponent that makes large ΔG absolute values much larger than small values; ii) S′=S/#of ΔG values in G; iii) H=T/S′, wherein T is a constant that makes H small for large values of S′ and H large for small values of S′; and iv) Fitness Score=H^{R}, wherein R is a weighting factor constant exponent that makes large values of H larger, and small values of H smaller.

In another aspect, described herein is a method of preparing a primer set for multiplex amplification or genotyping, the method comprising: A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of: 1) identifying all possible primers of 17 to 35 nucleotides within 100 base pairs of each genomic target sequence variation in set N of variable genomic target sequences; 2) for the primers identified in step (1), randomly selecting a primer pair for each target in set N that satisfies the conditions for a primer selection algorithm and is predicted to be specific for its target in the genome, or providing a primer pair for each target in set N, that has been selected to reduce potential for primer:primer interactions with other primers in the set and is predicted to be specific for its target in the genome; 3) repeat step (2) to generate population Z, of size 2 or greater, of primer pair sets for each target in set N; 4) generating a Fitness Score for each member of population Z based upon ΔG for all possible interactions between candidate primers in each member of the population; 5) selecting the worst members of population Z based on Fitness Scores as set W; 6) replacing a primer for a single target from W with another primer from step (2), and generating a Fitness Score for the resulting set; wherein if the change results in an improved Fitness Score relative to the Fitness Score generated in step (4), the resulting new set W′ replaces set W, and if the change results in a no change in Fitness Score or a decreased Fitness Score, keeping set W; 7) iteratively repeating steps (4)-(6) on the set W or W′ retained in each iteration of step (6) until a set of primer pairs for target genes in set N is identified that has a Fitness Score at a predetermined threshold, or, if a predetermined threshold is not reached by iteratively repeating steps (4)-(6), beginning again at step (2) and iteratively repeating steps (4)-(6) until a set of primer pairs for target genes in set N is identified that has a Fitness Score at the predetermined threshold; and B) synthesizing the primer set selected in step (7) that reaches the predetermined Fitness Score threshold.

In one embodiment of the aspect, the Fitness Score is generated according to the rule: a) G=the set of ΔG's for all possible interactions determined for a given member of set P; and b) Fitness Score is calculated by: i) for each member of set P, calculating the sum, S, of |ΔG|^{Q} for each ΔG value in that member, wherein Q is a weighting factor constant exponent that makes large ΔG absolute values much larger than small values; ii) S′=S/#of ΔG values in G; iii) H=T/S′, wherein T is a constant that makes H small for large values of S′ and H large for small values of S′; and iv) Fitness Score=H^{R}, wherein R is a weighting factor constant exponent that makes large values of H larger, and small values of H smaller.

In another embodiment of the aspect, the step of providing a primer pair for each target in set N that has been selected to reduce potential for primer:primer interactions with other primers in the set provides primer sets selected using one or more of a Monte Carlo random or pseudo-random selection method, a golden section search, gradient descent, minima hopping, a genetic algorithm, neural networks, cluster analysis in which substitution is picked to minimize score, or cluster analysis to create bins.

In another aspect, described herein is a method of preparing a primer set for multiplex amplification or genotyping, the method comprising: A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of: 1) identifying all possible primers of 17 to 35 nucleotides within 100 base pairs of each genomic target sequence variation in set N of variable genomic target sequences; 2) generating primer set Z, including a primer pair for each member of set N either by: (a) randomly selecting from the primers identified in step (1) a primer pair for each target in set N that satisfies the conditions for a primer selection algorithm and is predicted to be specific for its target in the genome; or (b) providing a primer pair for each target in set N that is predicted to be specific for its target in the genome, and that has been selected to reduce potential for primer:primer interactions with other primers in the set; 3) generating a Fitness Score for primer set Z based upon ΔG for all possible interactions between candidate primers in each member of the population; 4) making a change to a primer for a single target from set Z to generate new set Z′, and generating a Fitness Score for set Z′, wherein if the change results in an improved Fitness Score relative to that generated in step (3), the resulting new set Z′ replaces set Z, and if the change results in no change in Fitness Score or a decreased Fitness Score, keeping set Z; and 5) repeating step (4) iteratively until further iterations do not improve fitness of set Z; and B) synthesizing the primer set selected in step (5).

In one embodiment of the aspect, the Fitness Score is generated according to the rule: a) G=the set of ΔG's for all possible interactions determined for members of primer set Z; and b) Fitness Score is calculated by: i) calculating the sum, S, of |ΔG|^{Q} for each ΔG value, wherein Q is a weighting factor constant exponent that makes large ΔG absolute values much larger than small values; ii) S′=S/# of ΔG values in G; iii) H=T/S′, wherein T is a constant that makes H small for large values of S′ and H large for small values of S′; and iv) Fitness Score=H^{R}, wherein R is a weighting factor constant exponent that makes large values of H larger, and small values of H smaller.

In another aspect, described herein is a method of preparing a primer set for multiplex amplification or genotyping, the method comprising: A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of: 1) identifying all possible primers of 17 to 35 nucleotides within 100 base pairs of each genomic target sequence variation in set N of variable genomic target sequences; 2) providing a set of optimized primer pairs for the amplification of target gene set N, where the optimal primer pairs are predicted to be specific for their target genes in the genome, and are selected to minimize primer-primer interactions with other primers in the set by iterative calculation of predicted ΔG for all possible interactions between primers to generate a Fitness Score and use of a Fitness Score optimization method selected from one or a combination of the group consisting of: a) a Monte Carlo random or pseudo-random selection method; b) a golden section search; c) gradient descent; d) minima hopping; e) genetic algorithm; f) neural networks; g) cluster analysis, in which substitution is picked to minimize score; and h) cluster analysis to create bins; 3) adding the set of optimized primers of step (2) to set M; 4) while maintaining a degree of dissimilarity from primer sets included in set M, selecting a primer pair for each target in set N from step (1) and designating it set Z, wherein the primer pairs satisfy the conditions for a primer selection algorithm, and are predicted to be specific for their target genes in the genome; 5) optimizing primer pairs of set Z for the amplification of target gene set N, to minimize primer-primer interactions with other primers in the set by iterative calculation of predicted ΔG for all possible interactions between primers to generate a Fitness Score and use of a Fitness Score optimization method selected from one or a combination of methods (a)-(h) of step (2); and 6) repeating steps (2)-(5) iteratively until a set of primer pairs for target gene set N identified in step (5) has a Fitness Score at a predetermined threshold; and B) synthesizing the optimized primer set selected in step (6).

In one embodiment of the aspect, the Fitness Score is generated according to the rule: a) G=the set of ΔG's for all possible interactions determined for members of primer set Z; and b) Fitness Score is calculated by: i) calculating the sum, S, of |ΔG|^{Q} for each ΔG value, wherein Q is a weighting factor constant exponent that makes large ΔG absolute values much larger than small values; ii) S′=S/# of ΔG values in G; iii) H=T/S′, wherein T is a constant that makes H small for large values of S′ and H large for small values of S′; and iv) Fitness Score=H^{R}, wherein R is a weighting factor constant exponent that makes large values of H larger, and small values of H smaller.

In another aspect, described herein is a method of preparing a primer set for multiplex amplification or genotyping, the method comprising: A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of: 1) identifying all possible primers of 17 to 35 nucleotides within 100 base pairs of each genomic target sequence variation in set N of variable genomic target sequences; 2) For each primer identified in step (1) creating a node P_{nz}, such node connected to a node for the corresponding target (Tₙ), wherein: (i) each node outputs its ID and a numeric value; (ii) each Tₙ produces ID of one of the P_{nz} nodes connected to it; (iii) each one of the Tₙ nodes is connected to all others; and (iv) each node Tₙ represents a multilayer neural network; 3) calculating a Fitness Score for output of the neural network, and on the basis of Fitness Score, the value produced by the network is compared to target, and neural network parameters for a plurality of the Tₙ are changed; 4) calculating Fitness Score again for output of the neural network with parameters changed in step (3); 5) determining if a change was beneficial or not to the fitness of the resulting set, wherein if the change was beneficial, the direction of change is maintained with smaller increments, and wherein if the change was not beneficial, either direction is reversed or the parameters revert to a previous state; 6) repeating steps (3)-(5) iteratively, wherein at a plurality of iterations random changes are made to the parameters of the network, and wherein when the rate of fitness improvement decreases, the frequency of such random changes is increased, until a set of primer pairs for target genes in set N is identified that has a fitness score at a predetermined threshold; and B) synthesizing the optimized primer set selected in step (6).

In one embodiment of the aspect, the Fitness Score is generated according to the rule: a) G=the set of ΔG's for all possible interactions determined for members of a primer set for targets N; and b) Fitness Score is calculated by: i) calculating the sum, S, of |ΔG|^{Q} for each ΔG value, wherein Q is a weighting factor constant exponent that makes large ΔG absolute values much larger than small values; ii) S′=S/#of ΔG values in G; iii) H=T/S′, wherein T is a constant that makes H small for large values of S′ and H large for small values of S′; and iv) Fitness Score=H^{R}, wherein R is a weighting factor constant exponent that makes large values of H larger, and small values of H smaller.

In another aspect, described herein is a method of preparing a primer set for multiplex amplification or genotyping, the method comprising: A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of: 1) identifying all possible primers of 17 to 35 nucleotides within 100 base pairs of each genomic target sequence variation in set N of variable genomic target sequences; 2) picking a target at random, as well as a primer for such target, and placing it in set R; 3) picking an additional target, and calculating a Fitness Score evaluating all primers for this target in combination with primers already in set R on the basis of ΔG for all potential interactions, wherein the primer that results in the best Fitness Score is added to set R; 4) if fitness of set R is below a predetermined threshold T, removing one of the primers from R according to the following: calculating a Fitness Score for set Ri, wherein the i^{th} target with its primer is removed from set R, and the set with the best Fitness Score determines the target with its primer to be removed from set R and placed back into the pool of primers of step (1); and 5) repeating steps (3) and (4) until all targets are assigned primers; and B) synthesizing the optimized primer set selected in step (5). In this embodiment, if one is trying to pick a next primer to add to set R and any primer that is picked does not decrease fitness to such level that it is below T, options include restarting the process with a different starting point (primer) or decreasing the initial threshold T.

In one embodiment of the aspect, the Fitness Score is generated according to the rule: a) G=the set of ΔG's for all possible interactions determined for members of a primer set; and b) Fitness Score is calculated by: i) calculating the sum, S, of |ΔG|^{Q} for each ΔG value, wherein Q is a weighting factor constant exponent that makes large ΔG absolute values much larger than small values; ii) S′=S/#of ΔG values in G; iii) H=T/S′, wherein T is a constant that makes H small for large values of S′ and H large for small values of S′; and iv) Fitness Score=H^{R}, wherein R is a weighting factor constant exponent that makes large values of H larger, and small values of H smaller.

In another aspect, described herein is a method of preparing a primer set for multiplex amplification or genotyping, the method comprising: A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of: 1) identifying all possible primers of 17 to 35 nucleotides within 100 base pairs of each genomic target sequence variation in set N of variable genomic target sequences; 2) picking a target at random, as well as a primer for such target, and placing it in set R; 3) picking an additional target, and calculating a Fitness Score evaluating all primers for this target in combination with primers already in set R on the basis of ΔG for all potential interactions, wherein the primer that results in the best Fitness Score is added to set R; 4) if fitness of set R is below a predetermined threshold T, removing one of the primers from R according to the following: calculating a Fitness Score for set Ri, wherein the i^{th} target with its primer is removed from set R, and the set with the best Fitness Score determines the target with its primer to be removed from set R and placed back into the pool of primers of step (1); 5) repeating steps (3) and (4) until all targets are assigned primers; 6) once all targets are assigned primers, designating set R as R₁, and its fitness as F₁; 7) creating empty set R_{z+1}, where Z is the number of sets, with fitness F_{z+1}; 8) for each set Rz, where z is an index from 1 to number of sets R, determining the element that is worst for the set's fitness, and removing this element, designated Target E; 9) recalculating Fz after removal of E; 10) for all Rz, determining where E can be added so as to maximize Fz and maximize the minimum of Fz; and 11) if the minimum of Fz is below the predetermined threshold, repeating steps (7)-(10) until the standard deviation of Fz is below the predetermined threshold, thereby designing a multiplex primer set; and B) synthesizing the optimized primer set designed in step (11). In one embodiment, the step of determining the element in step (8) that is worst for fitness is performed in a method analogous to step (4).

In another aspect, described herein is a method of preparing a primer set for multiplex amplification or genotyping, the method comprising: A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of: 1) identifying all possible primers of 17 to 35 nucleotides within 100 base pairs of each genomic target sequence variation in set N of variable genomic target sequences; 2) for each member of set N, selecting from the set of primers in step (1) a subset of primer pairs that satisfies the conditions for a primer selection algorithm and is predicted to be specific for its target; 3) repeating step (2) to generate set P, a population of randomly selected primer sets for each target gene in set N; 4) calculating a Fitness Score for each member of the population P; 5) placing members of population P into a pool of candidate primer sets on the basis of Fitness Scores; 6) randomly selecting a plurality of “parent” sets of candidate primers from the pool of step (5), each parent set including a different pair of candidate primer sets, parent A and parent B; 7) for each parent set of candidate primers, creating a crossover set of candidate primers by replacing a subset of candidate primer pairs of parent A with the corresponding subset of primer pairs of parent B; 8) randomly replacing one primer pair in crossover set A with a different primer pair for the corresponding target sequence generated in step (2) to create a Generation 2 population of primer sets for each target gene in set N; and 9) repeating steps (4)-(8) iteratively until a set of primer pairs for target genes in set N is identified that has a Fitness Score at a predetermined threshold, and runs for an additional set amount of iterations with no measurable improvement in the fitness of the best member, whereby an optimized primer set is designed; and B) synthesizing the optimized primer set designed in step (9).

In one embodiment of the aspect, the Fitness Score is generated according to the rule: a) G=the set of ΔG's for all possible interactions determined for members of a primer set; and b) Fitness Score is calculated by: i) calculating the sum, S, of |ΔG|^{Q} for each ΔG value, wherein Q is a weighting factor constant exponent that makes large ΔG absolute values much larger than small values; ii) S′=S/#of ΔG values in G; iii) H=T/S′, wherein T is a constant that makes H small for large values of S′ and H large for small values of S′; and iv) Fitness Score=H^{R}, wherein R is a weighting factor constant exponent that makes large values of H larger, and small values of H smaller.

In another aspect, described herein is a method of preparing a primer set for multiplex amplification or genotyping, the method comprising: A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of: 1) identifying all possible primers of 17 to 35 nucleotides within 100 base pairs of each genomic target sequence variation in set N of variable genomic target sequences; 2) selecting a primer set for the multiplex amplification and genotyping of the members of set N comprising: a) from the set of all possible primers for each genomic target sequence variation of step (1), randomly selecting set P, a population of sets of candidate primers, each individual set of candidate primers in population P including a primer pair for the amplification of each member of set N of variable genomic target sequences to be genotyped; b) calculating a fitness score for each member of the population of set P by calculating ΔG for all possible interactions between candidate primers in each member of the population of set P, and assigning each member of set P a Fitness Score according to the rule: i) G=the set of ΔG's for all possible interactions determined for a given member of set P; ii) Number of top scorers to go into next generation=1 . . . N, Number of distinct populations sets=1 . . . N, and Population size=1 . . . N such that number of top scorers to go into next generation is greater or equal to population size; wherein fitness score is calculated by: iii) for each member of set P, calculating the sum, S, of |ΔG|^{Q} for each ΔG value in that member, wherein Q is a weighting factor constant exponent that makes large ΔG absolute values much larger than small values; iv) S′=S/#of ΔG values in G; v) H=T/S′, wherein T is a constant that makes H small for large values of S′ and H large for small values of S′; vi) Fitness Score=H^{R}, wherein R is a weighting factor constant exponent that makes large values of H larger, and small values of H smaller; c) selecting a set of primers for the multiplex amplification and genotyping of members of set N by: i) randomly selecting a plurality of sets of “parent” sets of candidate primers, each having parent set A and parent set B, from set P based upon Fitness Scores; ii) for each member of the plurality of sets of parents, creating a crossover set of candidate primers by replacing a subset of candidate primers in parent set A with a corresponding subset of candidate primers in parent set B, resulting in two crossover sets, crossover set A and crossover set B; and iii) randomly replacing one primer pair in crossover set A with a different primer pair for the corresponding variable genomic target sequence to create a next generation population of candidate sets of primers, Generation 2; and d) iteratively repeating steps (a)-(c), whereby a primer set for the multiplex amplification and genotyping of set N of variable genomic target sequences is selected; and B) synthesizing the primer set designed in step (A).

In another aspect, described herein is a method for multiplex amplification, sequencing, and/or genotyping, the method comprising using a primer set designed according to any one of the preceding aspects.

In another aspect, described herein is a method for equalization of reads in a next generation sequencing method, the method comprising using anti-sense oligos in concentrations sufficient to inhibit high-performing amplicons from forming and thereby allowing poorer performing amplicons to produce more data.

In another aspect, described herein is a method for performing multiplex PCR that results in even reads produced by NGS instruments for each amplicon (for 2-10,000 amplicons) and each sample (for 1-100,000 DNA samples). Embodiments of this method benefit from improved primer design methods as described herein and, for example, the use of antisense oligonucleotides targeting a subset of amplicons that are high-performing or highly efficiently amplified to permit amplicons that amplify less efficiently to produce more data. Embodiments include the use of primers designed according to the methods described herein in multiplex PCR, high throughput genotyping, and other approaches using NGS.

In another aspect, described herein is a variation of NGS (e.g., PlexSeq™ sequencing) that permits single base multiplexed sequencing on an NGS platform in order to avoid sequencing additional regions, e.g., when this is beneficial. One embodiment comprises designing a multiplex genotyping primer set as described herein, and adding random nucleotides to the 5′ end of the primers in the set that are shorter than the longest primer in the set, such that each primer in the set is the same length, N, and performing only N+1 cycles in an NGS sequencing run.

In another aspect, described herein is a method to rapidly analyze NGS data (e.g., PlexSeq™ sequencing data) using personalized software (e.g., PlexCall™ software) to automatically provide genotype or sequencing results.

In another aspect, described herein is a method of using NGS methodology (e.g., PlexSeq™ technology) to identify and quantify low abundance rare variants in clinically relevant genes in a minority of tumor cells from a complex mixture of cells.

### BRIEF DESCRIPTION OF THE DRAWINGS

FIG. 1 is a PlexForm™ Fitness graph example demonstrating optimization of primer mix.

FIG. 2 is a bar graph showing a decrease in variance in read numbers in TP53 sequencing multiplex by addition of blockers to p7 and p15.

FIG. 3 is a schematic showing primary amplification using a 2×PCR mix and DNA samples.

FIG. 4 is a schematic showing secondary amplification and barcoding reaction mixing, cleanup and NGS run.

FIG. 5 is a series of box plots showing control DNA analysis of 9 multiplexed amplicons barcoded in 20,682 PlexSeq™ reactions and analyzed simultaneously on one NGS run. Box plots demonstrate even amplification across amplicon targets and 20,682 samples based on read depth (on Y-axis).

FIG. 6 is an image showing the PlexCall™ front-end, demonstrating simplicity of selecting output files from sequencer and simple sample sheet indicating locations to correlate with barcode positions for analysis.

FIG. 7 is a scatterplot showing mutation frequency versus fold dilution from data in TABLE 4, demonstrating sensitivity of assay down to 1% or lower.

### DETAILED DESCRIPTION

Described herein are methods that dramatically increase the degree of multiplexing achievable for multiplex PCR, based, in part, on improvements in primer design for multiplex amplification. The following sets out various approaches for design of primer sets for high multiplex amplification that avoid or minimize primer:primer interactions. Methods of using the primers so designed in, e.g., multiplex amplification, sequencing and genotyping are also provided and follow directly from the improved primer designs.

### Multiplexed Genotyping

Described herein are improved methods of designing primers for multiplex PCR amplification (see e.g., Examples 1-11). As used herein, the term “primer” denotes a single-stranded nucleic acid that hybridizes to a nucleic acid region of interest and provides a starting point for nucleic acid synthesis, i.e. for enzymatic synthesis of a nucleic acid strand complementary to a template. In some embodiments of any of the aspects, the primer can be DNA, RNA, modified DNA, modified RNA, synthetic DNA, synthetic RNA, or another synthetic nucleic acid. In some embodiments, the primer is about 17-35 nucleotides long. As a non-limiting example, the primer is 17 nucleotides (nt) long, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, or 35 nt long.

In some embodiments of any of the aspects, the primer exhibits reverse complementarity to the coding strand of a nucleic acid region of interest. In some embodiments, the primer has 90-100% identity with the non-coding strand of a nucleic acid region of interest or the reverse complement of a nucleic acid region of interest. In some embodiments, the primer has about 90%, about 91%, about 92%, about 93%, about 94%, about 95%, about 96%, about 97%, about 98%, about 99%, or about 100% identity with the non-coding strand of a nucleic acid region of interest or the reverse complement of a nucleic acid region of interest.

In some embodiments of any of the aspects, the primer is within 100 base pairs (bp) of the nucleic acid region of interest. As a non-limiting example, the primer is within (i.e., hybridizes to a region) about 0 bp, about 1 bp, about 2 bp, about 3 bp, about 4 bp, about 5 bp, about 6 bp, about 7 bp, about 8 bp, about 9 bp, about 10 bp, about 11 bp, about 12 bp, about 13 bp, about 14 bp, about 15 bp, about 16 bp, about 17 bp, about 18 bp, about 19 bp, about 20 bp, about 21 bp, about 22 bp, about 23 bp, about 24 bp, about 25 bp, about 26 bp, about 27 bp, about 28 bp, about 29 bp, about 30 bp, about 31 bp, about 32 bp, about 33 bp, about 34 bp, about 35 bp, about 36 bp, about 37 bp, about 38 bp, about 39 bp, about 40 bp, about 41 bp, about 42 bp, about 43 bp, about 44 bp, about 45 bp, about 46 bp, about 47 bp, about 48 bp, about 49 bp, about 50 bp, about 51 bp, about 52 bp, about 53 bp, about 54 bp, about 55 bp, about 56 bp, about 57 bp, about 58 bp, about 59 bp, about 60 bp, about 61 bp, about 62 bp, about 63 bp, about 64 bp, about 65 bp, about 66 bp, about 67 bp, about 68 bp, about 69 bp, about 70 bp, about 71 bp, about 72 bp, about 73 bp, about 74 bp, about 75 bp, about 76 bp, about 77 bp, about 78 bp, about 79 bp, about 80 bp, about 81 bp, about 82 bp, about 83 bp, about 84 bp, about 85 bp, about 86 bp, about 87 bp, about 88 bp, about 89 bp, about 90 bp, about 91 bp, about 92 bp, about 93 bp, about 94 bp, about 95 bp, about 96 bp, about 97 bp, about 98 bp, about 99 bp, or about 100 bp of the nucleic acid region of interest. In some embodiments of any of the aspects, the primer is within about 80-90 bp, about 90-100 bp, about 100-110 bp, about 110-120 bp, about 120-130 bp, about 130-140 bp, about 140-150 bp, about 150-160 bp, about 160-170 bp, about 170-180 bp, about 180-190 bp, or about 190-200 bp of the nucleic acid region of interest.

In some embodiments of any of the aspects, the nucleic acid region of interest can also be referred to as the target, the target gene, the DNA target of interest, or the genomic target sequence. The nucleic acid region of interest can be DNA, RNA, genomic DNA (gDNA), complementary DNA (cDNA), messenger RNA (mRNA), or any nucleic acid of at least 20 bp. As a non-limiting example, the nucleic acid region of interest can comprise a gene, as known in the art. As a non-limiting example, the nucleic acid region of interest can be present in a non-coding region of the genome or larger nucleic acid. As a non-limiting example, the nucleic acid region of interest can comprise a single-nucleotide polymorphism (SNP). A SNP is a substitution of a single nucleotide that occurs at a specific position in the nucleic acid of interest, where each variation is present to some appreciable degree within a population. For example, a SNP can be an A/T, A/G, A/C, C/G, C/T, G/T, A/C/G, A/C/T, A/G/T, C/G/T, or A/C/G/T polymorphism, where A indicates adenosine, C indicates cytosine, G indicates guanine, T indicates thymine, and the groupings indicate the bases found at a specific SNP.

In some embodiments of any of the aspects, the nucleic acid region of interest can comprise a nucleotide deletion or a nucleotide insertion. As a non-limiting example, the nucleotide deletion or nucleotide insertion can be about 1 bp, about 2 bp, about 3 bp, about 4 bp, about 5 bp, about 6 bp, about 7 bp, about 8 bp, about 9 bp, about 10 bp, about 11 bp, about 12 bp, about 13 bp, about 14 bp, about 15 bp, about 16 bp, about 17 bp, about 18 bp, about 19 bp, about 20 bp, about 21 bp, about 22 bp, about 23 bp, about 24 bp, about 25 bp, about 26 bp, about 27 bp, about 28 bp, about 29 bp, or about 30 bp long. The specific genetic variation (e.g., SNP, insertion, deletion) found in an organism can be referred to as an allele.

In some embodiments of any of the aspects, the nucleic acid region of interest can be from a human, a mammal, a bird, a reptile, an amphibian, a fish, an animal, a plant, a fungus, a protist, a multicellular organism, a unicellular organism, a eukaryote, a bacterium, or an archaebacterium.

As a non-limiting example, the nucleic acid region of interest can be from one of the following plant species: corn (e.g., Zea mays), soybean (e.g., Glycine max), tomato (e.g., Solanum lycopersicum), squash (e.g., Cucurbita argyrosperma, Cucurbita maxima, Cucurbita moschata, Cucurbita pepo), cotton (e.g., Gossypium hirsutum, Gossypium barbadense, Gossypium arboreum, Gossypium herbaceum), wheat (e.g., Triticum aestivum, Triticum aethiopicum, Triticum araraticum, Triticum boeoticum, Triticum carthlicum, Triticum compactum, Triticum dicoccoides, Triticum dicoccon, Triticum durum, Triticum ispahanicum, Triticum karamyschevii, Triticum macha, Triticum militinae, Triticum monococcum, Triticum polonicum, Triticum spelta, Triticum sphaerococcum, Triticum timopheevii, Triticum turanicum, Triticum turgidum, Triticum Urartu, Triticum vavilovii, Triticum zhukovskyi), sunflower (e.g., Helianthus annuus, Helianthis agrestis, Helianthus angustifolius, Helianthus anomalus, Helianthus argophyllus, Helianthus arizonensis, Helianthus atrorubens, Helianthus bolanderi, Helianthus californicus, Helianthus carnosus, Helianthus ciliaris, Helianthus cinereus, Helianthus cusickii, Helianthus debilis, Helianthus decapetalus, Helianthus deserticola, Helianthus divaricatus, Helianthus eggertii, Helianthus floridanus, Helianthus giganteus, Helianthus glaucophyllus, Helianthus gracilentus, Helianthus grosseserratus, Helianthus heterophyllus, Helianthus hirsutus, Helianthus laciniatus, Helianthus laetiflorus, Helianthus laevigatus, Helianthus longifolius, Helianthus maximiliani, Helianthus microcephalus, Helianthus mollis, Helianthus multiflorus, Helianthus neglectus, Helianthus niveus, Helianthus nuttallii, Helianthus occidentalis, Helianthus paradoxus, Helianthus pauciflorus, Helianthus petiolaris, Helianthus porter, Helianthus praecox, Helianthus praetermissus, Helianthus pumilus, Helianthus radula, Helianthus resinosus, Helianthus salicifolius, Helianthus schweinitzii, Helianthus silphioides, Helianthus simulans, Helianthus smithii, Helianthus strumosus, Helianthus tuberosus), grape (e.g., Vitis vinifera, Vitis vinifera, Vitis labrusca, Vitis riparia, Vitis rotundifolia, Vitis rupestris, Vitis aestivalis, Vitis mustangensis, or any multi-species hybrids), cowpea (e.g., Vigna unguiculata), Chrysanthemum (e.g., Chrysanthemum indicum), Eucalyptus (e.g., Eucalyptus obliqua or any of the approximately 700 other species in the Eucalyptus genus), flax (e.g., Phormium tenax, Phormium cookianum), sesame (e.g., Sesamum radiatum), pepper (e.g., Capsicum annuum, Capsicum baccatum, Capsicum chinense, Capsicum frutescens, Capsicum pubescens), rice (e.g., Oryza sativa, including any one of the more than 40,000 varieties of this species), potato (e.g., Solanum tuberosum), cassava (e.g., Manihot esculenta), rye (e.g., Secale cereale), barley (e.g., Hordeum vulgare), alfalfa (e.g., Medicago sativa), or rapeseed (e.g., Brassica napus). A plant species can include any subspecies, cultivars, multi-species hybrids, strains, or any other variations or varieties that are known in the art.

As a non-limiting example, the nucleic acid region of interest can be from one of the following animal species: Homo sapiens, chicken (e.g., Gallus gallus domesticus), pig (e.g., Sus ahoenobarbus, Sus amyus, Sus cebifrons, Sus barbatus, Sus celebensis, Sus oliveri, Sus philippensis, Sus scrofa, Sus verrucosus) bovine (Bos taurus), sheep (e.g., Ovis aries), goat (e.g., Capra aegagrus hircus) deer (e.g. Odocoileus virginianus, Odocoileus hemionus), salmon (e.g., Salmo salar, Oncorhynchus tshawytscha, Oncorhynchus keta, Oncorhynchus kisutch, Oncorhynchus masou, Oncorhynchus gorbuscha, Oncorhynchus nerka, Arripis trutta, Hucho hucho, Elagatis bipinnulata, Eleutheronema tetradactylum) or other fish species (e.g., Acetes japonicas, Ammodytes personatus, Anadara granosa, Brevoortia patronus, Brevoortia tyrannus, Carassius carassius, Catla catla, Cetengraulis mysticetus, Channa argus, Chanos chanos, Cirrhinus mrigala, Clupea bentincki, Clupea harengus, Clupea pallasii, Cololabis saira, Crassostrea gigas, Ctenopharyngodon idellus, Cyprinus carpio, Dosidicus gigas, Engraulis capensis, Engraulis encrasicolus, Engraulis japonicus, Engraulis ringens, Eriocheir sinensis, Ethmalosa fimbriata, Euphausia superba, Euthynnus affinis, Gadus macrocephalus, Gadus morhua, Harpadon nehereus, Hypophthalmichthys molitrix, Hypophthalmichthys nobilis, Ictalurus punctatus, Illex argentinus, Katsuwonus pelamis, Labeo rohita, Larimichthys polyactis, Lates niloticus, Mallotus villosus, Megalobrama amblycephala, Melanogrammus aeglefinus, Merluccius hubbsi, Merluccius productus, Micromesistius poutassou, Misgurnus anguillicaudatus, Monopterus albus, Muraenesox cinereus, Mylopharyngodon piceus, Oncorhynchus mykiss, Opisthonema libertate, Oreochromis niloticus, Oreochromis niloticus, Pandalus borealis, Pangasius hypophthalmus, Patinopecten yessoensis, Pelodiscus sinensis, Penaeus monodon, Penaeus monodon, Penaeus vannamei, Placopecten magellanicus, Pollachius virens, Portunus pelagicus, Portunus trituberculatus, Procambarus clarkii, Rastrelliger brachysoma, Rastrelliger kanagurta, Rastrineobola argentea, Sardina pilchardus, Sardinella aurita, Sardinella gibbosa, Sardinella longiceps, Sardinella maderensis, Sardinops caeruleus, Sardinops melanostictus, Scomber japonicus, Scomber scombrus, Scomberomorus commerson, Selar crumenophthalmus, Selaroides leptolepis, Silurus asotus, Siniperca chuatsi, Sinonovacula constricta, Sprattus sprattus, Tachysurus fulvidraco, Tenualosa ilisha, Theragra chalcogramma, Thunnus alalunga, Thunnus albacares, Thunnus obesus, Thunnus tonggol, Todarodes pacificus, Trachurus capensis, Trachurus japonicus, Trachurus murphyi, Trachurus trachurus, Trachysalambria curvirostris, Trichiurus lepturus, Venerupis philippinarum). An animal species can include any subspecies, strains, breeds, or any other variations or varieties that are known in the art.

Described herein are improved methods of multiplex PCR amplification. Polymerase chain reaction (PCR) is a method to make copies of a specific nucleic acid region of interest. As well known in the art, PCR functions by using a pair of primers to amplify a specific amplicon. As used here, an “amplicon” is the segment of DNA or RNA that is the source and/or product of an amplification, replication, and/or PCR event. The primer pair comprises a forward primer and a reverse primer that anneals a certain distance from the forward primer; the reverse primer anneals to the opposite strand to and opposite orientation to the forward primer, such that polymerization from one primer progresses towards the other primer.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and, optionally, (iii) screening the PCR products for a band or bands of the correct size(s). As described herein, the primers used are oligonucleotides of sufficient length and appropriate sequence to provide initiation of polymerization, i.e. each primer is specifically designed to be complementary to a strand of the genomic locus to be amplified. Unless noted otherwise, all PCR reactions are carried out using standard PCR reaction conditions and reagents, as well known to those of skill in the art.

In some embodiments of any of the aspects, the forward primer and the reverse primer are about 20-100 bp apart. As a non-limiting example, the forward primer and the reverse primer are about 20 bp, about 21 bp, about 22 bp, about 23 bp, about 24 bp, about 25 bp, about 26 bp, about 27 bp, about 28 bp, about 29 bp, about 30 bp, about 31 bp, about 32 bp, about 33 bp, about 34 bp, about 35 bp, about 36 bp, about 37 bp, about 38 bp, about 39 bp, about 40 bp, about 41 bp, about 42 bp, about 43 bp, about 44 bp, about 45 bp, about 46 bp, about 47 bp, about 48 bp, about 49 bp, about 50 bp, about 51 bp, about 52 bp, about 53 bp, about 54 bp, about 55 bp, about 56 bp, about 57 bp, about 58 bp, about 59 bp, about 60 bp, about 61 bp, about 62 bp, about 63 bp, about 64 bp, about 65 bp, about 66 bp, about 67 bp, about 68 bp, about 69 bp, about 70 bp, about 71 bp, about 72 bp, about 73 bp, about 74 bp, about 75 bp, about 76 bp, about 77 bp, about 78 bp, about 79 bp, about 80 bp, about 81 bp, about 82 bp, about 83 bp, about 84 bp, about 85 bp, about 86 bp, about 87 bp, about 88 bp, about 89 bp, about 90 bp, about 91 bp, about 92 bp, about 93 bp, about 94 bp, about 95 bp, about 96 bp, about 97 bp, about 98 bp, about 99 bp, or about 100 bp apart. In some embodiments of any of the aspects, the forward primer and the reverse primer are about 80-90 bp, about 90-100 bp, about 100-110 bp, about 110-120 bp, about 120-130 bp, about 130-140 bp, about 140-150 bp, about 150-160 bp, about 160-170 bp, about 170-180 bp, about 180-190 bp, about 190-200 bp apart, about 200-210 bp apart, about 210-220 bp apart, about 220-230 bp apart, about 230-240 bp apart, or about 240-250 bp apart.

The length of the amplicon is the sum of the distance between the primers and the lengths of both primers. As a non-limiting example, the amplicon can be about 35 bp, about 36 bp, about 37 bp, about 38 bp, about 39 bp, about 40 bp, about 41 bp, about 42 bp, about 43 bp, about 44 bp, about 45 bp, about 46 bp, about 47 bp, about 48 bp, about 49 bp, about 50 bp, about 51 bp, about 52 bp, about 53 bp, about 54 bp, about 55 bp, about 56 bp, about 57 bp, about 58 bp, about 59 bp, about 60 bp, about 61 bp, about 62 bp, about 63 bp, about 64 bp, about 65 bp, about 66 bp, about 67 bp, about 68 bp, about 69 bp, about 70 bp, about 71 bp, about 72 bp, about 73 bp, about 74 bp, about 75 bp, about 76 bp, about 77 bp, about 78 bp, about 79 bp, about 80 bp, about 81 bp, about 82 bp, about 83 bp, about 84 bp, about 85 bp, about 86 bp, about 87 bp, about 88 bp, about 89 bp, about 90 bp, about 91 bp, about 92 bp, about 93 bp, about 94 bp, about 95 bp, about 96 bp, about 97 bp, about 98 bp, about 99 bp, or about 100 bp long. In some embodiments of any of the aspects, the amplicon can be about 80-90 bp, about 90-100 bp, about 100-110 bp, about 110-120 bp, about 120-130 bp, about 130-140 bp, about 140-150 bp, about 150-160 bp, about 160-170 bp, about 170-180 bp, about 180-190 bp, about 190-200 bp long, about 200-210 bp long, about 210-220 bp long, about 220-230 bp long, about 230-240 bp long, or about 240-250 bp long.

The amplicon can be detected by any method known to those of skill in the art. As a non-limiting example, the amplicon can be detected by gel electrophoresis, real time PCR, allele-specific PCR, an array-based method or next generation sequencing (NGS).

Described herein are methods of genotyping. As used herein, genotyping refers to the process of determining differences in the genetic make-up (e.g., genotype) of an individual by examining the individual's DNA sequence using biological assays and comparing it to another individual's sequence or a reference sequence. As used herein, the term “genotype calling” refers to the process of determining a subject's genotype with respect to members of a set of allelic markers, including but not limited to single nucleotide polymorphisms (see e.g., U.S. Pat. No. 8,428,886, which is incorporated herein by reference in its entirety). Biological assays used for genotyping can include but are not limited to PCR, restriction fragment length polymorphism identification (RFLPI) of genomic DNA, random amplified polymorphic detection (RAPD) of genomic DNA, amplified fragment length polymorphism detection (AFLPD), DNA sequencing, allele specific oligonucleotide (ASO) probes, or hybridization to DNA microarrays or beads. In some embodiments of any of the aspects, genotyping is performed on a nucleic acid region of interest, a gene, a non-coding region, a SNP, an insertion, a deletion, or any other region of nucleic acid. Genotyping can reveal the specific allele(s) present in an individual organism or population of organism. Genotyping can be used for in research of genes and gene variants associated with disease

Described herein are methods of multiplexed genotyping and/or multiplexed PCR. As used herein, “multiplex” refers to a reaction in which multiple targets and/or targets in or from multiple samples are amplified or interrogated in the same reaction. In some embodiments of any of the aspects, a multiplexed genotyping reaction can comprise 1 to 100,000 samples. As a non-limiting example, a multiplexed genotyping reaction can comprise about 1 sample, about 2 samples, about 3 samples, about 4 samples, about 5 samples, about 6 samples, about 7 samples, about 8 samples, about 9 samples, about 10 samples, about 20 samples, about 30 samples, about 40 samples, about 50 samples, about 60 samples, about 70 samples, about 80 samples, about 90 samples, about 100-200 samples, about 200-300 samples, about 300-400 samples, about 400-500 samples, about 500-600 samples, about 600-700 samples, about 700-800 samples, about 800-900 samples, about 900-1,000 samples, about 1,000-10,000 samples, about 10,000-20,000 samples, about 20,000-30,000 samples, about 30,000-40,000 samples, about 40,000-50,000 samples, about 50,000-60,000 samples, about 60,000-70,000 samples, about 70,000-80,000 samples, about 80,000-90,000 samples, or about 90,000-100,000 samples.

In some embodiments of any of the aspects, a multiplexed genotyping reaction can comprise 2 to 10,000 amplicons from the same or different nucleic acid regions of interest. As a non-limiting example, the multiplexed genotyping reaction can comprise about 2 amplicons, about 3 amplicons, about 4 amplicons, about 5 amplicons, about 6 amplicons, about 7 amplicons, about 8 amplicons, about 9 amplicons, about 10 amplicons, about 20 amplicons, about 30 amplicons, about 40 amplicons, about 50 amplicons, about 60 amplicons, about 70 amplicons, about 80 amplicons, about 90 amplicons, about 100-200 amplicons, about 200-300 amplicons, about 300-400 amplicons, about 400-500 amplicons, about 500-600 amplicons, about 600-700 amplicons, about 700-800 amplicons, about 800-900 amplicons, about 900-1,000 amplicons, about 1,000-2,000 amplicons, about 2,000-3,000 amplicons, about 3,000-4,000 amplicons, about 4,000-5,000 amplicons, about 5,000-6,000 amplicons, about 6,000-7,000 amplicons, about 7,000-8,000 amplicons, about 8,000-9,000 amplicons, or about 9,000-10,000 amplicons from the same or different nucleic acid regions of interest.

As described herein, to generate a primer set, all possible primers (e.g., 17-35 nucleotides) are identified within about 100 base pairs of each target gene. Primers are chosen that satisfy standard PCR conditions for a primer selection algorithm (e.g., Primer 3™, Oligo Analyzer™, NetPrimer™, or Oligo Calculator™). See e.g., Rozen et al. Methods Mol Biol. 2000, 132:365-86; Owczarzy et al., Nucleic Acids Res. 2008 Jul. 1, 36(Web Server issue):W163-9; each of which is incorporated by reference herein in its entirety.

As well known to those of skill in the art, standard PCR conditions or parameters can comprise preferred values for product (e.g., amplicon) size, primer size, primer Tₘ, Tₘ difference, product Tₘ, and/or primer GC % (e.g., the percentage of G or C bases compared to total bases). As a non-limiting example, primer Tₘ and/or product Tₘ can be about 57° C., about 58° C., about 59° C., about 60° C., about 61° C., about 62° C., or about 63° C., with a preferred primer Tₘ of about 60° C. As a non-limiting example, the max difference between the Tₘ's of the forward primer, the reverse primer, and/or the amplicon can be about 0° C., about 1° C., about 2° C., about 3° C., about 4° C., about 5° C., about 6° C., about 7° C., about 8° C., about 9° C., or about 10° C. As a non-limiting example, GC % can be about 20%, about 25%, about 30%, about 35%, about 40%, about 45%, about 50%, about 55%, about 60%, about 65%, about 70%, about 75%, or about 80%. Methods for calculating Tₘ are well known to those of skill in the art (see e.g., Panjkovich and Melo, Bioinformatics, Volume 21, Issue 6, 15 Mar. 2005, Pages 711-722, which is incorporated by reference herein in its entirety).

Additional PCR conditions that can be considered when necessary or desired during primer selection include but are not limited to primer self complementarity, primer 3′ self complementarity, primer #N's (e.g., consecutive repeated nucleotides), primer mispriming similarity, primer sequence quality, primer 3′ sequence quality, and/or primer 3′ stability. Preferred values for each of the aforementioned conditions can be set or determined by one of skill in the art or by the specific primer selection algorithm (e.g., Primer 3™, Oligo Analyzer™, NetPrimer™, or Oligo Calculator™).

In some embodiments of any of the aspects, primers are compared for specificity versus the genome using alignment software (e.g., primer blast (NCBI™); isPCR (UCSC)). Only those primers predicted to be specific for their respective targets (e.g., hybridizing only to a single sequence or a single set of allelic sequences in the subject genome) are kept. While hybridization is influenced by GC content as well as overall complementarity, in general a primer that is specific for a single target in the genome should have no more than about 80% sequence identity with sequences that are not target sequences in the genome of interest. As a non-limiting example, the primer can have about 0%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% or less sequence identity with a non-target sequence in the genome of interest.

Described herein, are methods of minimizing primer:primer interactions. As used herein, the term “primer-primer interactions” refers to inappropriate non-specific binding or non-specific hybridization of a primer with another primer in a reaction mixture, including a multiplex reaction mixture, as opposed to appropriate specific binding or specific hybridization with each primer's target of interest. Where primers are included in a reaction at many times the concentration of any given target sequence, even relatively inefficient primer-primer interactions can impact performance of the amplification. As a non-limiting example, primer-primer dimers are a potential byproduct in PCR, comprising primer molecules that have hybridized to each other due to regions of complementary bases. Primer-primer interactions reduce the availability of the interacting primers to hybridize productively with their intended targets and can lead to a competition for reaction reagents and potential inhibition of amplification of the targets of interest. In multiplex PCR and/or genotyping reactions, at least 2 pairs of primers are present in the same sample. As a non-limiting example, at least 3 pairs of primers, at least 4 pairs of primers, at least 5 pairs of primers, at least 6 pairs of primers, at least 7 pairs of primers, at least 8 pairs of primers, at least 9 pairs of primers, at least 10 pairs of primers, at least 20 pairs of primers, at least 30 pairs of primers, at least 40 pairs of primers, at least 50 pairs of primers, at least 60 pairs of primers, at least 70 pairs of primers, at least 80 pairs of primers, at least 90 pairs of primers, at least 100 pairs of primers, at least 110 pairs of primers, at least 120 pairs of primers, at least 130 pairs of primers, at least 140 pairs of primers, at least 150 pairs of primers, at least 160 pairs of primers, at least 170 pairs of primers, at least 180 pairs of primers, at least 190 pairs of primers, at least 200 pairs of primers, at least 250 pairs of primers, at least 300 pairs of primers, at least 350 pairs of primers, at least 400 pairs of primers, at least 450 pairs of primers, or least 500 pairs of primers are present in a reaction mixture, allowing for hundreds or thousands of potential inappropriate primer-primer interactions. The primer-design methods described herein can dramatically improve throughput in NGS-based multiplex genotyping methods.

### Fitness Score

In some embodiments, a fitness score is calculated for a primer set, chromosome, or individual (see e.g., Example 2). As used in the context of a genetic algorithm, the terms “chromosome” or “individual” refer to a set of “X” primer pairs, where X is the number of targets being interrogated in a multiplex reaction. A primer set can comprise one primer pair for each target gene or nucleic acid region of interest in a multiplex set of target genes or nucleic acid regions of interest. The initial primer pairs for each primer set can be selected randomly or guided by a selection algorithm. As a non-limiting example, a primer set can comprise at least 2 primer pairs, at least 3 primer pairs, at least 4 primer pairs, at least 5 primer pairs, at least 6 primer pairs, at least 7 primer pairs, at least 8 primer pairs, at least 9 primer pairs, at least 10 primer pairs, at least 20 primer pairs, at least 30 primer pairs, at least 4 primer pairs, at least 50 primer pairs, at least 60 primer pairs, at least 70 primer pairs, at least 80 primer pairs, at least 90 primer pairs, at least 100 primer pairs, at least 110 primer pairs, at least 120 primer pairs, at least 130 primer pairs, at least 140 primer pairs, at least 150 primer pairs, at least 160 primer pairs, at least 170 primer pairs, at least 180 primer pairs, at least 190 primer pairs, at least 200 primer pairs, at least 250 primer pairs, at least 300 primer pairs, at least 350 primer pairs, at least 400 primer pairs, at least 450 primer pairs, or least 500 primer pairs.

In some embodiments, e.g., in the context of a genetic algorithm, a fitness score is calculated for a population (i.e. set) of individuals. As a non-limiting example, a population can comprise at least 2 individuals, at least 3 individuals, at least 4 individuals, at least 5 individuals, at least 6 individuals, at least 7 individuals, at least 8 individuals, at least 9 individuals, at least 10 individuals, at least 20 individuals, at least 30 individuals, at least 4 individuals, at least 50 individuals, at least 60 individuals, at least 70 individuals, at least 80 individuals, at least 90 individuals, at least 100 individuals, at least 110 individuals, at least 120 individuals, at least 130 individuals, at least 140 individuals, at least 150 individuals, at least 160 individuals, at least 170 individuals, at least 180 individuals, at least 190 individuals, at least 200 individuals, at least 250 individuals, at least 300 individuals, at least 350 individuals, at least 400 individuals, at least 450 individuals, or at least 500 individuals.

In some embodiments, the primers within each primer set are analyzed for ΔG. In this context, “ΔG” (or “delta G”) refers to the change in Gibbs free energy, a measure of spontaneity. In some embodiments, ΔG represents the quantity of energy needed to fully break a secondary DNA structure (e.g., primer-primer interaction). ΔG can be measured in units of kilocalorie per mole (kcal/mol) or kilojoule per mole (KJ/mol). When ΔG is negative (i.e. less than zero), a process (e.g., primer-primer interactions) proceeds spontaneously and is referred to as exergonic. When ΔG is positive (i.e. greater than zero), a process (e.g., primer-primer interactions) does not proceed spontaneously and is referred to as endergonic. When ΔG is equal to zero, a process is referred to as in equilibrium. In some embodiments, ΔG is calculated using FORMULA 1:

FORMULA 1: ΔG=ΔH−T ΔS, where “ΔH” (or “delta H”) refers to the change in enthalpy, “T” refers to the temperature of the reaction in degrees Kelvin (K), and “ΔS” (or “delta S”) refers to the change in entropy of the reaction.

When ΔH is negative (i.e. less than zero), a process (e.g., primer-primer interactions) releases heat and is referred to as exothermic. When ΔH is positive (i.e. greater than zero), a process (e.g., primer-primer interactions) absorbs heat and is referred to as endothermic. When ΔH is equal to zero, no heat is released or absorbed in a process (e.g., primer-primer interactions). ΔH can be measured in units of kilojoule per mole (KJ/mol) or kilocalorie per mole (kcal/mol). ΔH can be determined experimentally or calculated or closely approximated using various software packages, which, e.g., incorporate or account for enthalpy change for various short sequences hybridizing with their complementary sequences (for a discussion of nearest neighbor, see below). As well known to those of skill in the art, in some embodiments ΔH can be determined using the heats of formation for each compound in a reaction; for a nucleic acid duplex, the heat of formation refers to the heat of duplex formation.

As used herein, “ΔS” (or “delta S”) refers to the change in entropy and can be used as a measure of disorder and randomness. When ΔS is negative (i.e. less than zero), there is a decrease of disorder in a process (e.g., primer-primer interactions). When ΔS is positive (i.e. greater than zero), there is an increase of disorder in a process (e.g., primer-primer interactions). When ΔS is equal to zero, there is no change in disorder in a process (e.g., primer-primer interactions). ΔS can be measured in units of kilojoule per mole (kJ/mol) per Kelvin or kilocalorie per mole (kcal/mol) per Kelvin. ΔS can be determined experimentally or calculated or closely approximated by using various software packages, e.g., as for ΔH.

As well known to those of skill in the art, in some embodiments ΔG, ΔH, and/or ΔS are known for each possible “nearest neighbor” nucleotide interaction (e.g., AA/TT, AT/TA, TA/AT, CA/GT, GT/CA, CT/GA, GA/CT, CG/GC, GC/CG, GG/CC; see e.g., TABLE 1). See e.g., SantaLucia. A unified view of polymer, dumbbell, and oligonucleotide DNA nearest-neighbor thermodynamics. Proc. Natl Acad. Sci. USA 95, 1460-1465 (1998); Sugimoto et al. Thermodynamic parameters to predict stability of RNA/DNA hybrid duplexes. Biochemistry 34, 11211-11216 (1995); each of which is incorporated by reference herein in its entirety.

TABLE 1
ΔH, ΔG, and ΔS for each possible nearest
neighbor DNA nucleotide interaction (T ≈ 300 K).

| Interaction | dH° (kcal/mol) | dS° (cal/mol per K) | dG° (kcal/mol) |
|---|---|---|---|
| AA/TT | −9.1 | −24.0 | −1.9 |
| AT/TA | −8.6 | −23.9 | −1.5 |
| TA/AT | −6.0 | −16.9 | −1.0 |
| CA/GT | −5.8 | −12.9 | −2.0 |
| GT/CA | −6.5 | −17.3 | −1.3 |
| CT/GA | −7.8 | −20.8 | −1.6 |
| GA/CT | −5.6 | −13.5 | −1.6 |
| CG/GC | −11.9 | −27.8 | −3.6 |
| GC/CG | −11.1 | −26.7 | −3.1 |
| GG/CC | −11.0 | −26.6 | −3.1 |

In some embodiments, the following Fitness Score (e.g., F) is calculated via FORMULA 2.

$$F = {\{ C / [ ( Σ ( {\vert Δ G \vert}^{A} ) ) / {G}_{n} ] \}}^{B} \qquad FORMULA 2$$

In this context, “G” equals the set of ΔG's for primer interactions determined for that primer set. ΔG is calculated for all possible primer-primer interactions determined in an primer set or a subset of every possible primer-primer interaction determined in an individual or set of primers. “A” and “B” are weighting factors; A and/or B can be an exponential weighting factor constant that makes large values much larger than small values. In some embodiments of Fitness Score calculations, the weighting factor “A” is alternatively referred to as “Q,” and/or the weighting factor “B” is alternatively referred to as “R.” A and/or B can be a non-zero, positive number greater than 1. A and/or B can be an integer or a fraction/decimal. As a non-limiting example, A and/or B can equal 1, 2, 3, 4, 5, 6, 7, 8, 9, 10, 15, 20, 25, 30, 35, 40, 45, 50, 55, 60, 65, 70, 75, 80, 85, 90, 95, 100, or any integer or fraction/decimal from 1 to 100 or 100 to 1,000. “C” is a factor to create reciprocal scaled values. C causes large numbers to become small numbers. C causes small numbers to become large numbers. In some embodiments of Fitness Score calculations, the scaling factor is alternatively referred to as “T.” In the context of Fitness score calculation, T should not be construed as referring to temperature, but to a scaling factor analogous to C described herein. As a non-limiting example, C can equal 1, 2, 3, 4, 5, 6, 7, 8, 9, 10, 15, 20, 25, 30, 35, 40, 45, 50, 55, 60, 65, 70, 75, 80, 85, 90, 95, 100, or any non-zero integer or fraction/decimal from 0 to 100 or 100 to 1,000. As a non-limiting example, A equals 5, B equals 2, and C equals 200.

In this Fitness Score F formula, the absolute value of each ΔG value is raised to the “A” power, and all such values are summed for a primer set, wherein Σ denotes a summation. The “A” exponent has the effect of spreading out the data values. In some embodiments, A (or Q) is a weighting factor constant exponent that makes large ΔG absolute values much larger than small values. The “A” exponent in effect “weights” or penalizes values proportional to the magnitude of “A”. This sum is “S”. See e.g., FORMULA 3: S=Σ(|ΔG|^{A})

In FORMULA 2, some embodiments, the sum is divided by the total number of ΔG values determined for that primer set or “Gₙ.” The division by Gₙ normalizes S, such that the sum is not inherently larger for larger numbers of primer sets or smaller for smaller numbers of primer sets. This normalized S value is referred to as “S′” (or “S prime”). See e.g., FORMULA 4: S′=S/Gₙ.

In calculating the Fitness Score F according to FORMULA 2, factor C (or T) is divided by S′ resulting in the value referred to herein as “H” (not to be confused with DH). See e.g., FORMULA 5: H=C/S′.

In calculating the Fitness Score F, H is raised to the “B” power. B (or R), similar to A, has the effect of further spreading out the data, making the difference between large and small values even bigger. The value is referred to as the “Fitness Score” or F. See e.g., FORMULA 6: F=H^{B}.

In some embodiments, each primer set is assigned a Fitness Score (e.g., “F”). “F” can be calculated using FORMULA 2, which simplifies using FORMULAE 3, 4, and 5 to FORMULA 6.

In some embodiments, an alternative calculation or algorithm can be used to calculate a fitness score. As a non-limiting example, a fitness score can comprise any one of the values described herein, including but not limited to ΔG, ΔH, T, and/or AS for each primer interaction; any one of the constants described herein, including but not limited to A, B, C, Q, R, T, and/or other constants; and/or any permutation of these values and/or constants, including but not limited to addition, subtraction, multiplication, division, absolute value, summation, exponential power, logarithm, and the like.

### Selection Methods

Provided herein are methods for primer design that permit large amplicon number multiplexing and genotype calling. In some embodiments, a set of primers is selected for each target through a specific selection method. In some embodiments, the selected primer set is referred to as “optimal.” As used herein “optimal primer set” can refer to a primer set with a minimization of primer-primer interactions. An optimal primer set can be selected using any of several mathematical algorithms described herein.

In some embodiments, optimal primers or an optimal primer set are selected for each target based on the minimization of primer-primer interactions. In some embodiments, primer set selection is based on the maximization of a Fitness Score F for a primer set (see e.g., Example 2). In other embodiments, primer set selection is based on the set meeting a threshold Fitness Score. As non-limiting examples, a threshold Fitness Score can be at least 1, at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900, at least 2000, at least 3000, at least 4000, at least 5000, at least 6000, at least 7000, at least 8000, at least 9000, or at least 10,000.

In some embodiments, a primer set is selected using any of several mathematical algorithms described herein and/or incorporated into the PlexForm™ process as described herein. In some embodiments, each sample point in the search space is a collection of primer pairs (e.g., one for each target) in one or more sets, where either highest chance of primer interaction or number of primers below a threshold of primer interaction within a set (e.g., or maximum value for multiple sets) provides a value that is being minimized. In some embodiments, the techniques use a model that can accurately predict the possibility of primer interaction between members of a collection of primer pairs.

In some embodiments, the search space for this task is too large to search for best solution (e.g., global minima). In some embodiments, the following techniques are employed iteratively, together, and/or separately to find an acceptable solution: Genetic Algorithm (see e.g., Example 3); a Monte Carlo random or pseudo-random selection method (see e.g., Example 4); Golden-section search (see e.g., Example 5); Gradient descent (see e.g., Example 6), Minima hopping (see e.g., Example 7); Neural Networks (see e.g., Example 8); Cluster analysis, in which substitution is picked to minimize score (see e.g., Example 9); or Cluster Analysis to create bins (see e.g., Example 10). Examples and detailed descriptions of each of these algorithmic approaches is described further herein.

In some embodiments, a Monte Carlo method can be used alone or iteratively, together, and/or separately with at least one other method to select an optimal primer set. In some embodiments, using random and/or pseudo random selection, one can achieve with a Monte Carlo method a uniform distribution of the sample points in the search space. Such a distribution can be augmented or replaced by selection of inputs near points of interest, that can be determined. See e.g., METROPOLIS et al., J Am Stat Assoc. 1949 September, 44(247):335-41; Rubinstein and Kroese, Simulation and the Monte Carlo method, 3^{rd} Ed., John Wiley & Sons, Inc. Hoboken, New Jersey, ISBN: 978-1-118-63216-1, October 2016; each of which is incorporated herein by reference in its entirety.

In some embodiments, a Golden-section search method can be used alone or iteratively, together, and/or separately with at least one other method to select an optimal primer set. In some embodiments, using a Golden Section Search algorithm it is possible to pick two points (e.g., where some of the targets contain primer pairs that are the same and do not change during the progression of the search) and using the assumption that minima located between two of those pick a third and fourth point between first two and evaluate the model of each point. In some embodiments, using the assumption that in between two original points the function produced by the model is unimodal, one removes an outside point that has an additional point between it and a point with minimum value. A replacement for the removed point can be picked in-between the two outermost points. This process can be repeated until the distance between two outer most points is small enough to be within a previously determined margin.

As a non-limiting example, the previously determined margin can less than 1, less than 100, less than 200, less than 300, less than 400, less than 500, less than 600, less than 700, less than 800, less than 900, less than 1000, less than 1100, less than 1200, less than 1300, less than 1400, less than 1500, less than 1600, less than 1700, less than 1800, less than 1900, less than 2000, less than 3000, less than 4000, less than 5000, less than 6000, less than 7000, less than 8000, less than 9000, or less than 10,000. For an example involving a Golden Section search method, see e.g., Chang et al., N-Dimension Golden Section Search: Its Variants and Limitations, 2009 2nd International Conference on Biomedical Engineering and Informatics, 17-19 Oct. 2009, which is incorporated herein by reference in its entirety.

In some embodiments, a Gradient descent method can be used alone or iteratively, together, and/or separately with at least one other method to select an optimal primer set. In some embodiments, using a gradient descent algorithm it is possible to determine a gradient (e.g., that represents an increase or decrease in change of interaction between primers) in a given point by measuring a change in interaction with the change of a single primer pair for a given target. In some embodiments, measured change in primer interaction points to a change in primer pairs that produces the greatest reduction in primer interaction. The process is repeated until a point is achieved where any change increases primer interaction score (e.g., decreases the fitness score). Such a point can be referred to as a minima (e.g., where primer-primer interaction is least likely). See e.g., Ruder, An overview of gradient descent optimization algorithms, 2016, available on the world wide web at arxiv.org/abs/1609.04747; Mandic et al. IEEE Signal Processing Letters (Volume: 11, Issue: 2, February 2004), pp. 115-118; each of which is incorporated by reference herein in its entirety.

In some embodiments, a minima hopping method can be used alone or iteratively, together, and/or separately with at least one other method to select an optimal primer set. In some embodiments, to ensure that minima (e.g., where primer interaction is least likely) that are found are not local minima, minima hopping can be employed. Upon locating a minima, it is noted, and a new point in the search space is picked. The new point can be picked randomly or via deterministic method(s) that may be based on variety of factors such as completeness of the data, historical accuracy of predictions, coverage during present search, human guided suggestion, and random and pseudo random number generation, or any combination of those methods as well as others. See e.g., Goedecker, J. Chem. Phys. 120, 9911 (2004), which is incorporated by reference herein in its entirety.

In some embodiments, a genetic algorithm method can be used alone or iteratively, together, and/or separately with at least one other method to select an optimal primer set. In some embodiments, using a genetic algorithm it is possible to pick several valid points and evaluate points in such cohort. Few points that produce lowest chance of primer interaction move on to next iteration with rest of the population being replaced via combining points with lowest chance of primer interaction, random, pseudo random, and/or guided changes. Magnitude of the change can be correlated to fitness of the best point, average fitness, predetermined function, rate of improvement or other metrics. In some embodiments, this process comprising a genetic algorithm can be repeated. In some embodiments, repetition can be stopped when model of high enough fitness is obtained (e.g., a threshold fitness scores as described herein). In some embodiments, time limit and/or changes are insignificant. See e.g., Davis, Handbook of genetic algorithms, 1991, ISBN-13:978-0442001735; Whitley, Statistics and Computing, June 1994, Volume 4, Issue 2, pp 65-85; each of which is incorporated herein by reference in its entirety.

In some embodiments, a neural network method can be used alone or iteratively, together, and/or separately with at least one other method to select an optimal primer set. In some embodiments, using a neural network it is possible to create a function (e.g., one or more per target) that gets inputs of all possible primer pairs. Such a function produces a recommendation based on internal values and/or functions. Once a recommendation has been made, an output of functions is fed back into the initial set of functions, with some causing output values to change. After the function settles on certain point, a primer interaction score (e.g., fitness score) is calculated. If this score is not satisfactory, changes are made to internal values and/or functions, and the process is repeated. Change to the internal values can be guided by a variety of algorithms. In some embodiments, it is possible to have internal values for the function to be picked in advance as well as adjusted or created just for the set of targets. In some embodiments, the neural network comprises a node. As used herein, “node” refers to the basic unit of computation in a neural network. A node can also be referred to as a neuron or unit. See e.g., Hansen et al., IEEE Transactions on Pattern Analysis & Machine Intelligence, October 1990, pp. 993-1001, vol. 12; Specht et al., IEEE Transactions on Neural Networks (Volume: 2, Issue: 6, November 1991), pp. 568-576; each of which is incorporated by reference herein in its entirety.

In some embodiments, a cluster analysis method can be used alone or iteratively, together, and/or separately with at least one other method to select an optimal primer set. In some embodiments, using a cluster analysis algorithm where all primer pair interactions are placed in a cluster, some members of the cluster that are outer most are candidates for substitution (e.g., either one of the primer pairs is replaced, or both). In some embodiments, substitution is picked in such way to minimize resulting interaction score (e.g., maximize the fitness score). See e.g., Scott et al., Biometrics Vol. 30, No. 3 (September, 1974), pp. 507-512; Caliński et al., Communications in Statistics Volume 3, 1974-Issue 1, pp. 1-27; Fraley et al., The Computer Journal, Volume 41, Issue 8, 1998, Pages 578-588; each of which is incorporated by reference herein in its entirety.

In some embodiments, Cluster Analysis to create bins method can be used alone or iteratively, together, and/or separately with at least one other method to select an optimal primer set. In some embodiments, if there is a situation where it becomes evident that the solution to produce a set of primers where primer interaction is below a certain threshold is not attainable, cluster analysis can be used to separate targets into two or more sets where targets that have primer pairs with high likelihood of primer interactions will be placed into separate sub set(s) once they are extracted. New subset is optimized using approaches listed above (e.g., a Monte Carlo random or pseudo-random selection method; a golden section search; gradient descent; minima hopping; genetic algorithm; neural networks; cluster analysis). In some embodiments, if a score for the new set is significantly lower, as compared to an original set, new elements may be added to even out the chance of interaction between multiple sets. See e.g., Nugent and Stuetzle, Clustering with Confidence: A Binning Approach, 2008, available on the world wide web at stat.cmu.edu/tr/tr870/tr870.pdf, which is incorporated by reference herein in its entirety.

### Primer Synthesis

Described herein are methods of selecting and/designing a primer set, e.g., for large amplicon number multiplexing. In some embodiments, the selected primer set is synthesized. Methods of oligonucleotide synthesis are well known to those of skill in the art. As used herein, “oligonucleotide synthesis” refers to the chemical synthesis of relatively short fragments of nucleic acids with defined chemical structure. As a non-limiting example, methods of oligonucleotide synthesis include phosphoramidite solid-phase synthesis, phosphoramidite synthesis, phosphodiester synthesis, phosphotriester synthesis, or phosphite triester synthesis. See e.g., Beaucage et al. Tetrahedron Volume 48, Issue 12, 20 Mar. 1992, Pages 2223-2311; Caruthers, J Biol Chem. 2013 Jan. 11, 288(2): 1420-7.

In some embodiments, each primer is synthesized separately. In some embodiments, the entire primer set is synthesized in one reaction. In some embodiments, a subset of the entire primer set is synthesized in one reaction. In some embodiments, the entire primer set is synthesized in multiple, separate reactions. In some embodiments, reaction products are isolated, e.g., by high-performance liquid chromatography (HPLC), to obtain the desired oligonucleotides in high purity.

### Equalization of Reads

It has been recognized that uneven amplification of genotyping targets reduces the efficiency, and therefore the throughput, of NGS-based multiplex genotyping approaches. Accordingly, described herein are methods for equalization of reads in these approaches (see e.g., Example 12). In some embodiments, anti-sense oligos are used to equalize reads. As used herein, the term “anti-sense oligo” (also referred to as a “blocker” or an “attenuator oligo”) refers to an oligo that is complementary (i.e. anti-sense) to at least one amplicon in a reaction mixture.

In some embodiments the anti-sense oligo is at least 10 nucleotides (nt) long. As a non-limiting example, the anti-sense oligo is at least 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, or at least 35 nt long. In some embodiments of any of the aspects, the anti-sense oligo is between 10 and 35 nucleotides in length, e.g., 10-30 nucleotides, 10-25 nucleotides, 10-20 nucleotides, 10-15 nucleotides, 15-35 nucleotides, 15-30 nucleotides, 15-25 nucleotides, 15-20 nucleotides, 20-35 nucleotides, 20-30 nucleotides, or 20-25 nucleotides long.

In some embodiments of any of the aspects, the anti-sense oligo exhibits reverse complementarity (i.e. is anti-sense) to an amplicon or part of an amplicon. The anti-sense oligo used must be capable of hybridizing to the target amplicon and thereby inhibiting amplification. In one embodiment, the anti-sense oligos lack a 3′ hydroxyl moiety that permits extension by a polymerase. In another embodiment, the anti-sense oligos are fully complementary to at least a portion of the target amplicon. Alternatively, the anti-sense oligos can include some degree of non-complementarity, as long as they are still capable of hybridizing to the target amplicon under the conditions for the amplification reaction. The degree of non-complementarity tolerated can vary with the length and composition of the oligo and/or target, in that longer oligos tend to hybridize more efficiently than shorter ones, and G/C content affects efficiency as well. Keeping these and other variables in mind, in some embodiments, the anti-sense oligos have at least about 90%, about 91%, about 92%, about 93%, about 94%, about 95%, about 96%, about 97%, about 98%, about 99%, or about 100% identity with at least one target amplicon. In some embodiments, the anti-sense oligo hybridizes to at least one primer, especially during the early PCR cycles when the amplicon level is minimal.

In some embodiments, at least one anti-sense oligo is added to a reaction mixture. As a non-limiting example, 1 anti-sense oligo, 2 anti-sense oligos, 3 anti-sense oligos, 4 anti-sense oligos, 5 anti-sense oligos, 6 anti-sense oligos, 7 anti-sense oligos, 8 anti-sense oligos, 9 anti-sense oligos, or at least 10 anti-sense oligos are added or included in a reaction mixture. In some embodiments, each anti-sense oligo can be directed at a different amplicon. In some embodiments, a set of anti-sense oligo binds to and/or inhibits at least 1, at least 2, at least 3, at least 4, at least 5, at least 6, at least 7, at least 8, at least 9, or at least 10 primers and/or amplicons.

In some embodiments, the anti-sense oligo or set of anti-sense oligos is added to inhibit high-performing amplicons from forming and thereby allow poorer performing amplicons to produce more data. As used herein, “high performing” refers to an amplicon that has a higher number of reads than the average number of reads for all amplicons. As a non-limiting example, a high-performing amplicon can have at least 400 reads, at least 410 reads, at least 420 reads, at least 430 reads, at least 440 reads, at least 450 reads, at least 460 reads, at least 470 reads, at least 480 reads, at least 490 reads, at least 500 reads, at least 510 reads, at least 520 reads, at least 530 reads, at least 540 reads, at least 550 reads, at least 560 reads, at least 570 reads, at least 580 reads, at least 590 reads, at least 600 reads, at least 610 reads, at least 620 reads, at least 630 reads, at least 640 reads, at least 650 reads, at least 660 reads, at least 670 reads, at least 680 reads, at least 690 reads, or at least 700 reads in a sequencing reaction.

In some embodiments, a high-performing amplicon can have a number of reads that is at least 50% greater than the average number of reads for all amplicons. As a non-limiting example, a high-performing amplicon can have a number of reads that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% greater than the average number of reads for all amplicons.

As used herein, “low performing amplicon” (or “poorer performing amplicon) refers to an amplicon that has a lower number of reads than the average number of reads for all amplicons. As a non-limiting example, a low-performing amplicon can have at most 100 reads, at most 110 reads, at most 120 reads, at most 130 reads, at most 140 reads, at most 150 reads, at most 160 reads, at most 170 reads, at most 180 reads, at most 190 reads, at most 200 reads, at most 210 reads, at most 220 reads, at most 230 reads, at most 240 reads, at most 250 reads, at most 260 reads, at most 270 reads, at most 280 reads, at most 290 reads, at most 300 reads, at most 310 reads, at most 320 reads, at most 330 reads, at most 340 reads, at most 350 reads, at most 360 reads, at most 370 reads, at most 380 reads, at most 390 reads, or at most 400 reads in a sequencing reaction.

In some embodiments, a low-performing amplicon can have a number of reads that is at least 50% less than the average number of reads for all amplicons. As a non-limiting example, a low-performing amplicon can have a number of reads that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% less than the average number of reads for all amplicons.

In some embodiments, an anti-sense oligo or a set of anti-sense oligos is added at a specific concentration. In some embodiments, an anti-sense oligo or a set of anti-sense oligos can be added at a concentration that is higher than the concentration of the other primers or oligos. In some embodiments, an anti-sense oligo or a set of anti-sense oligos can be added at a concentration that is lower than the concentration of the other primers or oligos. In some embodiments, an anti-sense oligo or a set of anti-sense oligos can be added at a concentration that is the same as the concentration of the other primers or oligos. As a non-limiting example, an anti-sense oligo or a set of anti-sense oligos can be added at a concentration of about 0.01 μM, about 0.02 μM, about 0.03 μM, about 0.04 μM, about 0.05 μM, about 0.06 μM, about 0.07 μM, about 0.08 μM, about 0.09 μM, about 0.1 μM, about 0.2 μM, about 0.3 μM, about 0.4 μM, about 0.5 μM, about 0.6 μM, about 0.7 μM, about 0.8 μM, about 0.9 μM, or about 1.0 μM.

In some embodiments, anti-sense oligos can be used for performing multiplex PCR that results in even reads produced by NGS instruments for each amplicon (e.g., for 2-10,000 amplicons) and each sample (e.g., for 1-100,000 DNA samples).

In some embodiments, the anti-sense oligo further comprises an additional sequence on the 5′-end that permanently inactivate primers in solution once annealed and extended. Such anti-sense oligos with 5′ primer-binding sequences are referred to as an inactivator oligos. Other embodiments use anti-sense oligos with reversible binding characteristics having only complementary sequences (e.g., attenuator oligos).

In some embodiments, the additional 5′ sequence on an inactivator oligo can be at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, or at least 35 nt long. In some embodiments, the inactivator oligo has about 90%, about 91%, about 92%, about 93%, about 94%, about 95%, about 96%, about 97%, about 98%, about 99%, or about 100% identity with at least one primer. At least one inactivator oligo can be added to a reaction mixture.

### Barcode Primer Design

Described herein are methods of designing a primer set, e.g., for large amplicon number multiplexing. In some embodiments, the primers comprise barcodes that allow for large number multiplexing (see e.g., Example 13). As used herein, a “barcode” or “index” is a nucleotide sequence that can be used to identify a sample (e.g., a sample from a specific subject). As a non-limiting example, a barcode can be 5-10 nucleotides long. As a non-limiting example, barcode can be 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, or at least 35 nt long.

In some embodiments, a barcode can be unique for each primer or amplicon. In some embodiments, a set of barcodes can comprise at least 2 unique barcode sequences. As a non-limiting example, a set of barcodes can comprise at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 250, at least 300, at least 350, at least 400, at least 450, or least 500 unique barcode sequences. As a non-limiting example, a set of barcodes can comprise 144 unique barcode sequences that are each 10 nucleotides long.

In some embodiments, a primer can comprise at least 2 barcodes. As a non-limiting example, a primer can comprise 1 barcode, 2 barcodes, 3 barcodes, 4 barcodes, 5 barcodes, 6 barcodes, 7 barcodes, 8 barcodes, 9 barcodes, or 10 barcodes. In some embodiments, one set of barcodes can comprise Illumina™ I5 indexes, Illumina™ I7 indexes, and/or Ion Torrent barcode sequences. In some embodiments, barcodes be incorporated “in-line” (meaning sequentially or in the same primer) into I5 Illumina™ primers, I7 Illumina™ primers, Ion Torrent™ primers, or the primers for any other sequencing platform, as described herein. As a non-limiting example, a combination of in-line and I5/I7 barcodes on the Illumina™ platform can be used for greater than 100 samples, 500 samples, 1,000 samples, 10,000 samples, 20,000 samples, 30,000 samples, 40,000 samples, 50,000 samples, 60,000 samples, 70,000 samples, 80,000 samples, 90,000 samples, or 100,000 samples in the same sequencing run.

In some embodiments, the barcodes that allow for large number multiplexing (e.g., “in-line” barcodes) are used during the primary amplification. In some embodiments, the barcodes that allow for large number multiplexing are used during the secondary amplification. In some embodiments, the Illumina™, Ion Torrent, or other NGS barcodes or indexes are used during the primary amplification. In some embodiments, the Illumina™, Ion Torrent, or other NGS barcodes or indexes are used during the secondary amplification. In some embodiments, the barcodes that allows for large number multiplexing are used during a different amplification as the Illumina™, Ion Torrent, or other NGS barcodes or indexes. In some embodiments, the barcodes that allow for large number multiplexing are used during the same amplification as the Illumina™, Ion Torrent, or other NGS barcodes or indexes.

### PCR Amplification Method

Described herein is a non-limiting example of a PCR amplification method (see e.g., Example 14). In some embodiments, the PCR amplification method comprises a primary (i.e. first) PCR amplification and a secondary PCR amplification. In some embodiments, the primary PCR amplification and the secondary PCR amplification are performed sequentially. In some embodiments, the primary amplification is performed with the primer mix or primer set designed using any one or combination of the methods described herein. In some embodiments, primers in the primary PCR amplification further comprise a 5′ tail sequence, wherein “tail” denotes that the sequence does not hybridize to the target. In some embodiments, the 5′ tail sequence of the primary PCR amplification primers comprises an appropriate number of bases such that all primers in the primer set are the same length (e.g., length N). In some embodiments, the 5′ tail sequence of the primary PCR amplification primers comprises at 1 barcode or at least 2 barcodes.

In some embodiments, a secondary PCR amplification can be performed using the primary amplification as the source or template. In some embodiments, the secondary PCR amplification is performed using a different set of primers as the primary PCR amplification. In some embodiments, the secondary PCR amplification is performed using the same set of primers as the primary PCR amplification. In some embodiments, the secondary PCR amplification is performed using primers (e.g., from the primary PCR amplification, from a unique set of primers) further comprising a 5′ tail sequence. In some embodiments, the 5′ tail sequence of the secondary PCR amplification primers comprises an appropriate number of bases such that all primers in the primer set are the same length (e.g., length N). In some embodiments, the 5′ tail sequence of the secondary PCR amplification primers comprises at 1 barcode or at least 2 barcodes.

In some embodiments, the primary PCR amplification reaction mixture is transferred into the secondary PCR amplification reaction mixture. As a non-limiting example, about 10 nanoliters (nL), about 20 nL, about 30 nL, about 40 nL, about 50 nL, about 60 nL, about 70 nL, about 80 nL, about 90 nL, about 100 nL, about 200 nL, about 300 nL, about 400 nL, about 500 nL, about 600 nL, about 700 nL, about 800 nL, about 900 nL, or about 1000 nL of the primary PCR amplification reaction mixture is transferred into the secondary PCR amplification reaction mixture. In some embodiments, the transfer can performed using a disposable plastic pintool, a pipette, an automated pintool, an automated pipette, or any other device or machine appropriate for this task. In some embodiments, the secondary amplicons are mixed into one sample and loaded onto a Next Generation Sequencer.

### Single Base Multiplexed Sequencing

The methods described herein permit efficient sequencing and genotype calling in high multiplex. Thus, described herein are methods for performing multiplex PCR that results in even reads produced by NGS instruments for each amplicon (for 2-10,000 amplicons) and each sample (for 1-100,000 DNA samples). In one embodiment, a variation is described herein that permits single base multiplexed sequencing on an NGS platform, in order to avoid sequencing additional regions, e.g., when this is beneficial. In some embodiments, primers can be designed using an N+1 approach, meaning that all primers in a set end at the same number of nucleotides, N, away from a sequence of interest, e.g., a SNP. In this design, the NGS sequencing platform can be run for a maximum of N+1 cycles to interrogate the SNPs without reading beyond the SNP itself.

In some embodiments, a primer set is designed such that all primers in the primer set (e.g., of the primary PCR amplification and/or the secondary PCR amplification) are the same length (e.g., length N). As a non-limiting example, all primers in a primer set are 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, or 35 nt long. In some embodiments, an additional set of bases are added to the 5′ end of the primers (e.g., used in the primary amplification and/or the secondary PCR amplification) such that all are of length N. In some embodiments, the additional set of bases added to the 5′ end of the primer can be referred to as a “5′ tail”, as described supra.

In some embodiments, a primer or a primer set is designed such that the 3′ end of the primer binds or hybridizes a short distance from the nucleic acid region of interest (e.g., SNP). In some embodiments, a primer or a primer set is designed such that the 3′ end of the primer binds or hybridizes immediately preceding the nucleic acid region of interest (e.g., SNP). In other words, the 3′ end of the primer binds or hybridizes to a base and the nearest 3′ neighbor of that base is the nucleic acid region of interest (e.g., SNP). As a non-limiting example, a primer can be designed such that the 3′ end of the primer binds or hybridizes 1 bp, 2 bp, 3 bp, 4 bp, 5 bp, 6 bp, 7 bp, 8 bp, 9 bp, or 10 bp from the nucleic acid region of interest (e.g., SNP).

In some embodiments, single based multiplexed sequencing can be performed using a limited number of cycles on an NGS platform. As a non-limiting example, single based multiplexed sequencing can be performed using 1 cycle, 2 cycles, 3 cycles, 4 cycles, 5 cycles, 6 cycles, 7 cycles, 8 cycles, 9 cycles, 10 cycles, 11 cycles, 12 cycles, 13 cycles, 14 cycles, 15 cycles, 16 cycles, 17 cycles, 18 cycles, 19 cycles, 20 cycles, 21 cycles, 22 cycles, 23 cycles, 24 cycles, 25 cycles, 26 cycles, 27 cycles, 28 cycles, 29 cycles, 30 cycles, 31 cycles, 32 cycles, 33 cycles, 34 cycles, 35 cycles, 36 cycles, 37 cycles, 38 cycles, 39 cycles, 40 cycles, 41 cycles, 42 cycles, 43 cycles, 44 cycles, 45 cycles, 46 cycles, 47 cycles, 48 cycles, 49 cycles, or 50 cycles on an NGS platform.

### Personalized Software

Also described herein are methods to rapidly analyze NGS sequencing data using personalized software to automatically provide genotype or sequencing results (see e.g., Example 16). In some embodiments, a primer set is designed using methods comprising PlexForm™. In some embodiments, a sequencing reaction is performed using a PlexSeq™ assay, and in some embodiments primers are used designed by Plexform™. In some embodiments, data from the PlexSeq™ assay is analyzed using PlexCall™

In some embodiments, PlexCall™ provides automatic calling of genotypes and allele ratios. As a non-limiting example, PlexCall™ comprises a method of calling genotypes and allele ratios, comprising at least one of the following steps: determining a sample ID based on the barcode sequence, determining an amplicon ID based on the amplicon sequence, determining an allele ratio based on the number of reads per allele, and/or determining a genotype based on the allele ratio. In some embodiments, PlexCall™ can be personalized for each experiment and includes all barcode and SNP information. In some embodiments, PlexCall™ can provide information on primer dimer (e.g., primer-primer interactions).

### Low Abundance Rare Variants

Also described herein are methods to identify and quantify low abundance rare variants. As used herein, “low abundance rare variants” refers to variants or alleles for a specific gene that are present at a low level in a population. As a non-limiting example, a low abundance rare variant can represent at most 0.01%, at most 0.1%, at most 0.2%, at most 0.3%, at most 0.4%, at most 0.5%, at most 0.6%, at most 0.7%, at most 0.8%, at most 0.9%, at most 1.0%, at most 2.0%, at most 3.0%, at most 4.0%, at most 5.0%, at most 6.0%, at most 7.0%, at most 8.0%, at most 9.0%, at most 10.0%, at most 20.0%, at most 30.0%, at most 40.0% or at most 50.0% of the total variants or alleles for a specific gene in a population.

In some embodiments, low abundance rare variants can be identified and/or quantified in clinically relevant genes, as known in the art.

In some embodiments, genotyping (e.g., for low abundance rare variant(s)) can be performed on a minority of tumor cells from a complex mixture of cells. As used herein, “complex mixture of cells” denotes that many cells types other than the cell of interest (e.g., tumor cell) are present in the mixture. In some embodiments, the complex mixture of cells can comprise at most 0.01%, at most 0.1%, at most 0.2%, at most 0.3%, at most 0.4%, at most 0.5%, at most 0.6%, at most 0.7%, at most 0.8%, at most 0.9%, at most 1.0%, at most 2.0%, at most 3.0%, at most 4.0%, at most 5.0%, at most 6.0%, at most 7.0%, at most 8.0%, at most 9.0%, at most 10.0%, at most 20.0%, at most 30.0%, at most 40.0% or at most 50.0% tumor cells.

In some embodiments, the genotyping (e.g., PlexSeq™) analysis is performed in triplicate. As a non-limiting example, the genotyping analysis can be performed at least 1 time, at least 2 times, at least 3 times, at least 4 times, at least 5 times, at least 6 times, at least 7 times, at least 8 times, at least 9 times, or at least 10 times. In some embodiments, the mutant alleles are quantified and the quantification produces a mutant allele frequency. In some embodiments, the mutant allele frequency is used to determine or quantify the percent of tumor cells in the sample.

In some embodiments, such a method can be applied to the detection of cells collected from blood, urine, other body fluids, or an organ biopsy. In some embodiments, such a method can be applied to the detection of minimal residual disease immediately post-surgery or therapy. In some embodiments, such a method can be applied to follow-up weekly, monthly or yearly to determine success of therapy and track disease recurrence.

### Nucleic Acid Samples

Methods and compositions described herein can comprise nucleic acids. In some embodiments the nucleic acids are synthetic (e.g., oligonucleotides, primers, amplicons). A nucleic acid can include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications. The preparation of the modified nucleobases described above are well known in the art. Nucleic acids can be indicated using a one-letter code, e.g., the ambiguity code developed by International Union of Pure and Applied Chemistry (IUPAC).

TABLE 13
shows IUPAC Ambiguity Codes.

| IUPAC Code | Meaning | Complement |
|---|---|---|
| A | A | T |
| C | C | G |
| G | G | C |
| T/U | T | A |
| M | A or C | K |
| R | A or G | Y |
| W | A or T | W |
| S | C or G | S |
| Y | C or T | R |
| K | G or T | M |
| V | A or C or G | B |
| H | A or C or T | D |

In some embodiments, nucleic acids are isolated from a sample. Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In some embodiments, a nucleic acid of interest (e.g., SNP) can be quantified and compared to a reference level. A level which is less than a reference level can be a level which is less by at least about 10%, at least about 20%, at least about 50%, at least about 60%, at least about 80%, at least about 90%, or less relative to the reference level. In some embodiments of any of the aspects, a level which is less than a reference level can be a level which is statistically significantly less than the reference level.

A level which is more than a reference level can be a level which is greater by at least about 10%, at least about 20%, at least about 50%, at least about 60%, at least about 80%, at least about 90%, at least about 100%, at least about 200%, at least about 300%, at least about 500% or more than the reference level. In some embodiments of any of the aspects, a level which is more than a reference level can be a level which is statistically significantly greater than the reference level.

In some embodiments of any of the aspects, the reference can be a level of the target molecule in a population of subjects who do not have or are not diagnosed as having, and/or do not exhibit signs or symptoms of a specific disease (e.g., cancer). In some embodiments of any of the aspects, the reference can also be a level of expression of the target molecule in a control sample, a pooled sample of control individuals or a numeric value or range of values based on the same. In some embodiments of any of the aspects, the reference can be the level of a target molecule in a sample obtained from the same subject at an earlier point in time, e.g., the methods described herein can be used to determine if a subject's sensitivity or response to a given therapy is changing over time.

In some embodiments of the foregoing aspects, the quantification of a nucleic acid of interest (e.g., SNP) can be normalized relative to the expression level of one or more reference genes or reference proteins.

In some embodiments, the reference level can be the level in a sample of similar cell type, sample type, sample processing, and/or obtained from a subject of similar age, sex and other demographic parameters as the sample/subject for which the level of a nucleic acid region of interest (e.g., SNP) is to be determined. In some embodiments, the test sample and control reference sample are of the same type, that is, obtained from the same biological source, and comprising the same composition, e.g. the same number and type of cells.

The term “sample” or “test sample” as used herein denotes a sample taken or isolated from a biological organism, e.g., a blood or plasma sample from a subject. In some embodiments of any of the aspects, the present invention encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; sperm; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject. In some embodiments of any of the aspects, the test sample can be a tumor biopsy. In some embodiments, the test sample can be a plant sample, including but not limited to a leaf, stem, root, bark, fruit, flower, seed, or other plant product.

The test sample can be obtained by removing a sample from a subject, but can also be accomplished by using a previously isolated sample (e.g. isolated at a prior time point and isolated by the same or another person).

In some embodiments of any of the aspects, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments of any of the aspects, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments of any of the aspects, the test sample is a clarified test sample, for example, by centrifugation and collection of a supernatant comprising the clarified test sample. In some embodiments of any of the aspects, a test sample can be a pre-processed test sample, for example, supernatant or filtrate resulting from a treatment selected from the group consisting of centrifugation, filtration, thawing, purification, and any combinations thereof. In some embodiments of any of the aspects, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments of any of the aspects, the methods, assays, and systems described herein can further comprise a step of obtaining or having obtained a test sample from a subject. In some embodiments of any of the aspects, the subject can be a human subject. In some embodiments of any of the aspects, the subject can be a subject in need of treatment for (e.g. having or diagnosed as having) cancer or a subject at risk of or at increased risk of developing cancer as described elsewhere herein.

In some embodiments, a nucleic acid sample (e.g., an isolated nucleic acid) can be amplified using PCR. The PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. Unless noted otherwise, PCR reactions are conducted under standard conditions using standard reagents, as well known to those of skill in the art.

### Next Generation Sequencing

In some embodiments as described further herein, nucleic acid samples (e.g., amplified nucleic acid samples) can be sequenced. Sequencing is the process of determining the order of monomers in a polymer. For example, DNA or RNA sequencing is the process of determining a nucleic acid sequence—the order of nucleotides in DNA or RNA, respectively, from a sample. DNA or RNA sequencing can also be referred to herein as “nucleic acid sequencing” or simply “sequencing.”

Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore).

In some embodiments as described herein, nucleic acid sequence data can be obtained from a sequencing platform. The term “sequencing platform” refers not only to a particular machine or device used for sequencing, but also to the particular chemical and/or physical approaches applied to extract or derive the sequence information from a sample. Exemplary methods of sequencing include, but are not limited to, Sanger sequencing, dideoxy chain termination, high-throughput sequencing, next generation sequencing, pyrosequencing (e.g., 454), sequencing by ligation and detection (SOLID™), polony sequencing, sequencing by synthesis (e.g., Illumina™), ion semiconductor sequencing (e.g., Ion Torrent™), sequencing by hybridization, nanopore sequencing, HeliScope single molecule sequencing, single-molecule real-time sequencing (SMRT), RNAP sequencing, combinatorial probe anchor synthesis (cPAS), nanopore sequencing, chain termination sequencing, DNA nanoball sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

Early methods of DNA sequencing, or “first generation sequencing,” included Sanger sequencing (also known as chain terminator sequencing) and Maxam-Gilbert sequencing (also known as chemical sequencing). High-throughput sequencing methods have significantly reduced the cost and time to sequence nucleic acid samples. High-throughput sequencing can also be referred to herein as “next-generation sequencing”, “second-generation sequencing”, “third-generation sequencing”, or “massively parallel signature sequencing (MPSS)”.

Non-limiting examples of ion semiconductor sequencing platforms include Ion Torrent™ sequencing platforms comprising Ion S5™, Ion AmpliSeq™, Ion Proton™, Ion PGM™ (e.g., PGM 314™, PGM 316™, PGM 318™, PI™, or PII™), or Ion Chef™ platforms, from ThermoFisher™ (see e.g., U.S. Pat. Nos. 7,785,785, 8,552,771, 8,692,298B2, 8,731,847B2, 8,742,472B2, 8,841,217B1, 8,912,580B2, 8,912,005B1, 8,962,366B2, 8,963,216B2, 9,116,117B2, 9,128,044B2, 9,194,000B2, 9,239,313B2, 9,404,920B2, 9,841,398B2, 9,927,393B2, 9,944,981B2, 9,958,414B2, 9,960,253B2, which are incorporated herein by reference in their entireties).

Pyrosequencing, an example of sequencing by synthesis, can also be referred to as 454 Life Sciences™ sequencing, 454 sequencing, or 454 pyrosequencing. Non-limiting examples of 454 pyrosequencing platforms include Genome Sequencer FLX™, GS20™, or GS Junior™ sequencing platforms. Pyrosequencing can also be performed on any the following sequencing platforms from QIAGEN: PyroMark Q48 Autoprep™, PyroMark Q24 Advanced™, PyroMark Q24™, or PyroMark Q96 ID™ (see e.g., U.S. Pat. Nos. 6,210,891, 7,323,305, 8,748,102, 8,765,380, which are incorporated herein by reference in their entireties).

Sequencing by synthesis include, for example, Illumina™ sequencing or Solexa™ sequencing. Non-limiting examples of Illumina™ sequencing platforms include cBot™, Genome Analyzer (GA)™, MiniSeq™, NextSeq™, MiSeq™, HiSeq 2500™, HiSeq 3000™, HiSeq 4000™, HiSeq X™ (e.g., Hiseq Ten™), iSeq™ 100, HiScan™, and iScan™ Illumina platforms (see e.g., U.S. Pat. Nos. 7,414,116, 7,329,860, 7,589,315, 7,960,685, 8,039,817, 8,071,962, 8,158,926, 8,241,573, 8,778,848, 8,778,849, 8,244,479, 8,315,817, 8,412,467, 8,422,031, 8,446,573, 8,914,241, 8,965,076, 9,012,022, 9,068,220, 9,121,063, 9,365,898, 9,410,977, 9,512,422, 9,540,690, 9,670,535, 9,752,186, 9,777,325, 9,994,687, 10,005,083, 10,053,730, 10,152,776, which are incorporated herein by reference in their entireties).

Additional non-limiting example of sequencing by synthesis platforms can comprise GeneReader™ from QIAGEN or Mini-20™ from AZCO Biotech™, Inc.

Non-limiting examples of SMRT sequencing platforms include C1™, C2™, P4-XL™, P5-C3™, P6-C4™, RS™, RS II™, or Sequel™ platforms, all from PacBio™ sequencing. SMRT sequencing can also be referred to as PacBio™ sequencing.

Non-limiting examples of cPAS sequencing platforms include BGISEQ-50™, MGISEQ 200™, BGISEQ-500™, or MGISEQ-2000™ cPAS platforms. cPas sequencing platforms can also utilize DNA nanoball sequencing methods (e.g., BGISEQ-500™, or MGISEQ-2000™).

Non-limiting examples of SOLiD™ sequencing platforms include 5500xl SOLID™, 5500 SOLID™, SOLID 5500xl Wildfire™, or SOLID 5500 Wildfire™, from Thermo Fisher Scientific™.

Non-limiting examples of Nanopore sequencing platforms include SmidgeION™, MinION™, and PromethION™, all from Oxford Nanopore Technologies™.

Chain termination sequencing can also be referred to as Sanger sequencing. Non-limiting examples of chain termination sequencing platforms can comprise Microfluidic Sanger sequencing platforms or the Apollo 100™ platform (Microchip Biotechnologies™, Inc.).

Non-limiting examples of Polony sequencing platforms include a Polonator™ platform (Dover™) or fluorescence microscope and a computer controlled flowcell.

Non-limiting examples of HeliScope single molecule sequencing platforms include Helicos® Genetic Analysis System platform or the HeliScope™ Sequencer.

Additional non-limiting examples of sequencing methods include tunneling currents DNA sequencing, sequencing by hybridization, sequencing with mass spectrometry, microscopy-based techniques, RNA polymerase (RNAP) sequencing, or in vitro virus high-throughput sequencing.

### Kits

Another aspect of the technology described herein relates to kits for multiplex genotyping, among others. Described herein are kit components that can be included in one or more of the kits described herein.

In some embodiments, the kit comprises an effective amount of PCR reagents, primers, and/or sequencing reagents. PCR reagents can comprise a polymerase, dNTPs, and/or an appropriate reaction buffer. The PCR reagents can further comprise template DNA, such as including a reference template DNA. The kit can comprise a set of primers, e.g., primers for a multiplex genotyping reaction designed using methods as described herein. The sequencing reagents can comprise reagents sufficient for library preparation specific for the sequencing platform. As a non-limiting example, the sequencing reagents can comprise primers, adaptors, polymerase, ligase, blocking reagent, lysing reagent, an appropriate buffer, and any other reagents or enzymes necessary for the specific sequencing reaction. The kit can further comprise reagents necessary for nucleic acid isolation, as described further herein.

As will be appreciated by one of skill in the art, PCR reagents, primers, and/or sequencing reagents can be supplied in a lyophilized form or a concentrated form that can diluted prior to use with cultured cells. Preferred formulations include those that are non-toxic to the cells and/or does not affect growth rate or viability etc. PCR reagents, primers, and/or sequencing reagents can be supplied in aliquots or in unit doses.

In some embodiments, the components described herein can be provided singularly or in any combination as a kit. The kit includes the components described herein, e.g., a composition comprising PCR reagents, primers, and/or sequencing reagents. In addition, the kit optionally comprises informational material.

In some embodiments, the compositions in the kit can be provided in a watertight or gas tight container which in some embodiments is substantially free of other components of the kit. For example, a composition can be supplied in more than one container, e.g., it can be supplied in a container having sufficient reagent for a predetermined number of PCR and/or sequencing reactions, e.g., 1, 2, 3 or greater. One or more components as described herein can be provided in any form, e.g., liquid, dried or lyophilized form. It is preferred that the components described herein are substantially pure and/or sterile. When the components described herein are provided in a liquid solution, the liquid solution preferably is an aqueous solution, with a sterile aqueous solution being preferred.

The informational material can be descriptive, instructional, marketing or other material that relates to the methods described herein. The informational material of the kits is not limited in its form. In one embodiment, the informational material can include information about production of a primer set, concentration, date of expiration, batch or production site information, and so forth. In one embodiment, the informational material relates to methods for using or administering the components of the kit.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

### Cancer

As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

As used herein the term “neoplasm” refers to any new and abnormal growth of tissue, e.g., an abnormal mass of tissue, the growth of which exceeds and is uncoordinated with that of the normal tissues. Thus, a neoplasm can be a benign neoplasm, premalignant neoplasm, or a malignant neoplasm.

A subject that has a cancer or a tumor is a subject having objectively measurable cancer cells present in the subject's body. Included in this definition are malignant, actively proliferative cancers, as well as potentially dormant tumors or micrometastases. Cancers which migrate from their original location and seed other vital organs can eventually lead to the death of the subject through the functional deterioration of the affected organs.

Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice.

One of skill in the art can readily identify a chemotherapeutic agent of use (e.g. see Physicians' Cancer Chemotherapy Drug Manual 2014, Edward Chu, Vincent T. De Vita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In some embodiments, genotyping methods as described herein can be used to detect and/or quantify a cancer cell with a specific allele or set of alleles. In some embodiments, the detection or quantification of cancer cells can be used to provide or alter a treatment method.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” typically means a human, animal, or plant. Usually the animal is a vertebrate such as a primate, rodent, domestic animal or game animal. Primates include chimpanzees, cynomolgous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. In some embodiments, the subject is a plant, including but not limited to species of corn, soybean, tomato, squash, cotton, wheat, sunflower, grape, cowpea, Chrysanthemum, Eucalyptus, flax, sesame, pepper, and rice. In some embodiments, the subject is a fungus, bacteria, protest, archaea, virus, or other entity with a nucleic acid genome. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of cancer. A subject can be male or female.

A subject can be one who has been previously diagnosed with or identified as suffering from or having a condition in need of treatment (e.g. cancer) or one or more complications related to such a condition, and optionally, have already undergone treatment for cancer or the one or more complications related to cancer. Alternatively, a subject can also be one who has not been previously diagnosed as having cancer or one or more complications related to cancer. For example, a subject can be one who exhibits one or more risk factors for cancer or one or more complications related to cancer or a subject who does not exhibit risk factors.

A “subject in need” of treatment for a particular condition can be a subject having that condition, diagnosed as having that condition, or at risk of developing that condition.

In the various embodiments described herein, it is further contemplated that variants (naturally occurring or otherwise), alleles, homologs, conservatively modified variants, and/or conservative substitution variants of any of the particular nucleic acids described are encompassed. One of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer. The term “treating” includes reducing or alleviating at least one adverse effect or symptom of a condition, disease or disorder associated with a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

As used herein, the term “specific binding” (e.g., hybridize) refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:

1. A method for primer design that allows for large amplicon number multiplexing using PlexForm™ software

2. A method of preparing a primer set for multiplex genotyping, the method comprising:
A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of:
   1) identifying all possible primers of 17 to 35 nucleotides within 100 base pairs of each genomic target sequence variation in set N of variable genomic target sequences;
   2) for each member of set N, selecting a subset of primer pairs from the set of step (1) that satisfies the conditions for a primer selection algorithm;
   3) evaluating specificity of primer pairs chosen in step (2) in the genome, keeping only those pairs predicted to be specific for their respective targets;
   4) selecting a set of optimized primers for the amplification of target gene set N, where the optimal primers are selected to minimize primer-primer interactions with other primers in the set by iterative calculation of predicted ΔG for interactions between primers to generate a fitness score and use of a fitness score optimization method selected from one or a combination of the group consisting of:
      a) a Monte Carlo random or pseudo-random selection method;
      b) a golden section search;
      c) gradient descent;
      d) minima hopping;
      e) genetic algorithm;
      f) neural networks;
      g) cluster analysis, in which substitution is picked to minimize score; and
      h) cluster analysis to create bins; and
   B) synthesizing the optimized primer set selected in step (4).

3. A method of preparing a primer set for multiplex genotyping, the method comprising:
A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of:
   1) identifying all possible primers of 17 to 35 nucleotides within 100 base pairs of each genomic target sequence variation in set N of variable genomic target sequences;
   2) for the primers identified in step (1), randomly selecting a primer pair for each target in set N that satisfies the conditions for a primer selection algorithm;
   3) evaluating specificity of primer pairs chosen in step (2) in the genome, keeping only those pairs predicted to be specific for their respective targets;
   4) repeat step (2) on the primer pairs kept from step (3) to generate set P, a population of randomly selected primer sets for each target in set N;
   5) generating a Fitness Score for each member of population P based upon ΔG for all possible interactions between candidate primers in each member of the population;
   6) picking acceptable member(s) of the population P based on Fitness Score;
   7) repeating steps (4)-(6) iteratively until a set of primer pairs for target genes identified in step (6) has a Fitness Score at a predetermined threshold; and
B) synthesizing the primer set identified in step (7).

4. The method of paragraph 3, wherein the Fitness Score is generated according to the rule:
a) G=the set of ΔG's for all possible interactions determined for a given member of set P; and
b) Fitness Score is calculated by:
   i) for each member of set P, calculating the sum, S, of |ΔG|^{Q} for each ΔG value in that member, wherein Q is a weighting factor constant exponent that makes large ΔG absolute values much larger than small values;
   ii) S′=S/#of ΔG values in G;
   iii) H=T/S′, wherein T is a constant that makes H small for large values of S′ and H large for small values of S′; and
   iv) Fitness Score=H^{R}, wherein R is a weighting factor constant exponent that makes large values of H larger, and small values of H smaller.

5. A method of preparing a primer set for multiplex genotyping, the method comprising:
A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of:
   1) identifying all possible primers of 17 to 35 nucleotides within 100 base pairs of each genomic target sequence variation in set N of variable genomic target sequences;
   2) for the primers identified in step (1), randomly selecting a primer pair for each target in set N that satisfies the conditions for a primer selection algorithm and is predicted to be specific for its target in the genome, or providing a primer pair for each target in set N, that has been selected to reduce potential for primer:primer interactions with other primers in the set and is predicted to be specific for its target in the genome;
   3) repeat step (2) to generate population Z, of size 2 or greater, of primer pair sets for each target in set N;
   4) generating a Fitness Score for each member of population Z based upon ΔG for all possible interactions between candidate primers in each member of the population;
   5) selecting the worst members of population Z based on Fitness Scores as set W;
   6) replacing a primer for a single target from W with another primer identified in step (2), and generating a Fitness Score for the resulting set; wherein if the change results in an improved Fitness Score relative to the Fitness Score generated in step (4), the resulting new set W′ replaces set W, and if the change results in a no change in Fitness Score or a decreased Fitness Score, keeping set W;
   7) iteratively repeating steps (4)-(6) on the set W or W′ retained in each iteration of step (6) until a set of primer pairs for target genes in set N is identified that has a Fitness Score at a predetermined threshold, or, if a predetermined threshold is not reached by iteratively repeating steps (4)-(6), beginning again at step (2) and iteratively repeating steps (4)-(6) until a set of primer pairs for target genes in set N is identified that has a Fitness Score at the predetermined threshold; and
B) synthesizing the primer set selected in step (7) that reaches the predetermined Fitness Score threshold.

6. The method of paragraph 5, wherein the Fitness Score is generated according to the rule:
a) G=the set of ΔG's for all possible interactions determined for a given member of set P; and
b) Fitness Score is calculated by:
   i) for each member of set P, calculating the sum, S, of |ΔG|^{Q} for each ΔG value in that member, wherein Q is a weighting factor constant exponent that makes large ΔG absolute values much larger than small values;
   ii) S′=S/#of ΔG values in G;
   iii) H=T/S′, wherein T is a constant that makes H small for large values of S′ and H large for small values of S′; and
   iv) Fitness Score=H^{R}, wherein R is a weighting factor constant exponent that makes large values of H larger, and small values of H smaller.

7. The method of paragraph 5, wherein the step of providing a primer pair for each target in set N that has been selected to reduce potential for primer:primer interactions with other primers in the set provides primer sets selected using one or more of a Monte Carlo random or pseudo-random selection method, a golden section search, gradient descent, minima hopping, a genetic algorithm, neural networks, cluster analysis in which substitution is picked to minimize score, or cluster analysis to create bins.

8. A method of preparing a primer set for multiplex genotyping, the method comprising:
A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of:
   1) identifying all possible primers of 17 to 35 nucleotides within 100 base pairs of each genomic target sequence variation in set N of variable genomic target sequences;
   2) generating primer set Z, including a primer pair for each member of set N either by: (a) randomly selecting from the primers identified in step (1) a primer pair for each target in set N that satisfies the conditions for a primer selection algorithm and is predicted to be specific for its target in the genome; or (b) providing a primer pair for each target in set N that is predicted to be specific for its target in the genome, and that has been selected to reduce potential for primer:primer interactions with other primers in the set;
   3) generating a Fitness Score for primer set Z based upon ΔG for all possible interactions between candidate primers in each member of the population;
   4) making a change to a primer for a single target from set Z to generate new set Z′, and generating a Fitness Score for set Z′, wherein if the change results in an improved Fitness Score relative to that generated in step (3), the resulting new set Z′ replaces set Z, and if the change results in no change in Fitness Score or a decreased Fitness Score, keeping set Z; and
   5) repeating step (4) iteratively until further iterations do not improve fitness of set Z; and
B) synthesizing the primer set selected in step (5).

9. The method of paragraph 8, wherein the Fitness Score is generated according to the rule:
a) G=the set of ΔG's for all possible interactions determined for members of primer set Z; and
b) Fitness Score is calculated by:
   i) calculating the sum, S, of |ΔG|^{Q} for each ΔG value, wherein Q is a weighting factor constant exponent that makes large ΔG absolute values much larger than small values;
   ii) S′=S/#of ΔG values in G;
   iii) H=T/S′, wherein T is a constant that makes H small for large values of S′ and H large for small values of S′; and
   iv) Fitness Score=H^{R}, wherein R is a weighting factor constant exponent that makes large values of H larger, and small values of H smaller.

10. A method of preparing a primer set for multiplex genotyping, the method comprising:
A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of:
   1) identifying all possible primers of 17 to 35 nucleotides within 100 base pairs of each genomic target sequence variation in set N of variable genomic target sequences;
   2) providing a set of optimized primer pairs for the amplification of target gene set N, where the optimal primer pairs are predicted to be specific for their target genes in the genome, and are selected to minimize primer-primer interactions with other primers in the set by iterative calculation of predicted ΔG for all possible interactions between primers to generate a Fitness Score and use of a Fitness Score optimization method selected from one or a combination of the group consisting of:
      a) a Monte Carlo random or pseudo-random selection method;
      b) a golden section search;
      c) gradient descent;
      d) minima hopping;
      e) genetic algorithm;
      f) neural networks;
      g) cluster analysis, in which substitution is picked to minimize score; and
      h) cluster analysis to create bins;
   3) adding the set of optimized primers of step (2) to set M;
   4) while maintaining a degree of dissimilarity from primer sets included in set M, selecting a primer pair for each target in set N from step (1) and designating it set Z, wherein the primer pairs satisfy the conditions for a primer selection algorithm, and are predicted to be specific for their target genes in the genome;
   5) optimizing primer pairs of set Z for the amplification of target gene set N, to minimize primer-primer interactions with other primers in the set by iterative calculation of predicted ΔG for all possible interactions between primers to generate a Fitness Score and use of a Fitness Score optimization method selected from one or a combination of methods (a)-(h) of step (2); and
   6) repeating steps (2)-(5) iteratively until a set of primer pairs for target gene set N identified in step (5) has a Fitness Score at a predetermined threshold; and
B) synthesizing the optimized primer set selected in step (6).

11. The method of paragraph 10, wherein the Fitness Score is generated according to the rule:
a) G=the set of ΔG's for all possible interactions determined for members of primer set Z; and
b) Fitness Score is calculated by:
   i) calculating the sum, S, of |ΔG|^{Q} for each ΔG value, wherein Q is a weighting factor constant exponent that makes large ΔG absolute values much larger than small values;
   ii) S′=S/#of ΔG values in G;
   iii) H=T/S′, wherein T is a constant that makes H small for large values of S′ and H large for small values of S′; and
   iv) Fitness Score=H^{R}, wherein R is a weighting factor constant exponent that makes large values of H larger, and small values of H smaller.

12. A method of preparing a primer set for multiplex genotyping, the method comprising:
A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of:
   1) identifying all possible primers of 17 to 35 nucleotides within 100 base pairs of each genomic target sequence variation in set N of variable genomic target sequences;
   2) For each primer identified in step (1) creating a node P_{nz}, such node connected to a node for the corresponding target (Tₙ), wherein:
      (i) each node outputs its ID and a numeric value;
      (ii) each Tₙ produces ID of one of the P_{nz} nodes connected to it;
      (iii) each one of the Tₙ nodes is connected to all others; and
      (iv) each node Tₙ represents a multilayer neural network;
   3) calculating a Fitness Score for output of the neural network, and on the basis of Fitness Score, the value produced by the network is compared to target, and neural network parameters for a plurality of the Tₙ are changed;
   4) calculating Fitness Score again for output of the neural network with parameters changed in step (3);
   5) determining if a change was beneficial or not to the fitness of the resulting set, wherein if the change was beneficial, the direction of change is maintained with smaller increments, and wherein if the change was not beneficial, either direction is reversed or the parameters revert to a previous state;
   6) repeating steps (3)-(5) iteratively, wherein at a plurality of iterations random changes are made to the parameters of the network, and wherein when the rate of fitness improvement decreases, the frequency of such random changes is increased, until a set of primer pairs for target genes in set N is identified that has a fitness score at a predetermined threshold; and
B) synthesizing the optimized primer set selected in step (6).

13. The method of paragraph 12, wherein the Fitness Score is generated according to the rule:
a) G=the set of ΔG's for all possible interactions determined for members of a primer set for targets N; and
b) Fitness Score is calculated by:
   i) calculating the sum, S, of |ΔG|^{Q} for each ΔG value, wherein Q is a weighting factor constant exponent that makes large ΔG absolute values much larger than small values;
   ii) S′=S/#of ΔG values in G;
   iii) H=T/S′, wherein T is a constant that makes H small for large values of S′ and H large for small values of S′; and
   iv) Fitness Score=H^{R}, wherein R is a weighting factor constant exponent that makes large values of H larger, and small values of H smaller.

14. A method of preparing a primer set for multiplex genotyping, the method comprising:
A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of:
   1) identifying all possible primers of 17 to 35 nucleotides within 100 base pairs of each genomic target sequence variation in set N of variable genomic target sequences;
   2) picking a target at random, as well as a primer for such target, and placing it in set R;
   3) picking an additional target, and calculating a Fitness Score evaluating all primers for this target in combination with primers already in set R on the basis of ΔG for all potential interactions, wherein the primer that results in the best Fitness Score is added to set R;
   4) if fitness of set R is below a predetermined threshold T, removing one of the primers from R according to the following:
      calculating a Fitness Score for set Ri, wherein the i^{th} target with its primer is removed from set R, and the set with the best Fitness Score determines the target with its primer to be removed from set R and placed back into the pool of primers of step (1); and
   5) repeating steps (3) and (4) until all targets are assigned primers; and
B) synthesizing the optimized primer set selected in step (5).

15. The method of paragraph 14, wherein the Fitness Score is generated according to the rule:
a) G=the set of ΔG's for all possible interactions determined for members of a primer set; and
b) Fitness Score is calculated by:
   i) calculating the sum, S, of |ΔG|^{Q} for each ΔG value, wherein Q is a weighting factor constant exponent that makes large ΔG absolute values much larger than small values;
   ii) S′=S/#of ΔG values in G;
   iii) H=T/S′, wherein T is a constant that makes H small for large values of S′ and H large for small values of S′; and
   iv) Fitness Score=H^{R}, wherein R is a weighting factor constant exponent that makes large values of H larger, and small values of H smaller.

16. A method of preparing a primer set for multiplex genotyping, the method comprising:
A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of:
   1) identifying all possible primers of 17 to 35 nucleotides within 100 base pairs of each genomic target sequence variation in set N of variable genomic target sequences;
   2) picking a target at random, as well as a primer for such target, and placing it in set R;
   3) picking an additional target, and calculating a Fitness Score evaluating all primers for this target in combination with primers already in set R on the basis of ΔG for all potential interactions, wherein the primer that results in the best Fitness Score is added to set R;
   4) if fitness of set R is below a predetermined threshold T, removing one of the primers from R according to the following:
      calculating a Fitness Score for set Ri, wherein the i^{th} target with its primer is removed from set R, and the set with the best Fitness Score determines the target with its primer to be removed from set R and placed back into the pool of primers of step (1);
   5) repeating steps (3) and (4) until all targets are assigned primers;
   6) once all targets are assigned primers, designating set R as R₁, and its fitness as F₁;
   7) creating empty set R_{z+1}, where Z is the number of sets, with fitness F_{z+1;}
   8) for each set Rz, where z is an index from 1 to number of sets R, determining the element that is worst for the set's fitness, and removing this element, designated Target E;
   9) recalculating Fz after removal of E;
   10) for all Rz, determining where E can be added so as to maximize Fz and maximize the minimum of Fz; and
   11) if the minimum of Fz is below the predetermined threshold, repeating steps (7)-(10) until the standard deviation of Fz is below the predetermined threshold, thereby designing a multiplex primer set; and
B) synthesizing the optimized primer set designed in step (11).

17. The method of paragraph 16, wherein the step of determining the element in step (8) that is worst for fitness is performed in a method analogous to step (4).

18. A method of preparing a primer set for multiplex genotyping, the method comprising:
A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of:
   1) identifying all possible primers of 17 to 35 nucleotides within 100 base pairs of each genomic target sequence variation in set N of variable genomic target sequences;
   2) for each member of set N, selecting from the set of primers in step (1) a subset of primer pairs that satisfies the conditions for a primer selection algorithm and is predicted to be specific for its target;
   3) repeating step (2) to generate set P, a population of randomly selected primer sets for each target gene in set N;
   4) calculating a Fitness Score for each member of the population P;
   5) placing members of population P into a pool of candidate primer sets on the basis of Fitness Scores;
   6) randomly selecting a plurality of “parent” sets of candidate primers from the pool of step (5), each parent set including a different pair of candidate primer sets, parent A and parent B;
   7) for each parent set of candidate primers, creating a crossover set of candidate primers by replacing a subset of candidate primer pairs of parent A with the corresponding subset of primer pairs of parent B;
   8) randomly replacing one primer pair in crossover set A with a different primer pair for the corresponding target sequence generated in step (2) to create a Generation 2 population of primer sets for each target gene in set N; and
   9) repeating steps (4)-(8) iteratively until a set of primer pairs for target genes in set N is identified that has a Fitness Score at a predetermined threshold, and runs for an additional set amount of iterations with no measurable improvement in the fitness of the best member, whereby an optimized primer set is designed; and

B) synthesizing the optimized primer set designed in step (9).

19. The method of paragraph 18, wherein the Fitness Score is generated according to the rule:
a) G=the set of ΔG's for all possible interactions determined for members of a primer set; and
b) Fitness Score is calculated by:
   i) calculating the sum, S, of |ΔG|^{Q} for each ΔG value, wherein Q is a weighting factor constant exponent that makes large ΔG absolute values much larger than small values;
   ii) S′=S/#of ΔG values in G;
   iii) H=T/S′, wherein T is a constant that makes H small for large values of S′ and H large for small values of S′; and
   iv) Fitness Score=H^{R}, wherein R is a weighting factor constant exponent that makes large values of H larger, and small values of H smaller.

20. A method of preparing a primer set for multiplex genotyping, the method comprising:
A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of:
1) identifying all possible primers of 17 to 35 nucleotides within 100 base pairs of each genomic target sequence variation in set N of variable genomic target sequences;
2) selecting a primer set for the multiplex amplification and genotyping of the members of set N comprising:
a) from the set of all possible primers for each genomic target sequence variation of step (1), randomly selecting set P, a population of sets of candidate primers, each individual set of candidate primers in population P including a primer pair for the amplification of each member of set N of variable genomic target sequences to be genotyped;
b) calculating a fitness score for each member of the population of set P by calculating ΔG for all possible interactions between candidate primers in each member of the population of set P, and assigning each member of set P a Fitness Score according to the rule:
   i) G=the set of ΔG's for all possible interactions determined for a given member of set P;
   ii) Number of top scorers to go into next generation=1 . . . N, Number of distinct populations sets=1 . . . N, and Population size=1 . . . N such that number of top scorers to go into next generation is greater or equal to population size;
      wherein fitness score is calculated by:
   iii) for each member of set P, calculating the sum, S, of |ΔG|^{Q} for each ΔG value in that member, wherein Q is a weighting factor constant exponent that makes large ΔG absolute values much larger than small values;
   iv) S′=S/#of ΔG values in G;
   v) H=T/S′, wherein T is a constant that makes H small for large values of S′ and H large for small values of S′;
   vi) Fitness Score=H^{R}, wherein R is a weighting factor constant exponent that makes large values of H larger, and small values of H smaller;
c) selecting a set of primers for the multiplex amplification and genotyping of members of set N by:
   i) randomly selecting a plurality of sets of “parent” sets of candidate primers, each having parent set A and parent set B, from set P based upon Fitness Scores;
   ii) for each member of the plurality of sets of parents, creating a crossover set of candidate primers by replacing a subset of candidate primers in parent set A with a corresponding subset of candidate primers in parent set B, resulting in two crossover sets, crossover set A and crossover set B; and
   iii) randomly replacing one primer pair in crossover set A with a different primer pair for the corresponding variable genomic target sequence to create a next generation population of candidate sets of primers, Generation 2; and
d) iteratively repeating steps (a)-(c), whereby a primer set for the multiplex amplification and genotyping of set N of variable genomic target sequences is selected; and
B) synthesizing the primer set designed in step (A).

21. A method of multiplex amplification, sequencing, and/or genotyping comprising using a primer set designed according to any one of the preceding paragraphs.

21. A method for equalization of NGS reads, the method comprising adding anti-sense oligonucleotides in concentrations sufficient to inhibit highly efficiently amplified amplicons from forming, thereby permitting less efficiently amplifying amplicons to produce more detectable data.

22. A method for performing multiplex PCR that results in substantially even reads produced by an NGS instrument for each of 2 to 10,000 amplicons and each of 1-100,000 samples in an NGS run.

23. A method for sequencing on an NGS instrument that permits single base multiplexed sequencing, without reading beyond a designated single base.

24. The method of paragraph 23, comprising designing a multiplex genotyping primer set as set out in any one of paragraphs 1-20, and adding random nucleotides to the 5′ end of the primers in the set that are shorter than the longest primer in the set, such that each primer in the set is the same length, N, and performing only N+1 cycles in an NGS sequencing run.

25. A method to rapidly analyze NGS sequencing data using software specific to the sample and/or target sequence or gene set to automatically provide genotype or sequencing results.

26. A method of using NGS sequencing methodology to identify and quantify low abundance, rare variants in clinically relevant genes in a minority of tumor cells from a complex mixture of cells.

### EXAMPLES

### Example 1: Large Amplicon Number Multiplexing

PlexForm™ software can be used for primer design that allows for large amplicon number multiplexing. As an example, the following general primer design scheme can be followed to design multiplex primer sets.

A target gene set N comprises Genes 1 to N (e.g., Gene 1, Gene 2, Gene 3, . . . Gene N). The target genes or DNA targets of interest can be a SNP or another region of interest. All possible primers (e.g., 17-35 nucleotides) within 100 base pairs of each target gene. Primers are chosen that satisfy standard PCR conditions for a primer selection algorithm (e.g., Primer 3™, Oligo Analyzer™, NetPrimer™, or Oligo Calculator™). Primers are compared for specificity versus the genome using alignment software (e.g., primer blast (NCBI™); isPCR (UCSC)). Only those primers predicted to be specific for their respective targets are kept. In some embodiments, a fitness score is calculated for a primer set (see e.g., Example 2).

Optimal primers are selected for each target based on the minimization of primer-primer interactions (e.g., maximization of Fitness Score) using any of several mathematical algorithms incorporated into the PlexForm™ process. Each sample point in the search space is a collection of primer pairs (one for each target) in one or more sets, where either highest chance of primer interaction or number of primers below a threshold of primer interaction within a set (or maximum value for multiple sets) provides a value that is being minimized. All techniques use a model that can accurately predict the possibility of primer interaction between members of a collection of primer pairs.

Since search space for this task is too large to search for best solution (global minima), the following techniques are employed iteratively, together, and/or separately to find an acceptable solution: Genetic Algorithm (see e.g., Example 3); a Monte Carlo random or pseudo-random selection method (see e.g., Example 4); Golden-section search (see e.g., Example 5); Gradient descent (see e.g., Example 6), Minima hopping (see e.g., Example 7); Neural Networks (see e.g., Example 8); Cluster analysis, in which substitution is picked to minimize score (see e.g., Example 9); or Cluster Analysis to create bins (see e.g., Example 10). Examples and detailed descriptions of each of these algorithmic approaches is described further herein. In some embodiments, the optimal primer set selected is then synthesized.

As a non-limiting example, the following steps can be applied as a general primer design scheme on a target gene set N {Genes 1, 2, 3, . . . N}.

Step 1: Identify all possible primers 17-35 nt, within 100 bp of each target.

Step 2: For each member of set N, elect a subset of primer pairs from the set of step 1 that satisfies the conditions for a primer selection algorithm (such as Primer 3, Oligo Analyzer, NetPrimer, or Oligo Calculator).

Step 3: Evaluate specificity of primer pairs chosen in step 2 in the genome, keeping only those pairs predicted to be specific for their respective targets.

Step 4: Select a set of optimal primers for the amplification of target gene set N, where the optimal primers are selected to minimize primer-primer interactions with other primers in the set by iterative use of one or more of the following: a) a Monte Carlo random or pseudo-random selection method; b) a golden section search; c) gradient descent; d) minima hopping; e) genetic algorithm; f) neural networks; g) cluster analysis, in which substitution is picked to minimize score; or h) cluster analysis to create bins;

Step 5: Synthesize the optimal primer set selected in step 4.

### Example 2: Fitness Score

In some embodiments, a fitness score is calculated for a primer set, chromosome, or individual. As used in the context of a genetic algorithm, a “chromosome” or “individual” is a set of “X” primer pairs. For example, if running analysis on 150 primer pairs, the chromosome or individual comprises 150 primer pairs. 100 different individuals are assembled randomly from the available primers. This set of 100 “individuals” comprises a “population”. The primers within each primer set or within each of the 100 individuals within the population are analyzed for ΔG for all possible interactions. A “score” is then calculated for each primer set or for each individual in the population.

In some embodiments, the following Fitness Score (e.g., F) in FORMULA 2 is used:

$$F = {\{ C / [ ( Σ ( {\vert Δ G \vert}^{A} ) ) / {G}_{n} ] \}}^{B} \qquad FORMULA 2$$

G equals the set of ΔG's for all possible primer interactions determined for that individual or primer set. A and B are weighting factors. C is a factor to create reciprocal scaled values; large numbers become small and vice versa. As a non-limiting example, A equals 5, B equals 2, and C equals 200.

The absolute value of each ΔG value is raised to the “A” power, and all such values are summed for an individual or primer set. The “A” exponent has the effect of spreading out the data values; “A” expands the scale, making large values much larger than small values. This in effect “weights” or penalizes values proportional to the magnitude of “A”. This sum is “S”. See e.g., FORMULA 3.

$$S = Σ ( {\vert Δ G \vert}^{A} ) \qquad FORMULA 3$$

This sum (e.g., “S”) is divided by the number of ΔG values (e.g., “Gₙ”). This division by Gₙ normalizes S, such that the sum is not inherently larger or smaller for larger or smaller numbers of SNPs, respectively. This normalized S value is “S′”. See e.g., FORMULA 4.

$${S}^{′} = S / {G}_{n} \qquad FORMULA 4$$

C (a constant) is divided by S′ resulting in value “H”. C is a factor to create reciprocal scaled values; large numbers become small, and small numbers become large. See e.g., FORMULA 5.

$$H = C / {S}^{′} \qquad FORMULA 5$$

Finally, H is raised to the “B” power. “B” once again has the effect of further spreading out the data, making the difference between large and small values even bigger. This value is the “Fitness Score” or F. See e.g., FORMULA 6.

$$F = {H}^{B} \qquad FORMULA 6$$

Each individual or primer set is assigned a fitness score (e.g., “F”). “F” can be calculated using FORMULA 1 or FORMULAS 3-6. FORMULA 2 is a combination of FORMULAS 3-6.

### Example 3: Genetic Algorithm

PlexForm™ software can be used for primer design that allows for large amplicon number multiplexing. Included herein is a description of a genetic algorithm used as one approach in PlexForm™ In a genetic algorithm, it is possible to pick several valid points and evaluate points in such cohort. Few points that produce lowest chance of primer interaction move on to next iteration with rest of the population being replaced via combining points with lowest chance of primer interaction, random, pseudo random, and/or guided changes. Magnitude of the change can be correlated to fitness of the best point, average fitness, predetermined function, rate of improvement or other metrics. This process is repeated. Repetition can be stopped when model of high enough fitness is obtained, time limit and/or changes are insignificant.

Such a genetic algorithm works on the input of multiple primers generated for each target (see e.g., Example 1). A fitness score for each individual can be calculated (see e.g., Example 2). An optimized set of primers is selected using a genetic algorithm (see below). The optimized primer set can then be synthesized. Described below is a non-limiting example using a genetic algorithm.

Each generation comprises a selection step and a mutation step. In the Selection step, the top 5 individuals (e.g., individuals with the 5 highest Fitness Scores, or “The Elite”) are automatically passed through to the next generation. This is referred to as Elitism. All individuals are represented in a theoretical “pool” proportional to their fitness score. In this way, those individuals with a higher fitness score are more highly represented in this “pool”, and those with very low fitness scores are minimally represented in this pool. Note that the Elite, though automatically passed to the next generation, remain represented in the pool. From this pool, two “parent” individuals are selected randomly. Note that the pool is not random itself as it is weighted to increase probability of selection of higher scoring individuals.

In the Mutation step, two “parent” individuals (or chromosomes) undergo a cross-over event. The result of one of those crossover events is retained. The resulting “child” individual then undergoes “mutation”. Each individual has a fixed probability of undergoing a mutation. Thus, for the individual as a whole, there can be from 0 mutations up to any number of mutations. As used herein, mutation denotes that another primer pair for the same target is used to replace the existing one. 100 pairs of parents are selected from the existing generation score-based pool. This results in 100 Child individuals, all of which result from a crossover of parents and a subsequent mutation event. The resulting 100 child individuals then comprise the next Generation population, and the process is repeated over and over again.

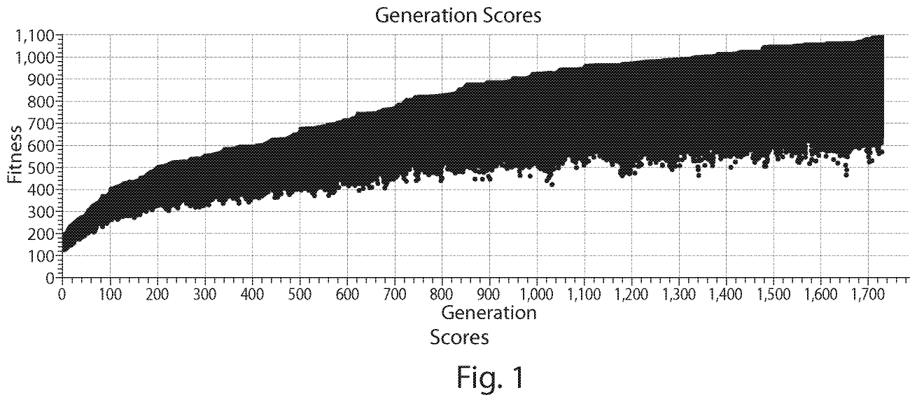

As an example, a genetic algorithm was used over multiple generations to optimize a primer set. As an output, two files were written every 25 generations or cycles to record the ΔG scores and primer pairs. One could thus monitor the process graphically and halt as needed (see e.g., FIG. 1). At that point, the final result was written to an output file. The output files contain data for the individual with the best Fitness Score at that point. One file contains the SNP name and the sequence of the associated primers which are then used in the PlexSeq™ assay. The other file contains the ΔG values for all possible interactions between those primers.

As shown in FIG. 1, the X axis displays each generation and the Y Axis displays the Fitness score. The data points at each generation are all of the individuals within that generation. Each individual in Generation X is plotted as a function of that individual's Fitness Score.

As a non-limiting example, the following steps can be applied as a primer design scheme based on a genetic algorithm for a target gene set N {Genes 1, 2, 3, . . . N}.

Step 1: Identify all possible primers 17-35 nt, within 100 bp of each target.

Step 2: A) from the primers identified in step (1), randomly select a primer pair for each target; B) repeat step (A) to generate set P, a population of randomly selected primer sets for each target gene in set N; C) calculate a fitness score for each member of the population P, according to the noted rule/algorithm (see e.g., Example 2); D) on the basis of fitness scores, place members of population P into a pool of candidate primer sets; E) randomly select a plurality of “parent” sets of candidate primers from the pool of (D), each parent set including a different pair of candidate primer sets, parent A and parent B; F) for each parent set of candidate primers, create a “crossover” set of candidate primers by replacing a subset of candidate primer pairs of parent A with the corresponding subset of primer pairs of parent B; G) randomly replace one primer pair in crossover set A with a different primer pair for the corresponding target sequence to create a Generation 2 population of primer sets for each target gene in set N; and H) repeat steps (C)-(G) iteratively until a set of primer pairs for target genes in set N is identified that has a fitness score at a desired threshold, and runs for and additional set amount of time with no measurable improvement in the fitness of the best member.

As another non-limiting example, described below is a method of preparing a primer set for multiplex genotyping, the method comprising:
A) for a given set N of variable genomic target sequences to be genotyped in a sample, designing a set of forward and reverse amplification primers that amplifies a sequence comprising each variable genomic target sequence in a multiplex amplification reaction, wherein the designing includes the steps of:
1) identifying all possible primers of 17 to 35 nucleotides within 100 base pairs of each genomic target sequence variation in set N of variable genomic target sequences;
2) selecting of a primer set for the multiplex amplification and genotyping of the members of set N comprising:
a) from the set of all possible primers for each genomic target sequence variation of step (1), randomly selecting set P, a population of sets of candidate primers, each individual set of candidate primers in population P including a primer pair for the amplification of each member of set N of variable genomic target sequences to be genotyped;
b) calculating a fitness score for each member of the population of set P by calculating ΔG for all possible interactions between candidate primers in each member of the population of set P, and assigning each member of set P a Fitness Score according to the rules: i) G=the set of ΔG's for all possible interactions determined for a given member of set P; ii) Number of top scorers to go into next generation=1 . . . N, Number of distinct populations sets=1 . . . N, and Population size=1 . . . N such that number of top scorers to go into next generation is greater or equal to population size; Where iii) for each member of set P, calculate the sum, S, of |ΔG|^{A} for each ΔG value in that member; iv) S′=S/#of ΔG values in G; v) H=C/S′; vi) Fitness Score=H^{B}; Better fitness score improves chances of specific set to move onto next generation, and/or be one of the parents for one or more sets in next generation.
c) selecting a set of primers for the multiplex amplification and genotyping of members of set N by: i) randomly selecting a plurality of sets of “parent” sets of candidate primers, each having parent set A and parent set B, from set P based upon Fitness Scores; ii) for each member of the plurality of sets of parents, creating a crossover set of candidate primers by replacing a subset of candidate primers in parent set A with a corresponding subset of candidate primers in parent set B, resulting in two crossover sets, crossover set A and crossover set B; and iii) randomly replacing one primer pair in crossover set A with a different primer pair for the corresponding variable genomic target sequence to create a next generation population of candidate sets of primers, Generation 2; and
d) iteratively repeating steps (a)-(c) whereby a primer set for the multiplex amplification and genotyping of set N of variable genomic target sequences is selected; and
B) synthesizing the primer set designed in step (A).

### Example 4: Monte Carlo Algorithm

PlexForm™ software can be used for primer design that allows for large amplicon number multiplexing. Included herein is a description of a Monte Carlo algorithm used as one approach in PlexForm™. Using random and/or pseudo random selection, one can achieve with a Monte Carlo method a uniform distribution of the sample points in the search space. Such a distribution can be augmented or replaced by selection of inputs near points of interest, that can be determined.

Such a Monte Carlo algorithm works on the input of multiple primers generated for each target (see e.g., Example 1). A fitness score for each primer set can then be calculated (see e.g., Example 2). An optimized set of primers is selected using a Monte Carlo algorithm (see below). The optimized primer set can then be synthesized.

As a non-limiting example, the following steps can be applied as a primer design scheme based on a Monte Carlo method for a target gene set N {Genes 1, 2, 3, . . . N}.

Step 1: Identify all possible primers 17-35 nt, within 100 bp of each target.

Step 2: A) from the primers identified in step (1), randomly select a primer pair for each target; B) repeat step (A) to generate set P, a population of randomly selected primer sets for each target gene in set N; C) calculate a fitness score for each member of the population P, according to the noted rule/algorithm (see e.g., Example 2); D) based on the fitness score, pick the best (best fitness) member of the population; E) repeat steps (A)-(D) iteratively until a set of primer pairs for target genes identified in step D has a fitness score at a desired threshold.

### Example 5: Golden Section Search

PlexForm™ software can be used for primer design that allows for large amplicon number multiplexing. Included herein is a description of a Golden Section Search algorithm used as one approach in PlexForm™. Using a Golden Section Search algorithm, it is possible to pick two points (where some of the targets contain primer pairs that are the same and do not change during the progression of the search) and using the assumption that minima located between two of those pick a third and fourth point between first two and evaluate the model of each point. Using the assumption that in between two original points the function produced by the model is unimodal, one removes an outside point that has an additional point between it and a point with minimum value. A replacement for the removed point can be picked in-between the two outermost points. This process is repeated until the distance between two outer most points is small enough to be within a previously determined margin.

Such a Golden Section Search algorithm works on the input of multiple primers generated for each target (see e.g., Example 1). A fitness score for each primer set can then be calculated (see e.g., Example 2). An optimized set of primers is selected using a Golden Section Search algorithm (see below). The optimized primer set can then be synthesized.

As a non-limiting example, the following steps can be applied as a primer design scheme based on a Golden Section Search method for a target gene set N {Genes 1, 2, 3, . . . N}.

Step 1: Identify all possible primers 17-35 nt, within 100 bp of each target.

Step 2: A) from the primers identified in step (1), randomly or based on results of other scheme select a primer pair for each target; B) repeat step (A) to generate population Z of size 2 or greater, a population of randomly selected primer sets for each target gene in set N; C) calculate a fitness score for each member of the population Z, according to the noted rule/algorithm (see e.g., Example 2); D) based on fitness scores, pick the worst members (e.g., lowest fitness score) of the population Z: W; E) make a change to a primer for a single target from W and evaluate fitness of resulting set, and if the fitness of the new set is better than fitness of W, new set (W′) replaces W; F) repeat steps (C)-(E) iteratively until a set of primer pairs for target genes in set N is identified that has a fitness score at a desired threshold, or there is no improvement, or if threshold was not reached start again from step A.

### Example 6: Gradient Descent

PlexForm™ M software can be used for primer design that allows for large amplicon number multiplexing. Included herein is a description of a gradient descent algorithm used as one approach in PlexForm™. Using a gradient descent algorithm, it is possible to determine a gradient (that represents an increase or decrease in change of interaction between primers) in a given point by measuring a change in interaction with the change of a single primer pair for a given target. Measured change in primer interaction points to a change in primer pairs that produces the greatest reduction in primer interaction. The process is repeated until a point is achieved where any change increases primer interaction score. Such a point is declared minima (where interaction is least likely).

Such a gradient descent algorithm works on the input of multiple primers generated for each target (see e.g., Example 1). A fitness score for each primer set can then be calculated (see e.g., Example 2). An optimized set of primers is selected using a gradient descent algorithm (see below). The optimized primer set can then be synthesized.

As a non-limiting example, the following steps can be applied as a primer design scheme based on a gradient descent method for a target gene set N {Genes 1, 2, 3, . . . N}.

Step 1: Identify all possible primers 17-35 nt, within 100 bp of each target.

Step 2: A) from the primers identified in step (1), randomly or based on results of other scheme select a primer pair for each target calling such set of primers Z; B) calculate a fitness score for Z, according to the noted rule/algorithm (see e.g., Example 2); C) make a change to a primer for a single target from Z and evaluate the fitness of the resulting set (according to the noted rule/algorithm, see e.g., Example 2), if the fitness of the resulting set is better than fitness of Z, new set (Z′) replaces Z; D) repeat step (C) iteratively until there is no improvement to fitness of Z.

### Example 7: Minima Hopping

PlexForm™ software can be used for primer design that allows for large amplicon number multiplexing. Included herein is a description of a minima hopping algorithm used as one approach in PlexForm™. To ensure that minima (e.g., where primer interaction is least likely) that are found are not local minima, minima hopping can be employed. Upon locating a minima, it is noted, and a new point in the search space is picked. The new point can be picked randomly or via deterministic method(s) that may be based on variety of factors such as completeness of the data, historical accuracy of predictions, coverage during present search, human guided suggestion, and random and pseudo random number generation, or any combination of those methods as well as others.

Such a minima hopping algorithm works on the input of multiple primers generated for each target (see e.g., Example 1). A fitness score for each primer set can then be calculated (see e.g., Example 2). An optimized set of primers is selected using a minima hopping algorithm (see below). The optimized primer set can then be synthesized.

As a non-limiting example, the following steps can be applied as a primer design scheme based on a minima hopping method for a target gene set N {Genes 1, 2, 3, . . . N}.

Step 1: Identify all possible primers 17-35 nt, within 100 bp of each target.

Step 2: A) From the results of another scheme obtain a primer set where no marked improvement was achieved, add it to set M; B) Select a primer pair for each target, calling such set of primers Z, while maintaining a degree of dissimilarity from primers sets included in M; C) Use any other scheme to improve Z; D) Repeat step (A)-(C) iteratively until a set of primer pairs for target genes identified in step C has a fitness score at a desired threshold.

### Example 8: Neural Networks

PlexForm™ software can be used for primer design that allows for large amplicon number multiplexing. Included herein is a description of a neural networks algorithm used as one approach in PlexForm™. Using neural networks, it is possible to create a function (one or more per target) that gets inputs of all possible primer pairs. Such a function produces a recommendation based on internal values and/or functions. Once a recommendation has been made, an output of functions is fed back into the initial set of functions, with some causing output values to change. After the function settles on certain point, primer interaction score is calculated. If this score is not satisfactory, changes are made to internal values and/or functions, and the process is repeated. Change to the internal values can be guided by a variety of algorithms. It is possible to have internal values for the function to be picked in advance as well as adjusted or created just for the set of targets.

Such a neural networks algorithm works on the input of multiple primers generated for each target (see e.g., Example 1). A fitness score for each primer set can then be calculated (see e.g., Example 2). An optimized set of primers is selected using a neural networks algorithm (see below). The optimized primer set can then be synthesized.

As a non-limiting example, the following steps can be applied as a primer design scheme based on a neural networks method for a target gene set N {Genes 1, 2, 3, . . . N}.

Step 1: Identify all possible primers 17-35 nt, within 100 bp of each target.

Step 2: A) For each primer identified in step 1 a node Pnz is created, such node is connected to a node for the corresponding target (Tn); B) Each node outputs its ID and a numeric value; each Tn produces ID of one of the Pnz nodes connected to it. Each one of the Tn nodes is connected to all others. C) Each node Tn represents a multilayer neural network. D) Calculate a fitness score as the output of the neural network is evaluated on the basis of fitness scores. The produced value is compared to target, and neural network parameters for some of the Tn are changed. E) Fitness score is calculated again for output of the neural network. F) Determination is made if change was beneficial or not to the fitness of the result set. If change was beneficial direction of change is maintained with smaller increments. If change was not beneficial either direction is reversed to revert to previous state. G) From time to time there are random changes that are be made to the parameters of the network; when there are no marked improvements in fitness rate of such changes may be increased. H) Repeat steps (E)-(G) iteratively until a set of primer pairs for target genes in set N is identified that has a fitness score at a desired threshold.

### Example 9: Cluster Analysis

PlexForm™ software can be used for primer design that allows for large amplicon number multiplexing. Included herein is a description of a cluster analysis algorithm used as one approach in PlexForm™. Using a cluster analysis algorithm where all primer pair interactions are placed in a cluster, some members of the cluster that are outer most are candidates for substitution (either one of the primer pairs is replaced, or both). Substitution is picked in such way to minimize resulting score (or maximize the fitness score).

Such a cluster analysis algorithm works on the input of multiple primers generated for each target (see e.g., Example 1). A fitness score for each primer set can then be calculated (see e.g., Example 2). An optimized set of primers is selected using a cluster analysis algorithm (see below). The optimized primer set can then be synthesized.

As a non-limiting example, the following steps can be applied as a primer design scheme based on a cluster analysis method for a target gene set N {Genes 1, 2, 3, . . . N}.

Step 1: Identify all possible primers 17-35 nt, within 100 bp of each target.

Step 2: A) A target is picked at random, as well as a primer for such target. It is trivial that this target and primer produce a high fitness score. It is placed in set R. B) An additional target is picked, and all primers for this target are evaluated in combination with primers already in R. The primer that results in best fitness score (according to the noted rule/algorithm; see e.g., Example 2) is added to set R. C) If the fitness of set R is below threshold T, one of the primers is removed from R according to following: a fitness score is generated for set Rᵢ (where i^{th} target with its primer is removed from R). The set with the best fitness core determines target with its primer to be removed from R and be placed in the pool of unassigned targets. D) Steps (B)-(C) are repeated until all primers are assigned. If there is an attempt to add element to the set R and if after several attempts fitness of the set R is below threshold T, either threshold T can be lowered, or the process from Step (A) is restart starting with different initial target.

### Example 10: Cluster Analysis to Create Bins

PlexForm™ software can be used for primer design that allows for large amplicon number multiplexing. Included herein is a description of a cluster analysis to create bins used as one approach in PlexForm™. If there is a situation where it becomes evident that the solution to produce a set of primers where primer interaction is below a certain threshold is not attainable, cluster analysis can be used to separate targets into two or more sets where targets that have primer pairs with high likelihood of primer interactions will be placed into separate sub set(s) once they are extracted. New subset is optimized using approaches listed above (e.g., a Monte Carlo random or pseudo-random selection method; a golden section search; gradient descent; minima hopping; genetic algorithm; neural networks; cluster analysis). If a score for the new set is significantly lower, as compared to an original set, new elements may be added to even out the chance of interaction between multiple sets.

Such a cluster analysis to create bins works on the input of multiple primers generated for each target (see e.g., Example 1). A fitness score for each primer set can then be calculated (see e.g., Example 2). An optimized set of primers is selected using a cluster analysis to create bins (see below). The optimized primer set can then be synthesized.

As a non-limiting example, the following steps can be applied as a primer design scheme based on a cluster analysis to create bins method for a target gene set N {Genes 1, 2, 3, . . . N}.

Step 1: Identify all possible primers 17-35 nt, within 100 bp of each target.

Step 2: A) A target is picked at random, as well as a primer for such target. It is trivial that this produces a high fitness score. It is placed in set R. B) An additional target is picked, and all primers for this target are evaluated in combination with primers already in R. Primer that results in best fitness score (according to the noted rule/algorithm; see e.g., Example 2) is added to set R. C) If the fitness of set R is below threshold T, one of the primers is removed from R according to following: a fitness score is generated for set Rᵢ (where i^{th} target with its primer is removed from R). The set with the best fitness core determines target with its primer to be removed from R and be placed in the pool of unassigned targets. D) Steps (B)-(C) are repeated until all primers are assigned. If there is an attempt to add element to the R, after several unsuccessful attempts threshold T can be lowered. E) Once all primers are assigned set R is marked as R₁, and its fitness is F₁. F) Empty set R_{z+1} (where z is number of R sets) is created; its fitness is F_{z+1}. G) For each set R_{z} (where z is an index from 1 to number of sets R) determine element that is the worst for its fitness. This can be done in a similar manner to step C. Once this element (Target E) is removed, recalculate F_{z}. H) For all R_{z} determine where E can be added in such a way as to maximize F_{z} and maximize minimum of F_{z}. I) Once standard deviation of F_{z} is below threshold, this means that all bins have similar fitness level. If minimum of F_{z} is below threshold steps (F)-(H) are repeated.

### Example 11: Exemplary Primer Set #1

PlexForm™ software can be used for primer design that allows for large amplicon number multiplexing. Included herein is an exemplary primer set designed by PlexForm™ software for Homo sapiens SNP targets (see e.g., TABLE 2). TABLE 2 shows the input allelic target sequences for 81 SNP regions, comprising SEQ ID NOs: 1-167. SNP position indicated by bolded letters in TABLE 2. A lack of bolded nucleotide(s) in a target sequence in TABLE 2 indicates a deletion.

TABLE 2
Input target sequences for 81 SNP regions

| | Allele | SEQ ID | |
|---|---|---|---|
| Target | No. | NO: | Target Sequence |
| SNP001 | 1 | 1 | GCAAGGAATATATTAAATTTTTTCTTTCTTGCACAGAATTCAAT |
| | | | GTTAAACAAGTATGTTGCCATTCTGTGGAAGGCATTATTTTCCC |
| | | | CTTCCAAACTTTGAAACTCAAAAGTTTTCTAAGAAAAAAAATCA |
| | | | AATCC |
| SNP001 | 2 | 2 | GCAAGGAATATATTAAATTTTTTCTTTCTTGCACAGAATTCAAT |
| | | | GTTAAACAAGTATGTTGCCATTCTGTGGAATTAAAAAAAAAAA |
| | | | GGCATTATTTTCCCCTTCCAAACTTTGAAACTCAAAAGTTTTCTA |
| | | | AGAAAAAAAATCAAATCC |
| SNP002 | 1 | 3 | ATATAGTGTCAGGTTAAGCATTATTAGGTTAAGTTATAGCTATC |
| | | | TGTGGCAATAGCAAGCAGTTTCAAGGGATGAATCCATAGCTCA |
| | | | AAGCGGGGAATAGGACGTATAGCTCTCTCATTTTAATGTCTCTC |
| | | | TGGGCCTGATGATTTAAAAGACTCACACTCCTAAGATGAAAGTT |
| | | | ATTTTCTCATCTATGTAATGTGTTAA |
| SNP002 | 2 | 4 | ATATAGTGTCAGGTTAAGCATTATTAGGTTAAGTTATAGCTATC |
| | | | TGTGGCAATAGCAAGCAGTTTCAAGGGATGAATCCATAGCTCA |
| | | | AAGCGGGGAATAGGATGTATAGCTCTCTCATTTTAATGTCTCTC |
| | | | TGGGCCTGATGATTTAAAAGACTCACACTCCTAAGATGAAAGTT |
| | | | ATTTTCTCATCTATGTAATGTGTTAA |
| SNP003 | 1 | 5 | TAAGAACTGTTAGGTGACTGACACATCTAGGAGGAAAATGAGG |
| | | | GGTGTCCTGGCGCTTAGTTCTTCAAACCCGGTAGGAATAAGGCA |
| | | | AGCCTGGTCTACAGGAAACCATCTGTCCTGACTCCGGGAGGGT |
| | | | AAGATGGACAAGCAGGTCATTTTCAGCTCCTATTTCAGTTGCCC |
| | | | TATGGAACAGGGGTGATTCAAACTGTA |
| SNP003 | 2 | 6 | TAAGAACTGTTAGGTGACTGACACATCTAGGAGGAAAATGAGG |
| | | | GGTGTCCTGGCGCTTAGTTCTTCAAACCCGGTAGGAATAAGGCA |
| | | | AGCCTGGTCTACAGGGAACCATCTGTCCTGACTCCGGGAGGGT |
| | | | AAGATGGACAAGCAGGTCATTTTCAGCTCCTATTTCAGTTGCCC |
| | | | TATGGAACAGGGGTGATTCAAACTGTA |
| SNP004 | 1 | 7 | CTGCACAGGGCCGGGATCCCTGCCCTCTGGGAGTTGATGCTCTT |
| | | | GGGGTGGGAGGACACAGATGCTTCAGGATCCCTTAGTGCTTCA |
| | | | GGATTCTAGAGTCTCAGAATTTCCAAGCCAAGGCTTGGAGTGCC |
| | | | TCAGCTGATGTCACAGTGGAGGTTCTAGCAGAGTGGGTAGCAC |
| | | | ATATGTGTCATGTCCCTCTGGTCTG |
| SNP004 | 2 | 8 | CTGCACAGGGCCGGGATCCCTGCCCTCTGGGAGTTGATGCTCTT |
| | | | GGGGTGGGAGGACACAGATGCTTCAGGATCCCTTAGTGCTTCA |
| | | | GGATTCTAGAGTCTCTTAGAATTTCCAAGCCAAGGCTTGGAGTG |
| | | | CCTCAGCTGATGTCACAGTGGAGGTTCTAGCAGAGTGGGTAGC |
| | | | ACATATGTGTCATGTCCCTCTGGTCTG |
| SNP005 | 1 | 9 | GTGAGACTGCGTGTGCAGGTGTGTGTGGAGGGCTGTGGGGAGC |
| | | | TGTGTGTGGGGGGGGTGTGTGTGTGAGGTTGGGGGCTGTGTGG |
| | | | GGTGTGTGTGAGGCTGCGTGTGGGGAGTGTGAGGCTGTGTGTG |
| | | | TGCGAGGGGGACTATGTGTGTCGGATGATGTCCCTGGCTGTGTG |
| | | | TGGGGATGTGTGTGTGTGTGTGTGTGTG |
| SNP005 | 2 | 10 | GTGAGACTGCGTGTGCAGGTGTGTGTGGAGGGCTGTGGGGAGC |
| | | | TGTGTGTGGGGGGGGTGTGTGTGTGAGGTTGGGGGCTGTGTGG |
| | | | GGTGTGTGTGAGGCTGTGTGTGGGGAGTGTGAGGCTGTGTGTGT |
| | | | GCGAGGGGGACTATGTGTGTCGGATGATGTCCCTGGCTGTGTGT |
| | | | GGGGATGTGTGTGTGTGTGTGTGTGTG |
| SNP006 | 1 | 11 | CCAATACAGGAGCACTCAGATTCATAAAGCAAGTCCTTAGAGA |
| | | | CCTACAAAGAGACTTAGAACTCCCACACAATAATAATGGGAGA |
| | | | CTTCAACACCTCACTGCCAACATTAGACAGATAGAGACAGAAA |
| | | | GTTAACAAGGATATCCAGGAATTGAACTCAGGAATTGAACTCA |
| | | | GCTCTGCACCAAGCGGACCTAATAGACAT |
| SNP006 | 2 | 12 | CCAATACAGGAGCACTCAGATTCATAAAGCAAGTCCTTAGAGA |
| | | | CCTACAAAGAGACTTAGAACTCCCACACAATAATAATGGGAGA |
| | | | CTTCAACACCTCACTGTCAACATTAGACAGATAGAGACAGAAA |
| | | | GTTAACAAGGATATCCAGGAATTGAACTCAGGAATTGAACTCA |
| | | | GCTCTGCACCAAGCGGACCTAATAGACAT |
| SNP007 | 1 | 13 | CAAGTGAAACAACCAACTATGGCTGTAAGGATCATGAAAAACA |
| | | | GGGAATTCCCCCCCAGTTTACACAGACAACAAAAACTAAGTGT |
| | | | AGGTCACTATCTCATTGTACCCATGGATTTTAATTTATAGAGGT |
| | | | GACTGAGTGATGACATAGAAAGACCAATGCCATCGAAAGAATA |
| | | | ATTTATTACTTACAAGTCCTGGGAGAAG |
| SNP007 | 2 | 14 | CAAGTGAAACAACCAACTATGGCTGTAAGGATCATGAAAAACA |
| | | | GGGAATTCCCCCCCAGTTTACACAGACAACAAAAACTAAGTGT |
| | | | AGGTCACTATCTCATTTTACCCATGGATTTTAATTTATAGAGGT |
| | | | GACTGAGTGATGACATAGAAAGACCAATGCCATCGAAAGAATA |
| | | | ATTTATTACTTACAAGTCCTGGGAGAAG |
| SNP008 | 1 | 15 | CTTTTGTCTCCCAGGCAAGATGCTATTAGGGGTCACTTCCCATG |
| | | | TACGGAGAAAACAATCTTCATGGATAATAATGATAAAACCTTA |
| | | | TGGAATGCAAAAACAACCAAAATATGTATTCTCGGATGACTGC |
| | | | ATTAGGGCCAAGTCAATATTAGTCCCACTTCACCCACGCATTGC |
| | | | ATAGTCTAAAAATGCTGTCAGCCTGAT |
| SNP008 | 2 | 16 | CTTTTGTCTCCCAGGCAAGATGCTATTAGGGGTCACTTCCCATG |
| | | | TACGGAGAAAACAATCTTCATGGATAATAATGATAAAACCTTA |
| | | | TGGAATGCAAAAACAGCCAAAATATGTATTCTCGGATGACTGC |
| | | | ATTAGGGCCAAGTCAATATTAGTCCCACTTCACCCACGCATTGC |
| | | | ATAGTCTAAAAATGCTGTCAGCCTGAT |
| SNP009 | 1 | 17 | CAACGTTTTTTAGATTCCTCATATGAGTGAGATCATGCTGTGAA |
| | | | TCTGTTTCTGGCTGATTTCACTTAGGAGGATGTCCTCCAAGCTC |
| | | | ATCCATGCTGTCCCAAAGGCTGAATAATATTCCATTGTATATAT |
| | | | CTCTCACATTTTCTTTATCCATTCATTCATCAACTTAGATTTTTTT |
| | | | CA |
| SNP009 | 2 | 18 | CAACGTTTTTTAGATTCCTCATATGAGTGAGATCATGCTGTGAA |
| | | | TCTGTTTCTGGCTGATTTCACTTAGGAGGATGTCCTCCAAGCTC |
| | | | ATCCATGCTGTCCCAAAGGGCAAGATCTCCTTTTTAAAGGCTG |
| | | | AATAATATTCCATTGTATATATCTCTCACATTTTCTTTATCCATT |
| | | | CATTCATCAACTTAGATTTTTTTCA |
| SNP010 | 1 | 19 | AGAGCAAGATAAGTAGAATCCAAAGCAATGATCTGACTGCTCA |
| | | | AAATCACCGATATTGACAACTGACTCCCAAATCCCTGCTTCATC |
| | | | TAACATATATTGCTAATACCATGCCCAGATAGAACACAAAGCA |
| | | | ATATTTATTATATGACAAATTCTCTCCATAATTTTAGAGAGTTTT |
| | | | CCCTAAGGAAAGAAAGGACTTTTTAA |
| SNP010 | 2 | 20 | AGAGCAAGATAAGTAGAATCCAAAGCAATGATCTGACTGCTCA |
| | | | AAATCACCGATATTGACAACTGACTCCCAAATCCCTGCTTCATC |
| | | | TAACATATATTGCTACTACCATGCCCAGATAGAACACAAAGCA |
| | | | ATATTTATTATATGACAAATTCTCTCCATAATTTTAGAGAGTTTT |
| | | | CCCTAAGGAAAGAAAGGACTTTTTAA |
| SNP011 | 1 | 21 | ATATTAGTAGACATAAAAATCACCTGGGGAGAGTGTAAAAAAA |
| | | | TAAAAATTCCCAGAAATTCTGGTTCAGTCATTTTGGGGCCAACC |
| | | | TAGTCATTTGCATTAATCAGCATGCACTCCCTGATGATTCTGAT |
| | | | ATAAATGAATCACATGTTGCAAAATGCTTTAGCCTGTTCCCTAA |
| | | | TCTAAATCTTATCTCTCTCCTCTCAG |
| SNP011 | 2 | 22 | ATATTAGTAGACATAAAAATCACCTGGGGAGAGTGTAAAAAAA |
| | | | TAAAAATTCCCAGAAATTCTGGTTCAGTCATTTTGGGGCCAACC |
| | | | TAGTCATTTGCATTAGTCAGCATGCACTCCCTGATGATTCTGAT |
| | | | ATAAATGAATCACATGTTGCAAAATGCTTTAGCCTGTTCCCTAA |
| | | | TCTAAATCTTATCTCTCTCCTCTCAG |
| SNP012 | 1 | 23 | ACTCAAGTGATCCTCCTGCCTTGGCCTCCCAAAGTGCTAGGATT |
| | | | ACAGGCATGAGCCACTGCGCCTGGCCCAGTTACTTATTTTAGAA |
| | | | GTTATATTTGAGCACCTATTCTGTGCCGAGCCCTGGCATGAGCT |
| | | | GTGAACAGGCCATATCTATCCTAGATGTGCACTAATGGGGCTTT |
| | | | GGAGGGTGGCAACAGGAGGCCCGGT |
| SNP012 | 2 | 24 | ACTCAAGTGATCCTCCTGCCTTGGCCTCCCAAAGTGCTAGGATT |
| | | | ACAGGCATGAGCCACTGCGCCTGGCCCAGTTACTTATTTTAGAA |
| | | | GTTATATTTGAGCATCTATTCTGTGCCGAGCCCTGGCATGAGCT |
| | | | GTGAACAGGCCATATCTATCCTAGATGTGCACTAATGGGGCTTT |
| | | | GGAGGGTGGCAACAGGAGGCCCGGT |
| SNP013 | 1 | 25 | CTTAAATGCAAATGGTAAATCTGAGGCAGCAGTAAGGTAGAGT |
| | | | GGAAAGGCTTTAGGGGAAAAAAAATAAAGTCTGAGAAACTGTC |
| | | | ACAGCAAGAGGACCCTAAGGAGACATGACAAGTAAATGTAATA |
| | | | TGGTGTCCTGGATGGCATCCTGGAAAAGAAAAGGGACATTAGA |
| | | | TAAAAACAAACCATGGACTTCAATAATAA |
| SNP013 | 2 | 26 | CTTAAATGCAAATGGTAAATCTGAGGCAGCAGTAAGGTAGAGT |
| | | | GGAAAGGCTTTAGGGGAAAAAAAATAAAGTCTGAGAAACTGTC |
| | | | ACAGCAAGAGGACCCTGAGGAGACATGACAAGTAAATGTAATA |
| | | | TGGTGTCCTGGATGGCATCCTGGAAAAGAAAAGGGACATTAGA |
| | | | TAAAAACAAACCATGGACTTCAATAATAA |
| SNP014 | 1 | 27 | GCTAGCAAAAGGTTCATTGTCAGAGATAACCTAGGTCTGAGGA |
| | | | AGCAGAGGCCAATAGTTTTTACATGCATCAGAGTGTTAGACAAT |
| | | | AAGATGCAGTGTAAAAGTGTTTGTTCTCACTGCTAACCAAAGAC |
| | | | AGGCAAATTAAGACTATTAAAGTATCCTTTTCTACCCACTAAAA |
| | | | TAATTTTAGAGAAAATTTAAGATTAC |
| SNP014 | 2 | 28 | GCTAGCAAAAGGTTCATTGTCAGAGATAACCTAGGTCTGAGGA |
| | | | AGCAGAGGCCAATAGTTTTTACATGCATCAGAGTGTTAGACAAT |
| | | | AAGATGCAGTGTAAAGGTGTTTGTTCTCACTGCTAACCAAAGAC |
| | | | AGGCAAATTAAGACTATTAAAGTATCCTTTTCTACCCACTAAAA |
| | | | TAATTTTAGAGAAAATTTAAGATTAC |
| SNP015 | 1 | 29 | ATTTCTTAAATGGGTACGTTTTGTTTGTACCCATCTCAAGACCTT |
| | | | TGAGATAGCCATTCCACTTGATTTTTTTTTTTTTTTTGAGATGGA |
| | | | GTCTTGCCCTGTCGCCAGGCTGGAGTGCGGTGGCGTGATCTCAA |
| | | | CTCAATGCAACCGCTGATTCCCTGGTTCAAGTGATTCTCCTGCC |
| | | | TCAGCCTCCCAAGTAGCTGGGAT |
| SNP015 | 2 | 30 | ATTTCTTAAATGGGTACGTTTTGTTTGTACCCATCTCAAGACCTT |
| | | | TGAGATAGCCATTCCACTTGATTTTTTTTTTTTTTTTGAGATGGA |
| | | | GTCTTGCCCTGTTGCCAGGCTGGAGTGCGGTGGCGTGATCTCAA |
| | | | CTCAATGCAACCGCTGATTCCCTGGTTCAAGTGATTCTCCTGCC |
| | | | TCAGCCTCCCAAGTAGCTGGGAT |
| SNP016 | 1 | 31 | ATAAAGAAGCTTACTGTGGTCCTGTAGGGCCTACCATCCTGTGT |
| | | | TGGTATTGCTCCAAAGATAAGCATTTTTGATTAAATTGGAGATT |
| | | | GCCCTCATTTGGGGCAGGGAGGGGGGTGCTTAGTCCAGTGATTT |
| | | | CACAAGCATTTTGGTCTCAGGACCCTTTTTTAACTCTTAAAATTT |
| | | | GTTGAGAACAGCAAATAAATTGTT |
| SNP016 | 2 | 32 | ATAAAGAAGCTTACTGTGGTCCTGTAGGGCCTACCATCCTGTGT |
| | | | TGGTATTGCTCCAAAGATAAGCATTTTTGATTAAATTGGAGATT |
| | | | GCCCTCATTTGGGGTAGGGAGGGGGGTGCTTAGTCCAGTGATTT |
| | | | CACAAGCATTTTGGTCTCAGGACCCTTTTTTAACTCTTAAAATTT |
| | | | GTTGAGAACAGCAAATAAATTGTT |
| SNP017 | 1 | 33 | CTCCCGGAGTAGCTCTGGGAGCAAGGCCCTCCCTGGATCCCTCC |
| | | | CTCTTTCCCGGTGTGCCTGTCTTTCCCGCTCCATCCTCCAGCCTC |
| | | | CACCGGGAGGAACACGGCTGCATTGTTCCTCAGGAGGAGGCCC |
| | | | TGCCTCGGGGCCTGGCCCAGTGCCCAGGTGGGGGGCCAGGAAC |
| | | | AAAACTCTGAGATCGAGGGGCGGGGT |
| SNP017 | 2 | 34 | CTCCCGGAGTAGCTCTGGGAGCAAGGCCCTCCCTGGATCCCTCC |
| | | | CTCTTTCCCGGTGTGCCTGTCTTTCCCGCTCCATCCTCCAGCCTC |
| | | | CACCGGGAGGAACGCGGCTGCATTGTTCCTCAGGAGGAGGCCC |
| | | | TGCCTCGGGGCCTGGCCCAGTGCCCAGGTGGGGGGCCAGGAAC |
| | | | AAAACTCTGAGATCGAGGGGCGGGGT |
| SNP018 | 1 | 35 | AGAATCTGATTAATAATTGCCCAGTAGGGACCAGTTCCTGGGTC |
| | | | CTGCTGAGCGGAGGGGAGGGAGTTCTGGGCTGGTTCTGGCTGG |
| | | | CCAGCCGAGGAGAGGAGAGGAGCAAAGGTGACTTTGAGCTGCA |
| | | | GAGGGTGGCCGCTGGGGCCCAGAGCTGGCAGATCCCAGCGGCT |
| | | | GGCCTCCTTCCATGCTTCCCTGGTGGCT |
| SNP018 | 2 | 36 | AGAATCTGATTAATAATTGCCCAGTAGGGACCAGTTCCTGGGTC |
| | | | CTGCTGAGCGGAGGGGAGGGAGTTCTGGGCTGGTTCTGGCTGG |
| | | | CCAGCCGAGGAGAGGGGAGGAGCAAAGGTGACTTTGAGCTGC |
| | | | AGAGGGTGGCCGCTGGGGCCCAGAGCTGGCAGATCCCAGCGGC |
| | | | TGGCCTCCTTCCATGCTTCCCTGGTGGCT |
| SNP019 | 1 | 37 | ACCAAAAGTATGCTCTGGCTGGATTGCAGCTACTCAAAAGGAG |
| | | | AAGGAACTGGGATGAGGCTAGAGATGTAGTCAGACCCTATAAA |
| | | | TTTTTTTTTTTTTTTTGAGCATTGAGACCTCAGGAACAGGTTTTA |
| | | | ATGGGTGTTTGCTGGTGGAGGAGAGGGTGGAGTGACTTAACAT |
| | | | GATGAGACTTACTGTGTTG |
| SNP019 | 2 | 38 | ACCAAAAGTATGCTCTGGCTGGATTGCAGCTACTCAAAAGGAG |
| | | | AAGGAACTGGGATGAGGCTAGAGATGTAGTCAGACCCTATAAA |
| | | | TTTTTTTTTTTTTTTTTGAGCATTGAGACCTCAGGAACAGGTTTT |
| | | | AATGGGTGTTTGCTGGTGGAGGAGAGGGTGGAGTGACTTAACA |
| | | | TGATGAGACTTACTGTGTTG |
| SNP019 | 3 | 39 | ACCAAAAGTATGCTCTGGCTGGATTGCAGCTACTCAAAAGGAG |
| | | | AAGGAACTGGGATGAGGCTAGAGATGTAGTCAGACCCTATAAA |
| | | | TTTTTTTTTTTTTTTTTTGAGCATTGAGACCTCAGGAACAGGTTT |
| | | | TAATGGGTGTTTGCTGGTGGAGGAGAGGGTGGAGTGACTTAAC |
| | | | ATGATGAGACTTACTGTGTTG |
| SNP019 | 4 | 40 | ACCAAAAGTATGCTCTGGCTGGATTGCAGCTACTCAAAAGGAG |
| | | | AAGGAACTGGGATGAGGCTAGAGATGTAGTCAGACCCTATAAA |
| | | | TTTTTTTTTTTTTTTTTTTGAGCATTGAGACCTCAGGAACAGGTT |
| | | | TTAATGGGTGTTTGCTGGTGGAGGAGAGGGTGGAGTGACTTAA |
| | | | CATGATGAGACTTACTGTGTTG |
| SNP020 | 1 | 41 | GGACCTGAGCGGTAGCCTTGGTCCACTGCAGACATCAGCAAGT |
| | | | TCTTAGATCAGGCGGGGAACCGGTCCACCCACTCATGCTCTGGT |
| | | | TTAGCTCCCTACTCTGCTTTCTGAGAGTCAGTTTGGAAGGGAAC |
| | | | CTAGCGGAGCTCCTCAGAGTCTCAGGGTCCTTGTCTTTGATGAA |
| | | | CTTTCTGCTTGGTCATAGGCAGCCTC |
| SNP020 | 2 | 42 | GGACCTGAGCGGTAGCCTTGGTCCACTGCAGACATCAGCAAGT |
| | | | TCTTAGATCAGGCGGGGAACCGGTCCACCCACTCATGCTCTGGT |
| | | | TTAGCTCCCTACTCTTCTTTCTGAGAGTCAGTTTGGAAGGGAAC |
| | | | CTAGCGGAGCTCCTCAGAGTCTCAGGGTCCTTGTCTTTGATGAA |
| | | | CTTTCTGCTTGGTCATAGGCAGCCTC |
| SNP021 | 1 | 43 | AAAATGCGTAGCATGGCTCCTGTAACTACGAGAGCTCCACAAA |
| | | | TGCAAACTATATGACAAGTGAGGAACATTAGATGACAGTCATG |
| | | | CTTCGATTTAAGTAAAGTTAATTGTAAAAATATAAACTTCCTAG |
| | | | AGGAAGTTGTTCTTCGTTCCTGCGGCTGTTGTTCTAACAAGTTA |
| | | | ATTCCTTTGGTGGATTTCCAGTAAGAT |
| SNP021 | 2 | 44 | AAAATGCGTAGCATGGCTCCTGTAACTACGAGAGCTCCACAAA |
| | | | TGCAAACTATATGACAAGTGAGGAACATTAGATGACAGTCATG |
| | | | CTTCGATTTAAGTAAATTTAATTGTAAAAATATAAACTTCCTAG |
| | | | AGGAAGTTGTTCTTCGTTCCTGCGGCTGTTGTTCTAACAAGTTA |
| | | | ATTCCTTTGGTGGATTTCCAGTAAGAT |
| SNP022 | 1 | 45 | CTGTCCAGTCAGGCAGCGCGCCAGTCAGCCTTCCTCCGTTTGCT |
| | | | GTTTCACCTTTCCAATCCCTCATTTTCACCCATTGGCTCCTAATG |
| | | | TGGGACACTGCTGAGGGCTTTGGTTTTCTACCTCAGTGAGCGTT |
| | | | CAACAGTCTGTTTTGACGCCTTGCTTTTAGAGCCAGGTTTAACA |
| | | | AAGCAAGGCATGGAATCATTTTCC |
| SNP022 | 2 | 46 | CTGTCCAGTCAGGCAGCGCGCCAGTCAGCCTTCCTCCGTTTGCT |
| | | | GTTTCACCTTTCCAATCCCTCATTTTCACCCATTGGCTCCTAATG |
| | | | TGGGACACTGCTGTGGGCTTTGGTTTTCTACCTCAGTGAGCGTT |
| | | | CAACAGTCTGTTTTGACGCCTTGCTTTTAGAGCCAGGTTTAACA |
| | | | AAGCAAGGCATGGAATCATTTTCC |
| SNP023 | 1 | 47 | TTTGGATTTCCTCTTGGCTGTGTTCATATCAATTTATCTCACCCC |
| | | | ACACCCCACAGTACTTGGTGTACCAGTTCTGGAACTACTGGTAT |
| | | | TTATTGCTGTGCCAAGGAGGCCCATTGCACAAGTCCTCCTAAAA |
| | | | GAGGCCATTGTCTCGATCACTTTAACATCTCCGCCCTGGGGAGC |
| | | | TTCAGGCTGAGAGATGGGCCTAAC |
| SNP023 | 2 | 48 | TTTGGATTTCCTCTTGGCTGTGTTCATATCAATTTATCTCACCCC |
| | | | ACACCCCACAGTACTTGGTGTACCAGTTCTGGAACTACTGGTAT |
| | | | TTATTGCTGTGCCGAGGAGGCCCATTGCACAAGTCCTCCTAAAA |
| | | | GAGGCCATTGTCTCGATCACTTTAACATCTCCGCCCTGGGGAGC |
| | | | TTCAGGCTGAGAGATGGGCCTAAC |
| SNP024 | 1 | 49 | ATTTTTGCTTTTTTTTTTTTTTTTTTTTTTTGTGGAGAGGGTGTTT |
| | | | CTCCATGTTGCCCAGGCTGGTCTCAAACTCCTGAGCTCAAGAGA |
| | | | TCTGCCTGCCTCAGCCTCCCAAAGTGCTAGGATTATAGGCATGA |
| | | | GCTACTGCGCCTGCCCAGAGCCTGCATTCTTTACCTTTACAGTCT |
| | | | AGACCCTGCTCCTATAGATCCC |
| SNP024 | 2 | 50 | ATTTTTGCTTTTTTTTTTTTTTTTTTTTTTTGTGGAGAGGGTGTTT |
| | | | CTCCATGTTGCCCAGGCTGGTCTCAAACTCCTGAGCTCAAGAGA |
| | | | TCTGCCTGCCTCGGCCTCCCAAAGTGCTAGGATTATAGGCATGA |
| | | | GCTACTGCGCCTGCCCAGAGCCTGCATTCTTTACCTTTACAGTCT |
| | | | AGACCCTGCTCCTATAGATCCC |
| SNP025 | 1 | 51 | TGGAACAAAGGTGGTGCTTATGGGATGTATGGAGCTGTGGGAT |
| | | | GTGGGAGAGGACAGGTTTAGCAGAGTGGCGTGGTGGAAAGTTT |
| | | | CCTCTCGCCGAGGAGGACAACTCCCCAGCGCCTGAGACAGCGG |
| | | | GAGCTCAGAGCGCCTAGAATGTGCCCAGATTCTAAAGTGAAGC |
| | | | AGGCTGTGGTGTGATGATGGCCCCCGAGG |
| SNP025 | 2 | 52 | TGGAACAAAGGTGGTGCTTATGGGATGTATGGAGCTGTGGGAT |
| | | | GTGGGAGAGGACAGGTTTAGCAGAGTGGCGTGGTGGAAAGTTT |
| | | | CCTCTCGCCGAGGAGGCCAACTCCCCAGCGCCTGAGACAGCGG |
| | | | GAGCTCAGAGCGCCTAGAATGTGCCCAGATTCTAAAGTGAAGC |
| | | | AGGCTGTGGTGTGATGATGGCCCCCGAGG |
| SNP026 | 1 | 53 | TGCCTGTCTTCTGCAGGGGCCTCTGCACCCACAGGCTTGGTCCA |
| | | | CAGCTGCCTCTTGGTTGTCCCTCCACCTCCCTGGCCTTTGAGACT |
| | | | CCCTCAGTGGCTTCGTCAGAGTTCTCTGAGCCCAGCTGTGGAGG |
| | | | AGAGTCTGAAACAGCTGCTCTGGGAGGCGGCAGCAGGAGTGTC |
| | | | CCAGCGCCGTGGGCTGGGCTGGTGC |
| SNP026 | 2 | 54 | TGCCTGTCTTCTGCAGGGGCCTCTGCACCCACAGGCTTGGTCCA |
| | | | CAGCTGCCTCTTGGTTGTCCCTCCACCTCCCTGGCCTTTGAGACT |
| | | | CCCTCAGTGGCTTTGTCAGAGTTCTCTGAGCCCAGCTGTGGAGG |
| | | | AGAGTCTGAAACAGCTGCTCTGGGAGGCGGCAGCAGGAGTGTC |
| | | | CCAGCGCCGTGGGCTGGGCTGGTGC |
| SNP027 | 1 | 55 | AGGCACTGAAGGGTGAGGCTGTGTGCCACTCCTTGGGCTGGCTC |
| | | | CAGCTGACAGGGTTGTCCACAGTAGAAAATGTGCCTGTGGGCA |
| | | | GTGGGGTCGGCCCCCAGCCCCACGTGGGAGGATGAACAACCCT |
| | | | CGGCACCATGCCATGCGCTTTGCTCAGATTCCCCTTCAGGAAAT |
| | | | TACTGATTTGGTTTCTTAGGAATTGGC |
| SNP027 | 2 | 56 | AGGCACTGAAGGGTGAGGCTGTGTGCCACTCCTTGGGCTGGCTC |
| | | | CAGCTGACAGGGTTGTCCACAGTAGAAAATGTGCCTGTGGGCA |
| | | | GTGGGGTCGGCCCCCGGCCCCACGTGGGAGGATGAACAACCCT |
| | | | CGGCACCATGCCATGCGCTTTGCTCAGATTCCCCTTCAGGAAAT |
| | | | TACTGATTTGGTTTCTTAGGAATTGGC |
| SNP028 | 1 | 57 | GGAGTCTTTCCCCCATTGCTTGCTTTTAATTGGCTTTGTTGAAGA |
| | | | TGAGATGGTTATAGTTGTGTTTTCTGAGTTCTTTATTCTGTTTCA |
| | | | TTGGTCTATGTTTTTTTTTTGTTTTTTTTTTTTTTGAAAGTGTCTC |
| | | | ACTCTTTACCAAGGCTGGAGTGTAGTGGCATGATCACAGCTCAC |
| | | | TGAAGCCTCAGCCTTCAGGG |
| SNP028 | 2 | 58 | GGAGTCTTTCCCCCATTGCTTGCTTTTAATTGGCTTTGTTGAAGA |
| | | | TGAGATGGTTATAGTTGTGTTTTCTGAGTTCTTTATTCTGTTTCA |
| | | | TTGGTCTATGTTGTTTTTTTTGTTTTTTTTTTTTTTGAAAGTGTCT |
| | | | CACTCTTTACCAAGGCTGGAGTGTAGTGGCATGATCACAGCTCA |
| | | | CTGAAGCCTCAGCCTTCAGGG |
| SNP029 | 1 | 59 | CCCACTCATGAGAACTCTACCCCCATCCAATCACCTCCCACTTG |
| | | | GTCCCACCTCCAACATGGGGGATTATAACTGGACATGAGATTTA |
| | | | GTGGGGACAGAGATACAAATTATATCATTCCACCCCCAGCCCCT |
| | | | CCAAATTTCATGTCCTTCTCATATTGCAAAATACAATCATCCCTT |
| | | | CTCAACAGTCCCCCAAAGTCTTAT |
| SNP029 | 2 | 60 | CCCACTCATGAGAACTCTACCCCCATCCAATCACCTCCCACTTG |
| | | | GTCCCACCTCCAACATGGGGGATTATAACTGGACATGAGATTTA |
| | | | GTGGGGACAGAGATCCAAATTATATCATTCCACCCCCAGCCCCT |
| | | | CCAAATTTCATGTCCTTCTCATATTGCAAAATACAATCATCCCTT |
| | | | CTCAACAGTCCCCCAAAGTCTTAT |
| SNP030 | 1 | 61 | TTTGACGAGTTGAGAGAAGAAGGCTTCAGATGATCAAACTACT |
| | | | CCGAGCTACAGGAGGAAATTCAAACCAAAGGCAAAGAAGTTCA |
| | | | AAACTTTGAAAAAAATGTAGACGAATGTATAATTAGAATAACC |
| | | | AATACAGAAAAGTGCTTAAAGGAGCTGATGGAGCTGAAAGCCA |
| | | | AGGCTGGAGAACTACGTGAAGAATGCAGA |
| SNP030 | 2 | 62 | TTTGACGAGTTGAGAGAAGAAGGCTTCAGATGATCAAACTACT |
| | | | CCGAGCTACAGGAGGAAATTCAAACCAAAGGCAAAGAAGTTCA |
| | | | AAACTTTGAAAAAAATTTAGACGAATGTATAATTAGAATAACC |
| | | | AATACAGAAAAGTGCTTAAAGGAGCTGATGGAGCTGAAAGCCA |
| | | | AGGCTGGAGAACTACGTGAAGAATGCAGA |
| SNP031 | 1 | 63 | GATCCAGATTGTGAGGTCATGAAATGCTTATGAACAGAGAGCT |
| | | | TAAGAGTAACTAGACAGAAGAAGAAAGAATGTTACTTCTGTTA |
| | | | ATATGAATAGCATGTGAGAAAGCCCTGGGTCCATATGGAGCCT |
| | | | GACCTAATCAAGACACATAAGAAAGACCTATGTGACAGGAGCG |
| | | | CAAAAAAGTGAAGGAGAAAATGGTTGG |
| SNP031 | 2 | 64 | GATCCAGATTGTGAGGTCATGAAATGCTTATGAACAGAGAGCT |
| | | | TAAGAGTAACTAGACAGAAGAAGAAAGAATGTTACTTCTGTTA |
| | | | ATATGAATAGCATGTGCGAAAGCCCTGGGTCCATATGGAGCCT |
| | | | GACCTAATCAAGACACATAAGAAAGACCTATGTGACAGGAGCG |
| | | | CAAAAAAGTGAAGGAGAAAATGGTTGG |
| SNP031 | 3 | 65 | GATCCAGATTGTGAGGTCATGAAATGCTTATGAACAGAGAGCT |
| | | | TAAGAGTAACTAGACAGAAGAAGAAAGAATGTTACTTCTGTTA |
| | | | ATATGAATAGCATGTGTGAAAGCCCTGGGTCCATATGGAGCCT |
| | | | GACCTAATCAAGACACATAAGAAAGACCTATGTGACAGGAGCG |
| | | | CAAAAAAGTGAAGGAGAAAATGGTTGG |
| SNP032 | 1 | 66 | TGGTCATCAGGGGTCCAAGCTTCTTCATTCTGCCTCACCATCTC |
| | | | GCTTGCAGCTTCTGCCTAATGTTGACTTACAGTTCAAGATGGCT |
| | | | TCTGGAGTGCTACCATTACATCCATGTTGTAGGCTAGAAGGAAA |
| | | | AGGGCAATGGCCTGAAGAGGAAGGGAGAGTTCCTGTTAACTCA |
| | | | GCTTCCTTTAAACAGCCTCCCCAAAA |
| SNP032 | 2 | 67 | TGGTCATCAGGGGTCCAAGCTTCTTCATTCTGCCTCACCATCTC |
| | | | GCTTGCAGCTTCTGCCTAATGTTGACTTACAGTTCAAGATGGCT |
| | | | TCTGGAGTGCTACCGTTACATCCATGTTGTAGGCTAGAAGGAAA |
| | | | AGGGCAATGGCCTGAAGAGGAAGGGAGAGTTCCTGTTAACTCA |
| | | | GCTTCCTTTAAACAGCCTCCCCAAAA |
| SNP033 | 1 | 68 | CAGCCTTGGACTCCTGGTCCAAAGCAATCCTCCTGCTTCAGCCT |
| | | | CCTAAGTGGCTGGGAGCACAGGAGCAAGCCATCACACTTGACT |
| | | | AATTTTTTTTTTTTTGAGACAGAGTTTCACTCTTGTTGCCCAGGC |
| | | | TGCAGTGCAATGGTGCCATCTCAGCTCACTGAAACCTCTACTTC |
| | | | CCAGATTCGAGCGATTCTCTTGCC |
| SNP033 | 2 | 69 | CAGCCTTGGACTCCTGGTCCAAAGCAATCCTCCTGCTTCAGCCT |
| | | | CCTAAGTGGCTGGGAGCACAGGAGCAAGCCATCACACTTGACT |
| | | | AATTTTTTTTTTTTTTGAGACAGAGTTTCACTCTTGTTGCCCAGG |
| | | | CTGCAGTGCAATGGTGCCATCTCAGCTCACTGAAACCTCTACTT |
| | | | CCCAGATTCGAGCGATTCTCTTGCC |
| SNP034 | 1 | 70 | ATCATTTGAACATAAATCAGAGTCTCAGTACAAACAGAGTGCTC |
| | | | AGGACATCAAGATGGTTAACCAGAGAGCCTGGCCAGAATATCT |
| | | | GCGGTGGAGAGAAACAATCTTGTTGGGAGAAGGATGACAATAA |
| | | | TTGGGGACTTAGAATAAAGGCTAAAAATGATTCAAAGAGAATG |
| | | | CAAAAAGAATCAGGCACACATCCTTTAC |
| SNP034 | 2 | 71 | ATCATTTGAACATAAATCAGAGTCTCAGTACAAACAGAGTGCTC |
| | | | AGGACATCAAGATGGTTAACCAGAGAGCCTGGCCAGAATATCT |
| | | | GCGGTGGAGAGAAACGATCTTGTTGGGAGAAGGATGACAATAA |
| | | | TTGGGGACTTAGAATAAAGGCTAAAAATGATTCAAAGAGAATG |
| | | | CAAAAAGAATCAGGCACACATCCTTTAC |
| SNP035 | 1 | 72 | TAAGAAGCATCCTCAAGCTCCCAGTTAAGTAACTTGACTACTTT |
| | | | TATTTGGGAATTTCAGACTATAGAAGCTCTCTTATGTCCAGATT |
| | | | CTGTGACCACTAGTTACTGTATCAGAACTCATCAGGTACCCACT |
| | | | TATAAATAGCACTGATCTGGC |
| SNP035 | 2 | 73 | TAAGAAGCATCCTCAAGCTCCCAGTTAAGTAACTTGACTACTTT |
| | | | TATTTGGGAATTTCAGACTATAGAAGCTCTCTTATGTTTTATGT |
| | | | CCAGATTCTGTGACCACTAGTTACTGTATCAGAACTCATCAGGT |
| | | | ACCCACTTATAAATAGCACTGATCTGGC |
| SNP036 | 1 | 74 | CTCTGCTCCAGGCTCTGGGCCGGGCACCAGCCTCTGGGAAAATG |
| | | | GAGGGGGTGGTGGTGAGGGCTCGGACAAGGAGCAGTGACTCCA |
| | | | TTCCAGGGACTCTGTCCAGAGGGACTGTCAGCTTAGGACGTGC |
| | | | GCGAAACACTCGGTTCACAGGGTTTAACACACTTTAGGGTAAA |
| | | | ACCTGGGAGAGCTTCCTAAGGAGGTGAC |
| SNP036 | 2 | 75 | CTCTGCTCCAGGCTCTGGGCCGGGCACCAGCCTCTGGGAAAATG |
| | | | GAGGGGGTGGTGGTGAGGGCTCGGACAAGGAGCAGTGACTCCA |
| | | | TTCCAGGGACTCTGTTCAGAGGGACTGTCAGCTTAGGACGTGCG |
| | | | CGAAACACTCGGTTCACAGGGTTTAACACACTTTAGGGTAAAA |
| | | | CCTGGGAGAGCTTCCTAAGGAGGTGAC |
| SNP037 | 1 | 76 | ACAAAAGAAACAAAAGACTGCTACTCCATAGGCAGAGCAGTCC |
| | | | TGAGAGCTGCTCGTGGCCTATTTTTATGGTTTTTTTTTTAAATTT |
| | | | TTATTTTAGGTTTGGGGGTACATGTGAAGGTTTTACATCGGTAA |
| | | | ACTTGTGCCACAGGGGTTTGTTGTACACATTGTTTCATTACCCA |
| | | | GGTATTAAGCCCAGTATCCGATAGT |
| SNP037 | 2 | 77 | ACAAAAGAAACAAAAGACTGCTACTCCATAGGCAGAGCAGTCC |
| | | | TGAGAGCTGCTCGTGGCCTATTTTTATGGTTTTTTTTTTAAATTT |
| | | | TTATTTTAGGTTTGTGGGTACATGTGAAGGTTTTACATCGGTAA |
| | | | ACTTGTGCCACAGGGGTTTGTTGTACACATTGTTTCATTACCCA |
| | | | GGTATTAAGCCCAGTATCCGATAGT |
| SNP038 | 1 | 78 | TTTCAAAAACGTGGCCACATCCATTTTCCCGCCATCAGCTTTCC |
| | | | AGACCACAGAATGCTCTGCTTTTGAGCTGCTCCTCAGCTGACAC |
| | | | CCTCCTTGAATCCACTTTGAGTTGCTCTTCCCTGGCCCCATTAGA |
| | | | AAATGTCGGCTCTGACTACACCATGCTTGCGAACAAAGGTGCA |
| | | | GAACAATTTTGGCTGCTTCGTCCAG |
| SNP038 | 2 | 79 | TTTCAAAAACGTGGCCACATCCATTTTCCCGCCATCAGCTTTCC |
| | | | AGACCACAGAATGCTCTGCTTTTGAGCTGCTCCTCAGCTGACAC |
| | | | CCTCCTTGAATCCATTTTGAGTTGCTCTTCCCTGGCCCCATTAGA |
| | | | AAATGTCGGCTCTGACTACACCATGCTTGCGAACAAAGGTGCA |
| | | | GAACAATTTTGGCTGCTTCGTCCAG |
| SNP039 | 1 | 80 | CTTCTAGAAGCGTAAGGTAACACTGGCATTCCTCTAGCCTCTGC |
| | | | TGGAGTGCAGTGAGGATTTTCTAGCATGTTGCTGCACTGTTCCC |
| | | | ATGCACATTATTCTAACTTTTTAGTAACTCACACGTGCATTCTTT |
| | | | TTTCAACGCTATCCTTAGAGTGAAAGTCAGAAAAAAATACTAG |
| | | | AAACTAACTCAGGGCTGAGCGTGGT |
| SNP039 | 2 | 81 | CTTCTAGAAGCGTAAGGTAACACTGGCATTCCTCTAGCCTCTGC |
| | | | TGGAGTGCAGTGAGGATTTTCTAGCATGTTGCTGCACTGTTCCC |
| | | | ATGCACATTATTCTGACTTTTTAGTAACTCACACGTGCATTCTTT |
| | | | TTTCAACGCTATCCTTAGAGTGAAAGTCAGAAAAAAATACTAG |
| | | | AAACTAACTCAGGGCTGAGCGTGGT |
| SNP040 | 1 | 82 | CTGAGCTCCCATCTCACACTGACATCTACAGAGTCCTATAGCTT |
| | | | CCATCTTGGAGTCCCACTCTGCCTTCTCAGAAAGCCACAGGTCA |
| | | | AATGAGGCTCCGCCGCACGCAGAACAGGGGACCTCCTGGACAG |
| | | | GAGTGGCTTTTATCCATCCCCACACCCACAGCTCCCAGCGCAGA |
| | | | CCCCGAAGAATTCATCCCAGGTGAGT |
| SNP040 | 2 | 83 | CTGAGCTCCCATCTCACACTGACATCTACAGAGTCCTATAGCTT |
| | | | CCATCTTGGAGTCCCACTCTGCCTTCTCAGAAAGCCACAGGTCA |
| | | | AATGAGGCTCCGCCTCACGCAGAACAGGGGACCTCCTGGACAG |
| | | | GAGTGGCTTTTATCCATCCCCACACCCACAGCTCCCAGCGCAGA |
| | | | CCCCGAAGAATTCATCCCAGGTGAGT |
| SNP041 | 1 | 84 | TTCTGACCTCAAGTGATCCGCCCGCCTCGGCCTCTGAAAGTGCT |
| | | | AGGATTGTAGGCATGAGCCACCGCGCCCGGCCTCGTACGGTAA |
| | | | TTCTGTGTGATGTTTAGGGACACGTCTCGGAGCTGGCGAACTGG |
| | | | ACTTGGGGTGGGAGGGAAAGGAAGCATTAAAG |
| SNP041 | 2 | 85 | TTCTGACCTCAAGTGATCCGCCCGCCTCGGCCTCTGAAAGTGCT |
| | | | AGGATTGTAGGCATGAGCCACCGCGCCCGGCCTCGTACGGTAA |
| | | | TTCTGTGTGATGTTTTGAGGAATTGCCACAATTTTTTCCTGCG |
| | | | CCTGCACCAGGGACACGTCTCGGAGCTGGCGAACTGGACTTGG |
| | | | GGTGGGAGGGAAAGGAAGCATTAAAG |
| SNP042 | 1 | 86 | AACTGGCTGCCTCTATTCCAAAAATTATTTAGAAATTTTCAGAA |
| | | | TTTAAACTCATTAGCATGGCTTGGAACTTTCTCATCCCTAACGC |
| | | | AATCCCTGTGACCGATATAATGATGGTAATACTAAGAGTAAAG |
| | | | GGGAGAGACAGATCCTACTGATTATTAAAAAGTTATAGTCTGAT |
| | | | AATGAATGAGTGTTGTCAGGAATAGA |
| SNP042 | 2 | 87 | AACTGGCTGCCTCTATTCCAAAAATTATTTAGAAATTTTCAGAA |
| | | | TTTAAACTCATTAGCATGGCTTGGAACTTTCTCATCCCTAACGC |
| | | | AATCCCTGTGACCGGTATAATGATGGTAATACTAAGAGTAAAG |
| | | | GGGAGAGACAGATCCTACTGATTATTAAAAAGTTATAGTCTGAT |
| | | | AATGAATGAGTGTTGTCAGGAATAGA |
| SNP043 | 1 | 88 | AAACCCTGCGCACTGTGGTTCACGCCAGCAATCCCAGCCCTTTG |
| | | | GGAGGCTAAGGCAGGTGGATCACCTGAGTCCAGGAGTTCAAGA |
| | | | CCAGCCAGGATGACACAGCAAAACACCATCTCTACTAATAATA |
| | | | CAAAAACCAGCTGTGAATGGTGACACACAGCTGAAGTAGCAGC |
| | | | TACTAGGGAGACTGAAGCAGGAGGACTG |
| SNP043 | 2 | 89 | AAACCCTGCGCACTGTGGTTCACGCCAGCAATCCCAGCCCTTTG |
| | | | GGAGGCTAAGGCAGGTGGATCACCTGAGTCCAGGAGTTCAAGA |
| | | | CCAGCCAGGATGACATAGCAAAACACCATCTCTACTAATAATA |
| | | | CAAAAACCAGCTGTGAATGGTGACACACAGCTGAAGTAGCAGC |
| | | | TACTAGGGAGACTGAAGCAGGAGGACTG |
| SNP044 | 1 | 90 | TGCTATATCCATCTACATATATAAAGCCACCGGGAGAACTAGTC |
| | | | CACTTGGTGCAGTCTTCTATACTGTCCTTCACAGCTTAGATTCAA |
| | | | TCTTTCCTTAAAGTGTAGCCGGGATACACAGGAGTGTGATTCTG |
| | | | GGCTGACTCAAAGTTCTTCTCTTGAAGGCTTTTTCCTGTGGCACT |
| | | | GGCAGATGGCTGTGCTATCTTC |
| SNP044 | 2 | 91 | TGCTATATCCATCTACATATATAAAGCCACCGGGAGAACTAGTC |
| | | | CACTTGGTGCAGTCTTCTATACTGTCCTTCACAGCTTAGATTCAA |
| | | | TCTTTCCTTAAAGATGTAGCCGGGATACACAGGAGTGTGATTCT |
| | | | GGGCTGACTCAAAGTTCTTCTCTTGAAGGCTTTTTCCTGTGGCA |
| | | | CTGGCAGATGGCTGTGCTATCTTC |
| SNP045 | 1 | 92 | ATTTGCTGGAGATTGATGCTGGGAAAGGAAGCAAAATCTTTTGC |
| | | | AATCTGTGGTTCCTTGGTTGGACAAGAAAGAGTCTTCTGCCAGG |
| | | | CCTGAGGATCTTCCAGACACTCACAGTACTCATGGTACACTGGT |
| | | | CCTAGGGAAGGAAAACATGAAAAAGGCCGCACTCCATTAGCAA |
| | | | GCACCACAACACAGGGAGTCACTTCT |
| SNP045 | 2 | 93 | ATTTGCTGGAGATTGATGCTGGGAAAGGAAGCAAAATCTTTTGC |
| | | | AATCTGTGGTTCCTTGGTTGGACAAGAAAGAGTCTTCTGCCAGG |
| | | | CCTGAGGATCTTCCGGACACTCACAGTACTCATGGTACACTGGT |
| | | | CCTAGGGAAGGAAAACATGAAAAAGGCCGCACTCCATTAGCAA |
| | | | GCACCACAACACAGGGAGTCACTTCT |
| SNP046 | 1 | 94 | AATGACTGACACTCTCAAATTCCCCTCTGCATCATGGGCACTCA |
| | | | GCACTGTGCCTAGTGCATAGTAAGACTTCAACAAATATGTGCTG |
| | | | TTGTTATAATTCGGAATGACGATGGAGGTGCAGAGGTTTACCTG |
| | | | TGTTTTTATTATCTCTGGTTGACAAGGCGGCCACACCCAGGTTG |
| | | | CCTGTTCTGAAGCTGTCTCAAGACA |
| SNP046 | 2 | 95 | AATGACTGACACTCTCAAATTCCCCTCTGCATCATGGGCACTCA |
| | | | GCACTGTGCCTAGTGCATAGTAAGACTTCAACAAATATGTGCTG |
| | | | TTGTTATAATTCGGCATGACGATGGAGGTGCAGAGGTTTACCTG |
| | | | TGTTTTTATTATCTCTGGTTGACAAGGCGGCCACACCCAGGTTG |
| | | | CCTGTTCTGAAGCTGTCTCAAGACA |
| SNP047 | 1 | 96 | GGCATGCACCACCACGCCCAGCTAAATTTTTTTATTTTTAGTAG |
| | | | AGATGGGGTTTCACCATGTTGGCAAGGCTGGTCTCAAACTCCTG |
| | | | ACTTCATGATCCACCCGCCTCGGCCTCTCAAAGTGCTGGGATTA |
| | | | CAGGTGTGAGCCACTGCAACCAGCCTGTTTTTTGTTTTTTTTGAG |
| | | | TAGGATGTGATCCGCTTATGTTTT |
| SNP047 | 2 | 97 | GGCATGCACCACCACGCCCAGCTAAATTTTTTTATTTTTAGTAG |
| | | | AGATGGGGTTTCACCATGTTGGCAAGGCTGGTCTCAAACTCCTG |
| | | | ACTTCATGATCCACTCGCCTCGGCCTCTCAAAGTGCTGGGATTA |
| | | | CAGGTGTGAGCCACTGCAACCAGCCTGTTTTTTGTTTTTTTTGAG |
| | | | TAGGATGTGATCCGCTTATGTTTT |
| SNP048 | 1 | 98 | TTCACAGGTGTTTAGAAAAATTAGATTGTCACCTCTTGTTGGTC |
| | | | ACAGAATGATTACAATACTTTGCATTCGTGCCACAATAGTTTTT |
| | | | AGAGGGTTTTTGTACGTTATGTAGCTGAGCATTCCATTTGGTCT |
| | | | TTGGAGCCTGGGGGAAGAGGACCTTTAATGAGGACAAGAAGAT |
| | | | AGGAAAGTGCAAAAATACAAATGGAG |
| SNP048 | 2 | 99 | TTCACAGGTGTTTAGAAAAATTAGATTGTCACCTCTTGTTGGTC |
| | | | ACAGAATGATTACAATACTTTGCATTCGTGCCACAATAGTTTTT |
| | | | AGAGGGTTTTTGTATGTTATGTAGCTGAGCATTCCATTTGGTCTT |
| | | | TGGAGCCTGGGGGAAGAGGACCTTTAATGAGGACAAGAAGATA |
| | | | GGAAAGTGCAAAAATACAAATGGAG |
| SNP049 | 1 | 100 | ACTGTGTGACTCTAGTGATCTTTAACATACACAGAATGATCTAC |
| | | | AGTGATCTTTAACATACTCAGAAATATGAAAAATGTTTGAATAT |
| | | | GATCTTTAGGGACTGCTAATGAAAAGGGTATATGAAATGGGAA |
| | | | CAATAAATTCTGTACATGTATACAGTCCATATACACATTAAGTG |
| | | | TTTGTCATTTGGACAAATTGAAAACT |
| SNP049 | 2 | 101 | ACTGTGTGACTCTAGTGATCTTTAACATACACAGAATGATCTAC |
| | | | AGTGATCTTTAACATACTCAGAAATATGAAAAATGTTTGAATAT |
| | | | GATCTTTAGGGACTTCTAATGAAAAGGGTATATGAAATGGGAA |
| | | | CAATAAATTCTGTACATGTATACAGTCCATATACACATTAAGTG |
| | | | TTTGTCATTTGGACAAATTGAAAACT |
| SNP050 | 1 | 102 | ACTAGCCATGGACATGCAAATTTTAAAAACAATGAGCTACTGTT |
| | | | GCTCTCAATTGGGCAATATTTTTAGAAAACTGATAGCATCTAGG |
| | | | CCAGCCCTTCCCAACCGTCTGCACCTGGGAATCGCCTGGGGACC |
| | | | TTCAAGTAACTACTGATCCCCAGCTACCAATTTAATTGGTTTGG |
| | | | GGTATGGCCTGGGGTTCTACATTTT |
| SNP050 | 2 | 103 | ACTAGCCATGGACATGCAAATTTTAAAAACAATGAGCTACTGTT |
| | | | GCTCTCAATTGGGCAATATTTTTAGAAAACTGATAGCATCTAGG |
| | | | CCAGCCCTTCCCAATCGTCTGCACCTGGGAATCGCCTGGGGACC |
| | | | TTCAAGTAACTACTGATCCCCAGCTACCAATTTAATTGGTTTGG |
| | | | GGTATGGCCTGGGGTTCTACATTTT |
| SNP051 | 1 | 104 | ATCAGTGTTACAGTGAAACAAAGTTATTCAAGGACCTGCTGCTG |
| | | | TACATACTTTTGCTAAAAATCAGTTTCCAAGAACCTATTGTGGA |
| | | | TGTTAGGAGAGGAGCTACCATGCCACAATGACTCTGGGAGATG |
| | | | AAGCCATTTTATTCCCATGCTTGTTAACCTTGTGCAGGTGCGGG |
| | | | AATGCAGATGGCTGAGTAGGTCAGAT |
| SNP051 | 2 | 105 | ATCAGTGTTACAGTGAAACAAAGTTATTCAAGGACCTGCTGCTG |
| | | | TACATACTTTTGCTAAAAATCAGTTTCCAAGAACCTATTGTGGA |
| | | | TGTTAGGAGAGGAGTTACCATGCCACAATGACTCTGGGAGATG |
| | | | AAGCCATTTTATTCCCATGCTTGTTAACCTTGTGCAGGTGCGGG |
| | | | AATGCAGATGGCTGAGTAGGTCAGAT |
| SNP052 | 1 | 106 | CCTCCCAAAGTGTTGGGATTACAGGTATGAGCCACCACACCCA |
| | | | GCTGCATGTGGATTCTTAAGTGCAACAGTCAGCAGTAATCTCAC |
| | | | ATCTGTTAGCAGACACTTGCTGTAGTCACAACAATGCTTTCTTC |
| | | | TTCCCTGAACAGATACTCCACTTCTTGAAATATACTTAAGTAGG |
| | | | CACTGTATTTATACAGCTCTGAAAGC |
| SNP052 | 2 | 107 | CCTCCCAAAGTGTTGGGATTACAGGTATGAGCCACCACACCCA |
| | | | GCTGCATGTGGATTCTTAAGTGCAACAGTCAGCAGTAATCTCAC |
| | | | ATCTGTTAGCAGACATTTGCTGTAGTCACAACAATGCTTTCTTC |
| | | | TTCCCTGAACAGATACTCCACTTCTTGAAATATACTTAAGTAGG |
| | | | CACTGTATTTATACAGCTCTGAAAGC |
| SNP053 | 1 | 108 | AGGAAAGATTGATTCCAGGAAAAGGGACCAAATGTCCTAACAC |
| | | | TTTTAAATGCCTAACAGAAAAGTTTTTACCACAGACTACCATTT |
| | | | TTTTCTTTCTAAAGGCTGCTACCTTTGAGGCTTCATCTGCATAAC |
| | | | AAGACAGCTTTTGCTCACCATGCCTTTCCTCCCCTCTCCCTCCCA |
| | | | TAAAGCTGTTGCCACACTCCAAGC |
| SNP053 | 2 | 109 | AGGAAAGATTGATTCCAGGAAAAGGGACCAAATGTCCTAACAC |
| | | | TTTTAAATGCCTAACAGAAAAGTTTTTACCACAGACTACCATTT |
| | | | TTTTCTTTCTAAAGGTTGCTACCTTTGAGGCTTCATCTGCATAAC |
| | | | AAGACAGCTTTTGCTCACCATGCCTTTCCTCCCCTCTCCCTCCCA |
| | | | TAAAGCTGTTGCCACACTCCAAGC |
| SNP054 | 1 | 110 | TCTGTAACTACCAGATTTTACTCGCCTTCCTGGACTCTGTTCACA |
| | | | GGAAAGAAAGGAAACAAACAAACCTCATACATTATGAAGCATA |
| | | | GGGTATCAATGGCCCTGCTGATTATAGTGGGTGGGGGTGGCAT |
| | | | AAATACATGTACACCCCCACTGCTGCCCCATCCCCACTCCTCTG |
| | | | AGCACTGGGGTCAAGGAATATATATT |
| SNP054 | 2 | 111 | TCTGTAACTACCAGATTTTACTCGCCTTCCTGGACTCTGTTCACA |
| | | | GGAAAGAAAGGAAACAAACAAACCTCATACATTATGAAGCATA |
| | | | GGGTATCAATGGCCTTGCTGATTATAGTGGGTGGGGGTGGCAT |
| | | | AAATACATGTACACCCCCACTGCTGCCCCATCCCCACTCCTCTG |
| | | | AGCACTGGGGTCAAGGAATATATATT |
| SNP055 | 1 | 112 | ACACCTGAGGAAGGCTGGGCAGAATGGATCGGGGGTGTGTATT |
| | | | GGCTGCAGTCACCTCCCCTCTGCTCGTCTGTGTCCACATTCTGTC |
| | | | GTGGTTGAGACCGGATCCTGTGTGGACCGGGTGGGCTGGTGTG |
| | | | GAGTCCTGTCAGGAGACCTGGGGCGGTTTTGAGGCAAGCTCAC |
| | | | GGAGGCCTGCCGCAGGGCCCTGCGCTG |
| SNP055 | 2 | 113 | ACACCTGAGGAAGGCTGGGCAGAATGGATCGGGGGTGTGTATT |
| | | | GGCTGCAGTCACCTCCCCTCTGCTCGTCTGTGTCCACATTCTGTC |
| | | | GTGGTTGAGACCGGCTCCTGTGTGGACCGGGTGGGCTGGTGTG |
| | | | GAGTCCTGTCAGGAGACCTGGGGCGGTTTTGAGGCAAGCTCAC |
| | | | GGAGGCCTGCCGCAGGGCCCTGCGCTG |
| SNP056 | 1 | 114 | TTATCTTCCTAAGACATAAGGCAATTGCTGACATTTGCCTTCCTC |
| | | | AAGGGCCAAACAGCCAACCAACAGTGTCCTTGGGAGCAGAGCT |
| | | | GAGTTTCTAAACCTACGGCTAGAAACATGGAGATCCAAATCCA |
| | | | TATATGGAGATACTTCACAGAAGGAAAAAAAGCAGAAATAAAC |
| | | | TCTTGGGAAAGAAAGAATCAGACCCAC |
| SNP056 | 2 | 115 | TTATCTTCCTAAGACATAAGGCAATTGCTGACATTTGCCTTCCTC |
| | | | AAGGGCCAAACAGCCAACCAACAGTGTCCTTGGGAGCAGAGCT |
| | | | GAGTTTCTAAACCTGCGGCTAGAAACATGGAGATCCAAATCCA |
| | | | TATATGGAGATACTTCACAGAAGGAAAAAAAGCAGAAATAAAC |
| | | | TCTTGGGAAAGAAAGAATCAGACCCAC |
| SNP057 | 1 | 116 | CTTCACAGGTGAAATTGTTTGACCCATGAAAATCTGAAACATAT |
| | | | GAGGTCTCTTGTTTTCATTCAGCATATGCCAGTTAAGTGCCTAA |
| | | | TTGTTCACCCAGCAACCAGATCCTGACTCACAATTAATTTTATTT |
| | | | ATAGAGCCTCACTGCTTTGCTGCTTCCCTACTTGTTATTTTGACT |
| | | | GTGGGAGCAAAAAATGGTAAC |
| SNP057 | 2 | 117 | CTTCACAGGTGAAATTGTTTGACCCATGAAAATCTGAAACATAT |
| | | | GAGGTCTCTTGTTTTCATTCAGCATATGCCAGTTAAGTGCCTAA |
| | | | TTGTTCACCCAGCACCCAGATCCTGACTCACAATTAATTTTATTT |
| | | | ATAGAGCCTCACTGCTTTGCTGCTTCCCTACTTGTTATTTTGACT |
| | | | GTGGGAGCAAAAAATGGTAAC |
| SNP057 | 3 | 118 | CTTCACAGGTGAAATTGTTTGACCCATGAAAATCTGAAACATAT |
| | | | GAGGTCTCTTGTTTTCATTCAGCATATGCCAGTTAAGTGCCTAA |
| | | | TTGTTCACCCAGCATCCAGATCCTGACTCACAATTAATTTTATTT |
| | | | ATAGAGCCTCACTGCTTTGCTGCTTCCCTACTTGTTATTTTGACT |
| | | | GTGGGAGCAAAAAATGGTAAC |
| SNP058 | 1 | 119 | AGTATGTAAGGTGATGCTAAGAGAAGAGGCACAAAAGTATTCA |
| | | | CAATTGGGAGGGAGACAGATCCCAGGGCTCCTTGAAGAGGTTC |
| | | | TCCCTCACTACTCATGCCCTGCTTATCACACACTTGCACAGTTG |
| | | | GATTTCTTTTCAGGTGTGCAAATATATAAGCTTTTAAGTCATGA |
| | | | ATAGTATGTACCTTATCTGCACTGTTG |
| SNP058 | 2 | 120 | AGTATGTAAGGTGATGCTAAGAGAAGAGGCACAAAAGTATTCA |
| | | | CAATTGGGAGGGAGACAGATCCCAGGGCTCCTTGAAGAGGTTC |
| | | | TCCCTCACTACTCATGTCCTGCTTATCACACACTTGCACAGTTG |
| | | | GATTTCTTTTCAGGTGTGCAAATATATAAGCTTTTAAGTCATGA |
| | | | ATAGTATGTACCTTATCTGCACTGTTG |
| SNP059 | 1 | 121 | GAAGATAAGGGGAAACTGAAAAATGAGCATGGGAGGAAGTAG |
| | | | CCAGGGAGGGAGGAGGAAAACCAGGCGAACACTGTGTTCTAGA |
| | | | AGCCAAGCAAAGATGGACTTTCAAGAAGAAGAGAGTCATCAAC |
| | | | TGTGTCAAATGCTGCTTATAGGATGATAGAGGACCACTGATTGG |
| | | | ATTTAAGAAAGTGGAAGTCCTTGGCCACC |
| SNP059 | 2 | 122 | GAAGATAAGGGGAAACTGAAAAATGAGCATGGGAGGAAGTAG |
| | | | CCAGGGAGGGAGGAGGAAAACCAGGCGAACACTGTGTTCTAGA |
| | | | AGCCAAGCAAAGATGGAGTTTCAAGAAGAAGAGAGTCATCAAC |
| | | | TGTGTCAAATGCTGCTTATAGGATGATAGAGGACCACTGATTGG |
| | | | ATTTAAGAAAGTGGAAGTCCTTGGCCACC |
| SNP060 | 1 | 123 | TTTCCTGACTTAGTCCCTTACCCTCAGAGACTGAACAAGAGCTG |
| | | | TAATTTTTACATGGGTGCCCAGGATGTGGCCTTGTCCCCTGTAT |
| | | | CCTTTCCAACCTAGATTTGAGCTGCTGCCTTCTATTAACTGCCTT |
| | | | TTCTGGCTAAGGTGGGAGGCAGAGCCCAAGCCGATCCCAGGAT |
| | | | GATGGGAGACCCCAGCCATGTTCCT |
| SNP060 | 2 | 124 | TTTCCTGACTTAGTCCCTTACCCTCAGAGACTGAACAAGAGCTG |
| | | | TAATTTTTACATGGGTGCCCAGGATGTGGCCTTGTCCCCTGTAT |
| | | | CCTTTCCAACCTAGCTTTGAGCTGCTGCCTTCTATTAACTGCCTT |
| | | | TTCTGGCTAAGGTGGGAGGCAGAGCCCAAGCCGATCCCAGGAT |
| | | | GATGGGAGACCCCAGCCATGTTCCT |
| SNP061 | 1 | 125 | CCTTCTAAAGCCACCGAAAGTTAATTGGTAGGATATCACAGGG |
| | | | GTGTTTTAACTTTTCTTTGAATTTTCTCCAATCTTAGTAACACAG |
| | | | CTGACTGGGGCAATCGCATCTCACTTCTACTTGATTTCTAAATA |
| | | | TTTGTTAACTAATATTCTTCATTTATGCTAAGATAGACAGTTTTT |
| | | | ACATTTAATAATTTTAGAATAAGA |
| SNP061 | 2 | 126 | CCTTCTAAAGCCACCGAAAGTTAATTGGTAGGATATCACAGGG |
| | | | GTGTTTTAACTTTTCTTTGAATTTTCTCCAATCTTAGTAACACAG |
| | | | CTGACTGGGGCAATGGCATCTCACTTCTACTTGATTTCTAAATA |
| | | | TTTGTTAACTAATATTCTTCATTTATGCTAAGATAGACAGTTTTT |
| | | | ACATTTAATAATTTTAGAATAAGA |
| SNP062 | 1 | 127 | TAACAGCAAGCAATAGTATCTATTTGAATAGAAACCAACACCT |
| | | | GTTATTTGAAGTTGAAGGTATGTGAGTTTGACCGTGATCCTTAA |
| | | | ATAGCAGCACAGACCCATTTGCAAACCGTGGATTGATGTGGCC |
| | | | CCAGCGGAGCAGGGGAGACGGAGATGCAGGGGGGGTGTTGTGT |
| | | | GCTTGGTGGAGGGACTGGGTTCTGCAGG |
| SNP062 | 2 | 128 | TAACAGCAAGCAATAGTATCTATTTGAATAGAAACCAACACCT |
| | | | GTTATTTGAAGTTGAAGGTATGTGAGTTTGACCGTGATCCTTAA |
| | | | ATAGCAGCACAGACCTATTTGCAAACCGTGGATTGATGTGGCC |
| | | | CCAGCGGAGCAGGGGAGACGGAGATGCAGGGGGGGTGTTGTGT |
| | | | GCTTGGTGGAGGGACTGGGTTCTGCAGG |
| SNP063 | 1 | 129 | ATTAATCAAAGTCCATACTGCAGGTAAGTGGCAGAACTGAGGT |
| | | | CTGAACCAAGGAAGTCTGACTCCAGTGCCTACGATCATAATCAC |
| | | | AAGTACCTCGAATACATTGGTAAGATGGCACATGACTGGTAGC |
| | | | TTTGCTGTAGAGGAATCTTACCTTGTCATACAAATCAATATGCC |
| | | | TTGTGAAAAATTTTTCAAATGCTTGAA |
| SNP063 | 2 | 130 | ATTAATCAAAGTCCATACTGCAGGTAAGTGGCAGAACTGAGGT |
| | | | CTGAACCAAGGAAGTCTGACTCCAGTGCCTACGATCATAATCAC |
| | | | AAGTACCTCGAATACGTTGGTAAGATGGCACATGACTGGTAGC |
| | | | TTTGCTGTAGAGGAATCTTACCTTGTCATACAAATCAATATGCC |
| | | | TTGTGAAAAATTTTTCAAATGCTTGAA |
| SNP064 | 1 | 131 | TCCTGCCTGCTGAAAGGAGCCCAGCGACTCCAACACCAACGTC |
| | | | ATTCATTAGGAAAACAAAACCGAAGACTCGCATGCACGTATAT |
| | | | GTACACACACAAAATGAACAAATAGTGGGAAGAATTATTGTAA |
| | | | GTCTTAAGTCACAGGAAATTTGATTTGCTTCAACTAAAACACCC |
| | | | GAGGATAGGCCGGCGTGGCGGGTCGCCC |
| SNP064 | 2 | 132 | TCCTGCCTGCTGAAAGGAGCCCAGCGACTCCAACACCAACGTC |
| | | | ATTCATTAGGAAAACAAAACCGAAGACTCGCATGCACGTATAT |
| | | | GTACACACACAAAATGCACAAATAGTGGGAAGAATTATTGTAA |
| | | | GTCTTAAGTCACAGGAAATTTGATTTGCTTCAACTAAAACACCC |
| | | | GAGGATAGGCCGGCGTGGCGGGTCGCCC |
| SNP065 | 1 | 133 | CACGCTCTGGTAGGCACTGAGGTTGGTGGTGAAACCCAGCTGG |
| | | | GAGATGGAGGCGCCCTTGTCCCGCAGCACTCGGTACTCCTCCCA |
| | | | GCAGTAGTAGATGCCATATGCCAGCACGCCCAGCACTCCCAGG |
| | | | ATCAGCACCAGCACCAGGGGCCCAGCCACCAGGCGCAGAAGCA |
| | | | AGATAAACAGTAGGCTCAAGACCAGAGC |
| SNP065 | 2 | 134 | CACGCTCTGGTAGGCACTGAGGTTGGTGGTGAAACCCAGCTGG |
| | | | GAGATGGAGGCGCCCTTGTCCCGCAGCACTCGGTACTCCTCCCA |
| | | | GCAGTAGTAGATGCCGTATGCCAGCACGCCCAGCACTCCCAGG |
| | | | ATCAGCACCAGCACCAGGGGCCCAGCCACCAGGCGCAGAAGCA |
| | | | AGATAAACAGTAGGCTCAAGACCAGAGC |
| SNP066 | 1 | 135 | TATTTCCTATTTTAATTTCCCTAGTTGCCTCCAAAATACCTTTTA |
| | | | TAGCTATTTTTATTTTTTCCTGATCCAAGGTACAATCAAGACTCA |
| | | | TGCATTGCAAGGATAAATTTTTTTTTCCCTAAGTGATCTTCATTG |
| | | | GGAGAAGTAAAACAATTTTATGTTAAACTCTAAGTGAGGTGAT |
| | | | ATTTTGTTCTCAGATTTTGAACT |
| SNP066 | 2 | 136 | TATTTCCTATTTTAATTTCCCTAGTTGCCTCCAAAATACCTTTTA |
| | | | TAGCTATTTTTATTTTTTCCTGATCCAAGGTACAATCAAGACTCA |
| | | | TGCATTGCAAGGGTAAATTTTTTTTTCCCTAAGTGATCTTCATTG |
| | | | GGAGAAGTAAAACAATTTTATGTTAAACTCTAAGTGAGGTGAT |
| | | | ATTTTGTTCTCAGATTTTGAACT |
| SNP067 | 1 | 137 | ATCAAGATCTGCAGCTCCAGAGGTTGCCATTTCCCATGCCCAGA |
| | | | TAGTTGGCTTACAAGCCTAGCTTCAAAGCATGCCTTGGCTCACA |
| | | | GAGTCATCTCTTTTAGGGATGTCCCCACCCTGTACTCATCTCAA |
| | | | AGCCATCGAGAACCATCTCTAAATGTCATATCTGGCAGTGATCT |
| | | | CTCTTTTCTCTGATCCGTTGTCGCA |
| SNP067 | 2 | 138 | ATCAAGATCTGCAGCTCCAGAGGTTGCCATTTCCCATGCCCAGA |
| | | | TAGTTGGCTTACAAGCCTAGCTTCAAAGCATGCCTTGGCTCACA |
| | | | GAGTCATCTCTTTTTGGGATGTCCCCACCCTGTACTCATCTCAA |
| | | | AGCCATCGAGAACCATCTCTAAATGTCATATCTGGCAGTGATCT |
| | | | CTCTTTTCTCTGATCCGTTGTCGCA |
| SNP068 | 1 | 139 | CCTATGGACAACAAACTGTGCCTAATGAATTCTGGCCAGAGCC |
| | | | AAAACAATGAAATTATTTATCTCCACCTCCCCTATTGATGCACA |
| | | | GCAGAAATAAATATAAGGATCACCACCTTCTGTGCAAATGCAA |
| | | | ATAAGTATACTCGCAGAAACAAAAATTTCAACCTACAATTTCAG |
| | | | TTTTCCTCACCTTTTGCTTACACTCTA |
| SNP068 | 2 | 140 | CCTATGGACAACAAACTGTGCCTAATGAATTCTGGCCAGAGCC |
| | | | AAAACAATGAAATTATTTATCTCCACCTCCCCTATTGATGCACA |
| | | | GCAGAAATAAATATAGGGATCACCACCTTCTGTGCAAATGCAA |
| | | | ATAAGTATACTCGCAGAAACAAAAATTTCAACCTACAATTTCAG |
| | | | TTTTCCTCACCTTTTGCTTACACTCTA |
| SNP069 | 1 | 141 | AGGCATGAAGTAAGGGTCGAGGTCCAAGGGTGTGTGACACAAC |
| | | | ATTGCTACCATGTTATAGAGGGATATTCTAAACAAAATCTCTGC |
| | | | ATTCTTACCCCATGAACCCTATCTTCAGCCTTTACCACTGGAAA |
| | | | GCATCTTTCTAAATTCAAATCCTTGATTTGCTTCTGGTTTTGTAA |
| | | | TAAAGTCATGAGCAATAGGAATGCA |
| SNP069 | 2 | 142 | AGGCATGAAGTAAGGGTCGAGGTCCAAGGGTGTGTGACACAAC |
| | | | ATTGCTACCATGTTATAGAGGGATATTCTAAACAAAATCTCTGC |
| | | | ATTCTTACCCCATGAGCCCTATCTTCAGCCTTTACCACTGGAAA |
| | | | GCATCTTTCTAAATTCAAATCCTTGATTTGCTTCTGGTTTTGTAA |
| | | | TAAAGTCATGAGCAATAGGAATGCA |
| SNP070 | 1 | 143 | TCAGACCTCTAGGCCCTCCTTCCTTAGACTCCGGAGTCCCTCCT |
| | | | GCCTCTGACACTCACGAGGTCCAGACCCCAAGATAGCCCAGCC |
| | | | AGCAGGGACAGGGACAGGTAAGCCTCACATGCTGGGCTCTGCA |
| | | | GGAGAATGAGAGGGGCTGAAGCTGGGCCCCTCTCGCTTTCCTCT |
| | | | TTCTCTCTCCTCCCCTTCACACCTGAA |
| SNP070 | 2 | 144 | TCAGACCTCTAGGCCCTCCTTCCTTAGACTCCGGAGTCCCTCCT |
| | | | GCCTCTGACACTCACGAGGTCCAGACCCCAAGATAGCCCAGCC |
| | | | AGCAGGGACAGGGACGGGTAAGCCTCACATGCTGGGCTCTGCA |
| | | | GGAGAATGAGAGGGGCTGAAGCTGGGCCCCTCTCGCTTTCCTCT |
| | | | TTCTCTCTCCTCCCCTTCACACCTGAA |
| SNP071 | 1 | 145 | TAATGAACAGAATATTTACTAAATGCTAGAGAGTAAAAATCCTT |
| | | | TCTATCCTTTCACCAAATCCCTAGATTAGTTGTATGGGGAGGGG |
| | | | GCAGGAAGCAGTACATGGAAGAAAAGCTCTTACTAGGCTTTTC |
| | | | AGTAAGCAGAAAACATGAGCTCCTAGATAGGACGGCAAATTCA |
| | | | TTTTTAAATCAACGGCAATTCCTATGA |
| SNP071 | 2 | 146 | TAATGAACAGAATATTTACTAAATGCTAGAGAGTAAAAATCCTT |
| | | | TCTATCCTTTCACCAAATCCCTAGATTAGTTGTATGGGGAGGGG |
| | | | GCAGGAAGCAGTACGTGGAAGAAAAGCTCTTACTAGGCTTTTC |
| | | | AGTAAGCAGAAAACATGAGCTCCTAGATAGGACGGCAAATTCA |
| | | | TTTTTAAATCAACGGCAATTCCTATGA |
| SNP072 | 1 | 147 | GATCTATTAGGATTGAATATGCTTTGTAAACAGTAGCATAACCA |
| | | | GATATTTCTGACTCCATTCTATAGTATGTAGTCTTCAGTTATCAG |
| | | | GACAAGTATCTTTCATGCTGTAACTCAGCCTTGAGAACTCGTTG |
| | | | GCACATTGCTCGGTCAGTGATCTGGGAGTCCAGTCATTGCAACG |
| | | | GGGATGCACTGAGCACCTGCTGAT |
| SNP072 | 2 | 148 | GATCTATTAGGATTGAATATGCTTTGTAAACAGTAGCATAACCA |
| | | | GATATTTCTGACTCCATTCTATAGTATGTAGTCTTCAGTTATCAG |
| | | | GACAAGTATCTTTTATGCTGTAACTCAGCCTTGAGAACTCGTTG |
| | | | GCACATTGCTCGGTCAGTGATCTGGGAGTCCAGTCATTGCAACG |
| | | | GGGATGCACTGAGCACCTGCTGAT |
| SNP073 | 1 | 149 | TTGAAAGGGTGTTTAATAAGATAATTGGGCCGGGCACAGTGGC |
| | | | TCATGCCTGTAATCCCAGCATTTTGGGAGGCTGAGGAGGGCAG |
| | | | ATCATGAGGTCAGGAGGTTGAGACCAGCCTGACCAACATGGTG |
| | | | AAACCCCGTCTCTACTAAAAATACAAAAATTAGCCAGGCGTGG |
| | | | TGGCACACACCTGTAATCCCAGCTACTCA |
| SNP073 | 2 | 150 | TTGAAAGGGTGTTTAATAAGATAATTGGGCCGGGCACAGTGGC |
| | | | TCATGCCTGTAATCCCAGCATTTTGGGAGGCTGAGGAGGGCAG |
| | | | ATCATGAGGTCAGGAGTTTGAGACCAGCCTGACCAACATGGTG |
| | | | AAACCCCGTCTCTACTAAAAATACAAAAATTAGCCAGGCGTGG |
| | | | TGGCACACACCTGTAATCCCAGCTACTCA |
| SNP074 | 1 | 151 | GGGCCACAGCAAGCAAGGGGCAGCGGCTTTTGCCTCCCCACCC |
| | | | TGCCCTGGCCCCGTCACCTCCCAAGGAGGGAAAGGTGATGCAT |
| | | | ACGTGCCCGAAGAAACCGACCGCATAGGTTATTTTCACGCAGC |
| | | | CCCTCCAAGGCAGGCACTAACTGGACACCTGCTTTGCGTCTCAG |
| | | | CTGTTGAAATGCCATCCCCTGCCCCCAG |
| SNP074 | 2 | 152 | GGGCCACAGCAAGCAAGGGGCAGCGGCTTTTGCCTCCCCACCC |
| | | | TGCCCTGGCCCCGTCACCTCCCAAGGAGGGAAAGGTGATGCAT |
| | | | ACGTGCCCGAAGAAACTGACCGCATAGGTTATTTTCACGCAGC |
| | | | CCCTCCAAGGCAGGCACTAACTGGACACCTGCTTTGCGTCTCAG |
| | | | CTGTTGAAATGCCATCCCCTGCCCCCAG |
| SNP075 | 1 | 153 | GGGCACAGAAAGGACCCTAGAGGGTCATCTGACCTGGGCCCAG |
| | | | ACACCCTGAGACCCATGACCCCTGGACTCTTGCAGATGCCAGTT |
| | | | CAATCCCCCATTTCCCCTTTTTATTTAATCAGCACTTTTCTGAGC |
| | | | ATCAGCAGTGCTGGAGGCCCTGTGCCAGGCGCTTCCCATCAGCA |
| | | | GCTCGTTTAGACCTCACAGCTGTTC |
| SNP075 | 2 | 154 | GGGCACAGAAAGGACCCTAGAGGGTCATCTGACCTGGGCCCAG |
| | | | ACACCCTGAGACCCATGACCCCTGGACTCTTGCAGATGCCAGTT |
| | | | CAATCCCCCATTTCCTCTTTTTATTTAATCAGCACTTTTCTGAGC |
| | | | ATCAGCAGTGCTGGAGGCCCTGTGCCAGGCGCTTCCCATCAGCA |
| | | | GCTCGTTTAGACCTCACAGCTGTTC |
| SNP076 | 1 | 155 | TTGGGAAAGAATTTGAACTCATCTTCCAATATATCCTACTAACA |
| | | | AAATTTTTTTGAGATGATGAATAGATTTGTTCTTATAACCAGTG |
| | | | TGGAAAGTTAACTCCATGGGGCTAAACAAATTCTCTTAACGTCA |
| | | | ACACACAACACAGGACAGGAGACAAAAAGTAATGTGAGATTAT |
| | | | ATTTTAGTATGCCATTAAAAATTTTG |
| SNP076 | 2 | 156 | TTGGGAAAGAATTTGAACTCATCTTCCAATATATCCTACTAACA |
| | | | AAATTTTTTTGAGATGATGAATAGATTTGTTCTTATAACCAGTG |
| | | | TGGAAAGTTAACTCTATGGGGCTAAACAAATTCTCTTAACGTCA |
| | | | ACACACAACACAGGACAGGAGACAAAAAGTAATGTGAGATTAT |
| | | | ATTTTAGTATGCCATTAAAAATTTTG |
| SNP077 | 1 | 157 | GGAAGGTGGGCTGAGCCTGTGGGCAGGTGTTGGTGCTCCCCTCC |
| | | | CCGACGGGGCACGATGGGGACAGAGCATGGGAGGGAATATGA |
| | | | AGCAGGAGCTCTGTCTCGTACACATGGAATCTGAGGAGCTGAC |
| | | | AGATGACCTGTGGGGAGGGTGGTCCCGTGCCAATGTGTGCTGG |
| | | | AAGGACATGCCTGTGCGTTTATCAGCTCT |
| SNP077 | 2 | 158 | GGAAGGTGGGCTGAGCCTGTGGGCAGGTGTTGGTGCTCCCCTCC |
| | | | CCGACGGGGCACGATGGGGACAGAGCATGGGAGGGAATATGA |
| | | | AGCAGGAGCTCTGTCTTGTACACATGGAATCTGAGGAGCTGAC |
| | | | AGATGACCTGTGGGGAGGGTGGTCCCGTGCCAATGTGTGCTGG |
| | | | AAGGACATGCCTGTGCGTTTATCAGCTCT |
| SNP078 | 1 | 159 | AAGAAAATGTATGTTGTTTCTTAAATATCATTTACAGCTGTAAA |
| | | | ATATTCTGTTGAGGATGCCACCTAATTCAATTAACCATTCTCCT |
| | | | GTCCTCAAATATTTACTTCCTCCTTCTTTTGGGGTTTTATAAAGA |
| | | | ACAATATGGTAAACATCTGTGTGCATGTAAGTTCTTGCTTGCTT |
| | | | TCTTTTTTTTTTTTTTTTTTTGAG |
| SNP078 | 2 | 160 | AAGAAAATGTATGTTGTTTCTTAAATATCATTTACAGCTGTAAA |
| | | | ATATTCTGTTGAGGATGCCACCTAATTCAATTAACCATTCTCCT |
| | | | GTCCTCAAATATTTCCTTCCTCCTTCTTTTGGGGTTTTATAAAGA |
| | | | ACAATATGGTAAACATCTGTGTGCATGTAAGTTCTTGCTTGCTT |
| | | | TCTTTTTTTTTTTTTTTTTTTGAG |
| SNP079 | 1 | 161 | TTGTAGATGGGTAACAGCCCAGAGATGGGAAGGGACGTGCACA |
| | | | AGATGGGAATGGGCGTGCCCATGGTTGCACCGTGTGGTGTGGC |
| | | | AGAGCAGGAACTGGAACACAGGCGGCTGGAAGTGAAAGTGGA |
| | | | GCTCAGGCTTTTTAGCAGTTACTATGTGTGATTTCCTTTTCATCA |
| | | | TCACATCAACCCCATTTTTTTTTTTCAG |
| SNP079 | 2 | 162 | TTGTAGATGGGTAACAGCCCAGAGATGGGAAGGGACGTGCACA |
| | | | AGATGGGAATGGGCGTGCCCATGGTTGCACCGTGTGGTGTGGC |
| | | | AGAGCAGGAACTGGAATACAGGCGGCTGGAAGTGAAAGTGGA |
| | | | GCTCAGGCTTTTTAGCAGTTACTATGTGTGATTTCCTTTTCATCA |
| | | | TCACATCAACCCCATTTTTTTTTTTCAG |
| SNP080 | 1 | 163 | TCTAAAGCTTCCCTCTGAATGCTGCTTTGGAGGATTGTGAGAGG |
| | | | TAGTGACTCTTCAAAGTTTGTTTGTTTTCTTGAAGCTTTTACCTC |
| | | | TATGCAAATATGCAGTTTGGAGCAGGGAAGAAAGGTTAACTGT |
| | | | GATGGCGCCGGCTCTTAACGTGGAATGTCCTGAATTAATGTGGG |
| | | | TTTCAGTCCTCTGGCTCAGGATC |
| SNP080 | 2 | 164 | TCTAAAGCTTCCCTCTGAATGCTGCTTTGGAGGATTGTGAGAGG |
| | | | TAGTGACTCTTCAAAGTTTGTTTGTTTTCTTGAAGCTTTTACCTC |
| | | | TATGCAAATATGCGGTTTGGAGCAGGGAAGAAAGGTTAACTGT |
| | | | GATGGCGCCGGCTCTTAACGTGGAATGTCCTGAATTAATGTGGG |
| | | | TTTCAGTCCTCTGGCTCAGGATC |
| SNP080 | 3 | 165 | TCTAAAGCTTCCCTCTGAATGCTGCTTTGGAGGATTGTGAGAGG |
| | | | TAGTGACTCTTCAAAGTTTGTTTGTTTTCTTGAAGCTTTTACCTC |
| | | | TATGCAAATATGCTGTTTGGAGCAGGGAAGAAAGGTTAACTGT |
| | | | GATGGCGCCGGCTCTTAACGTGGAATGTCCTGAATTAATGTGGG |
| | | | TTTCAGTCCTCTGGCTCAGGATC |
| SNP081 | 1 | 166 | CAGGAGGGAGGGAAGAAGGAAGCCCAGCTCACTCATCCAACCG |
| | | | GAAGGACCCCTGCTCCAGGCAGGACTGGAATAACGCCAGGACT |
| | | | CCAAATGAGCAAGATAATCCCAGCCTGGCCTGCCTGGGATAAG |
| | | | GAGCGGGCAGGGAAACCCAGAGGAGAAGCACTGACTTTGGCA |
| | | | GGATGGGGCATGTGAGGGCACCTGGAAGGC |
| SNP081 | 2 | 167 | CAGGAGGGAGGGAAGAAGGAAGCCCAGCTCACTCATCCAACCG |
| | | | GAAGGACCCCTGCTCCAGGCAGGACTGGAATAACGCCAGGACT |
| | | | CCAAATGAGCAAGATAGTCCCAGCCTGGCCTGCCTGGGATAAG |
| | | | GAGCGGGCAGGGAAACCCAGAGGAGAAGCACTGACTTTGGCA |
| | | | GGATGGGGCATGTGAGGGCACCTGGAAGGC |

TABLE 3 shows the primers selected by PlexForm™ for the targets in TABLE 2. “F” in the primer name denotes a forward primer. “R” in the primer name denotes a reverse primer. TABLE 3 comprises SEQ ID NO: 168-329. (See e.g., TABLE 13 for IUPAC nucleotide abbreviations.)

TABLE 3
PlexForm primers selected (for the target sequences in TABLE 2)

| | | SEQ ID | |
|---|---|---|---|
| Target | Primer Name | NO: | Primer Sequence |
| SNP001 | SNP001-F | 168 | ACAAGTATGTTGCCATTCTGTGGA |
| SNP001 | SNP001-R | 169 | AGTTTCAAAGTTTGGAAGGGGAAAATAA |
| SNP002 | SNP002-F | 170 | CAAGGGATGAATCCATAGCTCAAAGC |
| SNP002 | SNP002-R | 171 | AGGCCCAGAGAGACATTAAAATGAGA |
| SNP003 | SNP003-F | 172 | CCCGGTAGGAATAAGGCAAGCC |
| SNP003 | SNP003-R | 173 | GTCCATCTTACCCTCCCGGAG |
| SNP004 | SNP004-F | 174 | TCCCTTAGTGCTTCAGGATTCTAGAG |
| SNP004 | SNP004-R | 175 | CTGTGACATCAGCTGAGGCAC |
| SNP005 | SNP005-F | 176 | GCTGTGTGGGGTGTGTGTGAG |
| SNP005 | SNP005-R | 177 | CTCGCACACACACAGCCTC |
| SNP006 | SNP006-F | 178 | ATGGGAGACTTCAACACCTCAC |
| SNP006 | SNP006-R | 179 | TGGATATCCTTGTTAACTTTCTGTCTCTATC |
| SNP007 | SNP007-F | 180 | ACAGACAACAAAAACTAAGTGTAGGTC |
| SNP007 | SNP007-R | 181 | TCATCACTCAGTCACCTCTATAAATTAAAATC |
| SNP008 | SNP008-F | 182 | CATGGATAATAATGATAAAACCTTATGGAATGC |
| SNP008 | SNP008-R | 183 | CCCTAATGCAGTCATCCGAGAATAC |
| SNP009 | SNP009-F | 184 | YTCCAAGCTCATCCATGCTGTC |
| SNP009 | SNP009-R | 185 | TGGATAAAGAAAATGTGAGAGATATATACAATGG |
| SNP010 | SNP010-F | 186 | TCCCAAATCCCTGCTTCATCTAACATATATTG |
| SNP010 | SNP010-R | 187 | GTCATATAATAAATATTGCTTTGTGTTCTATCTGG |
| SNP011 | SNP011-F | 188 | TGGGGCCAACCTAGTCATTTGC |
| SNP011 | SNP011-R | 189 | TGTGATTCATTTATATCAGAATCATCAGGGAG |
| SNP012 | SNP012-F | 190 | CTGGCCCAGTTACTTATTTTAGAAGTTATATTTG |
| SNP012 | SNP012-R | 191 | ACAGCTCATGCCAGGGCTC |
| SNP013 | SNP013-F | 192 | AGAAACTGTCACAGCAAGAGGAC |
| SNP013 | SNP013-R | 193 | TGCCATCCAGGACACCATATTAC |
| SNP014 | SNP014-F | 194 | CATCAGAGTGTTAGACAATAAGATGCAG |
| SNP014 | SNP014-R | 195 | GCCTGTCTTTGGTTAGCAGTGAG |
| SNP015 | SNP015-F | 196 | TTTGAGATGGAGTCTTGCCCTG |
| SNP015 | SNP015-R | 197 | GAGTTGAGATCACGCCACCG |
| SNP016 | SNP016-F | 198 | AAATTGGAGATTGCCCTCATTTGG |
| SNP016 | SNP016-R | 199 | TGAAATCACTGGACTAAGCACCC |
| SNP017 | SNP017-F | 200 | ATCCTCCAGCCTCCACCG |
| SNP017 | SNP017-R | 201 | GCAGGGCCTCCTCCTGAG |
| SNP018 | SNP018-F | 202 | TCTGGGCTGGTTCTGGCTG |
| SNP018 | SNP018-R | 203 | CACCCTCTGCAGCTCAAAGTC |
| SNP019 | SNP019-F | 204 | AGGCTAGAGATGTAGTCAGACCCTA |
| SNP019 | SNP019-R | 205 | CCATTAAAACCTGTTCCTGAGGTCT |
| SNP020 | SNP020-F | 206 | GCTCTGGTTTAGCTCCCTACTC |
| SNP020 | SNP020-R | 207 | AGGTTCCCTTCCAAACTGACTC |
| SNP021 | SNP021-F | 208 | AGAYGACAGTCATGCTTCGATTTAAGT |
| SNP021 | SNP021-R | 209 | GGAACGAAGAACAACTTCCTCTAGGA |
| SNP022 | SNP022-F | 210 | TCACCCATTGGCTCCTAATGTG |
| SNP022 | SNP022-R | 211 | TGTTGAACGCTCACTGAGGTAG |
| SNP023 | SNP023-F | 212 | GTTCTGGAACTACTGGTATTTATTGCTG |
| SNP023 | SNP023-R | 213 | CTCTTTTAGGAGGACTTGTGCAATG |
| SNP024 | SNP024-F | 214 | GCTCAAGAGATCTGCCTGCCTC |
| SNP024 | SNP024-R | 215 | AAAGAATGCAGGCTCTGGGCAG |
| SNP025 | SNP025-F | 216 | AGTGGCGTGGTGGAAAGTTTC |
| SNP025 | SNP025-R | 217 | TCCCGCTGTCTCAGGCG |
| SNP026 | SNP026-F | 218 | CCTGGCCTTTGAGACTCCCTC |
| SNP026 | SNP026-R | 219 | TCCACAGCTGGGCTCAGAG |
| SNP027 | SNP027-F | 220 | CCACAGTAGAAAATGTGCCTGTGG |
| SNP027 | SNP027-R | 221 | TGCCGAGGGTTGTTCATCCTC |
| SNP028 | SNP028-F | 222 | GAGTTCTTTATTCYGTTTCATTGGTCTATGT |
| SNP028 | SNP028-R | 223 | CTTGGTAAAGAGTGAGACACTTTCAAAA |
| SNP029 | SNP029-F | 224 | ACATGAGATTTAGTGGGGACAGAG |
| SNP029 | SNP029-R | 225 | TGAAATTTGGAGGGGCTGGGG |
| SNP030 | SNP030-F | 226 | ACCAAAGGCAAAGAAGTTCAAAACT |
| SNP030 | SNP030-R | 227 | CAGCTCCTTTAAGCACTTTTCTGTAT |
| SNP031 | SNP031-F | 228 | GAAAGAATGTTACTTCTGTTAATATGAATAGCATG |
| SNP031 | SNP031-R | 229 | AGGTCAGGCTCCATATGGACC |
| SNP032 | SNP032-F | 230 | AAGATGGCTTCTGGAGTGCTAC |
| SNP032 | SNP032-R | 231 | TTGCCCTTTTCCTTCTAGCCTAC |
| SNP033 | SNP033-F | 232 | AGGAGCAAGCCATCACACTTGAC |
| SNP033 | SNP033-R | 233 | GCCTGGGCAACAAGAGTGAAAC |
| SNP034 | SNP034-F | 234 | AGCCTGGCCAGAATATCTGCG |
| SNP034 | SNP034-R | 235 | CCCCAATTATTGTCATCCTTCTCCC |
| SNP035 | SNP035-F | 236 | TGGGAATTTCAGACTATAGAAGCTCTC |
| SNP035 | SNP035-R | 237 | TGATGAGTTCTGATACAGTAACTAGTGGT |
| SNP036 | SNP036-F | 238 | AGGAGCAGTGACTCCACTCCAG |
| SNP036 | SNP036-R | 239 | CGCACGTCCTAAGCTGACAG |
| SNP037 | SNP037-F | 240 | ACAAGTTTACCGATGTAAAACCTTCAC |
| SNP037 | SNP037-R | 241 | GCTGCTCGTGGCCTATTTTTATG |
| SNP038 | SNP038-F | 242 | CAGCTGACACCCTCCTTGAATC |
| SNP038 | SNP038-R | 243 | TTTTCTAATGGGGCCAGGGAAG |
| SNP039 | SNP039-F | 244 | ATGTTGCTGCACTGTTCCCATG |
| SNP039 | SNP039-R | 245 | GAAAAAAGAATGCACGTSTGAGTTAC |
| SNP040 | SNP040-F | 246 | CCACAGGTCAAATGAGGCTCC |
| SNP040 | SNP040-R | 247 | GGATAAAAGCCACTCCTGTCCAG |
| SNP041 | SNP041-F | 248 | CCGGCCTCGTACGGTAATTC |
| SNP041 | SNP041-R | 249 | AGTTCGCCAGCTCCGAGAC |
| SNP042 | SNP042-F | 250 | TCCCTAACGCAATCCCTGTGAC |
| SNP042 | SNP042-R | 251 | GGATCTGTCTCTCCCCTTTACTCTTAG |
| SNP043 | SNP043-F | 252 | TTCAAGACCAGCCAGGATGAC |
| SNP043 | SNP043-R | 253 | CTGTGTGTCACCATTCACAGCTG |
| SNP044 | SNP044-F | 254 | TGTCCTTCACAGCTTAGATTCAATCTTTC |
| SNP044 | SNP044-R | 255 | GAGTCAGCCCAGAATCACACTC |
| SNP045 | SNP045-F | 256 | TGCCAGGCCTGAGGATCTTC |
| SNP045 | SNP045-R | 257 | TTCCCTAGGACCAGTGTACCATG |
| SNP046 | SNP046-F | 258 | AGACTTCAACAAATATGTGCTGTTGT |
| SNP046 | SNP046-R | 259 | TGTCAACCAGAGATAATAAAAACACAGGTA |
| SNP047 | SNP047-F | 260 | AGGCTGGTCTCAAACTCCTGAC |
| SNP047 | SNP047-R | 261 | ACCTGTAATCCCAGCACTTTGAGAG |
| SNP048 | SNP048-F | 262 | ACTTTGCATTCGTGCCACAATAG |
| SNP048 | SNP048-R | 263 | GGCTCCAAAGACCAAATGGAATG |
| SNP049 | SNP049-F | 264 | ATGAAAAATGTTTGAATATGATCTTTAGGGAC |
| SNP049 | SNP049-R | 265 | CATGTACAGAATTTATTGTTCCCATTTCATATAC |
| SNP050 | SNP050-F | 266 | CATCTAGGCCAGCCCTTCCC |
| SNP050 | SNP050-R | 267 | TACTTGAAGGTCCCCAGGCG |
| SNP051 | SNP051-F | 268 | AGAACCTATTGTGGATGTTAGGAGAG |
| SNP051 | SNP051-R | 269 | GGCTTCATCTCCCAGAGTCATTG |
| SNP052 | SNP052-F | 270 | ACAGTCAGCAGTAATCTCACATCTG |
| SNP052 | SNP052-R | 271 | GGGAAGAAGAAAGCATTGTTGTGAC |
| SNP053 | SNP053-F | 272 | ACAGAAAAGTTTTTACCACAGACTACCA |
| SNP053 | SNP053-R | 273 | TCTTGTTATGCAGATGAAGCCTCAA |
| SNP054 | SNP054-F | 274 | ACCTCAYACATTATGMAGCATAGGGTATC |
| SNP054 | SNP054-R | 275 | GGTGTACATGTATTTATGCCACCCC |
| SNP055 | SNP055-F | 276 | CCACATTCTGTCGTGGTTGAGAC |
| SNP055 | SNP055-R | 277 | CTCCTGACAGGACTCCACACC |
| SNP056 | SNP056-F | 278 | GGGAGCAGAGCTGAGTTTCTAAAC |
| SNP056 | SNP056-R | 279 | TCCTTCTGTGAAGTATCTCCAYATATGGATTTG |
| SNP057 | SNP057-F | 280 | AGTGCCTAATTGTTCACCCAGC |
| SNP057 | SNP057-R | 281 | AVCAGTGAGGCTYTATAAATAAAATTAATTGTG |
| SNP058 | SNP058-F | 282 | GATCCCAGGGCTCCTTGAAGAG |
| SNP058 | SNP058-R | 283 | AAGAAATCCAACTGTGCAAGTGTG |
| SNP059 | SNP059-F | 284 | GTGTTCTAGAAGCCAAGCAAAGATG |
| SNP059 | SNP059-R | 285 | AGCAGCATTTGACACAGTTGATG |
| SNP060 | SNP060-F | 286 | GCCTTGTCCCCTGTATCCTTTC |
| SNP060 | SNP060-R | 287 | CCAGAAAAGGCAGTTAATAGAAGGC |
| SNP061 | SNP061-F | 288 | TTYTCTCCAATCTTAGTAACACAGCTGAC |
| SNP061 | SNP061-R | 289 | ACAAATATTTAGAAATCAAGTAGAAGTGAGATRC |
| SNP062 | SNP062-F | 290 | CCGTGATCCTTAAATAGCAGCACAG |
| SNP062 | SNP062-R | 291 | CTGGGGCCACATCAATCCACG |
| SNP063 | SNP063-F | 292 | CCTACGATCATAATCACAAGTACCTCG |
| SNP063 | SNP063-R | 293 | GCAAAGCTACCAGTCATGTGCC |
| SNP064 | SNP064-F | 294 | ACTCGCATGCACGTATATGTACAC |
| SNP064 | SNP064-R | 295 | CCTGTGACTTAAGACTTACAATAATTCTTCC |
| SNP065 | SNP065-F | 296 | ACTCCTCCCAGCAGTAGTAGATG |
| SNP065 | SNP065-R | 297 | TGCTGATCCTGGGAGTGCTG |
| SNP066 | SNP066-F | 298 | ACAATCAAGACTCATGCATTGCAAG |
| SNP066 | SNP066-R | 299 | ACTTCTCCCAATGAAGATCACTTAGG |
| SNP067 | SNP067-F | 300 | CCTTGGCTCACAGAGTCATCTC |
| SNP067 | SNP067-R | 301 | GCTTTGAGATGAGTACAGGGTGG |
| SNP068 | SNP068-F | 302 | CCTCCCCTATTGATGCACAGC |
| SNP068 | SNP068-R | 303 | TTCTGCGAGTATACTTATTTGCATTTGC |
| SNP069 | SNP069-F | 304 | CAAAATCTCTGCATTCTTACCCCATG |
| SNP069 | SNP069-R | 305 | AGATGCTTTCCAGTGGTAAAGGC |
| SNP070 | SNP070-F | 306 | CAAGATAGCCCAGCCAGCAG |
| SNP070 | SNP070-R | 307 | TGCAGAGCCCAGCATGTG |
| SNP071 | SNP071-F | 308 | GGGAGGGGGCAGGAAGC |
| SNP071 | SNP071-R | 309 | TCTAGGAGCTCATGTTTTCTGCTTAC |
| SNP072 | SNP072-F | 310 | TGTAGTCTTCAGTTATCAGGACAAGTATC |
| SNP072 | SNP072-R | 311 | TGTGCCAAYGAGTTCTCAAGG |
| SNP073 | SNP073-F | 312 | GAGGGCAGATCATGAGGTCAG |
| SNP073 | SNP073-R | 313 | ACGGGGTTTCACCATGTTGG |
| SNP074 | SNP074-F | 314 | AAGGAGGGAAAGGTGATGCATAC |
| SNP074 | SNP074-R | 315 | GGAGGGGCTGCGTGAAAATAAC |
| SNP075 | SNP075-F | 316 | TGCCAGTTCAATCCCCCATTT |
| SNP075 | SNP075-R | 317 | GGGCCTCCAGCACTGCT |
| SNP076 | SNP076-F | 318 | TTGTTCTTATAACCAGTGTGGAAAGTTAAC |
| SNP076 | SNP076-R | 319 | TCCTGTGTTGTGTGTTGACGTTAAG |
| SNP077 | SNP077-F | 320 | TGGGAGGGAATATGAAGCAGGAG |
| SNP077 | SNP077-R | 321 | CATCTGYCAGCTCCTCAGATTCC |
| SNP078 | SNP078-F | 322 | TCAATTAACCAKTCTCCTGTCCTCAA |
| SNP078 | SNP078-R | 323 | ACCATATTGTTCTTTATAAAACCCCAAAAGAA |
| SNP079 | SNP079-F | 324 | GGTGTGGCAGAGCAGGAAC |
| SNP079 | SNP079-R | 325 | GCCTGAGCTCCACTTTCACTTC |
| SNP080 | SNP080-F | 326 | TCTTGAAGCTTTTACCTCTATGCAAATAYG |
| SNP080 | SNP080-R | 327 | CGCCATCACAGTTAACCTTTCTTC |
| SNP081 | SNP081-F | 328 | GCCAGGACTCCAAATGAGCAAG |
| SNP081 | SNP081-R | 329 | CGCTCCTTATCCCAGGCAG |

The primers from TABLE 3 were synthesized and tested using NGS. TABLE 4 shows the number of total reads obtained on an Illumina sequencer, using the primers from TABLE 3. The data in TABLE 4 demonstrate even multiplexing in one reaction (average of 427, standard Deviation of 230).

TABLE 4
Number of total reads obtained on an Illumina
sequencer (using the primers from TABLE 3)

| | PlexCall ™ | | PlexCall ™ | | PlexCall ™ |
|---|---|---|---|---|---|
| Target | Total Read # | Target | Total Read # | Target | Total Read # |
| SNP001 | 228.0 | SNP028 | 20.0 | SNP055 | 673.0 |
| SNP002 | 670.0 | SNP029 | 594.0 | SNP056 | 265.0 |
| SNP003 | 696.0 | SNP030 | 28.0 | SNP057 | 178.0 |
| SNP004 | 357.0 | SNP031 | 282.0 | SNP058 | 689.0 |
| SNP005 | 415.0 | SNP032 | 770.0 | SNP059 | 533.0 |
| SNP006 | 541.0 | SNP033 | 89.0 | SNP060 | 463.0 |
| SNP007 | 201.0 | SNP034 | 663.0 | SNP061 | 384.0 |
| SNP008 | 207.0 | SNP035 | 463.0 | SNP062 | 568.0 |
| SNP009 | 40.0 | SNP036 | 670.0 | SNP063 | 650.0 |
| SNP010 | 489.0 | SNP037 | 31.0 | SNP064 | 376.0 |
| SNP011 | 686.0 | SNP038 | 580.0 | SNP065 | 692.0 |
| SNP012 | 566.0 | SNP039 | 569.0 | SNP066 | 162.0 |
| SNP013 | 579.0 | SNP040 | 107.0 | SNP067 | 625.0 |
| SNP014 | 665.0 | SNP041 | 80.0 | SNP068 | 390.0 |
| SNP015 | 591.0 | SNP042 | 723.0 | SNP069 | 754.0 |
| SNP016 | 837.0 | SNP043 | 524.0 | SNP070 | 314.0 |
| SNP017 | 217.0 | SNP044 | 591.0 | SNP071 | 499.0 |
| SNP018 | 363.0 | SNP045 | 674.0 | SNP072 | 223.0 |
| SNP019 | 89.0 | SNP046 | 264.0 | SNP073 | 48.0 |
| SNP020 | 702.0 | SNP047 | 36.0 | SNP074 | 83.0 |
| SNP021 | 165.0 | SNP048 | 282.0 | SNP075 | 370.0 |
| SNP022 | 626.0 | SNP049 | 262.0 | SNP076 | 396.0 |
| SNP023 | 628.0 | SNP050 | 722.0 | SNP077 | 521.0 |
| SNP024 | 476.0 | SNP051 | 669.0 | SNP078 | 104.0 |
| SNP025 | 494.0 | SNP052 | 596.0 | SNP079 | 706.0 |
| SNP026 | 522.0 | SNP053 | 175.0 | SNP080 | 143.0 |
| SNP027 | 245.0 | SNP054 | 589.0 | SNP081 | 461.0 |

### Example 12: Method for Equalization of Reads Using Anti-Sense Oligos

Due at least in part to differences in amplification efficiency, some sequence elements tend to be over-represented in multiplex amplification. This phenomenon reduces the dynamic range and accuracy of sequencing and genotyping based on NGS.

Described herein is a method for equalization of reads using anti-sense oligos in various concentrations to inhibit high-performing amplicons from forming and thereby allow poorer performing amplicons to produce more data. Anti-sense oligos can be produced with reversible binding characteristics using complementary sequences (e.g., attenuator oligos). Also described herein is a method for performing multiplex PCR that results in even reads produced by NGS instruments for each amplicon (for 2-10,000 amplicons) and each sample (for 1-100,000 DNA samples).

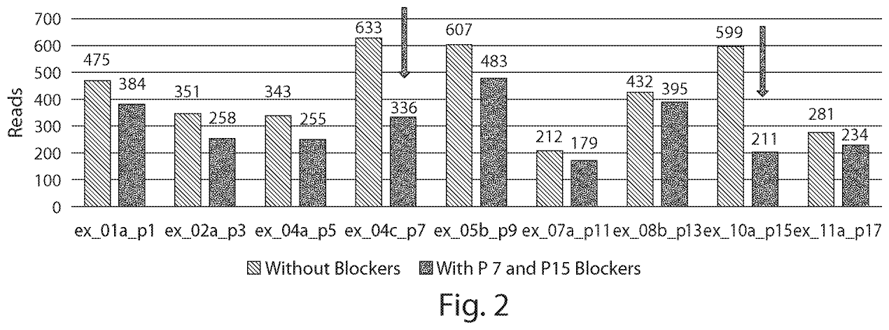

An exemplary process for equalization of reads is as follows: A) Initial multiplex is analyzed on a control sample and reads for each amplicon in the mix are obtained from PlexCall™ software; B) Anti-sense oligos targeting highly represented or over-represented amplicons in the reaction are added at a concentration needed to equalize number of reads for each amplicon (or target); and steps (A) and (B) are repeated as needed to improve evenness of assay. Amplicons that are represented at more than 2-fold greater than the average or median abundance for a set of amplicons can be targeted for reduction by anti-sense oligos. In one embodiment, the evenness of amplification and subsequent NGS reads can be improved by iteratively adding anti-sense oligos to reduce those amplicons with the highest representations until even reads are achieved. By “even reads” in this regard is meant that either the representation of all sequences is within 50% higher or lower relative to all others in a multiplex reaction. As a non-limiting example, see e.g., FIG. 2, which shows a decrease in variance in read numbers in TP53 sequencing multiplex by addition of blockers (e.g., anti-sense oligos) to targets p7 and p15.

### Example 13: Barcode Primer Design

Described herein is a non-limiting example of barcode primer design. As a non-limiting example, two sets of 144 barcodes with a length of 10 bases each are used in every possible combination (20,736 combinations or 1442), which allows for simultaneous analysis of 20,736 sample DNAs. These 10-base barcodes can be incorporated into custom Illumina™ primers for 15 and I7. These 10-base barcodes can also be used in-line with other platforms such as Ion Torrent™. A combination of in-line and I5/I7 barcodes on the Illumina™ platform can be used for greater than 100,000 samples in the same sequencing run.

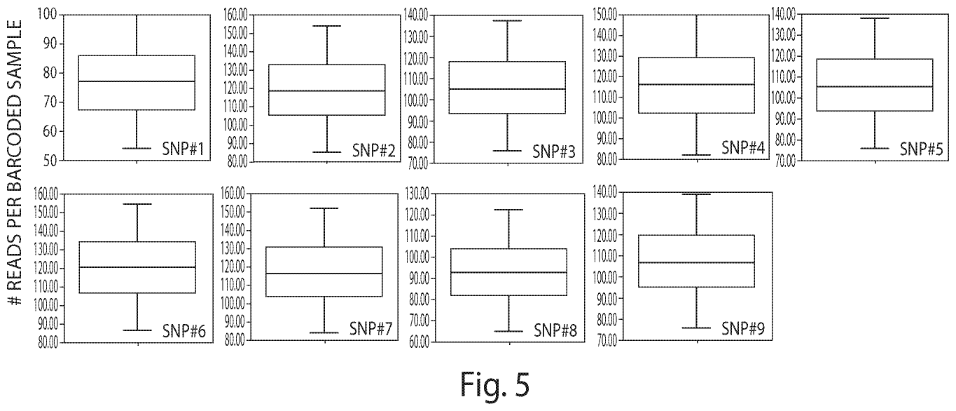

A non-limiting example showing barcode primer design is shown in FIG. 5. Control DNA was analyzed on 9 multiplexed amplicons barcoded in 20,682 PlexSeq reactions and analyzed simultaneously on one NGS run. Box plots demonstrate even amplification across amplicon targets and 20,682 samples based on read depth (see e.g., FIG. 5).

### Example 14: PCR Amplification Method

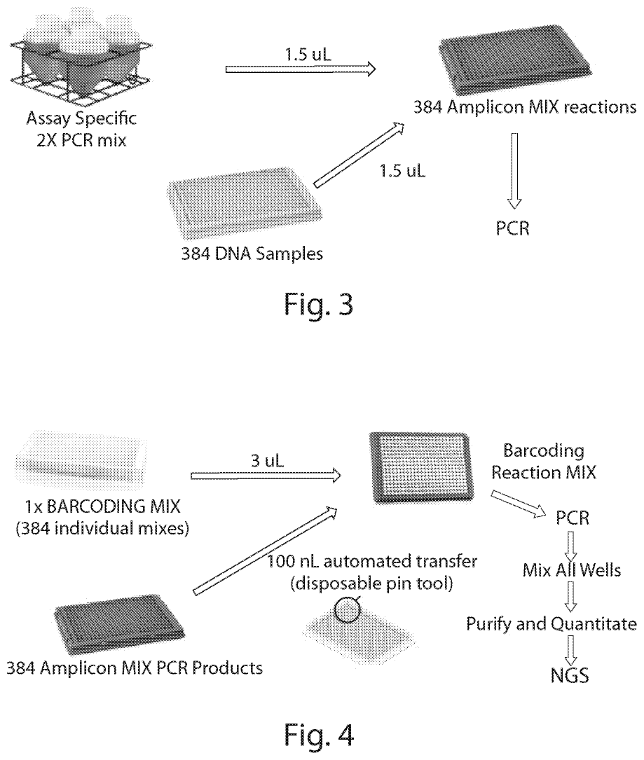

Described herein is a non-limiting example of a PCR amplification method. A primary amplification with the primer mix designed by PlexForm™ that are extended with a tail sequence on the 5′ end is first performed on each DNA sample using standard PCR conditions (see e.g., FIG. 3).

A universal secondary amplification is then performed using primers and the primary amplification as the source. 100 nanoliters are transferred from primary to secondary reaction via a disposable plastic pintool (see e.g., FIG. 4). All secondary amplicons are mixed and loaded onto a Next Generation Sequencer.

### Example 15: Single Base Sequencing

Described herein is a non-limiting example of single base sequencing. A variation of PlexSeq™ allows single base multiplexed sequencing on an NGS platform in order to avoid sequencing additional regions when this is beneficial. When data is needed for only one base pair, special primers are designed. An additional set of random bases are added to the 5′ end of the primers used in the primary amplification such that all are of length N. The sequencer is set to sequence only N+1 thus insuring that no additional sequence except the target base is analyzed. Single base sequencing can be performed with the procedures of any one of or any combination of Examples 1-14.

### Example 16: PlexCall™

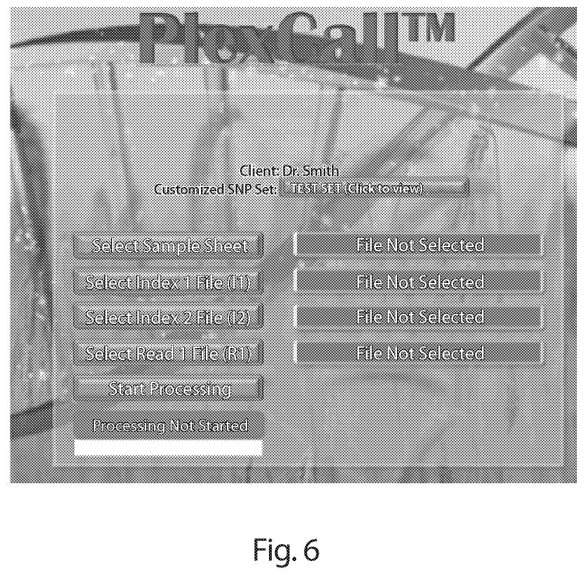

Described herein is a method to rapidly analyze PlexSeq™ data using personalized PlexCall™ software to automatically provide genotype or sequencing results. As a non-limiting example, a PlexSeq™ experiment is run on a sequencer, and direct analysis is performed automatically. PlexCall™ provides automatic calling of genotypes and allele ratios. PlexCall™ is personalized for each experiment and includes all barcode and SNP information. PlexCall™ can provide information on primer dimer composition to assist with refinement of assay during development. FIG. 6 shows the front-end screen of PlexCall™, demonstrating simplicity of selecting output files from sequencer and simple sample sheet indicating locations to correlate with barcode positions for analysis.

Described herein is a non-limiting example of using PlexCall™ to analyze PlexSeq™ data, using an exemplary set of SNPs (e.g., SMITH_1, SMITH_2, SMITH_3, SMITH_4, SMITH_5), which each comprise 2 alleles.

TABLE 5 shows the specific nucleotides for Allele 1 and Allele 2 for each of the SMITH SNPs. In TABLE 6, the allele ratio output from PlexCall™ demonstrates simple allele counts for easy determination of genotype. TABLE 7 shows the final genotype call output from PlexCall™, using the information from TABLE 5 and TABLE 6.

TABLE 5
Alleles for the SMITH SNPs

| SNP name | SMITH_1 | SMITH_2 | SMITH_3 | SMITH_4 | SMITH_5 |
|---|---|---|---|---|---|
| PROJECT | Dr. Smith | Dr. Smith | Dr. Smith | Dr. Smith | Dr. Smith |
| Allele_1 | C | T | C | A | C |
| Allele_2 | G | C | T | T | T |

TABLE 6
Allele ratio output from PlexCall ™

| | Index | | SMITH_1 | SMITH_2 | SMITH_3 | SMITH_4 | SMITH_5 |
|---|---|---|---|---|---|---|---|
| Sample | Plate | Well | Allele_1/ | Allele_1/ | Allele_1/ | Allele_1/ | Allele_1/ |
| Name | Number | Position | Allele_2 | Allele_2 | Allele_2 | Allele_2 | Allele_2 |
| SAMPLE_1 | PL001 | A01 | 0/313 | 289/0 | 0/206 | 261/0 | 253/0 |
| SAMPLE_2 | PL001 | B01 | 0/262 | 255/0 | 0/179 | 268/0 | 235/0 |
| SAMPLE_3 | PL001 | CO1 | 0/295 | 248/0 | 90/80 | 0/253 | 230/0 |
| SAMPLE_4 | PL001 | D01 | 0/301 | 244/0 | 134/110 | 0/230 | 263/0 |
| SAMPLE_5 | PL001 | E01 | 0/446 | 329/0 | 275/0 | 0/357 | 0/251 |
| SAMPLE_6 | PL001 | F01 | 0/285 | 0/246 | 126/140 | 213/0 | 236/0 |
| SAMPLE_7 | PL001 | GO1 | 0/295 | 0/243 | 106/0 | 0/249 | 233/0 |
| SAMPLE_8 | PL001 | H01 | 0/363 | 284/0 | 170/0 | 0/280 | 223/0 |
| SAMPLE_9 | PL001 | A02 | 0/251 | 0/154 | 94/0 | 211/200 | 194/0 |
| SAMPLE_10 | PL001 | B02 | 0/317 | 207/0 | 130/0 | 240/220 | 215/0 |

TABLE 7
Final genotype call output from PlexCall ™

| | Index | | SMITH_1 | SMITH_2 | SMITH_3 | SMITH_4 | SMITH_5 |
|---|---|---|---|---|---|---|---|
| Sample | Plate | Well | Allele_1/ | Allele_1/ | Allele_1/ | Allele_1/ | Allele_1/ |
| Name | Number | Position | Allele_2 | Allele_2 | Allele_2 | Allele_2 | Allele_2 |
| SAMPLE_1 | PL001 | A01 | G | T | T | A | C |
| SAMPLE_2 | PL001 | B01 | G | T | T | A | C |
| SAMPLE_3 | PL001 | CO1 | G | T | C/T | T | C |
| SAMPLE_4 | PL001 | D01 | G | T | C/T | T | C |
| SAMPLE_5 | PL001 | E01 | G | T | C | T | T |
| SAMPLE_6 | PL001 | F01 | G | C | C/T | A | C |
| SAMPLE_7 | PL001 | GO1 | G | C | C | T | C |
| SAMPLE_8 | PL001 | H01 | G | T | C | T | C |
| SAMPLE_9 | PL001 | A02 | G | C | C | A/T | C |
| SAMPLE_10 | PL001 | B02 | G | T | C | A/T | C |

### Example 17: Identification and Quantification of Low Abundance Rare Variants

As described herein, PlexSeq™ methodology can be used to identify and quantify low abundance rare variants in clinically relevant genes in a minority of tumor cells from a complex mixture of cells.

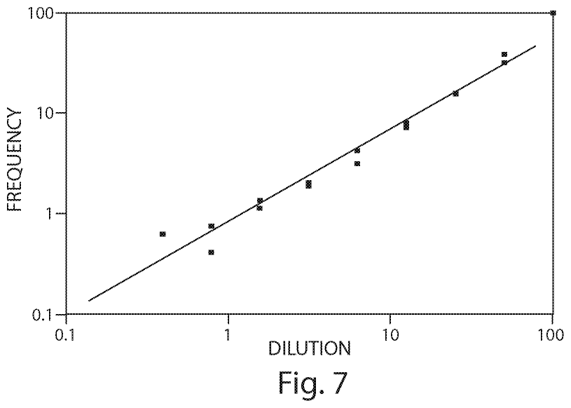

As a non-limiting example, TP53 analysis of the Arg273Cys mutation was performed in triplicate from a serial dilution of wild type DNA spiked with mutant DNA extracted from a cell line containing the mutation. TABLE 8 shows that the frequency of sequencing reads containing the Arg273Cys mutation is directly proportional to the amount of mutant DNA material present in the dilution series. The data from TABLE 8 is graphed in FIG. 7. As shown in FIG. 7, this process is linear down to 1% or lower.

TABLE 8
TP53 analysis of Arg273Cys mutation

| Object Label | DILUTION | FREQUENCY | MUTANT READS | TOTAL READS |
|---|---|---|---|---|
| CLINE-001-R0 | 100.00 | 99.14 | 1,720.00 | 1,735.00 |
| CLINE-001-R1 | 100.00 | 99.18 | 1,940.00 | 1,956.00 |
| CLINE-001-R2 | 100.00 | 99.21 | 2,005.00 | 2,021.00 |
| CLINE-002-R0 | 50.00 | 38.88 | 666.00 | 1,713.00 |
| CLINE-002-R1 | 50.00 | 32.04 | 837.00 | 2,612.00 |
| CLINE-002-R2 | 50.00 | 36.73 | 926.00 | 2,521.00 |
| CLINE-004-R0 | 25.00 | 15.47 | 318.00 | 2,056.00 |
| CLINE-004-R1 | 25.00 | 16.09 | 398.00 | 2,473.00 |
| CLINE-004-R2 | 25.00 | 15.08 | 409.00 | 2,713.00 |
| CLINE-008-R0 | 12.50 | 7.04 | 124.00 | 1,762.00 |
| CLINE-008-R1 | 12.50 | 8.20 | 170.00 | 2,074.00 |
| CLINE-008-R2 | 12.50 | 7.56 | 182.00 | 2,409.00 |
| CLINE-016-R0 | 6.25 | 4.13 | 75.00 | 1,815.00 |
| CLINE-016-R1 | 6.25 | 4.32 | 113.00 | 2,617.00 |
| CLINE-016-R2 | 6.25 | 3.09 | 77.00 | 2,493.00 |
| CLINE-032-R0 | 3.12 | 1.89 | 35.00 | 1,848.00 |
| CLINE-032-R1 | 3.12 | 1.88 | 44.00 | 2,337.00 |
| CLINE-032-R2 | 3.12 | 2.06 | 50.00 | 2,430.00 |
| CLINE-064-R0 | 1.56 | 1.13 | 21.00 | 1,855.00 |
| CLINE-064-R1 | 1.56 | 1.35 | 33.00 | 2,436.00 |
| CLINE-128-R0 | 0.78 | 0.76 | 14.00 | 1,838.00 |
| CLINE-128-R1 | 0.78 | 0.42 | 11.00 | 2,635.00 |
| CLINE-256-R2 | 0.39 | 0.63 | 15.00 | 2,381.00 |

As an example, the following method can be followed to identify and quantify low abundance rare variants, especially with regards to cancer variants. PlexSeq™ analysis is performed in triplicate on sets of clinical genes of interest. The mutant alleles are quantified. The mutant allele frequency is used to determine the percent of tumor cells in the sample.

Such a method can be applied to the detection of cells collected from blood, urine, other body fluids, or an organ biopsy. Such a method can be applied to the detection of minimal residual disease immediately post-surgery or therapy. Such a method can be applied to follow-up weekly, monthly or yearly to determine success of therapy and track disease recurrence.

### Example 18: Exemplary Primer Set #2

PlexForm™ software can be used for primer design that allows for large amplicon number multiplexing. Included herein is an exemplary primer set designed by PlexForm™ software for SNP targets from the tomato plant, Solanum lycopersicum (see e.g., TABLE 9). TABLE 9 shows the input allelic target sequences for 300 SNP regions (e.g., SNP100-SNP399), comprising SEQ ID NOs: 330-929. SNP position indicated by bolded letters in TABLE 9

TABLE 9
Input target sequences for 300 SNP regions

| | Allele | SEQ ID | |
|---|---|---|---|
| Target | # | NO: | Target Sequence |
| SNP100 | 1 | 330 | ACAATATACTCAGTGTATTAGAAGTGTGGCCTGGAGAGGGTAGGATGTACAC |
| | | | GACCTTACCTCTACCTTTGAAGGGTGGAGAGGTTGTTTCCGATAGACCC |
| SNP100 | 2 | 331 | ACAATATACTCAGTGTATTAGAAGTGTGGCCTGGAGAGGGTAGGATGTACGC |
| | | | GACCTTACCTCTACCTTTGAAGGGTGGAGAGGTTGTTTCCGATAGACCC |
| SNP101 | 1 | 332 | TGAATTCGGATAATGGATCTGTTGTACTTCTTTCTTGCAGCTTTTGCCTGTTTT |
| | | | TCACGAAAAGATTTAGGAATCAATAATTTCGACTCCTCTAGTTTCTT |
| SNP101 | 2 | 333 | TGAATTCGGATAATGGATCTGTTGTACTTCTTTCTTGCAGCTTTTGCCTGCTTT |
| | | | TCACGAAAAGATTTAGGAATCAATAATTTCGACTCCTCTAGTTTCTT |
| SNP102 | 1 | 334 | TGTTTCTAAGTGATTGTGGAGGAAAAAGATACCCTTTGTTGCAACATCCAATT |
| | | | GCGCCAGATGGACCAGTTCTTTTTTTGCCATTTTTGAAGGGATGCAAG |
| SNP102 | 2 | 335 | TGTTTCTAAGTGATTGTGGAGGAAAAAGATACCCTTTGTTGCAACATCCAGT |
| | | | TGCGCCAGATGGACCAGTTCTTTTTTTGCCATTTTTGAAGGGATGCAAG |
| SNP103 | 1 | 336 | TCTCTGTTGCATTTGTAGATGGTGGTGAGGCTGGAAATGTAATCCCTGAAAG |
| | | | TGTGAAGTTTGGTGGAACATTCCGGTTCTTGACGTTTGAGGGTCATTCC |
| SNP103 | 2 | 337 | TCTCTGTTGCATTTGTAGATGGTGGTGAGGCTGGAAATGTAATCCCTGAAGG |
| | | | TGTGAAGTTTGGTGGAACATTCCGGTTCTTGACGTTTGAGGGTCATTCC |
| SNP104 | 1 | 338 | GGTGAGAAAGTTGCGGAGCCACTATACAAGATGGAGGCTGGTAAGACGTAT |
| | | | AGGTATAGATTTTGCAATGTAGGTATGAGGACATCAGTGAATGTTAGGAT |
| SNP104 | 2 | 339 | GGTGAGAAAGTTGCGGAGCCACTATACAAGATGGAGGCTGGTAAGACGTAC |
| | | | AGGTATAGATTTTGCAATGTAGGTATGAGGACATCAGTGAATGTTAGGAT |
| SNP105 | 1 | 340 | TTTGTTCTATCTCTTCGCGTAACATTTGTGTCCAACGAAATCTTTTTGCCTCGC |
| | | | CACTTAAAAAGCCAAGCTATCATTGTACTGTTGTCTGTCTTGCGCTT |
| SNP105 | 2 | 341 | TTTGTTCTATCTCTTCGCGTAACATTTGTGTCCAACGAAATCTTTTTGCCGCGC |
| | | | CACTTAAAAAGCCAAGCTATCATTGTACTGTTGTCTGTCTTGCGCTT |
| SNP106 | 1 | 342 | TTACCGATATGTCAGACAGTAAGATGGAGAATTTTGTTCCTGCTTATGAAATC |
| | | | GTCAAATTTTACCTGTTTTTCGAGAAATGGAGGCGTGGAGAGATAGAG |
| SNP106 | 2 | 343 | TTACCGATATGTCAGACAGTAAGATGGAGAATTTTGTTCCTGCTTATGAATTC |
| | | | GTCAAATTTTACCTGTTTTTCGAGAAATGGAGGCGTGGAGAGATAGAG |
| SNP107 | 1 | 344 | TTTGACGCGGAAAAGTGAAGATAGCCTCCAAGCATTTCAATGCCGTCTCCAC |
| | | | TTTTGCTTGGAGTGAGATTACCCCCGAACATAAGAAGAGAATAATCGGA |
| SNP107 | 2 | 345 | TTTGACGCGGAAAAGTGAAGATAGCCTCCAAGCATTTCAATGCCGTCTCCTC |
| | | | TTTTGCTTGGAGTGAGATTACCCCCGAACATAAGAAGAGAATAATCGGA |
| SNP108 | 1 | 346 | GGAGGTAGCAATAACAACAACAACAACAACAACGGGGCCCACCACCACCAT |
| | | | CATCAGAACCAGTTTGATAACAATAACAACAACAACCTCATTGGCTCGTC |
| SNP108 | 2 | 347 | GGAGGTAGCAATAACAACAACAACAACAACAACGGGGCCCACCACCACCAC |
| | | | CATCAGAACCAGTTTGATAACAATAACAACAACAACCTCATTGGCTCGTC |
| SNP109 | 1 | 348 | ATGCCCAGCCCATTGAACAGCATGGCAATGCTGTGGCAATGAAATCTGAAAT |
| | | | CACTGACGCAATGATGGAAATTCCTGCTAAGGCTGTGCTAGTCAGTCC |
| SNP109 | 2 | 349 | ATGCCCAGCCCATTGAACAGCATGGCAATGCTGTGGCAATGAAATCTGAAGT |
| | | | CACTGACGCAATGATGGAAATTCCTGCTAAGGCTGTGCTAGTCAGTCCC |
| SNP110 | 1 | 350 | CCTTTATTTGTGTGATTATTTTATGGTTTTCAATTAGGTCTCATGATGCCAATG |
| | | | AGTTATGGATATTACTGANGCTTTGTGATTAGGTAAAGGTGATCACACTTTGT |
| | | | CAATCAAGAGACAAGGATGGCGAAGATGGTAATAAAAGCAACCATTTCTTTT |
| | | | TTCGAGTGCTTTCTTCCAGTTTAGATTATAAAAGTTCCTCTT |
| SNP110 | 2 | 351 | CCTTTATTTGTGTGATTATTTTATGGTTTTCAATTAGGTCTCATGATGCCAATG |
| | | | AGTTATGGATATTACTGANGCTTTGTGATTAGGTAAAGGTGATCACTCTTTGT |
| | | | CAATCAAGAGACAAGGATGGCGAAGATGGTAATAAAAGCAACCATTTCTTTT |
| | | | TTCGAGTGCTTTCTTCCAGTTTAGATTATAAAAGTTCCTCTT |
| SNP111 | 1 | 352 | AATCGTTGTACATGCTATATTTTCGCTTGTGCCTCTTCTCCCATGGACTTAGA |
| | | | AAGTATGTTTTTGCTAGTGTAAGAGGAATGCAACGGGATCGTTTCGTT |
| SNP111 | 2 | 353 | AATCGTTGTACATGCTATATTTTCGCTTGTGCCTCTTCTCCCATGGACTTGGA |
| | | | AAGTATGTTTTTGCTAGTGTAAGAGGAATGCAACGGGATCGTTTCGTT |
| SNP112 | 1 | 354 | CAAATGCATGTAATTTGCAGAAACAACTGCATCCACTGAAGCTCTATTACTG |
| | | | ATACAGAAATATTAGGGGTAGAGAATATTCCAGTATTAATTAAATTGAC |
| SNP112 | 2 | 355 | CAAATGCATGTAATTTGCAGAAACAACTGCATCCACTGAAGCTCTATTACCG |
| | | | ATACAGAAATATTAGGGGTAGAGAATATTCCAGTATTAATTAAATTGAC |
| SNP113 | 1 | 356 | GATGATATCGGTCGTCTCATGGATGATGATATCTCTAAGGTTTCTTCTGTTCT |
| | | | CTCTGTTTCCAGAACTGATGCATCTGCTTTACTCCGTCGGTATAACTG |
| SNP113 | 2 | 357 | GATGATATCGGTCGTCTCATGGATGATGATATCTCTAAGGTTTCTTCTGTCCT |
| | | | CTCTGTTTCCAGAACTGATGCATCTGCTTTACTCCGTCGGTATAACTG |
| SNP114 | 1 | 358 | TCAAGCGCTGAACTTCTTGCCACCTTCTGTGACAACATTCTCAAAAAAGGAG |
| | | | GGAGTGAGAAATTGAGTGATGAAGCTATTGAAGAAACGTTGGAAAAGGT |
| SNP114 | 2 | 359 | TCAAGCGCTGAACTTCTTGCCACCTTCTGTGACAACATTCTCAAAAAAGGCG |
| | | | GGAGTGAGAAATTGAGTGATGAAGCTATTGAAGAAACGTTGGAAAAGGT |
| SNP115 | 1 | 360 | CAAAATTTGGGAGAGCTGAAGCAGAGTTTCCCACTCAAGGTAAATGTATATA |
| | | | GCTAGTCAAAAGTATGCCAGTTGTGTCCTGTTGCTTGTGTATATAGTTC |
| SNP115 | 2 | 361 | CAAAATTTGGGAGAGCTGAAGCAGAGTTTCCCACTCAAGGTAAATGTATAGA |
| | | | GCTAGTCAAAAGTATGCCAGTTGTGTCCTGTTGCTTGTGTATATAGTTC |
| SNP116 | 1 | 362 | GTAGAGTTATGTTGGTGGAAGGAAGTACAGAGAAGAAGCATAGAGATGTTT |
| | | | GAGAATTGGGTGGGAGATTGTTTTTTCCAGAGCTCCAACTATATGATATA |
| SNP116 | 2 | 363 | GTAGAGTTATGTTGGTGGAAGGAAGTACAGAGAAGAAGCATAGAGATGTTC |
| | | | GAGAATTGGGTGGGAGATTGTTTTTTCCAGAGCTCCAACTATATGATATA |
| SNP117 | 1 | 364 | CTGCTTTGGCTTTTCCTCTACAACAGGAACATCGTCAACACCTTGAAAACTTG |
| | | | TGTCTGTGGAGGAACTATTTTCGCCAGAAATCTGAATGATCTGCTTCA |
| SNP117 | 2 | 365 | CTGCTTTGGCTTTTCCTCTACAACAGGAACATCGTCAACACCTTGAAAACCTG |
| | | | TGTCTGTGGAGGAACTATTTTCGCCAGAAATCTGAATGATCTGCTTCA |
| SNP118 | 1 | 366 | GTACTGGTTTTGGTTTAAAAAAATGAAGACATCAATGATTGACAGTGCTCAT |
| | | | CCTACTATCATGCTCATTACCAGGGTGGGAAGAAGCTCCTATCATCAGA |
| SNP118 | 2 | 367 | GTACTGGTTTTGGTTTAAAAAAATGAAGACATCAATGATTGACAGTGCTCGT |
| | | | CCTACTATCATGCTCATTACCAGGGTGGGAAGAAGCTCCTATCATCAGA |
| SNP119 | 1 | 368 | GTGAAATTGCACTGTTTCCCCTTTCATCTTGAGGGAGCTCACATGCTTCATTT |
| | | | ATAGACCCCGATTTCAGATCAGCTGGTGGGATGAAGCAGTCTACTGAT |
| SNP119 | 2 | 369 | GTGAAATTGCACTGTTTCCCCTTTCATCTTGAGGGAGCTCACATGCTTCACTT |
| | | | ATAGACCCCGATTTCAGATCAGCTGGTGGGATGAAGCAGTCTACTGAT |
| SNP120 | 1 | 370 | TAAGTTTTTGACTCTATTAACTCTGTGTGGCCTGACAATGTAATTTGTCTTAG |
| | | | CTAGAGAAAAGTTCAGTCAGAAAATAAATTTCCCATCTCTCTCATTGT |
| SNP120 | 2 | 371 | TAAGTTTTTGACTCTATTAACTCTGTGTGGCCTGACAATGTAATTTGTCTCAG |
| | | | CTAGAGAAAAGTTCAGTCAGAAAATAAATTTCCCATCTCTCTCATTGT |
| SNP121 | 1 | 372 | AATAAAATGGTGAAATGGTGTCCAAGTATCCCTCATTGTGGGAATGCTATAC |
| | | | GAGTAGAGACCGATGAGTTCTGTGAAGTAGAATGTTCATGTGGTTTACA |
| SNP121 | 2 | 373 | AATAAAATGGTGAAATGGTGTCCAAGTATCCCTCATTGTGGGAATGCTATTC |
| | | | GAGTAGAGACCGATGAGTTCTGTGAAGTAGAATGTTCATGTGGTTTACA |
| SNP122 | 1 | 374 | TACATTGACAATGGCAATTTGGAGCAATGGCTACACGGTGATGTAGGGTCAG |
| | | | TTAGTCCTCTAACTTGGGAAATCAGACTGAGAATTGCAATTGGAACTGC |
| SNP122 | 2 | 375 | TACATTGACAATGGCAATTTGGAGCAATGGCTACACGGTGATGTAGGGTCTG |
| | | | TTAGTCCTCTAACTTGGGAAATCAGACTGAGAATTGCAATTGGAACTGC |
| SNP123 | 1 | 376 | GGAGACACTGTGAAGAGAATATGATGAGAGGAGTGCTCCGGAAACTTGGGT |
| | | | CGATATGCACATTGACATTATTTGCTAGAGCATTCACATGCACTTGGCAT |
| SNP123 | 2 | 377 | GGAGACACTGTGAAGAGAATATGATGAGAGGAGTGCTCCGGAAACTTGGGG |
| | | | CGATATGCACATTGACATTATTTGCTAGAGCATTCACATGCACTTGGCAT |
| SNP124 | 1 | 378 | GTATGCAGCTCACAACTACTAAACGGTCAATTTTAAGTGAGCATAGTGCCAT |
| | | | TGCCAATTCCCATTTCTCTAAATATCAAACCAGAAAACATATTTAACGT |
| SNP124 | 2 | 379 | GTATGCAGCTCACAACTACTAAACGGTCAATTTTAAGTGAGCATAGTGCCGT |
| | | | TGCCAATTCCCATTTCTCTAAATATCAAACCAGAAAACATATTTAACGT |
| SNP125 | 1 | 380 | GAGGAACATAAACCACTGAAGCATTAGCCTTTGTCTCTACTTTTGCCTCTACA |
| | | | ACAGTATTAAAAACAGGTAGCCCCAAATGTTCTGTTCCACCCTTCTTG |
| SNP125 | 2 | 381 | GAGGAACATAAACCACTGAAGCATTAGCCTTTGTCTCTACTTTTGCCTCTGCA |
| | | | ACAGTATTAAAAACAGGTAGCCCCAAATGTTCTGTTCCACCCTTCTTG |
| SNP126 | 1 | 382 | CCTCTTTGAAAATGTTTAAGATCTCGGCGAGCTTGGTGACCTCTAAAACAACT |
| | | | TTGAACACATAAGATGCCATGGAGAGTGCGATTTCTTGTATCTTCAAG |
| SNP126 | 2 | 383 | CCTCTTTGAAAATGTTTAAGATCTCGGCGAGCTTGGTGACCTCTAAAACAGC |
| | | | TTTGAACACATAAGATGCCATGGAGAGTGCGATTTCTTGTATCTTCAAG |
| SNP127 | 1 | 384 | TCAGCTTATTGGTGATTCCTGTTCATAGATTTGGCTGTATTTTATGTCTTCTAT |
| | | | TGCCTTAAATTCGTGTATGGACTATAAAAAACTGATTGTTTGTTGGA |
| SNP127 | 2 | 385 | TCAGCTTATTGGTGATTCCTGTTCATAGATTTGGCTGTATTTTATGTCTTGTAT |
| | | | TGCCTTAAATTCGTGTATGGACTATAAAAAACTGATTGTTTGTTGGA |
| SNP128 | 1 | 386 | AACGATAATCTTCTTACTTCTTCAATAAACCTCAGGAATTCTTTGGAGAATGG |
| | | | GACGCCATTGACGTTGATTCCGATGATGATAGCCATGGCTCCTTGAAT |
| SNP128 | 2 | 387 | AACGATAATCTTCTTACTTCTTCAATAAACCTCAGGAATTCTTTGGAGAACGG |
| | | | GACGCCATTGACGTTGATTCCGATGATGATAGCCATGGCTCCTTGAAT |
| SNP129 | 1 | 388 | AGGTTGAGATGGATGATTGGTTGTTCGAGTTTGCTCAGTTATTCAGGACTTAT |
| | | | GTTGGCATTGATCCGGATGCCCACATTGACCTGCACGAGCTTGGGATG |
| SNP129 | 2 | 389 | AGGTTGAGATGGATGATTGGTTGTTCGAGTTTGCTCAGTTATTCAGGACTCAT |
| | | | GTTGGCATTGATCCGGATGCCCACATTGACCTGCACGAGCTTGGGATG |
| SNP130 | 1 | 390 | CATATCCATTAAAGAAAGAGTTTAGATCCAAGACTGTGAATTAGGGCATTTA |
| | | | ATTACTCCACATGGCAAGATAGAAAGTATGTCACCCGGATTTAGAAGAT |
| SNP130 | 2 | 391 | CATATCCATTAAAGAAAGAGTTTAGATCCAAGACTGTGAATTAGGGCATTGA |
| | | | ATTACTCCACATGGCAAGATAGAAAGTATGTCACCCGGATTTAGAAGAT |
| SNP131 | 1 | 392 | TCAGATAGAAACAGTCAAGGCCAACAGGGAACAAGAGACTAAAGGCCTCAA |
| | | | TGATAAAATTTCTAGGATAGAGGCTGAACTTCAAGCTGCTGAATCTATCA |
| SNP131 | 2 | 393 | TCAGATAGAAACAGTCAAGGCCAACAGGGAACAAGAGACTAAAGGCCTCAG |
| | | | TGATAAAATTTCTAGGATAGAGGCTGAACTTCAAGCTGCTGAATCTATCA |
| SNP132 | 1 | 394 | CAACCGAGCCACCGGACTTGAGAATAAAGCGAAATTCCTCTTTGATTCACTG |
| | | | ATCAAAGAGCAGATTTCAACCTAACAGTTGAATCCATGCACAACTCAAT |
| SNP132 | 2 | 395 | CAACCGAGCCACCGGACTTGAGAATAAAGCGAAATTCCTCTTTGATTCACCG |
| | | | ATCAAAGAGCAGATTTCAACCTAACAGTTGAATCCATGCACAACTCAAT |
| SNP133 | 1 | 396 | TTCATTTTGTTGAAACCAAAATTATACATAGAATATCTGTCCAACATTTAATA |
| | | | GGCTCTCATGAGGGACATTGAAGATTACACTATTTTCCCTGCATATTT |
| SNP133 | 2 | 397 | TTCATTTTGTTGAAACCAAAATTATACATAGAATATCTGTCCAACATTTACTA |
| | | | GGCTCTCATGAGGGACATTGAAGATTACACTATTTTCCCTGCATATTT |
| SNP134 | 1 | 398 | TCCCTGTGTAGTATCATTTTGAAAGATACAAAGCAAACAAGATTGATTAGTC |
| | | | CCGCTTGCACGAATTTGTAAGATTTTTATATCTAGAACAGGATGATAGT |
| SNP134 | 2 | 399 | TCCCTGTGTAGTATCATTTTGAAAGATACAAAGCAAACAAGATTGATTAGCC |
| | | | CCGCTTGCACGAATTTGTAAGATTTTTATATCTAGAACAGGATGATAGT |
| SNP135 | 1 | 400 | ATTTAGAAAATCAGGATAGTAAACATTCCCTGGCCAAACAACCCCTTGGTAG |
| | | | GGCATATTATCGCGTTTTATGAAGACATCTGCTTCCATGCCTCTCCTAT |
| SNP135 | 2 | 401 | ATTTAGAAAATCAGGATAGTAAACATTCCCTGGCCAAACAACCCCTTGGTGG |
| | | | GGCATATTATCGCGTTTTATGAAGACATCTGCTTCCATGCCTCTCCTAT |
| SNP136 | 1 | 402 | TGAGAGAAGGAGTGGCGAAGCAGAGGAAGGATTTTGCGAAGGAGGCTAAAA |
| | | | AGTTTACTAATATTAGGCATCCTAATGTAGTAGGATTAAGAGGTTACTAC |
| SNP136 | 2 | 403 | TGAGAGAAGGAGTGGCGAAGCAGAGGAAGGATTTTGCGAAGGAGGCTAAA |
| | | | GAGTTTACTAATATTAGGCATCCTAATGTAGTAGGATTAAGAGGTTACTAC |
| SNP137 | 1 | 404 | CATATGACAAGATGGAGCAACAATTGTCAAAGACTCGTAACCTGCATTGTTA |
| | | | GATCAACTGTGAACAGGTGTTTTTTCTTCTCTTTTGCTTCATTTATTTA |
| SNP137 | 2 | 405 | CATATGACAAGATGGAGCAACAATTGTCAAAGACTCGTAACCTGCATTGTCA |
| | | | GATCAACTGTGAACAGGTGTTTTTTCTTCTCTTTTGCTTCATTTATTTA |
| SNP138 | 1 | 406 | GCCAAAGAGCAAGCTGAATCTGCTCAAGAGGAGGCAGAGGAGTGGAAACGT |
| | | | AAGTACGGCATTGCTGCCAAGGAAGCAAAGAATGCTCTTGAGAAGGCAGC |
| SNP138 | 2 | 407 | GCCAAAGAGCAAGCTGAATCTGCTCAAGAGGAGGCAGAGGAGTGGAAACGC |
| | | | AAGTACGGCATTGCTGCCAAGGAAGCAAAGAATGCTCTTGAGAAGGCAGC |
| SNP139 | 1 | 408 | ACCATGGACAGATGACCTTTTATGGCAGTACCACGAAGCTTGACAAGTTCAT |
| | | | GCAGCACAGTTTTCACCATTCTTAAAGGTTTATCATCGGCTCCCGCTCT |
| SNP139 | 2 | 409 | ACCATGGACAGATGACCTTTTATGGCAGTACCACGAAGCTTGACAAGTTCGT |
| | | | GCAGCACAGTTTTCACCATTCTTAAAGGTTTATCATCGGCTCCCGCTCT |
| SNP140 | 1 | 410 | GAAGATTTGTGGGTATTGATGCAAAAGAAGAATGTTGATGCTGACTTGGGAA |
| | | | GTTACACCATTAGATTACAAGGATTGGTTGCGAATAACCAGGTTAACGA |
| SNP140 | 2 | 411 | GAAGATTTGTGGGTATTGATGCAAAAGAAGAATGTTGATGCTGACTTGGGGA |
| | | | GTTACACCATTAGATTACAAGGATTGGTTGCGAATAACCAGGTTAACGA |
| SNP141 | 1 | 412 | AGACGTTATTGTGTTCTGAACCAGTGTAATTATGTTGTTCTTGATGAAGCTGA |
| | | | CCGTATGATTGACATGGGTTTTGAGCCTCAAGTTGTTGGTGTACTGGA |
| SNP141 | 2 | 413 | AGACGTTATTGTGTTCTGAACCAGTGTAATTATGTTGTTCTTGATGAAGCGGA |
| | | | CCGTATGATTGACATGGGTTTTGAGCCTCAAGTTGTTGGTGTACTGGA |
| SNP142 | 1 | 414 | CCCATATTGCTAATGCAGATCAAAGAGCGGGAGGATTAAGCTTCTATAGTAG |
| | | | AAGATATCCATCAAATGGAGTAGCAAACAAGCAATGTACCACAATATTA |
| SNP142 | 2 | 415 | CCCATATTGCTAATGCAGATCAAAGAGCGGGAGGATTAAGCTTCTATAGTCG |
| | | | AAGATATCCATCAAATGGAGTAGCAAACAAGCAATGTACCACAATATTA |
| SNP143 | 1 | 416 | TCACTTGATTTGATAGCAGAAGCAAAAGTAGATCCAGAATCCAGGTTAATAG |
| | | | AACATTTATTGCTTGTAAAAGATTTCTGGTTCTGACCAGAAGATGGATC |
| SNP143 | 2 | 417 | TCACTTGATTTGATAGCAGAAGCAAAAGTAGATCCAGAATCCAGGTTAATTG |
| | | | AACATTTATTGCTTGTAAAAGATTTCTGGTTCTGACCAGAAGATGGATC |
| SNP144 | 1 | 418 | GGAGGTCAGGCTGGGCAATTGGGAGTTGGGCCATTAAATGGATTCTTTTCAT |
| | | | GCAAGCTCAATGAATCTGAGATGATGCTCCGAAATATACCAGTGTTGGT |
| SNP144 | 2 | 419 | GGAGGTCAGGCTGGGCAATTGGGAGTTGGGCCATTAAATGGATTCTTTTCCT |
| | | | GCAAGCTCAATGAATCTGAGATGATGCTCCGAAATATACCAGTGTTGGT |
| SNP145 | 1 | 420 | GTGTAATACGATCTGCCAGACCGTGGTGGGCAGGGGATTCTGTTAGCTGATA |
| | | | ATGCTCCGTACGAAATGTAGTACTTGAATTTGTTCCACAATAGAGATCT |
| SNP145 | 2 | 421 | GTGTAATACGATCTGCCAGACCGTGGTGGGCAGGGGATTCTGTTAGCTGACA |
| | | | ATGCTCCGTACGAAATGTAGTACTTGAATTTGTTCCACAATAGAGATCT |
| SNP146 | 1 | 422 | CGTCGATCAAAATACTCTTCCAAACCTACCGCAAAAGATAATAGCAGGCAAT |
| | | | AACAACAAAGATTAATCTCCCCCCTATATATGACTTGAGTTGTCAGGAA |
| SNP146 | 2 | 423 | CGTCGATCAAAATACTCTTCCAAACCTACCGCAAAAGATAATAGCAGGCAGT |
| | | | AACAACAAAGATTAATCTCCCCCCTATATATGACTTGAGTTGTCAGGAA |
| SNP147 | 1 | 424 | CCAGGATCACCCCTCTAGTAGCCAGCCAAGTGAAAAAGCACACCTTCATCAG |
| | | | CACTTTGGAAATGTAAACTGCATCAAAACTTCCTCTTCCTAACCAAAAG |
| SNP147 | 2 | 425 | CCAGGATCACCCCTCTAGTAGCCAGCCAAGTGAAAAAGCACACCTTCATCGG |
| | | | CACTTTGGAAATGTAAACTGCATCAAAACTTCCTCTTCCTAACCAAAAG |
| SNP148 | 1 | 426 | ACTCCAAGGGACCAAATAAGCTTTGCCATTGTAAGAGATAAGATCATGTCAA |
| | | | AGACAAATTGGACTGTGAATATGTTCTTAGACTGCGAACGGCGTAACTT |
| SNP148 | 2 | 427 | ACTCCAAGGGACCAAATAAGCTTTGCCATTGTAAGAGATAAGATCATGTCCA |
| | | | AGACAAATTGGACTGTGAATATGTTCTTAGACTGCGAACGGCGTAACTT |
| SNP149 | 1 | 428 | CGGGTGTGCATGTTTCAATACTGACAAAAAAGGGCCTTCAATGGTGTTGATC |
| | | | GCGCTAGTGAGAAATGAATTTGGTGGTTCTCTGTGCCTTCTTTCCGCTA |
| SNP149 | 2 | 429 | CGGGTGTGCATGTTTCAATACTGACAAAAAAGGGCCTTCAATGGTGTTGACC |
| | | | GCGCTAGTGAGAAATGAATTTGGTGGTTCTCTGTGCCTTCTTTCCGCTA |
| SNP150 | 1 | 430 | AATTCCGCCAGCCATTTCTTCGCCGGCTTTTTCCCCCTCAACATCTCCGGTGA |
| | | | TGTCACGTTATCTATCAAATAGGGGAGGAACTCAAGTACAAAAGGTTC |
| SNP150 | 2 | 431 | AATTCCGCCAGCCATTTCTTCGCCGGCTTTTTCCCCCTCAACATCTCCGGCGA |
| | | | TGTCACGTTATCTATCAAATAGGGGAGGAACTCAAGTACAAAAGGTTC |
| SNP151 | 1 | 432 | ACAGCTCTACAACTATTGCTCCGTTCATTCCTTCCTAGCACTTTTGAGGCAAA |
| | | | GCTAGGAGGCTTAAGCTTCTCAACTTGACTCTGCAGAGATCCATGGCT |
| SNP151 | 2 | 433 | ACAGCTCTACAACTATTGCTCCGTTCATTCCTTCCTAGCACTTTTGAGGCTAA |
| | | | GCTAGGAGGCTTAAGCTTCTCAACTTGACTCTGCAGAGATCCATGGCT |
| SNP152 | 1 | 434 | ACTCTTGAACCATTTGAAAGAGACCACGCTTGTGTTGTTGGTGCCTATCGTGT |
| | | | ACCAAAGAAGCAAAAGGCTGCTGCCTAGAAAATTTAAGCTTATGATTT |
| SNP152 | 2 | 435 | ACTCTTGAACCATTTGAAAGAGACCACGCTTGTGTTGTTGGTGCCTATCGCGT |
| | | | ACCAAAGAAGCAAAAGGCTGCTGCCTAGAAAATTTAAGCTTATGATTT |
| SNP153 | 1 | 436 | AAACAGAAATTCGGGTTTCGGTTCAGAAAAACATCAATGAACAAAGTATCA |
| | | | ATCAAGCAATTAACTTGAATGGCACCCCTTTTGTTCTTCCAGAGTTTTTC |
| SNP153 | 2 | 437 | AAACAGAAATTCGGGTTTCGGTTCAGAAAAACATCAATGAACAAAGTATCG |
| | | | ATCAAGCAATTAACTTGAATGGCACCCCTTTTGTTCTTCCAGAGTTTTTC |
| SNP154 | 1 | 438 | CTCTCCTCTTTTGTTAATTCATCAATTTTTTCCTGCATCAGTTTCATTAGTGAT |
| | | | ACAACTTCTTCAGGCCTTCGCTTGAAGTTATCAATGCTGAATGCATA |
| SNP154 | 2 | 439 | CTCTCCTCTTTTGTTAATTCATCAATTTTTTCCTGCATCAGTTTCATTAGCGAT |
| | | | ACAACTTCTTCAGGCCTTCGCTTGAAGTTATCAATGCTGAATGCATA |
| SNP155 | 1 | 440 | GGGCGTGACTCAAACACTTGGGAAGATGCATTATCTTTCAGGCCAGAGCGAT |
| | | | TTCTCAACTCTAATGTGGATTTCAGGGGTCAAGATTTCGAGTTCATACC |
| SNP155 | 2 | 441 | GGGCGTGACTCAAACACTTGGGAAGATGCATTATCTTTCAGGCCAGAGCGGT |
| | | | TTCTCAACTCTAATGTGGATTTCAGGGGTCAAGATTTCGAGTTCATACC |
| SNP156 | 1 | 442 | GAAGAGTATGCTCTTCGACGTCTAAGGCTTTTGCAGCACATCTGCTAACGTG |
| | | | GAGCATATTTGTTCTCTCATCGCATCCCTGTTATTTGCATCAAGTGTAA |
| SNP156 | 2 | 443 | GAAGAGTATGCTCTTCGACGTCTAAGGCTTTTGCAGCACATCTGCTAACGCG |
| | | | GAGCATATTTGTTCTCTCATCGCATCCCTGTTATTTGCATCAAGTGTAA |
| SNP157 | 1 | 444 | TTCCATCAGCTTATGAACTCCAAACGTTGTTTGTCAATGAGTTGTGCTAAATA |
| | | | CTACCTCTTTGACCATCAGTTCATTTACCAGGCAAATCAACAGGGCAA |
| SNP157 | 2 | 445 | TTCCATCAGCTTATGAACTCCAAACGTTGTTTGTCAATGAGTTGTGCTAAGTA |
| | | | CTACCTCTTTGACCATCAGTTCATTTACCAGGCAAATCAACAGGGCAA |
| SNP158 | 1 | 446 | AAGTCCATTGAATCCAATATCGAGGCAGGGTTGCCCTGGGTGAGATCTACAG |
| | | | TAGTTATGGACTCTAAATTAGCAGACTCTGGTGAGAAGTTTGCAATATT |
| SNP158 | 2 | 447 | AAGTCCATTGAATCCAATATCGAGGCAGGGTTGCCCTGGGTGAGATCTACTG |
| | | | TAGTTATGGACTCTAAATTAGCAGACTCTGGTGAGAAGTTTGCAATATT |
| SNP159 | 1 | 448 | GAATGGAACTATCACCACAAGTACACAACTAAAGCTTTGATGAAAACACCA |
| | | | GAGCTGTTTCAGTCTTGACGAACATACCAACCGCATTCTAGTGCTTGAAA |
| SNP159 | 2 | 449 | GAATGGAACTATCACCACAAGTACACAACTAAAGCTTTGATGAAAACACCG |
| | | | GAGCTGTTTCAGTCTTGACGAACATACCAACCGCATTCTAGTGCTTGAAA |
| SNP160 | 1 | 450 | CCCTACTGATGTTGAGCTGATAATGTACTATCTAAAGCGGAAGATCATGGTG |
| | | | AAAAAGATCCTTTTTGAAGTCATATCAGAACTCAACATTTATAAGTTCT |
| SNP160 | 2 | 451 | CCCTACTGATGTTGAGCTGATAATGTACTATCTAAAGCGGAAGATCATGGGG |
| | | | AAAAAGATCCTTTTTGAAGTCATATCAGAACTCAACATTTATAAGTTCT |
| SNP161 | 1 | 452 | AGATTTTGAAGTTCACCCAACTCTTCAATTCTTGAATCACTAAATGCACCTAC |
| | | | GACAAATCTTGTTGAATAAGAAAGTAGGAGAATCTGTAACTTGCTTTT |
| SNP161 | 2 | 453 | AGATTTTGAAGTTCACCCAACTCTTCAATTCTTGAATCACTAAATGCACCCAC |
| | | | GACAAATCTTGTTGAATAAGAAAGTAGGAGAATCTGTAACTTGCTTTT |
| SNP162 | 1 | 454 | ATAATGCAAGTCTCACATTTTTCGTGCAGATAAAGTATACCCCTTGATATATC |
| | | | CAATGCAAGTCTCATCCTTTGCTCCCAAGAAGGTCTTGTTTCAGAACT |
| SNP162 | 2 | 455 | ATAATGCAAGTCTCACATTTTTCGTGCAGATAAAGTATACCCCTTGATATGTC |
| | | | CAATGCAAGTCTCATCCTTTGCTCCCAAGAAGGTCTTGTTTCAGAACT |
| SNP163 | 1 | 456 | TACCAATAGATTCTCCGATCAAAGAGTTTTCCTTGGGCTCAAGCACACCGTC |
| | | | GGACTTTGACAAGATCAAAAACAATTTTTTTGCATCGTCGTCAGTTAAC |
| SNP163 | 2 | 457 | TACCAATAGATTCTCCGATCAAAGAGTTTTCCTTGGGCTCAAGCACACCGCC |
| | | | GGACTTTGACAAGATCAAAAACAATTTTTTTGCATCGTCGTCAGTTAAC |
| SNP164 | 1 | 458 | GCTATCAACTGGTGTATATGCAAGGGTACAATTCCTATCCCTGGAATCAAAT |
| | | | CTGTAAAACAAACTGAAGAGAACCTAGGAGCCCTTGGTTGGCAACTCAG |
| SNP164 | 2 | 459 | GCTATCAACTGGTGTATATGCAAGGGTACAATTCCTATCCCTGGAATCAAGT |
| | | | CTGTAAAACAAACTGAAGAGAACCTAGGAGCCCTTGGTTGGCAACTCAG |
| SNP165 | 1 | 460 | TCATGCTGGCAGAGGAAGTGAGAGAAATTATGTCTCAACTTGGTTTCAGAAC |
| | | | ACTTACTGAAATGGTTGGCCGTTCAGACATGCTTGAAATGGACAATGAT |
| SNP165 | 2 | 461 | TCATGCTGGCAGAGGAAGTGAGAGAAATTATGTCTCAACTTGGTTTCAGAGC |
| | | | ACTTACTGAAATGGTTGGCCGTTCAGACATGCTTGAAATGGACAATGAT |
| SNP166 | 1 | 462 | AAAAGCCATCCTTAAGTTTCCGTTCGATGCCGGAAATTTAATACCGCAGGAG |
| | | | ACGATTGGCCGGAAGAGGAGAATTGATCAGTGATGCACTTAGTTGAGTG |
| SNP166 | 2 | 463 | AAAAGCCATCCTTAAGTTTCCGTTCGATGCCGGAAATTTAATACCGCAGGTG |
| | | | ACGATTGGCCGGAAGAGGAGAATTGATCAGTGATGCACTTAGTTGAGTG |
| SNP167 | 1 | 464 | CAAAACATAGAATATCATGCAAGTTGCTGGTTAAGTGCCAAGAACATGGATG |
| | | | TTCATTGCATGTTTAATTAACTCTTTCGGGTGAGATCAAAATCTCTGGT |
| SNP167 | 2 | 465 | CAAAACATAGAATATCATGCAAGTTGCTGGTTAAGTGCCAAGAACATGGAC |
| | | | GTTCATTGCATGTTTAATTAACTCTTTCGGGTGAGATCAAAATCTCTGGT |
| SNP168 | 1 | 466 | TCGCAACTCTTATACTATGATGGGTGTTCAATTATTGAGATGTGTACCAAATG |
| | | | CTTGTAACTGCTCACTTTAAGATGAAAAGCTGCCTTCACTGGTTAGTG |
| SNP168 | 2 | 467 | TCGCAACTCTTATACTATGATGGGTGTTCAATTATTGAGATGTGTACCAAGTG |
| | | | CTTGTAACTGCTCACTTTAAGATGAAAAGCTGCCTTCACTGGTTAGTG |
| SNP169 | 1 | 468 | TGATGTAAGCAAATTTTCAAGTCGCCATGTGGTGAGTGCTGCATATGATCAC |
| | | | ACAATAAAAGTTTGGGATCTGCAGAAGGGTTACTGTAACAACACTATCA |
| SNP169 | 2 | 469 | TGATGTAAGCAAATTTTCAAGTCGCCATGTGGTGAGTGCTGCATATGATCGC |
| | | | ACAATAAAAGTTTGGGATCTGCAGAAGGGTTACTGTAACAACACTATCA |
| SNP170 | 1 | 470 | AGGAGGTGGCATCTGGGGTGGGAAGTAGGGGAATGGTTCAATTGGGAAACA |
| | | | ACCGGGAGGAACTGGAGCTCCATAAGGTGGCCCTCCGGGTGGCCCTCTAT |
| SNP170 | 2 | 471 | AGGAGGTGGCATCTGGGGTGGGAAGTAGGGGAATGGTTCAATTGGGAAACC |
| | | | ACCGGGAGGAACTGGAGCTCCATAAGGTGGCCCTCCGGGTGGCCCTCTAT |
| SNP171 | 1 | 472 | AGCGGCTGAGATAAAGGATTAAATCTACAAAACGAAGCGGAACTGGACGGC |
| | | | TTGTGAGGCTTCTTAGGGAGTGAAATTGACGGAGAAAACGAGATTGCTGT |
| SNP171 | 2 | 473 | AGCGGCTGAGATAAAGGATTAAATCTACAAAACGAAGCGGAACTGGACGGG |
| | | | TTGTGAGGCTTCTTAGGGAGTGAAATTGACGGAGAAAACGAGATTGCTGT |
| SNP172 | 1 | 474 | CTCCAAATATTGCAGCACCTGTTGCATTGTAGGTCTATCATCTGGATTTGAAT |
| | | | CAGTGCATCTTGCAGCTATTTCTATAATTGCTTCTACTGTCTCTGCAT |
| SNP172 | 2 | 475 | CTCCAAATATTGCAGCACCTGTTGCATTGTAGGTCTATCATCTGGATTTGCAT |
| | | | CAGTGCATCTTGCAGCTATTTCTATAATTGCTTCTACTGTCTCTGCAT |
| SNP173 | 1 | 476 | GGTAACCTAACTCCGGCTGAGACTCATCAGAATCCGCCGTCAAATCCACCAC |
| | | | TTCCGTCTCCATCACCTTCGCCGGTGACGGAGAAGCTGTAATTTCTTCT |
| SNP173 | 2 | 477 | GGTAACCTAACTCCGGCTGAGACTCATCAGAATCCGCCGTCAAATCCACCGC |
| | | | TTCCGTCTCCATCACCTTCGCCGGTGACGGAGAAGCTGTAATTTCTTCT |
| SNP174 | 1 | 478 | AACGAATCGAAATCATGACTCGTGGTGCAAAACTCGGAGCTATCATCGTCAT |
| | | | CGGTGAGATCGTACAACAGATTCTCCTTGAGTTTCTTCACTTCCGGTGA |
| SNP174 | 2 | 479 | AACGAATCGAAATCATGACTCGTGGTGCAAAACTCGGAGCTATCATCGTCGT |
| | | | CGGTGAGATCGTACAACAGATTCTCCTTGAGTTTCTTCACTTCCGGTGA |
| SNP175 | 1 | 480 | CTAAAATCAATTATCTTGAGCTGTGAACATTGAGACAGTTGAGATGGAATTG |
| | | | GACCATAAAGTGAGTTATCAGGTGCTCTCAGCTCTTCCAATGATGAAGC |
| SNP175 | 2 | 481 | CTAAAATCAATTATCTTGAGCTGTGAACATTGAGACAGTTGAGATGGAATCG |
| | | | GACCATAAAGTGAGTTATCAGGTGCTCTCAGCTCTTCCAATGATGAAGC |
| SNP176 | 1 | 482 | ATCGAAGCCAGAAATTGTGATTATTGATTGTGGATTAAGGAATTTTGGTGTG |
| | | | GAGTTGAAATGGATGGGAATAATTGGAGGGCCGCTCAGGCTCAGGCTCC |
| SNP176 | 2 | 483 | ATCGAAGCCAGAAATTGTGATTATTGATTGTGGATTAAGGAATTTTGGTGCG |
| | | | GAGTTGAAATGGATGGGAATAATTGGAGGGCCGCTCAGGCTCAGGCTCC |
| SNP177 | 1 | 484 | TCTCCGGCGACCGGAGAATCCTACGCCTGTAAATCTATCGATAAAAACCTTC |
| | | | TCATTGATTCCACCGACCGTGAGTGTCTCGATAAAGAACCCAAAATTCT |
| SNP177 | 2 | 485 | TCTCCGGCGACCGGAGAATCCTACGCCTGTAAATCTATCGATAAAAACCTCC |
| | | | TCATTGATTCCACCGACCGTGAGTGTCTCGATAAAGAACCCAAAATTCT |
| SNP178 | 1 | 486 | GATCGAAGGTCAATTGAGAGAAGCAGAAGAGACTATGACAGGAGCAGGAGC |
| | | | CGTAGTAGGAGTAGAAGCCACAGCCGAAGCTTGCATGATCAAGGTACAAG |
| SNP178 | 2 | 487 | GATCGAAGGTCAATTGAGAGAAGCAGAAGAGACTATGACAGGAGCAGGAG |
| | | | GCGTAGTAGGAGTAGAAGCCACAGCCGAAGCTTGCATGATCAAGGTACAAG |
| SNP179 | 1 | 488 | CTATATGGGTCAATATTTCCCTGCTCATGATCTGCAAGAAGAAGAGCTTTAGT |
| | | | ACAATCAGTGGAAGGATGTTCAGACGATCCTGTGTCACATGTTATCTG |
| SNP179 | 2 | 489 | CTATATGGGTCAATATTTCCCTGCTCATGATCTGCAAGAAGAAGAGCTTTGG |
| | | | TACAATCAGTGGAAGGATGTTCAGACGATCCTGTGTCACATGTTATCTG |
| SNP180 | 1 | 490 | TGGCCCAAATGAAGATATGACACCAGAAAATCGATCTGTTCTGTGCGTGCCA |
| | | | CTAGAGGAAAACGGGATAAACTTACCCCCACCCGGAGAGCCACCAAAGG |
| SNP180 | 2 | 491 | TGGCCCAAATGAAGATATGACACCAGAAAATCGATCTGTTCTGTGCGTGCGA |
| | | | CTAGAGGAAAACGGGATAAACTTACCCCCACCCGGAGAGCCACCAAAGG |
| SNP181 | 1 | 492 | ATAGACTCGAAGCATTTCTTCGTGCCTTCAGGAAATGCAGCTGTTGAATTAAT |
| | | | AGGAGGAAGGGAAACAGGCATTGCACAGACGATACGTACAATCCCAAA |
| SNP181 | 2 | 493 | ATAGACTCGAAGCATTTCTTCGTGCCTTCAGGAAATGCAGCTGTTGAATTGA |
| | | | TAGGAGGAAGGGAAACAGGCATTGCACAGACGATACGTACAATCCCAAA |
| SNP182 | 1 | 494 | CTTCAGCAACTTTACCATCTTCATTCACTTTATTCTTCCCTTTGAAGCCACAAC |
| | | | CAAAAACAAATGTGTCATTCAGTTTTGGACCCTGATACTCTGGTTCC |
| SNP182 | 2 | 495 | CTTCAGCAACTTTACCATCTTCATTCACTTTATTCTTCCCTTTGAAGCCAGAA |
| | | | CCAAAAACAAATGTGTCATTCAGTTTTGGACCCTGATACTCTGGTTCC |
| SNP183 | 1 | 496 | CCATTCAGAAAATAGATTCTTAAAAGTTGTGAGCCATCAAATCTCCAGCTTTT |
| | | | CAGTTTTGGGGTTGTAGTTTTCGGACTCTACATGTTATAACTACAATA |
| SNP183 | 2 | 497 | CCATTCAGAAAATAGATTCTTAAAAGTTGTGAGCCATCAAATCTCCAGCTCT |
| | | | TCAGTTTTGGGGTTGTAGTTTTCGGACTCTACATGTTATAACTACAATA |
| SNP184 | 1 | 498 | TTTATAAAATGTGATCTTGTATACTTCCTGGACAACCATCAACCATCCATTTG |
| | | | TTCTGCCTTTGTGTAGCCCTCACCTGGTTTTTTATTAACCGCTCCCAG |
| SNP184 | 2 | 499 | TTTATAAAATGTGATCTTGTATACTTCCTGGACAACCATCAACCATCCATCTG |
| | | | TTCTGCCTTTGTGTAGCCCTCACCTGGTTTTTTATTAACCGCTCCCAG |
| SNP185 | 1 | 500 | GAGAGACCACACAGATGCCGACGGCGATAGACAGCCGAATACTTTGTGTAA |
| | | | GCGTGCTCTGAAGCTCTCATCGTCCATGCCTTACCACTTTCTACTTCCTG |
| SNP185 | 2 | 501 | GAGAGACCACACAGATGCCGACGGCGATAGACAGCCGAATACTTTGTGTAC |
| | | | GCGTGCTCTGAAGCTCTCATCGTCCATGCCTTACCACTTTCTACTTCCTG |
| SNP186 | 1 | 502 | CCCGACTGTCATCAGCCAAACGATAAATATTCCTTCGAGAAAAGAAATGGAG |
| | | | TTTGTAAACATTTTGCCTCGTCTGGCTATCTGAACCTAGAAGAAACTCC |
| SNP186 | 2 | 503 | CCCGACTGTCATCAGCCAAACGATAAATATTCCTTCGAGAAAAGAAATGGGG |
| | | | TTTGTAAACATTTTGCCTCGTCTGGCTATCTGAACCTAGAAGAAACTCC |
| SNP187 | 1 | 504 | GACGCTCATTGCATCTCGCACTGATGGTGGCAAATTCCTCAAGATCTTGGTA |
| | | | GATTTATGCTATCCAAAGTGGGCACTGGAAGCATTTGTCATTGCAAATG |
| SNP187 | 2 | 505 | GACGCTCATTGCATCTCGCACTGATGGTGGCAAATTCCTCAAGATCTTGGCA |
| | | | GATTTATGCTATCCAAAGTGGGCACTGGAAGCATTTGTCATTGCAAATG |
| SNP188 | 1 | 506 | GAGCTTCGTCAGAGAGGTACTTCTGTTGTACCTCCAGGTGAAGTGTATGGAA |
| | | | GATGGGGTGGCATGGAATTTAAAGATAAAGAAATTGTGTGGCCACCAAT |
| SNP188 | 2 | 507 | GAGCTTCGTCAGAGAGGTACTTCTGTTGTACCTCCAGGTGAAGTGTATGGGA |
| | | | GATGGGGTGGCATGGAATTTAAAGATAAAGAAATTGTGTGGCCACCAAT |
| SNP189 | 1 | 508 | GTATGATGATCGAGCCATTTGCAATGTTTTAGTTGGTGACCTAAGAATTCTTG |
| | | | GTCTTCTAAAAGGTGCAAGCATGCGTTGTCTAATTCAGAAGAAACAAC |
| SNP189 | 2 | 509 | GTATGATGATCGAGCCATTTGCAATGTTTTAGTTGGTGACCTAAGAATTCCTG |
| | | | GTCTTCTAAAAGGTGCAAGCATGCGTTGTCTAATTCAGAAGAAACAAC |
| SNP190 | 1 | 510 | GGATGAATGGAGAGGGACATGGTCAATATCGATGCATCGCCCAAATGTTCTG |
| | | | TTTTTCCCCTTGGTGATTGTTCAATTATGATTTATGTACAATATATATA |
| SNP190 | 2 | 511 | GGATGAATGGAGAGGGACATGGTCAATATCGATGCATCGCCCAAATGTTCG |
| | | | GTTTTTCCCCTTGGTGATTGTTCAATTATGATTTATGTACAATATATATA |
| SNP191 | 1 | 512 | AACTTAATCAGCAGACATCTTTATACAAGAAAATGTGGCTAGCATACAAGTA |
| | | | ATAAAGTCAAATAAATAACAAATTGGCACAACAAAACAATTTGCTTAAA |
| SNP191 | 2 | 513 | AACTTAATCAGCAGACATCTTTATACAAGAAAATGTGGCTAGCATACAAGCA |
| | | | ATAAAGTCAAATAAATAACAAATTGGCACAACAAAACAATTTGCTTAAA |
| SNP192 | 1 | 514 | AATTGGCCGCATCTACATAATGCCCAGTTTTTCGTCTATGCCGACTATAATCT |
| | | | GATACAAACCTAAAAGTCAGTCCACAACCCTCAACCTTGCACTTGTAT |
| SNP192 | 2 | 515 | AATTGGCCGCATCTACATAATGCCCAGTTTTTCGTCTATGCCGACTATAACCT |
| | | | GATACAAACCTAAAAGTCAGTCCACAACCCTCAACCTTGCACTTGTAT |
| SNP193 | 1 | 516 | GAGAAAGCTAGAGAAGCCAGAGCTTCCTGCTAGCATTATTGAGATGAGTATA |
| | | | TTGATGTCGATAGTATTTTCTTCAACTCTATTTACCTGACAGACTTTGA |
| SNP193 | 2 | 517 | GAGAAAGCTAGAGAAGCCAGAGCTTCCTGCTAGCATTATTGAGATGAGTAG |
| | | | ATTGATGTCGATAGTATTTTCTTCAACTCTATTTACCTGACAGACTTTGA |
| SNP194 | 1 | 518 | TCCCCCTATACATTAGCCTTGGAATGACTCCTCTTCATCTGGCAAGACAATAG |
| | | | CTAGTAAAGAGAACTTAACACGAAGAGAAATCTTAGCCTTGTCCACTA |
| SNP194 | 2 | 519 | TCCCCCTATACATTAGCCTTGGAATGACTCCTCTTCATCTGGCAAGACAACAG |
| | | | CTAGTAAAGAGAACTTAACACGAAGAGAAATCTTAGCCTTGTCCACTA |
| SNP195 | 1 | 520 | ATTCTCAAAGTGGAGCTTTCGAGTGCTTCAATTGTTCAAAAAAGAGAGTGTG |
| | | | GCTCTTATTGGCTGGCTCCCATCTGTTGTTGTTTGCCTAAGGACAAAAG |
| SNP195 | 2 | 521 | ATTCTCAAAGTGGAGCTTTCGAGTGCTTCAATTGTTCAAAAAAGAGAGTGCG |
| | | | GCTCTTATTGGCTGGCTCCCATCTGTTGTTGTTTGCCTAAGGACAAAAG |
| SNP196 | 1 | 522 | AAGTAGAAAGTACTGCCAATGAAGTTCCAAATGATCCTTCCAAGATCACAAA |
| | | | CATCAAAGAAATTGCCACCTATGACAGTAAAGACATGCCTTCACTTGAACTT |
| | | | AGTTTGAAGCAACTTCGAGATGTTGGAGAGAATGGGACTGGTGTGCAAGAG |
| | | | CGAAATATACTCAGGCATTCAGATCTGTCAGCGTTCTCTAGGCATG |
| SNP196 | 2 | 523 | AAGTAGAAAGTACTGCCAATGAAGTTCCAAATGATCCTTCCAAGATCACAAA |
| | | | CATCAAAGAAATTGCCACCTATGACAGTAAAGACATGCCTTCACTTGAGCTT |
| | | | AGTTTGAAGCAACTTCGAGATGTTGGAGAGAATGGGACTGGTGTGCAAGAG |
| | | | CGAAATATACTCAGGCATTCAGATCTGTCAGCGTTCTCTAGGCATG |
| SNP197 | 1 | 524 | TTTTCGCCTATAGAGTCAGGTATTAAGAAAGTGGCAAAGGATTTTGAGCATT |
| | | | GTTGGCCTGGTAAAGCTGAGAGTTGTACTAGTAGTGGGTATGGATTAGA |
| SNP197 | 2 | 525 | TTTTCGCCTATAGAGTCAGGTATTAAGAAAGTGGCAAAGGATTTTGAGCACT |
| | | | GTTGGCCTGGTAAAGCTGAGAGTTGTACTAGTAGTGGGTATGGATTAGA |
| SNP198 | 1 | 526 | CTGAACCAAAACAATGGACTGATGGTAAATTGAGCAAGAGAATCGGAAGGA |
| | | | GAGGAACTCCATTTGTATTGAGACTGCTAATTGCTGTTTTCCCATTCTTA |
| SNP198 | 2 | 527 | CTGAACCAAAACAATGGACTGATGGTAAATTGAGCAAGAGAATCGGAAGGG |
| | | | GAGGAACTCCATTTGTATTGAGACTGCTAATTGCTGTTTTCCCATTCTTA |
| SNP199 | 1 | 528 | TGACCATGAGAGGGAGAAATAATATTGTGATATATGAAGAAGAGCTTGGCT |
| | | | AATAGTGGTGGAAGTCACAGATGAACCAATCTTAGGCTTCAAAAGATTGT |
| SNP199 | 2 | 529 | TGACCATGAGAGGGAGAAATAATATTGTGATATATGAAGAAGAGCTTGGCG |
| | | | AATAGTGGTGGAAGTCACAGATGAACCAATCTTAGGCTTCAAAAGATTGT |
| SNP200 | 1 | 530 | ATAATAAGAATTGGGAAACAATTGGAGTACTTTGAACAATATCAAAGGAGA |
| | | | GTAAGTGGTTTAATTGGAGCAGCACAAACAGAGCAGCTAGTAAACAGTGC |
| SNP200 | 2 | 531 | ATAATAAGAATTGGGAAACAATTGGAGTACTTTGAACAATATCAAAGGAGG |
| | | | GTAAGTGGTTTAATTGGAGCAGCACAAACAGAGCAGCTAGTAAACAGTGC |
| SNP201 | 1 | 532 | TCATATCGATCTGCATGTCTAAGCTTAAGCCTAATTGACGAATTCCCATATGA |
| | | | TTTTGCAGTTGTGTGCCAGACACCGGTAACAGCATATCGACTCCCTGA |
| SNP201 | 2 | 533 | TCATATCGATCTGCATGTCTAAGCTTAAGCCTAATTGACGAATTCCCATACGA |
| | | | TTTTGCAGTTGTGTGCCAGACACCGGTAACAGCATATCGACTCCCTGA |
| SNP202 | 1 | 534 | ACAAGCAACAGCAGCATGTATTTAACAAACAAGGAACTATGTACGGTTAGA |
| | | | GTCCTTAATGCTGTCAGTCAAGATGAAGTCACTGTAAGTATATTAATGGT |
| SNP202 | 2 | 535 | ACAAGCAACAGCAGCATGTATTTAACAAACAAGGAACTATGTACGGTTAGG |
| | | | GTCCTTAATGCTGTCAGTCAAGATGAAGTCACTGTAAGTATATTAATGGT |
| SNP203 | 1 | 536 | TTTTTGTTTAATCAGATATTTTGAATTCGAGCTTTGTTCGTTAAAATGTTTTTG |
| | | | CTAGGGAACGTTTTATGCTAATGTATATTTGAAATGATGTGCTAGAA |
| SNP203 | 2 | 537 | TTTTTGTTTAATCAGATATTTTGAATTCGAGCTTTGTTCGTTAAAATGTTCTTG |
| | | | CTAGGGAACGTTTTATGCTAATGTATATTTGAAATGATGTGCTAGAA |
| SNP204 | 1 | 538 | CTAGAAGCAAAAGGGTAACTAGCAGCAACAGAAGCAGCAGCAGCTGTGGTA |
| | | | GCCAACTTGGCTGAGAAAATACCATAAGAATGAAGGGTTTGACCATTAAA |
| SNP204 | 2 | 539 | CTAGAAGCAAAAGGGTAACTAGCAGCAACAGAAGCAGCAGCAGCTGTGGTT |
| | | | GCCAACTTGGCTGAGAAAATACCATAAGAATGAAGGGTTTGACCATTAAA |
| SNP205 | 1 | 540 | GATGTAACCTGAATGTCTCCTTGAGCACTAGTTCTAAGTATTCCAACTTAAGT |
| | | | AAGTCATCTTCCTCAACCATTCTATCAAGTCCTACAATAGAAGTCAAC |
| SNP205 | 2 | 541 | GATGTAACCTGAATGTCTCCTTGAGCACTAGTTCTAAGTATTCCAACTTAGGT |
| | | | AAGTCATCTTCCTCAACCATTCTATCAAGTCCTACAATAGAAGTCAAC |
| SNP206 | 1 | 542 | CTGTGTCATTTTTTTTTCTAACACTATACTAGTCTTTTTGCCGCCGGCGGTATG |
| | | | AGTTTATTCAGGTAAAAGGGAAAAGGGTATTCATATATAAGCCTAAA |
| SNP206 | 2 | 543 | CTGTGTCATTTTTTTTTCTAACACTATACTAGTCTTTTTGCCGCCGGCGGGATG |
| | | | AGTTTATTCAGGTAAAAGGGAAAAGGGTATTCATATATAAGCCTAAA |
| SNP207 | 1 | 544 | AGGGCAATATTACTCCCCTTGATCGCCATTGTAGACCTTTGTGAAATAGAAC |
| | | | AGTTTCCACTCACTTTACGTTTTACTTCTGAGAAAGTAAGCGCCCATTT |
| SNP207 | 2 | 455 | AGGGCAATATTACTCCCCTTGATCGCCATTGTAGACCTTTGTGAAATAGAGC |
| | | | AGTTTCCACTCACTTTACGTTTTACTTCTGAGAAAGTAAGCGCCCATTT |
| SNP208 | 1 | 546 | AACTGCACATTGTTTGCATTCAGAAGGTTTTGGAATTCACTGTAGTCTATTCT |
| | | | ATGAGAATTTTCAGGATCCCACTCTGTCCCCTTCAGCTTTCCCTGCAT |
| SNP208 | 2 | 547 | AACTGCACATTGTTTGCATTCAGAAGGTTTTGGAATTCACTGTAGTCTATCCT |
| | | | ATGAGAATTTTCAGGATCCCACTCTGTCCCCTTCAGCTTTCCCTGCAT |
| SNP209 | 1 | 548 | GAAGCAAATAGTGTGCAGAGGAAGAGAAAATAGAGATGTCGAAGACGCTAC |
| | | | TTCAGCCTGTAGGCCAAAAGAGACTTACCAATGTTGCTGTTGTGCGTCTC |
| SNP209 | 2 | 549 | GAAGCAAATAGTGTGCAGAGGAAGAGAAAATAGAGATGTCGAAGACGCTAG |
| | | | TTCAGCCTGTAGGCCAAAAGAGACTTACCAATGTTGCTGTTGTGCGTCTC |
| SNP210 | 1 | 550 | GTTCCCATAGTACCCAAAGAAAAGAAGGTCCCTCCAACTGGTGACAAGGTAA |
| | | | AAAATGGAGATTTCTCAGGTGACAATAATAATGTCAAAAAATGAAAGTT |
| SNP210 | 2 | 551 | GTTCCCATAGTACCCAAAGAAAAGAAGGTCCCTCCAACTGGTGACAAGGTCA |
| | | | AAAATGGAGATTTCTCAGGTGACAATAATAATGTCAAAAAATGAAAGTT |
| SNP211 | 1 | 552 | TCAATACAATACCTTTACCACTATTATAAAGAAAAAGGACAACCAGGTGCAC |
| | | | GAAACATCACATGTTCACGCATGGTCTGAGGAAGGGCCACATTCCAAGG |
| SNP211 | 2 | 553 | TCAATACAATACCTTTACCACTATTATAAAGAAAAAGGACAACCAGGTGCGC |
| | | | GAAACATCACATGTTCACGCATGGTCTGAGGAAGGGCCACATTCCAAGG |
| SNP212 | 1 | 554 | GACTCTAGTGGAGTTGAAGTAGGAGCAATGTTGGTGATGGTTATTGGATATA |
| | | | GGGTGTTAGCCTACTTCCTCCTAAGAAAAATGAAACCAAGAACAAGCAA |
| SNP212 | 2 | 555 | GACTCTAGTGGAGTTGAAGTAGGAGCAATGTTGGTGATGGTTATTGGATACA |
| | | | GGGTGTTAGCCTACTTCCTCCTAAGAAAAATGAAACCAAGAACAAGCAA |
| SNP213 | 1 | 556 | TAATATTATTTGTTCATTTTAAGATGAATAAAGAATTAAGTCTGCAAGATCTT |
| | | | TATGGTCAAACCACTACCCTTGGAACTCATTGAGATTAGATCTTATAT |
| SNP213 | 2 | 557 | TAATATTATTTGTTCATTTTAAGATGAATAAAGAATTAAGTCTGCAAGATGTT |
| | | | TATGGTCAAACCACTACCCTTGGAACTCATTGAGATTAGATCTTATAT |
| SNP214 | 1 | 558 | ATGTTTTACCAAGTACTATCTGCTGCAAGTTGTTTTTTCTTTAATCTAGACACT |
| | | | CCAGATCAAGTTTATTTAGAACACAAAAAACCATAGAAGAAAAAGGT |
| SNP214 | 2 | 559 | ATGTTTTACCAAGTACTATCTGCTGCAAGTTGTTTTTTCTTTAATCTAGAGAC |
| | | | TCCAGATCAAGTTTATTTAGAACACAAAAAACCATAGAAGAAAAAGGT |
| SNP215 | 1 | 560 | TGATTACACGTGCAATCCTCTGGAGAAGCTCCAATACTAGAGAAGGCGACAA |
| | | | ATTTGTCCTAGATGTCGCAACAAAAAGCAGACCAACTACCTTCACATGG |
| SNP215 | 2 | 561 | TGATTACACGTGCAATCCTCTGGAGAAGCTCCAATACTAGAGAAGGCGACCA |
| | | | ATTTGTCCTAGATGTCGCAACAAAAAGCAGACCAACTACCTTCACATGG |
| SNP216 | 1 | 562 | TGGACTTCCAAACCAACTGGTGCCTTTATACTTGCTGTGTATGTTGCGTCTTC |
| | | | TTGTCCTACGTTAGTTACAGTTCTTGTTACTTTCTTGATTTCATTTTC |
| SNP216 | 2 | 563 | TGGACTTCCAAACCAACTGGTGCCTTTATACTTGCTGTGTATGTTGCGTCGTC |
| | | | TTGTCCTACGTTAGTTACAGTTCTTGTTACTTTCTTGATTTCATTTTC |
| SNP217 | 1 | 564 | TGTTTTTTTATCATATTGCTCTGAAAAACAAAATATGGAGTGATGGAGCATTG |
| | | | TGGACAAGTAAACTCCACCCCATCCAAATAATACACCAGAAAAGTACA |
| SNP217 | 2 | 565 | TGTTTTTTTATCATATTGCTCTGAAAAACAAAATATGGAGTGATGGAGCAGT |
| | | | GTGGACAAGTAAACTCCACCCCATCCAAATAATACACCAGAAAAGTACA |
| SNP218 | 1 | 566 | AGATATTTTTAAAATTGGATGATTTTTTTTCGAGTGATTTTGGGCGTTCCATTT |
| | | | TCCTGGGTTTATTGAGTTTCTTCCTCTGTTTGATCTGACCAATGCAC |
| SNP218 | 2 | 567 | AGATATTTTTAAAATTGGATGATTTTTTTTCGAGTGATTTTGGGCGTTCCGTTT |
| | | | TCCTGGGTTTATTGAGTTTCTTCCTCTGTTTGATCTGACCAATGCAC |
| SNP219 | 1 | 568 | TCCAGGTAGTCTTTTGGTACTTCCATTTCAGATAGTGTGCTGGCATTGATTGC |
| | | | CTGAGCTGCCTTGAAAATTTGATTTGTGCATTCCCTACACTGCCGCAA |
| SNP219 | 2 | 569 | TCCAGGTAGTCTTTTGGTACTTCCATTTCAGATAGTGTGCTGGCATTGATCGC |
| | | | CTGAGCTGCCTTGAAAATTTGATTTGTGCATTCCCTACACTGCCGCAA |
| SNP220 | 1 | 570 | CCGGTTCACATTTCGTCATTCAGCTAAATCTCTGGCCAAAGTTGACGCCATTC |
| | | | ACTCTCCTTTGCTCCTTCCGATCTCAATCTCCGGTGAACTCCTGCGTG |
| SNP220 | 2 | 571 | CCGGTTCACATTTCGTCATTCAGCTAAATCTCTGGCCAAAGTTGACGCCACTC |
| | | | ACTCTCCTTTGCTCCTTCCGATCTCAATCTCCGGTGAACTCCTGCGTG |
| SNP221 | 1 | 572 | ACCAAATAAACAAGAAAAAAAATGATTTTTGGAGCTCAGAAGAAAGAAGAA |
| | | | CCAAAAGGGTCGATTCCTTTTCCTTCTAGAAATGTAAAAACCTAATTATT |
| SNP221 | 2 | 573 | ACCAAATAAACAAGAAAAAAAATGATTTTTGGAGCTCAGAAGAAAGAAGAG |
| | | | CCAAAAGGGTCGATTCCTTTTCCTTCTAGAAATGTAAAAACCTAATTATT |
| SNP222 | 1 | 574 | ATTGGCTTGGTCTAAATGAGTTCTCTGATTTGAGCCATGATGAGTTTAAGAAA |
| | | | ATGTATTTAGGACTGAAAGTTGATCAAGAGTTGCTTAATAAAAGAGAG |
| SNP222 | 2 | 575 | ATTGGCTTGGTCTAAATGAGTTCTCTGATTTGAGCCATGATGAGTTTAAGGA |
| | | | AATGTATTTAGGACTGAAAGTTGATCAAGAGTTGCTTAATAAAAGAGAG |
| SNP223 | 1 | 576 | AACTTGCTATTAGGATTGTTCCAAGCAGGTGCAGCTGCTTTTAGAAAAAGAA |
| | | | AAACCCCTCTTTCATTGACTGGTTGTGATGACTACTATCATTCGCCATA |
| SNP223 | 2 | 577 | AACTTGCTATTAGGATTGTTCCAAGCAGGTGCAGCTGCTTTTAGAAAAAGGA |
| | | | AAACCCCTCTTTCATTGACTGGTTGTGATGACTACTATCATTCGCCATA |
| SNP224 | 1 | 578 | TCTGGAAGACATGTTGGAGGATCGTACGTGTATGGTTCAAAGGGATTGTCTA |
| | | | ACAGAAAATAACTGGACGACGGGCTGCATGAGTTATCACATTGATAGGA |
| SNP224 | 2 | 579 | TCTGGAAGACATGTTGGAGGATCGTACGTGTATGGTTCAAAGGGATTGTCGA |
| | | | ACAGAAAATAACTGGACGACGGGCTGCATGAGTTATCACATTGATAGGA |
| SNP225 | 1 | 580 | AGCGATTTCAGGGACGAAGAATGCTCAAAACAATAAGGGATGTTCTTGAGAT |
| | | | GCTGGCATCTTTTCAGTTCCAAGCGTTCAAGGCATGGAAAGGCATCATC |
| SNP225 | 2 | 581 | AGCGATTTCAGGGACGAAGAATGCTCAAAACAATAAGGGATGTTCTTGAGG |
| | | | TGCTGGCATCTTTTCAGTTCCAAGCGTTCAAGGCATGGAAAGGCATCATC |
| SNP226 | 1 | 582 | CCTTTGGCAAGTTATCTTACGATAGAACAGAAAACATTGCACTGAAAGAATT |
| | | | GCCAAGATATAGATCTCCCAAAATCAGCAATCTTCTCTGCAACTTCAAC |
| SNP226 | 2 | 583 | CCTTTGGCAAGTTATCTTACGATAGAACAGAAAACATTGCACTGAAAGAACT |
| | | | GCCAAGATATAGATCTCCCAAAATCAGCAATCTTCTCTGCAACTTCAAC |
| SNP227 | 1 | 584 | ATTCAACTATAGCATGGGTTGGTTGTGTGGGCAAAGGCAGAGTCCCTAACAT |
| | | | GAGCTACGCGTACAAGACAACAAGTTCAGTGGACTACATGTTCCGCGTC |
| SNP227 | 2 | 585 | ATTCAACTATAGCATGGGTTGGTTGTGTGGGCAAAGGCAGAGTCCCTAACGT |
| | | | GAGCTACGCGTACAAGACAACAAGTTCAGTGGACTACATGTTCCGCGTC |
| SNP228 | 1 | 586 | CAAAATCCCAAAATTACCCTTCCTCCTACACCGCCCTATCCTACACACTTATG |
| | | | TCCTCATTCGTCTTTTTACAATCTGAGTCTCACTGTCTCCAACTCACT |
| SNP228 | 2 | 587 | CAAAATCCCAAAATTACCCTTCCTCCTACACCGCCCTATCCTACACACTTGTG |
| | | | TCCTCATTCGTCTTTTTACAATCTGAGTCTCACTGTCTCCAACTCACT |
| SNP229 | 1 | 588 | ATTCTACTACTGTTCCATAACCCATTGTGTTCAATTGGACTCATTTTGAGATC |
| | | | CATGAATCGCCAGAGCTTTCGATTAGATCCTCTTTTTTTTCTTTGTTT |
| SNP229 | 2 | 589 | ATTCTACTACTGTTCCATAACCCATTGTGTTCAATTGGACTCATTTTGAGCTC |
| | | | CATGAATCGCCAGAGCTTTCGATTAGATCCTCTTTTTTTTCTTTGTTT |
| SNP230 | 1 | 590 | AATGAAGTTGAAGCAATATCTTTCGAGAATGATGAGATTTCTCCTGAATCAA |
| | | | TTGAGAAAGTTCTGTCGTTGGATCACTTATCTATCATTTTGAACTCTGA |
| SNP230 | 2 | 591 | AATGAAGTTGAAGCAATATCTTTCGAGAATGATGAGATTTCTCCTGAATCGA |
| | | | TTGAGAAAGTTCTGTCGTTGGATCACTTATCTATCATTTTGAACTCTGA |
| SNP231 | 1 | 592 | ATTTCCCGCCAAATCCTCATCGATCCGATCAAATCGACTACCGTCACCACTG |
| | | | GCGCGAACATCTCTATTTCCGTGCGGTTGAAAATTCCAGCCGAATTCGG |
| SNP231 | 2 | 593 | ATTTCCCGCCAAATCCTCATCGATCCGATCAAATCGACTACCGTCACCACCG |
| | | | GCGCGAACATCTCTATTTCCGTGCGGTTGAAAATTCCAGCCGAATTCGG |
| SNP232 | 1 | 594 | ATGTTTCAAAAGCTGCTGCTGAAGCAAGGAAAAAATCGAAAATTCTTCAGTC |
| | | | AGTGGTGAGAAACAAAGAACCTTACATTCTTGAAACAAATAGCAGTTTA |
| SNP232 | 2 | 595 | ATGTTTCAAAAGCTGCTGCTGAAGCAAGGAAAAAATCGAAAATTCTTCAGCC |
| | | | AGTGGTGAGAAACAAAGAACCTTACATTCTTGAAACAAATAGCAGTTTA |
| SNP233 | 1 | 596 | AAATACGAGTTTGCATATTTTCGTCCATGTTTTTAATCTTTTCAATGTGCTTGT |
| | | | TGTGATATGACTTTCTATGCAATCATTGGTTCTTGAGTGAATTCACT |
| SNP233 | 2 | 597 | AAATACGAGTTTGCATATTTTCGTCCATGTTTTTAATCTTTTCAATGTGCCTGT |
| | | | TGTGATATGACTTTCTATGCAATCATTGGTTCTTGAGTGAATTCACT |
| SNP234 | 1 | 598 | ATGACCAAGTTGTGGGTAGCTGAAGGGTTTGTACAAGCAAACAACGAAAAA |
| | | | GGACAAGAAGATACCGCACAAGGTTTCTTGGACGATCTTATTGGTAGGAA |
| SNP234 | 2 | 599 | ATGACCAAGTTGTGGGTAGCTGAAGGGTTTGTACAAGCAAACAACGAAAAT |
| | | | GGACAAGAAGATACCGCACAAGGTTTCTTGGACGATCTTATTGGTAGGAA |
| SNP235 | 1 | 600 | CAGGGGCAATGTAGCCTATATTTCCTCTCACTACTGTGCTAAGATGGGTTAG |
| | | | ATCAAGCTCGGGCATTGTCTTAGATATTCCAAAATCAGTAATTTTTGGC |
| SNP235 | 2 | 601 | CAGGGGCAATGTAGCCTATATTTCCTCTCACTACTGTGCTAAGATGGGTTTGA |
| | | | TCAAGCTCGGGCATTGTCTTAGATATTCCAAAATCAGTAATTTTTGGC |
| SNP236 | 1 | 602 | GTTAATGCCAGTGAAAAGTCTACTTCCACTTCTCCTGTGGATCTATTAGGTTT |
| | | | GGCTAACTATGCGTCAGATGATGAGGATGACAATGAAATCCAGAGTTC |
| SNP236 | 2 | 603 | GTTAATGCCAGTGAAAAGTCTACTTCCACTTCTCCTGTGGATCTATTAGGCTT |
| | | | GGCTAACTATGCGTCAGATGATGAGGATGACAATGAAATCCAGAGTTC |
| SNP237 | 1 | 604 | GAGCTGCTAACCAAGGCTCCACCATAGAAGCCCCAAATCCATCCACAATTTG |
| | | | CCTTCCCCTATGCTTCTCTTCATTAACAATCCTCTTATCCAAACTTGAC |
| SNP237 | 2 | 605 | GAGCTGCTAACCAAGGCTCCACCATAGAAGCCCCAAATCCATCCACAATTCG |
| | | | CCTTCCCCTATGCTTCTCTTCATTAACAATCCTCTTATCCAAACTTGAC |
| SNP238 | 1 | 606 | CGGTTATTGAAGAACAGTTCTTCAGCATTTTCAAATCTGGTGGCAGAATTTTT |
| | | | GCGGAGATCATCCAAGGGGTTAACCTAAAAGCATAGCACATTTGATAG |
| SNP238 | 2 | 607 | CGGTTATTGAAGAACAGTTCTTCAGCATTTTCAAATCTGGTGGCAGAATTCTT |
| | | | GCGGAGATCATCCAAGGGGTTAACCTAAAAGCATAGCACATTTGATAG |
| SNP239 | 1 | 608 | TGGGATGCTTCCAAACTATGTCGTCTATCATGAATTAATAGTTACATCACACC |
| | | | CATTTATGCGTAATGTATGTGCAGTTGAGATGCGATGGGTTGCACCAA |
| SNP239 | 2 | 609 | TGGGATGCTTCCAAACTATGTCGTCTATCATGAATTAATAGTTACATCACGCC |
| | | | CATTTATGCGTAATGTATGTGCAGTTGAGATGCGATGGGTTGCACCAA |
| SNP240 | 1 | 610 | ATGCCTCTGAGAGATTCTAACCCTGGTAATACAGCAAGTACGACTGGTTATG |
| | | | CAGTTCCTGGCATGATGCAAGTAATAGCTACCACTAGTGGAGATAGACC |
| SNP240 | 2 | 611 | ATGCCTCTGAGAGATTCTAACCCTGGTAATACAGCAAGTACGACTGGTTACG |
| | | | CAGTTCCTGGCATGATGCAAGTAATAGCTACCACTAGTGGAGATAGACC |
| SNP241 | 1 | 612 | TCACTTTCATAGATCTCCTCAATCGATTCGGGAATATCTGCTTGAAACTGTGG |
| | | | CCCAACAGGTATTCTCTTTCTGCGCTGCTTCCACCAAAAACCAATTGC |
| SNP241 | 2 | 613 | TCACTTTCATAGATCTCCTCAATCGATTCGGGAATATCTGCTTGAAACTGCGG |
| | | | CCCAACAGGTATTCTCTTTCTGCGCTGCTTCCACCAAAAACCAATTGC |
| SNP242 | 1 | 614 | TAATAAAATGGATTTGGAGACGAGTGAGATTTCAAATTACAAGTCATCAGTA |
| | | | GTTTTGTCTAAGTTGGCTAGTAACGAACAACATGGTGAAAACTCACCAT |
| SNP242 | 2 | 615 | TAATAAAATGGATTTGGAGACGAGTGAGATTTCAAATTACAAGTCATCAGCA |
| | | | GTTTTGTCTAAGTTGGCTAGTAACGAACAACATGGTGAAAACTCACCAT |
| SNP243 | 1 | 616 | GCGATTGCAAAATTTATGGAGAAAACAAGAGGTGGTAAGGTTAAGTTTGAT |
| | | | GCTAAACGTGTAGTAATGGCTGGTGGAGCTACTGGAGCTAATGAGACTCT |
| SNP243 | 2 | 617 | GCGATTGCAAAATTTATGGAGAAAACAAGAGGTGGTAAGGTTAAGTTTGAC |
| | | | GCTAAACGTGTAGTAATGGCTGGTGGAGCTACTGGAGCTAATGAGACTCT |
| SNP244 | 1 | 618 | CTGCGTTCTATGCACTTCCTTCATCACATTGTTGTGCAACATCGCTAAACACA |
| | | | GTTGGATTAATGCAACATTTTCGAAGAAAAGCCAACAACTCCTCTTTA |
| SNP244 | 2 | 619 | CTGCGTTCTATGCACTTCCTTCATCACATTGTTGTGCAACATCGCTAAACTCA |
| | | | GTTGGATTAATGCAACATTTTCGAAGAAAAGCCAACAACTCCTCTTTA |
| SNP245 | 1 | 620 | TAAGGTTTCCAGACAATTGTGGTCTCAGCAATCATGGATGTAACAGTGTAAG |
| | | | GATCCATGTTCGAAGCAGGCCTCCTGTCCTCGAAATATCCCTTCCCTGC |
| SNP245 | 2 | 621 | TAAGGTTTCCAGACAATTGTGGTCTCAGCAATCATGGATGTAACAGTGTAGG |
| | | | GATCCATGTTCGAAGCAGGCCTCCTGTCCTCGAAATATCCCTTCCCTGC |
| SNP246 | 1 | 622 | TTACTATGCAATTTCAAGAAAAGGGTTCATGAACTAGAAGCTGAAGTAGCAA |
| | | | ATAGACGATTAACGGAGTCCAAAATATTCGATTCGTTGGCCTCACAGAC |
| SNP246 | 2 | 623 | TTACTATGCAATTTCAAGAAAAGGGTTCATGAACTAGAAGCTGAAGTAGCGA |
| | | | ATAGACGATTAACGGAGTCCAAAATATTCGATTCGTTGGCCTCACAGAC |
| SNP247 | 1 | 624 | AATTCAATTGGTCTTGTTTGTTTGGCCCAATAGGCCAATGTGGAGTGTTAATA |
| | | | TAATTGGAGATTCGGGGTTTTTTCTTCTTCTTTTCAATTGTGGGGGAT |
| SNP247 | 2 | 625 | AATTCAATTGGTCTTGTTTGTTTGGCCCAATAGGCCAATGTGGAGTGTTACTA |
| | | | TAATTGGAGATTCGGGGTTTTTTCTTCTTCTTTTCAATTGTGGGGGAT |
| SNP248 | 1 | 626 | CAAAGTTATCCTCCCGGAGTGCAATCTCATAATAGTGCTCCTGTTCAATCTCT |
| | | | TCCTAGTTATGCCTATGGCAATTCCGTCGCTGCAATGCCACCCCATAC |
| SNP248 | 2 | 627 | CAAAGTTATCCTCCCGGAGTGCAATCTCATAATAGTGCTCCTGTTCAATCGCT |
| | | | TCCTAGTTATGCCTATGGCAATTCCGTCGCTGCAATGCCACCCCATAC |
| SNP249 | 1 | 628 | CTAATTCCCTGTTTAACTGAAAAATGGGGAGATTTACCACTAAAAGTCGATG |
| | | | ATTCCGAAGATATGGTAATTTACGGTCTATTAAAAGACGCTCTAAGCGT |
| SNP249 | 2 | 629 | CTAATTCCCTGTTTAACTGAAAAATGGGGAGATTTACCACTAAAAGTCGACG |
| | | | ATTCCGAAGATATGGTAATTTACGGTCTATTAAAAGACGCTCTAAGCGT |
| SNP250 | 1 | 630 | TCGGATGTTATCAGTGTGTGTACAGTAAAACCTCTGGTCTTATGTGTACTATG |
| | | | GAGTATCATTGTAATAGCAGTACAAGCTTTGAAATGGACTCTACTTGT |
| SNP250 | 2 | 631 | TCGGATGTTATCAGTGTGTGTACAGTAAAACCTCTGGTCTTATGTGTACTGTG |
| | | | GAGTATCATTGTAATAGCAGTACAAGCTTTGAAATGGACTCTACTTGT |
| SNP251 | 1 | 632 | TACACTTTGTGCTATTTTCAGATGAAATTTATGATGCATGGGTGACAGCAACG |
| | | | AATGAACAGTTGAATTGATTAGTATGTTTACGAGTATTTGCAAGACGG |
| SNP251 | 2 | 633 | TACACTTTGTGCTATTTTCAGATGAAATTTATGATGCATGGGTGACAGCAGC |
| | | | GAATGAACAGTTGAATTGATTAGTATGTTTACGAGTATTTGCAAGACGG |
| SNP252 | 1 | 634 | TAATAAACCTTAGCAAAGTTGCCTTGACCTAATAATCTCCCCAAATCGTATTT |
| | | | TTCCATCAGTACATTTCCTTTTTTCTCCATTTGAACCAACAATTCTTG |
| SNP252 | 2 | 635 | TAATAAACCTTAGCAAAGTTGCCTTGACCTAATAATCTCCCCAAATCGTACTT |
| | | | TTCCATCAGTACATTTCCTTTTTTCTCCATTTGAACCAACAATTCTTG |
| SNP253 | 1 | 636 | CAATTTTAACTCAATAACAGGAAATCACACCTAATCCAGGACACGACGCTTG |
| | | | TTCAGATGTATCTACGGAATAATAGCAACCACAGATAACACGACCCCAA |
| SNP253 | 2 | 637 | CAATTTTAACTCAATAACAGGAAATCACACCTAATCCAGGACACGACGCTCG |
| | | | TTCAGATGTATCTACGGAATAATAGCAACCACAGATAACACGACCCCAA |
| SNP254 | 1 | 638 | GGCCCTAGCAAATTATTCGCCATCTCCCTTAGCACATGCTTCAGTTCATTCCC |
| | | | ATCATTAATTACTGAACACCCGCTCAAAGGTGAACCATAAGTGTTGTC |
| SNP254 | 2 | 639 | GGCCCTAGCAAATTATTCGCCATCTCCCTTAGCACATGCTTCAGTTCATTGCC |
| | | | ATCATTAATTACTGAACACCCGCTCAAAGGTGAACCATAAGTGTTGTC |
| SNP255 | 1 | 640 | AAATTCCGGTTTTGTATACCGATGATGAAAACACTCTAGCTAGGTGTGTTATG |
| | | | GGGTTTAAACTTTTTTGGGGTATTTGATGTCCATTGTTGATTTTGAAC |
| SNP255 | 2 | 641 | AAATTCCGGTTTTGTATACCGATGATGAAAACACTCTAGCTAGGTGTGTTGT |
| | | | GGGGTTTAAACTTTTTTGGGGTATTTGATGTCCATTGTTGATTTTGAAC |
| SNP256 | 1 | 642 | TACTCTGTTGAACCTGCTGAAAGCAGGAGGCCTTTCCGTGCTCTCTTGGATGT |
| | | | CGGCCTCTTAAGAACTACTACAGGGAACCGTGTTTTTGGTGCTCTCAA |
| SNP256 | 2 | 643 | TACTCTGTTGAACCTGCTGAAAGCAGGAGGCCTTTCCGTGCTCTCTTGGACGT |
| | | | CGGCCTCTTAAGAACTACTACAGGGAACCGTGTTTTTGGTGCTCTCAA |
| SNP257 | 1 | 644 | ATATAGATCTGTGTGAAAGGCATCATATGTTAGCTCTCTGTTATTACGGAAA |
| | | | AGGAGATAATTTCACCGCCTTGAACTTGTTGCGGAAACTATTGAGTAGT |
| SNP257 | 2 | 645 | ATATAGATCTGTGTGAAAGGCATCATATGTTAGCTCTCTGTTATTACGGAGA |
| | | | AGGAGATAATTTCACCGCCTTGAACTTGTTGCGGAAACTATTGAGTAGT |
| SNP258 | 1 | 646 | CGGCGAAATTCCAGTAGACATTCAGGGTCTGAGTAATTTGAGAGCATTGAAC |
| | | | TTGGGGAGAAATAAGTTCATGGGTGAAATTCCAGATGAAATTGGAGGTT |
| SNP258 | 2 | 647 | CGGCGAAATTCCAGTAGACATTCAGGGTCTGAGTAATTTGAGAGCATTGAGC |
| | | | TTGGGGAGAAATAAGTTCATGGGTGAAATTCCAGATGAAATTGGAGGTT |
| SNP259 | 1 | 648 | ACAATACATGAGAGGAACAAATTAAGTAAGATCATCTTCCTCAAGCTCCTTC |
| | | | GCCTTCAATGTTTCCTTGACTCTTAGAAGTAGTGTTGTCCTCCAAGCAT |
| SNP259 | 2 | 649 | ACAATACATGAGAGGAACAAATTAAGTAAGATCATCTTCCTCAAGCTCCTCC |
| | | | GCCTTCAATGTTTCCTTGACTCTTAGAAGTAGTGTTGTCCTCCAAGCAT |
| SNP260 | 1 | 650 | ATAAGTGTCCACTTGACCATAATGCCCTTGTGAGATGGGTGTGGAGGATGAG |
| | | | TCCCACATGTTCAAGGGCATAGGTTGGTGATGCTGATGAGGTGGACCAA |
| SNP260 | 2 | 651 | ATAAGTGTCCACTTGACCATAATGCCCTTGTGAGATGGGTGTGGAGGATGGG |
| | | | TCCCACATGTTCAAGGGCATAGGTTGGTGATGCTGATGAGGTGGACCAA |
| SNP261 | 1 | 652 | CCCATTCTTTGTTTCTACACACAATTCAAAATCCCCTCCTCCCTCTCTTTTCCC |
| | | | CCCTTTGAACTCTGCAGCCGTACGCCACTCTCATTTTCCTGCGAATT |
| SNP261 | 2 | 653 | CCCATTCTTTGTTTCTACACACAATTCAAAATCCCCTCCTCCCTCTCTTTCCCC |
| | | | CCCTTTGAACTCTGCAGCCGTACGCCACTCTCATTTTCCTGCGAATT |
| SNP262 | 1 | 654 | CAGTAAAAATGCTTGCAAAGGGCAGAAGACCTCCCACCAGAGAAATAACAT |
| | | | ATGGTCTTAGGTACCATTTCTTCTCAGGGATAGGACGCGGAATATTCTTA |
| SNP262 | 2 | 655 | CAGTAAAAATGCTTGCAAAGGGCAGAAGACCTCCCACCAGAGAAATAACAG |
| | | | ATGGTCTTAGGTACCATTTCTTCTCAGGGATAGGACGCGGAATATTCTTA |
| SNP263 | 1 | 656 | ATCATCTTTCTTCTCCGGATTGGGAGAAAATACCCCTGCTGTAACAAGTGTG |
| | | | GTAAGTACAAGAGAAATGGCGAAAACATTCATCATCTTTTCCAATTAGT |
| SNP263 | 2 | 657 | ATCATCTTTCTTCTCCGGATTGGGAGAAAATACCCCTGCTGTAACAAGTGCG |
| | | | GTAAGTACAAGAGAAATGGCGAAAACATTCATCATCTTTTCCAATTAGT |
| SNP264 | 1 | 658 | TGGGTGCTCAATGGTCAGATGACCATCTGAGTGTGAGAAATATGCACTCCTT |
| | | | CACAAGCAGCAGGCCTATCAGAGTGCTGCTCATGCGTGGTTCAGCAGAG |
| SNP264 | 2 | 659 | TGGGTGCTCAATGGTCAGATGACCATCTGAGTGTGAGAAATATGCACTCCGT |
| | | | CACAAGCAGCAGGCCTATCAGAGTGCTGCTCATGCGTGGTTCAGCAGAG |
| SNP265 | 1 | 660 | GTGCATTTTTTCCATGATGGACAAGGTTTCATGTCTGTTGAGTTGACACCAAC |
| | | | AGAGGCTGAGATCAAATATTATGATGTTTTTGGTAGAATTAGACATAG |
| SNP265 | 2 | 661 | GTGCATTTTTTCCATGATGGACAAGGTTTCATGTCTGTTGAGTTGACACCGAC |
| | | | AGAGGCTGAGATCAAATATTATGATGTTTTTGGTAGAATTAGACATAG |
| SNP266 | 1 | 662 | AGTTAATTCTTGTGCTGCTTGCTATATTTTGAGGCCTGTTGGACGCAACAAAC |
| | | | TAGTAAAATAGGATCTTAGTCATGTATTGCCTCAAGAATTTGTGTTTC |
| SNP266 | 2 | 663 | AGTTAATTCTTGTGCTGCTTGCTATATTTTGAGGCCTGTTGGACGCAACATAC |
| | | | TAGTAAAATAGGATCTTAGTCATGTATTGCCTCAAGAATTTGTGTTTC |
| SNP267 | 1 | 664 | GGCCCGGATAAGATTAGTAAAATATATGGCGACTGGATTGATGACATCGAAT |
| | | | GAAGGAGTTGTCATAATTAGCTACTGATCTGTTTAGCTAGACACAAATA |
| SNP267 | 2 | 665 | GGCCCGGATAAGATTAGTAAAATATATGGCGACTGGATTGATGACATCGAGT |
| | | | GAAGGAGTTGTCATAATTAGCTACTGATCTGTTTAGCTAGACACAAATA |
| SNP268 | 1 | 666 | GGTCGGAAGCAATTGATGGGGCAAGAATCGGTCGGAAATGGAGTTCTAGGA |
| | | | CATTTATCTTCGTCGTCTTGATGAGAAGGTAGTGGATTTAAACCGTTTAA |
| SNP268 | 2 | 667 | GGTCGGAAGCAATTGATGGGGCAAGAATCGGTCGGAAATGGAGTTCTAGGG |
| | | | CATTTATCTTCGTCGTCTTGATGAGAAGGTAGTGGATTTAAACCGTTTAA |
| SNP269 | 1 | 668 | TATGTAGCTAGGAAGAGAGTTGAAAAGACCTAGCTTACAAAAGATGGGGGA |
| | | | AAAAAGGGGGAGATGATAAATAAGGTATAAGTTTTTGAGAGATGAATGAA |
| SNP269 | 2 | 669 | TATGTAGCTAGGAAGAGAGTTGAAAAGACCTAGCTTACAAAAGATGGGGGG |
| | | | AAAAAGGGGGAGATGATAAATAAGGTATAAGTTTTTGAGAGATGAATGAA |
| SNP270 | 1 | 670 | CAACACCTGTACAGGATACATCTGCAGAAGAATTGTTGTCCAGAAAGATGAC |
| | | | AGGCAATAGGTTGGCAGAATCTTTATGGCCCTCAACAATGAGGAGTCTG |
| SNP270 | 2 | 671 | CAACACCTGTACAGGATACATCTGCAGAAGAATTGTTGTCCAGAAAGATGGC |
| | | | AGGCAATAGGTTGGCAGAATCTTTATGGCCCTCAACAATGAGGAGTCTG |
| SNP271 | 1 | 672 | GTCTCCTATGCTCTTCCTGTCTCCTTTGTTTCAAGTGGTATCATGCGTTCAGAG |
| | | | ACAGAAGTTGAGGAGAAATCAATCGAAACAACCCAGACAACCACCAT |
| SNP271 | 2 | 673 | GTCTCCTATGCTCTTCCTGTCTCCTTTGTTTCAAGTGGTATCATGCGTTCGGAG |
| | | | ACAGAAGTTGAGGAGAAATCAATCGAAACAACCCAGACAACCACCAT |
| SNP272 | 1 | 674 | ATCTCAGGAACTTCTCTAAAGAACGTATCTTGTCTTCCAGGATTTGCATGATC |
| | | | TCACCAGGAAGTCGGCAGACAACTACTACATTGTCAAATGCTTCTGAA |
| SNP272 | 2 | 675 | ATCTCAGGAACTTCTCTAAAGAACGTATCTTGTCTTCCAGGATTTGCATGGTC |
| | | | TCACCAGGAAGTCGGCAGACAACTACTACATTGTCAAATGCTTCTGAA |
| SNP273 | 1 | 676 | GACAGCTCCACTTGCAGCAGAAAGTGTGACTCCAACAAAGCGAAACAGAAA |
| | | | ATCTTCAGCTTCTAAGAAGGACGTGAAAGACAAAAAAGAACAGGAAGAAA |
| SNP273 | 2 | 677 | GACAGCTCCACTTGCAGCAGAAAGTGTGACTCCAACAAAGCGAAACAGAAG |
| | | | ATCTTCAGCTTCTAAGAAGGACGTGAAAGACAAAAAAGAACAGGAAGAAA |
| SNP274 | 1 | 678 | CATGAAGTTGGGATACACGAATGCATATAAAGCTTTTGATCCAAGTCGATCA |
| | | | AAATCTTGGTCCTTCCCCGCACTACCAGTTTCACATCGTTGGATGCTGA |
| SNP274 | 2 | 679 | CATGAAGTTGGGATACACGAATGCATATAAAGCTTTTGATCCAAGTCGATGA |
| | | | AAATCTTGGTCCTTCCCCGCACTACCAGTTTCACATCGTTGGATGCTGA |
| SNP275 | 1 | 680 | TCTTTTGTCAGAAGGAATCAACTCGGGGGTTGATGGAGCCAGAAGCATCTAG |
| | | | TGGTGGTTAGCTAGGCTGTTAATCATCCAACAAAAGTTGTTAGATGATC |
| SNP275 | 2 | 681 | TCTTTTGTCAGAAGGAATCAACTCGGGGGTTGATGGAGCCAGAAGCATCTCG |
| | | | TGGTGGTTAGCTAGGCTGTTAATCATCCAACAAAAGTTGTTAGATGATC |
| SNP276 | 1 | 682 | CTACTCTTTCACAATACATTCAAGTGGTGAAGTTCACATAGTGTGCCTCATCA |
| | | | CTTTTATTCATGCTACATGCATTACTTAATTTTATTCATAAGTTACAC |
| SNP276 | 2 | 683 | CTACTCTTTCACAATACATTCAAGTGGTGAAGTTCACATAGTGTGCCTCACCA |
| | | | CTTTTATTCATGCTACATGCATTACTTAATTTTATTCATAAGTTACAC |
| SNP277 | 1 | 684 | ACCTTCCTCCATGCCACAAAGTTCCCAGAACCTCCACCACGATAAGTTCTAGT |
| | | | ATCACAGAAGTCGAAGTTGAAGAAATAACTATTTTGGGAGCTTATTGG |
| SNP277 | 2 | 685 | ACCTTCCTCCATGCCACAAAGTTCCCAGAACCTCCACCACGATAAGTTCTGG |
| | | | TATCACAGAAGTCGAAGTTGAAGAAATAACTATTTTGGGAGCTTATTGG |
| SNP278 | 1 | 686 | TTGATCTTTAACCCTGCAACCGAAGCTATTGACTGTGAAAACTTCACCAACA |
| | | | AGCATTTGATCAATGACCTCCTCCCTCTTCGACCCAGTCAACTTGTGCA |
| SNP278 | 2 | 687 | TTGATCTTTAACCCTGCAACCGAAGCTATTGACTGTGAAAACTTCACCAAGA |
| | | | AGCATTTGATCAATGACCTCCTCCCTCTTCGACCCAGTCAACTTGTGCA |
| SNP279 | 1 | 688 | CCTGAAGAAGCTGTTTCGACTGATACTATTAATGGTGGGGGGCAGAACCCAG |
| | | | TTTTCGATCAGAGTCTTCGACTTAATGTCAAGACTATTGAAACATCAGT |
| SNP279 | 2 | 689 | CCTGAAGAAGCTGTTTCGACTGATACTATTAATGGTGGGGGGCAGAACCCGG |
| | | | TTTTCGATCAGAGTCTTCGACTTAATGTCAAGACTATTGAAACATCAGT |
| SNP280 | 1 | 690 | CACTCCGAAACTTTGTCAGAACAGGGAAGTGTCAAATCAATTGGGACTACTC |
| | | | CGGTAACCCAACTGCCCAGGCTGCACAAGAATGTCAGCGACTCAATGTT |
| SNP280 | 2 | 691 | CACTCCGAAACTTTGTCAGAACAGGGAAGTGTCAAATCAATTGGGACTACCC |
| | | | CGGTAACCCAACTGCCCAGGCTGCACAAGAATGTCAGCGACTCAATGTT |
| SNP281 | 1 | 692 | TCAGGCTGGATTCTTGGTTTATTCGAAGCGGTTAGTCACAGTAAGATAAGTTT |
| | | | TGTTGTATAAGCGGTGGGTAAAGCGGTTGTCGGTTTGCTGAACATGCC |
| SNP281 | 2 | 693 | TCAGGCTGGATTCTTGGTTTATTCGAAGCGGTTAGTCACAGTAAGATAAGCT |
| | | | TTGTTGTATAAGCGGTGGGTAAAGCGGTTGTCGGTTTGCTGAACATGCC |
| SNP282 | 1 | 694 | CAACTTTAAGTCGAGGATGAAGGGTGGTGGTGATTTGGCTGTTGCATCTATT |
| | | | ACGAATGGAAAAGATAGATATGTTCCGTTTGATGTGGAGAACGGTTCTA |
| SNP282 | 2 | 695 | CAACTTTAAGTCGAGGATGAAGGGTGGTGGTGATTTGGCTGTTGCATCTACT |
| | | | ACGAATGGAAAAGATAGATATGTTCCGTTTGATGTGGAGAACGGTTCTA |
| SNP283 | 1 | 696 | GTGAGGCTACTGTCGGGTTACCCGGTGGGTGTGACATTGGGGCCCGACCCAT |
| | | | TGATTTTTACATTCATGGTCTACGTGCTCTTGGTGCTACGGTTGAGTTG |
| SNP283 | 2 | 697 | GTGAGGCTACTGTCGGGTTACCCGGTGGGTGTGACATTGGGGCCCGACCCGT |
| | | | TGATTTTTACATTCATGGTCTACGTGCTCTTGGTGCTACGGTTGAGTTG |
| SNP284 | 1 | 698 | CAGACGACAATACTGTTGGACCAACTTGCATTTTCGGGATCACCTTTGTCTGG |
| | | | CAAGCAACCTTTTCTGAAACTTGCTTAGGTGATTTTCTTCTTCCCTGT |
| SNP284 | 2 | 699 | CAGACGACAATACTGTTGGACCAACTTGCATTTTCGGGATCACCTTTGTCCG |
| | | | GCAAGCAACCTTTTCTGAAACTTGCTTAGGTGATTTTCTTCTTCCCTGT |
| SNP285 | 1 | 700 | CAAAAAGTTTTCAAACCGTGTGATTTCTGGTGAACCTGATCCAGATCGTCAT |
| | | | GTAGTTGCACCAATCAAGTCAGACAAAAAGTTTTCATACCCAGATCTTC |
| SNP285 | 2 | 701 | CAAAAAGTTTTCAAACCGTGTGATTTCTGGTGAACCTGATCCAGATCGTCCT |
| | | | GTAGTTGCACCAATCAAGTCAGACAAAAAGTTTTCATACCCAGATCTTC |
| SNP286 | 1 | 702 | CGGTGGCGGCAACAGCGGAGGAGAATCGGCGTGGTAGGGAGGGTTTGAAGA |
| | | | TTGGGTTTACCGTTTTGAGTGATGAAGGTTTTGGAATTCGCGGAAGCAGA |
| SNP286 | 2 | 703 | CGGTGGCGGCAACAGCGGAGGAGAATCGGCGTGGTAGGGAGGGTTTGAAGG |
| | | | TTGGGTTTACCGTTTTGAGTGATGAAGGTTTTGGAATTCGCGGAAGCAGA |
| SNP287 | 1 | 704 | GAGTTTTTTTTGCGGAGTTGAACGAAGTGCTTACAAGAGAGTTGGCGGAGAA |
| | | | TGGTTACTCGGGAGTTGAAGTTAGGGTTACTCCCGTGCGAACTGAAATC |
| SNP287 | 2 | 705 | GAGTTTTTTTTGCGGAGTTGAACGAAGTGCTTACAAGAGAGTTGGCGGAGGA |
| | | | TGGTTACTCGGGAGTTGAAGTTAGGGTTACTCCCGTGCGAACTGAAATC |
| SNP288 | 1 | 706 | AACAAAACTTGAAGAGCAATTTAAGGAAGTGAAGTTGGAAGAAAGAGCAGT |
| | | | CCGTAGGGAAGCCAGAAGGAAGATGTATGGTTGGTCACCAAAATCAGAGG |
| SNP288 | 2 | 707 | AACAAAACTTGAAGAGCAATTTAAGGAAGTGAAGTTGGAAGAAAGAGCAGC |
| | | | CCGTAGGGAAGCCAGAAGGAAGATGTATGGTTGGTCACCAAAATCAGAGG |
| SNP289 | 1 | 708 | CAGGACCAAGTCTCCGATCTTTCTGGATTAAACGGGAAACTGCATCCATAAC |
| | | | CTCATTACTAATGTCAAGAGGCTTAGATGCAGCCCGAACACTTGGGATA |
| SNP289 | 2 | 709 | CAGGACCAAGTCTCCGATCTTTCTGGATTAAACGGGAAACTGCATCCATAGC |
| | | | CTCATTACTAATGTCAAGAGGCTTAGATGCAGCCCGAACACTTGGGATA |
| SNP290 | 1 | 710 | TCTGTCATGCGTTAATCGTGTTAACATTTCCTTTAAGGTATGACGATGAGTTC |
| | | | TGGCATCCGATTTACATGCCCGGACTGTTTTTTGATGGCAAAGTTTCT |
| SNP290 | 2 | 711 | TCTGTCATGCGTTAATCGTGTTAACATTTCCTTTAAGGTATGACGATGAGCTC |
| | | | TGGCATCCGATTTACATGCCCGGACTGTTTTTTGATGGCAAAGTTTCT |
| SNP291 | 1 | 712 | CATATTAGTAAAGGCCTAATGGAAGGGACACGATAAACTTGCTCCTCAATTA |
| | | | GCTCGTTGCCTGTTGTGGTTCCAAGTTGGTCCCTGGTTGTCTCAGTCTC |
| SNP291 | 2 | 713 | CATATTAGTAAAGGCCTAATGGAAGGGACACGATAAACTTGCTCCTCAATCA |
| | | | GCTCGTTGCCTGTTGTGGTTCCAAGTTGGTCCCTGGTTGTCTCAGTCTC |
| SNP292 | 1 | 714 | TTAATAGGTGCTACTCCGAGGTTCTTCGGTAGGTATTGCTTGCTAGAGGATA |
| | | | AACTCGAACGGATTAAAATTTATTGCTACAGGCGCTGTGCGTCTATGTT |
| SNP292 | 2 | 715 | TTAATAGGTGCTACTCCGAGGTTCTTCGGTAGGTATTGCTTGCTAGAGGACA |
| | | | AACTCGAACGGATTAAAATTTATTGCTACAGGCGCTGTGCGTCTATGTT |
| SNP293 | 1 | 716 | CCATCCGCTAAATCCAAAAATTTCGTTGAATATTAGGTTACCTGCAAAAAAG |
| | | | CAAAAGGCAACCCATCTGAATCCCTTCGCGGAACTTTCTATATTTGGTA |
| SNP293 | 2 | 717 | CCATCCGCTAAATCCAAAAATTTCGTTGAATATTAGGTTACCTGCAAAAAGG |
| | | | CAAAAGGCAACCCATCTGAATCCCTTCGCGGAACTTTCTATATTTGGTA |
| SNP294 | 1 | 718 | TCATCGACTAATTCAAATTCACGCTCCTTGCCTCAGTCACATTAAGGACTAGG |
| | | | CCGGTGTCGATCCGCAAATCGATTAACCAAAGCAAGTGCATTACTAGT |
| SNP294 | 2 | 719 | TCATCGACTAATTCAAATTCACGCTCCTTGCCTCAGTCACATTAAGGACTTGG |
| | | | CCGGTGTCGATCCGCAAATCGATTAACCAAAGCAAGTGCATTACTAGT |
| SNP295 | 1 | 720 | ATCAACGCCGCCGACACTGAATGAAGCTCCGAGTGTGATTCGGGTCGGGTTG |
| | | | GAAACGACATTTCCGGTTCGAGAAAATCACCTAAACCAAAAACAGTGAA |
| SNP295 | 2 | 721 | ATCAACGCCGCCGACACTGAATGAAGCTCCGAGTGTGATTCGGGTCGGGTCG |
| | | | GAAACGACATTTCCGGTTCGAGAAAATCACCTAAACCAAAAACAGTGAA |
| SNP296 | 1 | 722 | GAACACTATGAAGATGAGACCCCAGATGACACCGAAGATGACGATGAGGGT |
| | | | GGAAAAGAAGCATCTCTTGGGCGTTATTGTGTCTTCTGTAGTAAACTTGA |
| SNP296 | 2 | 723 | GAACACTATGAAGATGAGACCCCAGATGACACCGAAGATGACGATGAGGGG |
| | | | GGAAAAGAAGCATCTCTTGGGCGTTATTGTGTCTTCTGTAGTAAACTTGA |
| SNP297 | 1 | 724 | ACTGCAAGTAAAAATGTTTCATATACGGATGCTGCAGCTGTTGAAGAAAAAA |
| | | | AATCCTGGTCTTCTTCTGCATGCCCATACAACCTGTAGAGGACACGCAA |
| SNP297 | 2 | 725 | ACTGCAAGTAAAAATGTTTCATATACGGATGCTGCAGCTGTTGAAGAAAAGA |
| | | | AATCCTGGTCTTCTTCTGCATGCCCATACAACCTGTAGAGGACACGCAA |
| SNP298 | 1 | 726 | GTTCAGTATCCCGAAATTCAAAGGTTTGCTTTTCGGCTCCTTAGTCAGACATG |
| | | | CAATGGTGCTTCACATTATAGGCTGAAAAGGAGCTTGGTCGAGACATT |
| SNP298 | 2 | 727 | GTTCAGTATCCCGAAATTCAAAGGTTTGCTTTTCGGCTCCTTAGTCAGACTTG |
| | | | CAATGGTGCTTCACATTATAGGCTGAAAAGGAGCTTGGTCGAGACATT |
| SNP299 | 1 | 728 | ACTATAGCGTGCATGGGAAACAGAAAATTGAGTTTGATCCTTGATGATTGAA |
| | | | AATGAAGGATCAGCAGAACCGTCTTTGCTCTGAGAAACAACAAAAGGTG |
| SNP299 | 2 | 729 | ACTATAGCGTGCATGGGAAACAGAAAATTGAGTTTGATCCTTGATGATTGGA |
| | | | AATGAAGGATCAGCAGAACCGTCTTTGCTCTGAGAAACAACAAAAGGTG |
| SNP300 | 1 | 730 | TAACCACAGCATCAGACAAAGACATAAAGAGGCTGCGATGAGGAGAAAAAA |
| | | | ATATGGAGTAGACACACTACTCCCAATATTCCACTTCAAATTATTAACCC |
| SNP300 | 2 | 731 | TAACCACAGCATCAGACAAAGACATAAAGAGGCTGCGATGAGGAGAAAAAC |
| | | | ATATGGAGTAGACACACTACTCCCAATATTCCACTTCAAATTATTAACCC |
| SNP301 | 1 | 732 | TGGTGTTCTAACATTTGAAAGCAAATTCTGCAACCTTTCCACACTCATTCTAG |
| | | | CACATGATGCGATGTCTTCTTTGGTTGGATGACGATTGCCTTGTCGAA |
| SNP301 | 2 | 733 | TGGTGTTCTAACATTTGAAAGCAAATTCTGCAACCTTTCCACACTCATTCCAG |
| | | | CACATGATGCGATGTCTTCTTTGGTTGGATGACGATTGCCTTGTCGAA |
| SNP302 | 1 | 734 | CGTTTGCATGACCGTCTGTGGCTGCATTCGTAGATGTTGAATTTGCTTTCAGT |
| | | | ACCAAACTTTTGTATGACAGACGACATAACTTCTGTCATATTTCCAGG |
| SNP302 | 2 | 735 | CGTTTGCATGACCGTCTGTGGCTGCATTCGTAGATGTTGAATTTGCTTTCGGT |
| | | | ACCAAACTTTTGTATGACAGACGACATAACTTCTGTCATATTTCCAGG |
| SNP303 | 1 | 736 | CCATCCCAGAAGACATAATTTGTAGCATTGGAGCATGTTCCGATGGACCTAG |
| | | | CATTACAAAGGAATGATGTTTCTAGTGTACCAGTCCCGCAGCAAGCCTT |
| SNP303 | 2 | 737 | CCATCCCAGAAGACATAATTTGTAGCATTGGAGCATGTTCCGATGGACCTGG |
| | | | CATTACAAAGGAATGATGTTTCTAGTGTACCAGTCCCGCAGCAAGCCTT |
| SNP304 | 1 | 738 | GAATTCTTGCATTTGAAATTGCAACTTTGATGTCTAAGGTGGTTAACTTGTGG |
| | | | CAGTGTCTAAGTGAAAGGCGANTCGACAAGTTAAGAGAAGAAATCTCAAGT |
| | | | TCACTTGGCATTCAGAAGCTTGTTGCTGAAGATGACAAATATCTTATGGATCT |
| | | | TGCTNTTGCTGAGATAATTGACAATTTGGGATCTCTGACGAAGT |
| SNP304 | 2 | 739 | GAATTCTTGCATTTGAAATTGCAACTTTGATGTCTAAGGTGGTTAACTTGTGG |
| | | | CAGTGTCTAAGTGAAAGGCGANTCGACAAGTTAAGAGAAGAAATCTCGAGT |
| | | | TCACTTGGCATTCAGAAGCTTGTTGCTGAAGATGACAAATATCTTATGGATCT |
| | | | TGCTNTTGCTGAGATAATTGACAATTTGGGATCTCTGACGAAGT |
| SNP305 | 1 | 740 | CCCACAAAATAGCTGGTAGAGAATCATTGATTGGCTCAATTTAGCCTCTATA |
| | | | ACACATTTTGCAAGAACTGAAAGATTGATAAGTAACCATCCCATCATAC |
| SNP305 | 2 | 741 | CCCACAAAATAGCTGGTAGAGAATCATTGATTGGCTCAATTTAGCCTCTACA |
| | | | ACACATTTTGCAAGAACTGAAAGATTGATAAGTAACCATCCCATCATAC |
| SNP306 | 1 | 742 | AATGATGTCATTGAGATCTTTAGTGGATAGTAAAATGGTGGGTTCTTAAGTA |
| | | | AAATGGTAAAGAGGTGCGCTGTTCGTGATGTGGGCTTGTAGATAAAGCT |
| SNP306 | 2 | 743 | AATGATGTCATTGAGATCTTTAGTGGATAGTAAAATGGTGGGTTCTTAAGGA |
| | | | AAATGGTAAAGAGGTGCGCTGTTCGTGATGTGGGCTTGTAGATAAAGCT |
| SNP307 | 1 | 744 | CAAAACCCGCATGCTGGTGACGTTTTGGTTGATTCTATGAAAAGGTATTATG |
| | | | GAAAGTTACCTGCAGTCGTTGAACTGTTTAGTCAAGTTGGAGCACAGGT |
| SNP307 | 2 | 745 | CAAAACCCGCATGCTGGTGACGTTTTGGTTGATTCTATGAAAAGGTATTACG |
| | | | GAAAGTTACCTGCAGTCGTTGAACTGTTTAGTCAAGTTGGAGCACAGGT |
| SNP308 | 1 | 746 | ACACTCATGAACATTGCTGATAACCCGACGAATGTCCAACTCCCCGGTATAT |
| | | | ACAACAAGCAAGAGAATGCCAGGGTACCTATTATTGTCACTGGTAACGA |
| SNP308 | 2 | 747 | ACACTCATGAACATTGCTGATAACCCGACGAATGTCCAACTCCCCGGTATGT |
| | | | ACAACAAGCAAGAGAATGCCAGGGTACCTATTATTGTCACTGGTAACGA |
| SNP309 | 1 | 748 | TTAATGCATCACAGACAGGATGTAAACCACACCCTACGCGTATAGATATTAT |
| | | | CAGCCCATTATCCAATTTGTTGAAGCGTTTGCAAGTTAGGGATACAGAG |
| SNP309 | 2 | 749 | TTAATGCATCACAGACAGGATGTAAACCACACCCTACGCGTATAGATATTCT |
| | | | CAGCCCATTATCCAATTTGTTGAAGCGTTTGCAAGTTAGGGATACAGAG |
| SNP310 | 1 | 750 | TCTTTTGGTAGAAGAGATGTGCCATCTTTCCAATTTTCATCAATATATTCAAG |
| | | | AATCACTAGAGACTCAGGAATTGGATTTCCCTTGTGCAAAAATACAGG |
| SNP310 | 2 | 751 | TCTTTTGGTAGAAGAGATGTGCCATCTTTCCAATTTTCATCAATATATTCCAG |
| | | | AATCACTAGAGACTCAGGAATTGGATTTCCCTTGTGCAAAAATACAGG |
| SNP311 | 1 | 752 | GAGTAGAAGAAGAATCAAAAAGCAAAAATGGTGAAAGACAGAACCATTGGT |
| | | | GTGGCTGTAGATTTTTCAAAGAGCAGCAAAACAGCTTTGAAATGGGCAAT |
| SNP311 | 2 | 753 | GAGTAGAAGAAGAATCAAAAAGCAAAAATGGTGAAAGACAGAACCATTGG |
| | | | CGTGGCTGTAGATTTTTCAAAGAGCAGCAAAACAGCTTTGAAATGGGCAAT |
| SNP312 | 1 | 754 | TCGCTCATCTACCCTTCTCCACTTAAATAGTTTACGGGCAAGACTCGTACAGT |
| | | | GCATCCCGCAGTGGGGACACAAGTATGTGTCTTTCATTTGGTTCTTAA |
| SNP312 | 2 | 755 | TCGCTCATCTACCCTTCTCCACTTAAATAGTTTACGGGCAAGACTCGTACCGT |
| | | | GCATCCCGCAGTGGGGACACAAGTATGTGTCTTTCATTTGGTTCTTAA |
| SNP313 | 1 | 756 | TACAGATCCTCTGTTTTCTTCAAACAACAAATGTCTCTTATTCCAAGCTTTTTT |
| | | | GGTGGTCGGAGGAGCAATATCTTCGACCCATTTTCCCTTGACTTATG |
| SNP313 | 2 | 757 | TACAGATCCTCTGTTTTCTTCAAACAACAAATGTCTCTTATTCCAAGCTTCTTT |
| | | | GGTGGTCGGAGGAGCAATATCTTCGACCCATTTTCCCTTGACTTATG |
| SNP314 | 1 | 758 | TCCAAATCCATAAGTGGTTGTTACTTCACTTGAACACCATTCCCCTTCTTAGC |
| | | | TGAATTTGTGTTGCATCACCTTTTTCCGTAGTGCATGCTCCTCTTGGT |
| SNP314 | 2 | 759 | TCCAAATCCATAAGTGGTTGTTACTTCACTTGAACACCATTCCCCTTCTTCGC |
| | | | TGAATTTGTGTTGCATCACCTTTTTCCGTAGTGCATGCTCCTCTTGGT |
| SNP315 | 1 | 760 | CAGGAAGACATAATCACAACACTTTTGAATCTGTCCATCCATGACAACAATA |
| | | | AGAAGCTTGTCGCGGAGACTCCAAAAGTTATTCCACTTCTTGTGGAGGC |
| SNP315 | 2 | 761 | CAGGAAGACATAATCACAACACTTTTGAATCTGTCCATCCATGACAACAACA |
| | | | AGAAGCTTGTCGCGGAGACTCCAAAAGTTATTCCACTTCTTGTGGAGGC |
| SNP316 | 1 | 762 | GATGGAATTACACACAACCTCAGATGAATATGAGAGACAGCTACATACCTAA |
| | | | TTGGTCTGCATCGCGTAATCCTGGGCATTATTCTGGCTATCGTGGTCCT |
| SNP316 | 2 | 763 | GATGGAATTACACACAACCTCAGATGAATATGAGAGACAGCTACATACCTG |
| | | | ATTGGTCTGCATCGCGTAATCCTGGGCATTATTCTGGCTATCGTGGTCCT |
| SNP317 | 1 | 764 | CCAGCATAAATTTAAGAATGGAGTAGAATCCAATTGACAAAAGAGAGCAGT |
| | | | TATTAACGCACCTAAACATCCTTACCCGCACTGCAATTAATGCATTTATC |
| SNP317 | 2 | 765 | CCAGCATAAATTTAAGAATGGAGTAGAATCCAATTGACAAAAGAGAGCAGC |
| | | | TATTAACGCACCTAAACATCCTTACCCGCACTGCAATTAATGCATTTATC |
| SNP318 | 1 | 766 | ACTGCTGGACCTGGCATTGACATGTCCATGGCACATAATCATGCTATTTTTCA |
| | | | GAGTCTCCCGGAAGCTACGAGGCAAAATTTACAGATGGCCGCAGCAGC |
| SNP318 | 2 | 767 | ACTGCTGGACCTGGCATTGACATGTCCATGGCACATAATCATGCTATTTTCCA |
| | | | GAGTCTCCCGGAAGCTACGAGGCAAAATTTACAGATGGCCGCAGCAGC |
| SNP319 | 1 | 768 | GATTTATTCACAGATAACGAAGAGGATGATATGGAAAATGCTGATATCAGTA |
| | | | TCAAGGGAAGGAGGAGAGAAGACGATGGCATCTTTTTACGACTCAGGAT |
| SNP319 | 2 | 769 | GATTTATTCACAGATAACGAAGAGGATGATATGGAAAATGCTGATATCAGCA |
| | | | TCAAGGGAAGGAGGAGAGAAGACGATGGCATCTTTTTACGACTCAGGAT |
| SNP320 | 1 | 770 | AAATCGGCAGAGAAGGAAGTCGAGATATTGACCAAATTTGCTCCACTCAAA |
| | | | GGATTCTCCATCTAATTGAACTTTCTGTTGTACAGTTTAGTTCAGTATCT |
| SNP320 | 2 | 771 | AAATCGGCAGAGAAGGAAGTCGAGATATTGACCAAATTTGCTCCACTCAAG |
| | | | GGATTCTCCATCTAATTGAACTTTCTGTTGTACAGTTTAGTTCAGTATCT |
| SNP321 | 1 | 772 | AGTTCAAGCAAGGTGACATACTAATCAACACGTTCGTGCAATCGTTGCTCAA |
| | | | TTGAGCAACAACTCCAGAACAGATCATCGCAAACATAATCGCGAAAAAA |
| SNP321 | 2 | 773 | AGTTCAAGCAAGGTGACATACTAATCAACACGTTCGTGCAATCGTTGCTCGA |
| | | | TTGAGCAACAACTCCAGAACAGATCATCGCAAACATAATCGCGAAAAAA |
| SNP322 | 1 | 774 | CAATACAGATATGGGAGAAAACTAAAGATCTGAAGGCACAAGTGGAGACGT |
| | | | ACTATAAATCCTTAAAATTCACTCCATCGCAATTCCCCACTGTTGGTGGA |
| SNP322 | 2 | 775 | CAATACAGATATGGGAGAAAACTAAAGATCTGAAGGCACAAGTGGAGACGC |
| | | | ACTATAAATCCTTAAAATTCACTCCATCGCAATTCCCCACTGTTGGTGGA |
| SNP323 | 1 | 776 | GATGAAAAGATTGCAACTCATTTTCAAGTTGCCGTCAGCTCAATTGCACAAT |
| | | | CTCTCAGAACTCAGATTATTAATAGGTCTTATGATGAAGTTTCTATATG |
| SNP323 | 2 | 777 | GATGAAAAGATTGCAACTCATTTTCAAGTTGCCGTCAGCTCAATTGCACAGT |
| | | | CTCTCAGAACTCAGATTATTAATAGGTCTTATGATGAAGTTTCTATATG |
| SNP324 | 1 | 778 | ACAAGTTCTTCTTTGAAACGGAGATATTCAGGAACCGTCAATTGTTCAACAC |
| | | | TCAGATCATATACGTTCACACGAACATATTCCCAAACACCTGGCCTTGG |
| SNP324 | 2 | 779 | ACAAGTTCTTCTTTGAAACGGAGATATTCAGGAACCGTCAATTGTTCAACGC |
| | | | TCAGATCATATACGTTCACACGAACATATTCCCAAACACCTGGCCTTGG |
| SNP325 | 1 | 780 | GCTCGAGTCCTCAAATGAGGAATCAGCTGATTCGAGGGGTCTCCCTTCAATC |
| | | | CACGTTCTAACAGACTTGTTCTTCAGTTGGTAGGTGTAGTACACTTCTT |
| SNP325 | 2 | 781 | GCTCGAGTCCTCAAATGAGGAATCAGCTGATTCGAGGGGTCTCCCTTCAACC |
| | | | CACGTTCTAACAGACTTGTTCTTCAGTTGGTAGGTGTAGTACACTTCTT |
| SNP326 | 1 | 782 | GAGTCAAGATGAGATTGCAAAAAAGGACTCTTCAACCAGGAAGAGACCGCT |
| | | | TTTTGTCAATCCTCAAAGGCCAATGAGACCAAGTACTATTGCTGCAGCTA |
| SNP326 | 2 | 783 | GAGTCAAGATGAGATTGCAAAAAAGGACTCTTCAACCAGGAAGAGACCGCC |
| | | | TTTTGTCAATCCTCAAAGGCCAATGAGACCAAGTACTATTGCTGCAGCTA |
| SNP327 | 1 | 784 | TGGAGATGTACTCTAGATCTTAACTGTGATGTTCTGAGCTGTAAAAGTACTA |
| | | | ACTCCGATCATCAGTCAGATCAACGTCCCTATTACTTTGTAACGATGTC |
| SNP327 | 2 | 785 | TGGAGATGTACTCTAGATCTTAACTGTGATGTTCTGAGCTGTAAAAGTACCA |
| | | | ACTCCGATCATCAGTCAGATCAACGTCCCTATTACTTTGTAACGATGTC |
| SNP328 | 1 | 786 | TGAAGATTGTGTATGGTGATACTAAAGTGGATTTAAAAGGCGAAAACGACAT |
| | | | AAACATGGGTGCAGGGGAAGTTGTTGGTTTTGTTCTGGAGAATAGGAAG |
| SNP328 | 2 | 787 | TGAAGATTGTGTATGGTGATACTAAAGTGGATTTAAAAGGCGAAAACGACGT |
| | | | AAACATGGGTGCAGGGGAAGTTGTTGGTTTTGTTCTGGAGAATAGGAAG |
| SNP329 | 1 | 788 | GGATATGAAAGGTAAGAAGTTAACCGTCATTGGTACAGTTGATCCAGTGAAC |
| | | | GTAGTGAGTAGGCTACGTAAGTTTTGGTGGACAGAGATACTCATAGTAG |
| SNP329 | 2 | 789 | GGATATGAAAGGTAAGAAGTTAACCGTCATTGGTACAGTTGATCCAGTGAGC |
| | | | GTAGTGAGTAGGCTACGTAAGTTTTGGTGGACAGAGATACTCATAGTAG |
| SNP330 | 1 | 790 | AAAGAGTAAACCGGTGCACAAAGTATCCTACGTCAACAAGCTTCGGGGAAA |
| | | | GCCGCACCCCAAGAAGCATAAGTAGATTCATAACCACAAAGTGATACTAT |
| SNP330 | 2 | 791 | AAAGAGTAAACCGGTGCACAAAGTATCCTACGTCAACAAGCTTCGGGGAAG |
| | | | GCCGCACCCCAAGAAGCATAAGTAGATTCATAACCACAAAGTGATACTAT |
| SNP331 | 1 | 792 | CTTTAGCTGTCTTGATCTTCTGAAGTCTCACTTACAGTGCGTCAACTTCACTC |
| | | | AGCTCTAGACTGCCCAGCTCGGGAAGACAAGATAATGCCAACAATAAG |
| SNP331 | 2 | 793 | CTTTAGCTGTCTTGATCTTCTGAAGTCTCACTTACAGTGCGTCAACTTCAGTC |
| | | | AGCTCTAGACTGCCCAGCTCGGGAAGACAAGATAATGCCAACAATAAG |
| SNP332 | 1 | 794 | CCATAAGAGAACAACAGATTGGTGGCATTGTTCAGGTGATTGATTGATTTAT |
| | | | TCCTGGATGTTTTAAACAACTTATCATGTCGATTTCTTGTGGATTACTC |
| SNP332 | 2 | 795 | CCATAAGAGAACAACAGATTGGTGGCATTGTTCAGGTGATTGATTGATTTGT |
| | | | TCCTGGATGTTTTAAACAACTTATCATGTCGATTTCTTGTGGATTACTC |
| SNP333 | 1 | 796 | AAACCCTTATCGGCGCAAGAATGGGAAAATCTAATCGACGATTACAACCACG |
| | | | GTGGTTCACGGCGGCTCCGGTGGACTTCCATCAACTACGCCGCCGTTCCTCTT |
| | | | CTTGACCTCACACTTTCATCACTTCTCCGGAAAGATATCCCTCACAATC |
| SNP333 | 2 | 797 | AAACCCTTATCGGCGCAAGAATGGGAAAATCTAATCGACGATTACAACCACG |
| | | | GCGGTTCACGGCGGCTCCGGTGGACTTCCATCAACTACGCCGCCGTTCCTCTT |
| | | | CTTGACCTCACACTTTCATCACTTCTCCGGAAAGATATCCCTCACAATC |
| SNP334 | 1 | 798 | GAAAGGGGGATTTTGGTAAGAGATTGGGCACCTCAATTGGAGATCTTGTCAC |
| | | | ATTGTTCGACTGGTGGATTCTTGAGTCACTGTGGGTGGAATTCATGCAT |
| SNP334 | 2 | 799 | GAAAGGGGGATTTTGGTAAGAGATTGGGCACCTCAATTGGAGATCTTGTCGC |
| | | | ATTGTTCGACTGGTGGATTCTTGAGTCACTGTGGGTGGAATTCATGCAT |
| SNP335 | 1 | 800 | TAACCAACCACTTTGGTATCTGTTAGTCCACCAACATGACTATGAGTAATTCG |
| | | | ACTATTGAAACTGAAGATACTTTTGCCAGCTTGCTTGAACTTGCTGCC |
| SNP335 | 2 | 801 | TAACCAACCACTTTGGTATCTGTTAGTCCACCAACATGACTATGAGTAATCC |
| | | | GACTATTGAAACTGAAGATACTTTTGCCAGCTTGCTTGAACTTGCTGCC |
| SNP336 | 1 | 802 | CCACGTTGGACTAACGAAATTTTAACCATCATAGAAATGACTTCCGCGCGTT |
| | | | CCTTCACTGGAATTCAATTTCTTATAGTGGGACCCACTTTCCGATCTGT |
| SNP336 | 2 | 803 | CCACGTTGGACTAACGAAATTTTAACCATCATAGAAATGACTTCCGCGCGCT |
| | | | CCTTCACTGGAATTCAATTTCTTATAGTGGGACCCACTTTCCGATCTGT |
| SNP337 | 1 | 804 | TTTGCTGAAGAGAGCAGGATTTTTATGTGGCAGTTGCTAGACTGCAGTATAA |
| | | | ACTCCTTTTGCAGTTTCTTTCGTTACATTTATGTTGCCGTCTTTATCAT |
| SNP337 | 2 | 805 | TTTGCTGAAGAGAGCAGGATTTTTATGTGGCAGTTGCTAGACTGCAGTATGA |
| | | | ACTCCTTTTGCAGTTTCTTTCGTTACATTTATGTTGCCGTCTTTATCAT |
| SNP338 | 1 | 806 | AAGGGAAGAGAATGTGCTTTGCTAGTAGTTTGAAGGAGTCTTTTTTGGTGTTT |
| | | | TGAGGGTTTGATTCTTTTTGTAATGGGCTGTGAATAAAGTGAAGTGCT |
| SNP338 | 2 | 807 | AAGGGAAGAGAATGTGCTTTGCTAGTAGTTTGAAGGAGTCTTTTTTGGTGCT |
| | | | TTGAGGGTTTGATTCTTTTTGTAATGGGCTGTGAATAAAGTGAAGTGCT |
| SNP339 | 1 | 808 | TTTTGAGTTAAATTCGAATAATTTCAAAGTTTACACAATAATTTTGATGTTGG |
| | | | TGGTTGTTCAGGTTCAATGTAACAATGAGGTAATTCAACAGCATTGTAACGG |
| | | | ACCTGTACAAAAATTAAAGCGATTTCTTATAAAGAAATTGAAACGTAATGTT |
| | | | TCTGTTGTTCGACAAAAGAAAGGTAATNAATCATGCAGCCGNTA |
| SNP339 | 2 | 809 | TTTTGAGTTAAATTCGAATAATTTCAAAGTTTACACAATAATTTTGATGTTGG |
| | | | TGGTTGTTCAGGTTCAATGTAACAATGAGGTAATTCAACAGCATTGTGACGG |
| | | | ACCTGTACAAAAATTAAAGCGATTTCTTATAAAGAAATTGAAACGTAATGTT |
| | | | TCTGTTGTTCGACAAAAGAAAGGTAATNAATCATGCAGCCGNTA |
| SNP340 | 1 | 810 | TCACCATATAACCACGAAATACCTTCTGGATCCTTAACGCTGAAGCAGATCT |
| | | | TCTATCAGGCGCCTCCGCCTGCGCCAACTCATCAGGTTTCTGTGACCGA |
| SNP340 | 2 | 811 | TCACCATATAACCACGAAATACCTTCTGGATCCTTAACGCTGAAGCAGATGT |
| | | | TCTATCAGGCGCCTCCGCCTGCGCCAACTCATCAGGTTTCTGTGACCGA |
| SNP341 | 1 | 812 | CCAGCTTGAAAAGCAGAGACATTAACGTTTCATTCACCCGTACCCTTTCCATT |
| | | | TCGTCTCTCCGAATCAACTCCGCCGTTTCGCCGAATAAAAGCTTCCGT |
| SNP341 | 2 | 813 | CCAGCTTGAAAAGCAGAGACATTAACGTTTCATTCACCCGTACCCTTTCCCTT |
| | | | TCGTCTCTCCGAATCAACTCCGCCGTTTCGCCGAATAAAAGCTTCCGT |
| SNP342 | 1 | 814 | GTTGCGCTCTATGTATTTTACTGTCTATATACACCATTCACTGCTGCTTCAAC |
| | | | GTGGACTGCGTTACCATCTTCCATGATCATCCCGTTGATTTGTGTTCT |
| SNP342 | 2 | 815 | GTTGCGCTCTATGTATTTTACTGTCTATATACACCATTCACTGCTGCTTCGAC |
| | | | GTGGACTGCGTTACCATCTTCCATGATCATCCCGTTGATTTGTGTTCT |
| SNP343 | 1 | 816 | ATTTGAGCTGTGAAAGGAGTTGCAGATTTGGGATAGTTAGGGCTTCTTGTAC |
| | | | CGAGGAGGTGGTGGTGGATGATCATGAGATTGATGACGTGGAGAGGAAG |
| SNP343 | 2 | 817 | ATTTGAGCTGTGAAAGGAGTTGCAGATTTGGGATAGTTAGGGCTTCTTGTGC |
| | | | CGAGGAGGTGGTGGTGGATGATCATGAGATTGATGACGTGGAGAGGAAG |
| SNP344 | 1 | 818 | CCTCTCTGCATCATACAGTTGATCGTGGGGGAACACACATATTTCTAGTTTCA |
| | | | GTAATATTCCTTTTGTATCTTGCATGCAGTCATTCAGAAAGAGATAGG |
| SNP344 | 2 | 819 | CCTCTCTGCATCATACAGTTGATCGTGGGGGAACACACATATTTCTAGTTCCA |
| | | | GTAATATTCCTTTTGTATCTTGCATGCAGTCATTCAGAAAGAGATAGG |
| SNP345 | 1 | 820 | GTCGATCGAACGTTATTGATAACGAGAACAACGATCTTTTCTCATGTGCATG |
| | | | CAAATCAGGCCTTGATATACGAGATGTGTCTATTTACGATGGTTTTCCT |
| SNP345 | 2 | 821 | GTCGATCGAACGTTATTGATAACGAGAACAACGATCTTTTCTCATGTGCACG |
| | | | CAAATCAGGCCTTGATATACGAGATGTGTCTATTTACGATGGTTTTCCT |
| SNP346 | 1 | 822 | ACTTAATCCAAGAAAGAGAGGAAATTGTGGCTTGTGGAATTTCAAATCAATA |
| | | | ATAACCCCAAAAAGAACTGCTAAAAACCGTAAGCAAGATACCACAGAGA |
| SNP346 | 2 | 823 | ACTTAATCCAAGAAAGAGAGGAAATTGTGGCTTGTGGAATTTCAAATCAACA |
| | | | ATAACCCCAAAAAGAACTGCTAAAAACCGTAAGCAAGATACCACAGAGA |
| SNP347 | 1 | 824 | GTGGAGCTCAATTTGGGGCTTATTTACAGAGGAGCACAGATACTCCTGCAAA |
| | | | TGGGAGTTGCGGTAGAATTGAAGCCACCGGAGAAAACCCAGTTTGGGAA |
| SNP347 | 2 | 825 | GTGGAGCTCAATTTGGGGCTTATTTACAGAGGAGCACAGATACTCCTGCACA |
| | | | TGGGAGTTGCGGTAGAATTGAAGCCACCGGAGAAAACCCAGTTTGGGAA |
| SNP348 | 1 | 826 | TCCTTGTCTTGAATCTTAGCTTTGACATTATCAATGGTGTCAGAACTCTCAAC |
| | | | CTCAAGAGTGATGGTCTTTCCGGTGAGTGTCTTAACAAATATCTGCAT |
| SNP348 | 2 | 827 | TCCTTGTCTTGAATCTTAGCTTTGACATTATCAATGGTGTCAGAACTCTCGAC |
| | | | CTCAAGAGTGATGGTCTTTCCGGTGAGTGTCTTAACAAATATCTGCAT |
| SNP349 | 1 | 828 | ATATCTTTGTTTTGCAAAACTCATGAATTTAAGCTTGTTCAAGCAAACGATGG |
| | | | AAGACGGAGGTCGTGAAATTAGAGTCCCTGTGGCATCAAGCTTCTCCA |
| SNP349 | 2 | 829 | ATATCTTTGTTTTGCAAAACTCATGAATTTAAGCTTGTTCAAGCAAACGACGG |
| | | | AAGACGGAGGTCGTGAAATTAGAGTCCCTGTGGCATCAAGCTTCTCCA |
| SNP350 | 1 | 830 | TTGAAGTTTGTGGATTTTGTCAGTAGGATGGTACTTGTTAGGACCCGAAATG |
| | | | CCAAGCAACTCGCGTCTCATGATCATAAGAGCAATATCGTCAATTATAA |
| SNP350 | 2 | 831 | TTGAAGTTTGTGGATTTTGTCAGTAGGATGGTACTTGTTAGGACCCGAAACG |
| | | | CCAAGCAACTCGCGTCTCATGATCATAAGAGCAATATCGTCAATTATAA |
| SNP351 | 1 | 832 | TAGTTTGATTGTTTGATCAAGCAGCAAGAACAAGTGCTTTTATTGTCCCTTGA |
| | | | CTGTTTGCTGTAACAATGGTTGGGCTGTCACGTTTCCAACAAACAGCA |
| SNP351 | 2 | 833 | TAGTTTGATTGTTTGATCAAGCAGCAAGAACAAGTGCTTTTATTGTCCCTCGA |
| | | | CTGTTTGCTGTAACAATGGTTGGGCTGTCACGTTTCCAACAAACAGCA |
| SNP352 | 1 | 834 | ATAGAGCAAAGAACCCTGACCATTCTAAGCAGCAACCACCTCTTGAGCACAT |
| | | | CTAGACTCCAACCATGATGTAGCTGCAACCTTCGTGGTCAAAAACCACC |
| SNP352 | 2 | 835 | ATAGAGCAAAGAACCCTGACCATTCTAAGCAGCAACCACCTCTTGAGCACGT |
| | | | CTAGACTCCAACCATGATGTAGCTGCAACCTTCGTGGTCAAAAACCACC |
| SNP353 | 1 | 836 | GCAACCTCAACTTCTAATTGTTTCTCTGTGTCACGGACTGAAGTAGTTGGTGA |
| | | | TGAGTCAAATTGCTCGTCCACATCTTGGACAACTGACACATCTGGGAT |
| SNP353 | 2 | 837 | GCAACCTCAACTTCTAATTGTTTCTCTGTGTCACGGACTGAAGTAGTTGGCGA |
| | | | TGAGTCAAATTGCTCGTCCACATCTTGGACAACTGACACATCTGGGAT |
| SNP354 | 1 | 838 | TCAAATGAGGAGCAAAAATTGGGTTTAAACACCAAAAAAATGCAGCAGCAA |
| | | | CAGCAGCCTTAAAAGGCCGAAACTGAAGAGCAAAAAATGGGTTTAAACTC |
| SNP354 | 2 | 839 | TCAAATGAGGAGCAAAAATTGGGTTTAAACACCAAAAAAATGCAGCAGCAG |
| | | | CAGCAGCCTTAAAAGGCCGAAACTGAAGAGCAAAAAATGGGTTTAAACTC |
| SNP355 | 1 | 840 | CCCATTACCAACACAGTGGCATCAGCATGCCTTGCAGCCACTTCTGCTAATC |
| | | | CAAAGTTTTGATTCCCTGGACAAGCTACTCCCATACATCCTTGTTGGTG |
| SNP355 | 2 | 841 | CCCATTACCAACACAGTGGCATCAGCATGCCTTGCAGCCACTTCTGCTAACC |
| | | | CAAAGTTTTGATTCCCTGGACAAGCTACTCCCATACATCCTTGTTGGTG |
| SNP356 | 1 | 842 | GCTTTAAAGGGTCCTAAAGATGTCTCTGATGGCCTAAGGAAACTTGATAAAC |
| | | | TAATAGAGAAAAGTGATGGACCAGAAAAGTTCCAGCTTGCTCGTGGACT |
| SNP356 | 2 | 843 | GCTTTAAAGGGTCCTAAAGATGTCTCTGATGGCCTAAGGAAACTTGATAAGC |
| | | | TAATAGAGAAAAGTGATGGACCAGAAAAGTTCCAGCTTGCTCGTGGACT |
| SNP357 | 1 | 844 | TTCTCAAGTCATCCCACTCATTATAGTTATCATTCCACACATCATCCAGGACA |
| | | | ACAAGAAACTTCTTCCCTTCGGGCTTTCTTTCAATTGACCTATAGCTT |
| SNP357 | 2 | 845 | TTCTCAAGTCATCCCACTCATTATAGTTATCATTCCACACATCATCCAGGGCA |
| | | | ACAAGAAACTTCTTCCCTTCGGGCTTTCTTTCAATTGACCTATAGCTT |
| SNP358 | 1 | 846 | TATTAGGAGACAGGTGTGATTTCACCTCTGGAATAATTTTCTTGTGAAACTGA |
| | | | CGATCATCATGAGATTCAACAAAACGCTGGGTCATATTGGAGGCTTGA |
| SNP358 | 2 | 847 | TATTAGGAGACAGGTGTGATTTCACCTCTGGAATAATTTTCTTGTGAAACCG |
| | | | ACGATCATCATGAGATTCAACAAAACGCTGGGTCATATTGGAGGCTTGA |
| SNP359 | 1 | 848 | GATATGTTGGAACAAAGTCGGCACATGTCCTATTACAGAGGAGAAGATGGTC |
| | | | ACTTTGAGAAATTGAAACAACTCTCTGAATCAGAGCAGCTGAGGACATT |
| SNP359 | 2 | 849 | GATATGTTGGAACAAAGTCGGCACATGTCCTATTACAGAGGAGAAGATGGC |
| | | | CACTTTGAGAAATTGAAACAACTCTCTGAATCAGAGCAGCTGAGGACATT |
| SNP360 | 1 | 850 | TGAGTGGATCTTTGTCGTGTGCATCTGTATGTGTGTGGTACATGCGTGCTTTT |
| | | | CTTCATCAATTTAGATGACAGTGAGACTACTCCAAAGATCATGCATTA |
| SNP360 | 2 | 851 | TGAGTGGATCTTTGTCGTGTGCATCTGTATGTGTGTGGTACATGCGTGCTCTT |
| | | | CTTCATCAATTTAGATGACAGTGAGACTACTCCAAAGATCATGCATTA |
| SNP361 | 1 | 852 | ATACTAGAGGGAGGTATATGGCCTATTGTCCAGGTGGCCAACTTCCGCTATA |
| | | | TACCAGTTAGGTATCAGCTCCTTTACGTCAATTTCTTCTGCTTGCTTGA |
| SNP361 | 2 | 853 | ATACTAGAGGGAGGTATATGGCCTATTGTCCAGGTGGCCAACTTCCGCTACA |
| | | | TACCAGTTAGGTATCAGCTCCTTTACGTCAATTTCTTCTGCTTGCTTGA |
| SNP362 | 1 | 854 | GGGTCTGGGGAGGACTTTACCCCTACCTTGGGAGGTAGAGAGTTGTTTTCAA |
| | | | TAGAACCTCGGCTCAAAACTAACTTGAAAAGATGCTTGGTATTACTGAG |
| SNP362 | 2 | 855 | GGGTCTGGGGAGGACTTTACCCCTACCTTGGGAGGTAGAGAGTTGTTTTCTA |
| | | | TAGAACCTCGGCTCAAAACTAACTTGAAAAGATGCTTGGTATTACTGAG |
| SNP363 | 1 | 856 | GAAAACTGAAGTTTGTCCTCCAAGTTTATGTGATAACCCAATTATATTATTGC |
| | | | TGTCCTCATCTTCTTCATAGTGTTGCAAACTGTGACCACTCAAACTAT |
| SNP363 | 2 | 857 | GAAAACTGAAGTTTGTCCTCCAAGTTTATGTGATAACCCAATTATATTATCGC |
| | | | TGTCCTCATCTTCTTCATAGTGTTGCAAACTGTGACCACTCAAACTAT |
| SNP364 | 1 | 858 | TCCAGGTCCTCAAGGCCTTTGCGAATACTGGAATTGAGCTTATGATTGGGATT |
| | | | CCAAACTCAGACTTGTTGGCGTTTTCTCAATTCGAGTCTAATGCCAAT |
| SNP364 | 2 | 859 | TCCAGGTCCTCAAGGCCTTTGCGAATACTGGAATTGAGCTTATGATTGGGGT |
| | | | TCCAAACTCAGACTTGTTGGCGTTTTCTCAATTCGAGTCTAATGCCAAT |
| SNP365 | 1 | 860 | AGAAAGCATTTCATATGGGCAAAGCTACGTTTGATTTTATATGTTCTGAAATA |
| | | | GAATCAGTAGTGACAAAAAAGGACACGATGTTACGTATGGCGATACCT |
| SNP365 | 2 | 861 | AGAAAGCATTTCATATGGGCAAAGCTACGTTTGATTTTATATGTTCTGAATTA |
| | | | GAATCAGTAGTGACAAAAAAGGACACGATGTTACGTATGGCGATACCT |
| SNP366 | 1 | 862 | ATGGCAAGGGTGGTTGTCGACCCATATTTCGTATTTATGGACAGGATCCATT |
| | | | CATAGTTTCTGATCGGTCTCCGAAAAGCTTGTTCTCAACACAAAAGAAA |
| SNP366 | 2 | 863 | ATGGCAAGGGTGGTTGTCGACCCATATTTCGTATTTATGGACAGGATCCACT |
| | | | CATAGTTTCTGATCGGTCTCCGAAAAGCTTGTTCTCAACACAAAAGAAA |
| SNP367 | 1 | 864 | TCTCTAATTGAAAAACCTTGTCCACCTCCACCTCCACCTCCACCACTTCCTGA |
| | | | GAAGCATGTGAAGGAAGATTTTTCTCTAACCGAAAAACTATGTCCACC |
| SNP367 | 2 | 865 | TCTCTAATTGAAAAACCTTGTCCACCTCCACCTCCACCTCCACCACTTCCCGA |
| | | | GAAGCATGTGAAGGAAGATTTTTCTCTAACCGAAAAACTATGTCCACC |
| SNP368 | 1 | 866 | GAGCTTCACGAAGTTATGGCAAGCAAAACGAAGATTTACTTCGCCATGGAAT |
| | | | ACGTTAAAGGCGGTGAATTGTTCGAAAAAGTAGCTAAAGGTAAGCTTAG |
| SNP368 | 2 | 867 | GAGCTTCACGAAGTTATGGCAAGCAAAACGAAGATTTACTTCGCCATGGAGT |
| | | | ACGTTAAAGGCGGTGAATTGTTCGAAAAAGTAGCTAAAGGTAAGCTTAG |
| SNP369 | 1 | 868 | TACTATATCTACTACTAATCTTGGTCCTTCATTCACTTGAGATGTCTTTGTGTA |
| | | | GACCTCCACTTCCTCGACTTCTGCTGAATAACGTCTCGTGTATGAGA |
| SNP369 | 2 | 869 | TACTATATCTACTACTAATCTTGGTCCTTCATTCACTTGAGATGTCTTTGCGTA |
| | | | GACCTCCACTTCCTCGACTTCTGCTGAATAACGTCTCGTGTATGAGA |
| SNP370 | 1 | 870 | CAACAATATAGGGACGAGGTCAATAACAACGAGCACAACAACAACAACAAT |
| | | | AATAGTAACGTTTGGGATCAGAGTGAAAAATACAAAGCGGATATTTTAAA |
| SNP370 | 2 | 871 | CAACAATATAGGGACGAGGTCAATAACAACGAGCACAACAACAACAACAAC |
| | | | AATAGTAACGTTTGGGATCAGAGTGAAAAATACAAAGCGGATATTTTAAA |
| SNP371 | 1 | 872 | ATAATAGGAGGCCAACAAGCCTTATAAGACGCAACTCGTGCTCTTGGTGATC |
| | | | CTCCCTTAGCTGTTCCATTGCCATATCCGAAAATGTTAGCTCCCTCGAC |
| SNP371 | 2 | 873 | ATAATAGGAGGCCAACAAGCCTTATAAGACGCAACTCGTGCTCTTGGTGACC |
| | | | CTCCCTTAGCTGTTCCATTGCCATATCCGAAAATGTTAGCTCCCTCGAC |
| SNP372 | 1 | 874 | CTTGCCAAACAAGAGTATAAGTTCCACAATGGAAATGCCAAGAGCAGGGGT |
| | | | CTTCTCACAGCAGCAAGGAACCAGGAATTTTCAATCTGGAAGTTCACCGC |
| SNP372 | 2 | 875 | CTTGCCAAACAAGAGTATAAGTTCCACAATGGAAATGCCAAGAGCAGGGGC |
| | | | CTTCTCACAGCAGCAAGGAACCAGGAATTTTCAATCTGGAAGTTCACCGC |
| SNP373 | 1 | 876 | ATTCAGATTTCGAGCTACAACTTTTCTTACTTTCCAGTATCCTCTACTGCTTGA |
| | | | ACCCGAGATTTAACGTCTGATTCGGACATTGGATCCCGAATTGATCC |
| SNP373 | 2 | 877 | ATTCAGATTTCGAGCTACAACTTTTCTTACTTTCCAGTATCCTCTACTGCCTG |
| | | | AACCCGAGATTTAACGTCTGATTCGGACATTGGATCCCGAATTGATCC |
| SNP374 | 1 | 878 | CATTTAGTTCACACTGATCTGATCAGCTGCTAAACACGCGAGTACAAGGAAG |
| | | | CAGAGGTAACATTTCAGATTATTCACTTTCGAGTACATTCTGTCTAAGA |
| SNP374 | 2 | 879 | CATTTAGTTCACACTGATCTGATCAGCTGCTAAACACGCGAGTACAAGGAGG |
| | | | CAGAGGTAACATTTCAGATTATTCACTTTCGAGTACATTCTGTCTAAGA |
| SNP375 | 1 | 880 | TTAACAATACCAGCATTAATGTTGAACAGATCATCACGGGTCATACCAGGTT |
| | | | TCCGTGGCACACCAGCTGGAATAATGACAACATCAGCTCCCTCCAAAGC |
| SNP375 | 2 | 881 | TTAACAATACCAGCATTAATGTTGAACAGATCATCACGGGTCATACCAGGCT |
| | | | TCCGTGGCACACCAGCTGGAATAATGACAACATCAGCTCCCTCCAAAGC |
| SNP376 | 1 | 882 | CAATCCAAGTTTACATTATAAAGATCTGGAAGAAGCCTACTTGTGGAAGTAA |
| | | | CATTTTCATCTTTACCAGGTACAGATACTGAGACTGAAGAATCACTCCA |
| SNP376 | 2 | 883 | CAATCCAAGTTTACATTATAAAGATCTGGAAGAAGCCTACTTGTGGAAGTTA |
| | | | CATTTTCATCTTTACCAGGTACAGATACTGAGACTGAAGAATCACTCCA |
| SNP377 | 1 | 884 | TTAAAATTTGTTCATTTTGCTTGGTAGTGAAGTTGAGGCCTCCGTTAATCTGT |
| | | | GGTCATCAAACCCGATTCTTAACATACCTCATATATTTGGCAGAAGAG |
| SNP377 | 2 | 885 | TTAAAATTTGTTCATTTTGCTTGGTAGTGAAGTTGAGGCCTCCGTTAATCCGT |
| | | | GGTCATCAAACCCGATTCTTAACATACCTCATATATTTGGCAGAAGAG |
| SNP378 | 1 | 886 | CAGGGTGGTTTGCATGGCTATTTTCCGTCATCTAAGGTTCTTGTTTGGTGTGA |
| | | | TTCCAAGTGATCACGGTGCAACCGAGACTACTGTGAACCTTGGAAGGA |
| SNP378 | 2 | 887 | CAGGGTGGTTTGCATGGCTATTTTCCGTCATCTAAGGTTCTTGTTTGGTGGGA |
| | | | TTCCAAGTGATCACGGTGCAACCGAGACTACTGTGAACCTTGGAAGGA |
| SNP379 | 1 | 888 | AATAAAAAAACAAATCAAATCGACAAACAATACATATCTGGTTAACTGAGT |
| | | | GAAGATGAGATGCCATTCTTGTTGCCACTGACAGACGAGGGTTTTCCAAC |
| SNP379 | 2 | 889 | AATAAAAAAACAAATCAAATCGACAAACAATACATATCTGGTTAACTGAGC |
| | | | GAAGATGAGATGCCATTCTTGTTGCCACTGACAGACGAGGGTTTTCCAAC |
| SNP380 | 1 | 890 | CAGAGAGACCGGCCAGCATTGGGAACCAAAAATGCTCTGTGACGTCACACC |
| | | | TCTCATCTTCAGTAGTGTCAACTGGCTTCAGAGCACCACCAGGTATAAGA |
| SNP380 | 2 | 891 | CAGAGAGACCGGCCAGCATTGGGAACCAAAAATGCTCTGTGACGTCACACG |
| | | | TCTCATCTTCAGTAGTGTCAACTGGCTTCAGAGCACCACCAGGTATAAGA |
| SNP381 | 1 | 892 | GAACTAGGAGCAGGACATCCAAGAAGAGCAAAGCTTTCAGTATTAGTGGTA |
| | | | TCAATAACTTCACTTGTAATTGGTGCATTATTGACAATATTACTCTTACT |
| SNP381 | 2 | 893 | GAACTAGGAGCAGGACATCCAAGAAGAGCAAAGCTTTCAGTATTAGTGGTG |
| | | | TCAATAACTTCACTTGTAATTGGTGCATTATTGACAATATTACTCTTACT |
| SNP382 | 1 | 894 | TAGAGGGTCTTCAGGAGATAAAGGTAGTCCGAGTCAAACTGTATCAAGCTTC |
| | | | CATGCAACACCATATGAAGTTCCATTACAAACCCAGAATAGATTTCTTT |
| SNP382 | 2 | 895 | TAGAGGGTCTTCAGGAGATAAAGGTAGTCCGAGTCAAACTGTATCAAGCTCC |
| | | | CATGCAACACCATATGAAGTTCCATTACAAACCCAGAATAGATTTCTTT |
| SNP383 | 1 | 896 | GAGGACATTATGGAGCGCATAACAGCCATGAACTTGATGAAACCTCAAAAA |
| | | | TGTAGGCTCAAACGAACACCAAGTCATCCTCGAAAACAGAAACAAGTAAG |
| SNP383 | 2 | 897 | GAGGACATTATGGAGCGCATAACAGCCATGAACTTGATGAAACCTCAAAAC |
| | | | TGTAGGCTCAAACGAACACCAAGTCATCCTCGAAAACAGAAACAAGTAAG |
| SNP384 | 1 | 898 | GATGAAAGCCCTGGCAGAAAATCAATTGTGTTCTCTCAATTCAGGAAGTTTT |
| | | | TGCTCCTACTTGAAGAGCCGCTTAAAGCAGCTGGTTTTAAGATATTGCG |
| SNP384 | 2 | 899 | GATGAAAGCCCTGGCAGAAAATCAATTGTGTTCTCTCAATTCAGGAAGTTGT |
| | | | TGCTCCTACTTGAAGAGCCGCTTAAAGCAGCTGGTTTTAAGATATTGCG |
| SNP385 | 1 | 900 | TAACATCTATGGTACGTTTCAAAGCACGCCAAAGAATTAGTGCAAAGACAAC |
| | | | TTTAGCACATCCATACTTTGATAGAGAAGGTCTTCTAGCCCTGTCCTTC |
| SNP385 | 2 | 901 | TAACATCTATGGTACGTTTCAAAGCACGCCAAAGAATTAGTGCAAAGACAGC |
| | | | TTTAGCACATCCATACTTTGATAGAGAAGGTCTTCTAGCCCTGTCCTTC |
| SNP386 | 1 | 902 | CTTTCTTGAGCCTAGCTGCCTGAATGTCATCAAACATGTGTGTCCTTCTCTCC |
| | | | TTCCTTGTTAGCATGAAATATATATGTATACATCTGTTTATTTTGTCT |
| SNP386 | 2 | 903 | CTTTCTTGAGCCTAGCTGCCTGAATGTCATCAAACATGTGTGTCCTTCTCCCC |
| | | | TTCCTTGTTAGCATGAAATATATATGTATACATCTGTTTATTTTGTCT |
| SNP387 | 1 | 904 | CAATAGGAACAGAAATTGATGGGCACTAAGCTGTCACATCAAATGTAATGA |
| | | | AAGCTGTCCAATGTTCAACGTGTTGGCCACTTATGCCTTATCAAGAAATA |
| SNP387 | 2 | 905 | CAATAGGAACAGAAATTGATGGGCACTAAGCTGTCACATCAAATGTAATGTA |
| | | | AGCTGTCCAATGTTCAACGTGTTGGCCACTTATGCCTTATCAAGAAATA |
| SNP388 | 1 | 906 | AGGGAGGTTGTTTCCGATAGTCCCATAGTTTATTCCGACACCATGAACATTGT |
| | | | AGTTAATAAGAGAGAGGAGAATGAAGAGGGATAAGACAAGAGTACAAA |
| SNP388 | 2 | 907 | AGGGAGGTTGTTTCCGATAGTCCCATAGTTTATTCCGACACCATGAACATCG |
| | | | TAGTTAATAAGAGAGAGGAGAATGAAGAGGGATAAGACAAGAGTACAAA |
| SNP389 | 1 | 908 | GAAAAGGAGCATCGAAGCAACAGTAAAGTTGTCTCTGAGTGAATTATAGGT |
| | | | AAGGGGTTTAAGCCGTCAGAATAGTCACTAATACTTGTAATTAGGATAGA |
| SNP389 | 2 | 909 | GAAAAGGAGCATCGAAGCAACAGTAAAGTTGTCTCTGAGTGAATTATAGGC |
| | | | AAGGGGTTTAAGCCGTCAGAATAGTCACTAATACTTGTAATTAGGATAGA |
| SNP390 | 1 | 910 | CAGCGGTTGATGAAACTGAACTGCTAGGAACATCTGACACTTCTCCTGCCAC |
| | | | AGTTCCCACATCTTTCTTTTTGGTCATGAAAAGATAAGCTACATAAATT |
| SNP390 | 2 | 911 | CAGCGGTTGATGAAACTGAACTGCTAGGAACATCTGACACTTCTCCTGCCTC |
| | | | AGTTCCCACATCTTTCTTTTTGGTCATGAAAAGATAAGCTACATAAATT |
| SNP391 | 1 | 912 | AACTTCACTGAAACTTCAATCAAAAAAACCATCTTCTTCGGTAACCCAACAA |
| | | | ACAAACTTCTCAGAGTATTGCCGGTGCATCTGCTTACTCACTTTAGCAT |
| SNP391 | 2 | 913 | AACTTCACTGAAACTTCAATCAAAAAAACCATCTTCTTCGGTAACCCAACGA |
| | | | ACAAACTTCTCAGAGTATTGCCGGTGCATCTGCTTACTCACTTTAGCAT |
| SNP392 | 1 | 914 | CACCTCTATACTGCTGTAGACAAGCAGAGTCCAATCCAGAGGATGTTTCTAA |
| | | | GAACACTTGTTAGACAATCAACATCAGATGACAGTTTACTAGCGTATCA |
| SNP392 | 2 | 915 | CACCTCTATACTGCTGTAGACAAGCAGAGTCCAATCCAGAGGATGTTTCTCA |
| | | | GAACACTTGTTAGACAATCAACATCAGATGACAGTTTACTAGCGTATCA |
| SNP393 | 1 | 916 | GTTCTTCAGCTTACGTTCTTCAGCTCAGCAACAGTTCGTCCGCAAGAGCTAAG |
| | | | CTCAGCTCAGCTCTTCAGTCTTCTGCTTCCTCTCTTCGATCTTCAGTA |
| SNP393 | 2 | 917 | GTTCTTCAGCTTACGTTCTTCAGCTCAGCAACAGTTCGTCCGCAAGAGCTGAG |
| | | | CTCAGCTCAGCTCTTCAGTCTTCTGCTTCCTCTCTTCGATCTTCAGTA |
| SNP394 | 1 | 918 | CCTGTTTTATTTGTTGGACATCTTTCGAAAACTTCCACCTTGATCATAGATAA |
| | | | ACCTTGGATTCAAGTGGTTAAAACTCTTGATGCTCAACCAGTTCACAG |
| SNP394 | 2 | 919 | CCTGTTTTATTTGTTGGACATCTTTCGAAAACTTCCACCTTGATCATAGACAA |
| | | | ACCTTGGATTCAAGTGGTTAAAACTCTTGATGCTCAACCAGTTCACAG |
| SNP395 | 1 | 920 | TATCCAGGGATTAAGGGCGAAGGTCCTGAATCAGGTGAGAAGTCTCTCCGAA |
| | | | GAGGCTGGAGGCAAAGGGTCTGCCAAGAAGGACCTAAACAGTCAAAGAA |
| SNP395 | 2 | 921 | TATCCAGGGATTAAGGGCGAAGGTCCTGAATCAGGTGAGAAGTCTCTCCGTA |
| | | | GAGGCTGGAGGCAAAGGGTCTGCCAAGAAGGACCTAAACAGTCAAAGAA |
| SNP396 | 1 | 922 | TCCGAAACTGTTGAAGTGTCTGCACTATTCATATTTTGATTCGAACCAACTGA |
| | | | ACCTTCAAGCCATACAGTTGGATTTCTTGACGCCTTTGTACTGGCACG |
| SNP396 | 2 | 923 | TCCGAAACTGTTGAAGTGTCTGCACTATTCATATTTTGATTCGAACCAACCGA |
| | | | ACCTTCAAGCCATACAGTTGGATTTCTTGACGCCTTTGTACTGGCACG |
| SNP397 | 1 | 924 | GCAATCGGGTATGGGCAACGAGTGCCCATACGATTTAAGGTTGCGGGGCGA |
| | | | GTGGATATAAATTTGGTCACGGTAACAATGGTTGCACCTGGATTTAACAC |
| SNP397 | 2 | 925 | GCAATCGGGTATGGGCAACGAGTGCCCATACGATTTAAGGTTGCGGGGCGG |
| | | | GTGGATATAAATTTGGTCACGGTAACAATGGTTGCACCTGGATTTAACAC |
| SNP398 | 1 | 926 | AACCAATGGCCCCAACCTAGGTGAGAGAACTCAGGCAAACCAGAGAGAGGA |
| | | | GATGGGACGTTTGCAGGATGTGAAGGATGATTTAGTGTTCCGGCAACTTC |
| SNP398 | 2 | 927 | AACCAATGGCCCCAACCTAGGTGAGAGAACTCAGGCAAACCAGAGAGAGGG |
| | | | GATGGGACGTTTGCAGGATGTGAAGGATGATTTAGTGTTCCGGCAACTTC |
| SNP399 | 1 | 928 | CAACAACACAAAAATGGTTTACGCACACGCCATGGGTTAGAAAATAATCAA |
| | | | GAATTTCAACATCAACCGTATAAAAATGGTTTAGGCCCACGTCACGGGTT |
| SNP399 | 2 | 929 | CAACAACACAAAAATGGTTTACGCACACGCCATGGGTTAGAAAATAATCAG |
| | | | GAATTTCAACATCAACCGTATAAAAATGGTTTAGGCCCACGTCACGGGTT |

TABLE 10 shows the primers selected by PlexForm™ for the targets in TABLE 9. “F” in the primer name denotes a forward primer. “R” in the primer name denotes a reverse primer. TABLE 10 comprises SEQ ID NOs: 930-1529.

TABLE 10
PlexForm primers selected (for the target sequences in TABLE 9)

| Target | Primer Name | SEQ ID NO: | Primer Sequence |
|---|---|---|---|
| SNP100 | SNP100-F | 930 | TGTGGCCTGGAGAGGGTAGG |
| SNP100 | SNP100-R | 931 | TCTCCACCCTTCAAAGGTAGAGG |
| SNP101 | SNP101-F | 932 | GATCTGTTGTACTTCTTTCTTGCAGC |
| SNP101 | SNP101-R | 933 | GTCGAAATTATTGATTCCTAAATCTTTTCGTG |
| SNP102 | SNP102-F | 934 | GGAGGAAAAAGATACCCTTTGTTGC |
| SNP102 | SNP102-R | 935 | CTTGCATCCCTTCAAAAATGGCAA |
| SNP103 | SNP103-F | 936 | GTGGTGAGGCTGGAAATGTAATCC |
| SNP103 | SNP103-R | 937 | ACGTCAAGAACCGGAATGTTCC |
| SNP104 | SNP104-F | 938 | CCACTATACAAGATGGAGGCTGGTAAG |
| SNP104 | SNP104-R | 939 | ATGTCCTCATACCTACATTGCAAAATC |
| SNP105 | SNP105-F | 940 | CGCGTAACATTTGTGTCCAACG |
| SNP105 | SNP105-R | 941 | GACAGACAACAGTACAATGATAGCTTGG |
| SNP106 | SNP106-F | 942 | GACAGTAAGATGGAGAATTTTGTTCCTG |
| SNP106 | SNP106-R | 943 | CTCCACGCCTCCATTTCTCG |
| SNP107 | SNP107-F | 944 | AGTGAAGATAGCCTCCAAGCATTTC |
| SNP107 | SNP107-R | 945 | ATGTTCGGGGGTAATCTCACTCC |
| SNP108 | SNP108-F | 946 | AACAACAACAACGGGGCCC |
| SNP108 | SNP108-R | 947 | GGTTGTTGTTGTTATTGTTATCAAACTGG |
| SNP109 | SNP109-F | 948 | CCCATTGAACAGCATGGCAATG |
| SNP109 | SNP109-R | 949 | TTAGCAGGAATTTCCATCATTGCG |
| SNP110 | SNP110-F | 950 | GCTTTGTGATTAGGTAAAGGTGATCAC |
| SNP110 | SNP110-R | 951 | ACCATCTTCGCCATCCTTGTCT |
| SNP111 | SNP111-F | 952 | TTTCGCTTGTGCCTCTTCTCCC |
| SNP111 | SNP111-R | 953 | CCCGTTGCATTCCTCTTACACTAG |
| SNP112 | SNP112-F | 954 | CAGAAACAACTGCATCCACTGAAG |
| SNP112 | SNP112-R | 955 | ACTGGAATATTCTCTACCCCTAATATTTCTG |
| SNP113 | SNP113-F | 956 | GTCGTCTCATGGATGATGATATCTCTAAG |
| SNP113 | SNP113-R | 957 | GCAGATGCATCAGTTCTGGAAAC |
| SNP114 | SNP114-F | 958 | GCTGAACTTCTTGCCACCTTCTG |
| SNP114 | SNP114-R | 959 | TCTTCAATAGCTTCATCACTCAATTTCTCAC |
| SNP115 | SNP115-F | 960 | GAGCTGAAGCAGAGTTTCCCAC |
| SNP115 | SNP115-R | 961 | CAGGACACAACTGGCATACTTTTG |
| SNP116 | SNP116-F | 962 | GTGGAAGGAAGTACAGAGAAGAAGC |
| SNP116 | SNP116-R | 963 | GCTCTGGAAAAAACAATCTCCCAC |
| SNP117 | SNP117-F | 964 | ACAGGAACATCGTCAACACCTTG |
| SNP117 | SNP117-R | 965 | TTCTGGCGAAAATAGTTCCTCCAC |
| SNP118 | SNP118-F | 966 | TGAAGACATCAATGATTGACAGTGCT |
| SNP118 | SNP118-R | 967 | TAGGAGCTTCTTCCCACCCTGG |
| SNP119 | SNP119-F | 968 | TTCATCTTGAGGGAGCTCACATG |
| SNP119 | SNP119-R | 969 | CACCAGCTGATCTGAAATCGGGG |
| SNP120 | SNP120-F | 970 | AACTCTGTGTGGCCTGACAATG |
| SNP120 | SNP120-R | 971 | GGGAAATTTATTTTCTGACTGAACTTTTCTC |
| SNP121 | SNP121-F | 972 | TGTCCAAGTATCCCTCATTGTGGG |
| SNP121 | SNP121-R | 973 | TTCTACTTCACAGAACTCATCGGTC |
| SNP122 | SNP122-F | 974 | ACAATGGCAATTTGGAGCAATGG |
| SNP122 | SNP122-R | 975 | TCAGTCTGATTTCCCAAGTTAGAGG |
| SNP123 | SNP123-F | 976 | CACTGTGAAGAGAATATGATGAGAGGA |
| SNP123 | SNP123-R | 977 | GCTCTAGCAAATAATGTCAATGTGCA |
| SNP124 | SNP124-F | 978 | CAACTACTAAACGGTCAATTTTAAGTGAGC |
| SNP124 | SNP124-R | 979 | TGTTTTCTGGTTTGATATTTAGAGAAATGGG |
| SNP125 | SNP125-F | 980 | CACTGAAGCATTAGCCTTTGTCTC |
| SNP125 | SNP125-R | 981 | GGAACAGAACATTTGGGGCTACC |
| SNP126 | SNP126-F | 982 | ATCTCGGCGAGCTTGGTGAC |
| SNP126 | SNP126-R | 983 | CGCACTCTCCATGGCATCTTATG |
| SNP127 | SNP127-F | 984 | TGGTGATTCCTGTTCATAGATTTGGC |
| SNP127 | SNP127-R | 985 | CAGTTTTTTATAGTCCATACACGAATTTAAGGC |
| SNP128 | SNP128-F | 986 | TTCTTACTTCTTCAATAAACCTCAGGAATTC |
| SNP128 | SNP128-R | 987 | ATCGGAATCAACGTCAATGGCG |
| SNP129 | SNP129-F | 988 | ATTGGTTGTTCGAGTTTGCTCAG |
| SNP129 | SNP129-R | 989 | GGGCATCCGGATCAATGCC |
| SNP130 | SNP130-F | 990 | AGAGTTTAGATCCAAGACTGTGAATTAGG |
| SNP130 | SNP130-R | 991 | TGACATACTTTCTATCTTGCCATGTGG |
| SNP131 | SNP131-F | 992 | GCCAACAGGGAACAAGAGACTAAAGG |
| SNP131 | SNP131-R | 993 | CAGCTTGAAGTTCAGCCTCTATCC |
| SNP132 | SNP132-F | 994 | GACTTGAGAATAAAGCGAAATTCCTCTTTG |
| SNP132 | SNP132-R | 995 | TGTGCATGGATTCAACTGTTAGGTTG |
| SNP133 | SNP133-F | 996 | TGTTGAAACCAAAATTATACATAGAATATCTGTCC |
| SNP133 | SNP133-R | 997 | GTGTAATCTTCAATGTCCCTCATGAGAG |
| SNP134 | SNP134-F | 998 | AGTATCATTTTGAAAGATACAAAGCAAACAAG |
| SNP134 | SNP134-R | 999 | TTCTAGATATAAAAATCTTACAAATTCGTGCAAG |
| SNP135 | SNP135-F | 1000 | TCCCTGGCCAAACAACCCC |
| SNP135 | SNP135-R | 1001 | AGGCATGGAAGCAGATGTCTTC |
| SNP136 | SNP136-F | 1002 | AAGGATTTTGCGAAGGAGGCTAAA |
| SNP136 | SNP136-R | 1003 | CCTCTTAATCCTACTACATTAGGATGCCT |
| SNP137 | SNP137-F | 1004 | CAATTGTCAAAGACTCGTAACCTGC |
| SNP137 | SNP137-R | 1005 | GCAAAAGAGAAGAAAAAACACCTGTTC |
| SNP138 | SNP138-F | 1006 | CAAGAGGAGGCAGAGGAGTGG |
| SNP138 | SNP138-R | 1007 | TTTGCTTCCTTGGCAGCAATGC |
| SNP139 | SNP139-F | 1008 | GGCAGTACCACGAAGCTTGAC |
| SNP139 | SNP139-R | 1009 | AAACCTTTAAGAATGGTGAAAACTGTGC |
| SNP140 | SNP140-F | 1010 | TGATGCAAAAGAAGAATGTTGATGCTG |
| SNP140 | SNP140-R | 1011 | TCGCAACCAATCCTTGTAATCTAATGG |
| SNP141 | SNP141-F | 1012 | GTTCTGAACCAGTGTAATTATGTTGTTCTTG |
| SNP141 | SNP141-R | 1013 | AGGCTCAAAACCCATGTCAATCATAC |
| SNP142 | SNP142-F | 1014 | ATCAAAGAGCGGGAGGATTAAGC |
| SNP142 | SNP142-R | 1015 | TTGCTTGTTTGCTACTCCATTTGATG |
| SNP143 | SNP143-F | 1016 | GAAGCAAAAGTAGATCCAGAATCCAG |
| SNP143 | SNP143-R | 1017 | GGTCAGAACCAGAAATCTTTTACAAGC |
| SNP144 | SNP144-F | 1018 | GGCAATTGGGAGTTGGGCC |
| SNP144 | SNP144-R | 1019 | GGAGCATCATCTCAGATTCATTGAGC |
| SNP145 | SNP145-F | 1020 | TGGTGGGCAGGGGATTCTG |
| SNP145 | SNP145-R | 1021 | TGTGGAACAAATTCAAGTACTACATTTCG |
| SNP146 | SNP146-F | 1022 | ACTCTTCCAAACCTACCGCAAAAGA |
| SNP146 | SNP146-R | 1023 | TCCTGACAACTCAAGTCATATATAGGGG |
| SNP147 | SNP147-F | 1024 | CCCCTCTAGTAGCCAGCCAAG |
| SNP147 | SNP147-R | 1025 | GGAAGTTTTGATGCAGTTTACATTTCC |
| SNP148 | SNP148-F | 1026 | ATAAGCTTTGCCATTGTAAGAGATAAGATC |
| SNP148 | SNP148-R | 1027 | AGTCTAAGAACATATTCACAGTCCAATTTG |
| SNP149 | SNP149-F | 1028 | ACTGACAAAAAAGGGCCTTCAATGG |
| SNP149 | SNP149-R | 1029 | GGCACAGAGAACCACCAAATTC |
| SNP150 | SNP150-F | 1030 | GCCAGCCATTTCTTCGCCG |
| SNP150 | SNP150-R | 1031 | GAGTTCCTCCCCTATTTGATAGATAACG |
| SNP151 | SNP151-F | 1032 | GCTCCGTTCATTCCTTCCTAGC |
| SNP151 | SNP151-R | 1033 | GCAGAGTCAAGTTGAGAAGCTTAAGC |
| SNP152 | SNP152-F | 1034 | CGCTTGTGTTGTTGGTGCC |
| SNP152 | SNP152-R | 1035 | TAAGCTTAAATTTTCTAGGCAGCAGC |
| SNP153 | SNP153-F | 1036 | CAGAAATTCGGGTTTCGGTTCAG |
| SNP153 | SNP153-R | 1037 | AAGGGGTGCCATTCAAGTTAATTG |
| SNP154 | SNP154-F | 1038 | ATTCATCAATTTTTTCCTGCATCAGTTTC |
| SNP154 | SNP154-R | 1039 | TCAAGCGAAGGCCTGAAGAAG |
| SNP155 | SNP155-F | 1040 | GCATTATCTTTCAGGCCAGAGCG |
| SNP155 | SNP155-R | 1041 | TGAACTCGAAATCTTGACCCCTGA |
| SNP156 | SNP156-F | 1042 | CTTCGACGTCTAAGGCTTTTGC |
| SNP156 | SNP156-R | 1043 | CAGGGATGCGATGAGAGAACAAATATG |
| SNP157 | SNP157-F | 1044 | ATCAGCTTATGAACTCCAAACGTTGTTTG |
| SNP157 | SNP157-R | 1045 | CTGGTAAATGAACTGATGGTCAAAGAG |
| SNP158 | SNP158-F | 1046 | ATCCAATATCGAGGCAGGGTTG |
| SNP158 | SNP158-R | 1047 | TCTCACCAGAGTCTGCTAATTTAGAGTC |
| SNP159 | SNP159-F | 1048 | ACCACAAGTACACAACTAAAGCTTTG |
| SNP159 | SNP159-R | 1049 | GTTGGTATGTTCGTCAAGACTGAAAC |
| SNP160 | SNP160-F | 1050 | TGAGCTGATAATGTACTATCTAAAGCGG |
| SNP160 | SNP160-R | 1051 | AATGTTGAGTTCTGATATGACTTCAAAAAGG |
| SNP161 | SNP161-F | 1052 | GTTCACCCAACTCTTCAATTCTTGAATC |
| SNP161 | SNP161-R | 1053 | ACAGATTCTCCTACTTTCTTATTCAACAAGATTTG |
| SNP162 | SNP162-F | 1054 | TCACATTTTTCGTGCAGATAAAGTATACC |
| SNP162 | SNP162-R | 1055 | GGGAGCAAAGGATGAGACTTGC |
| SNP163 | SNP163-F | 1056 | AGAGTTTTCCTTGGGCTCAAGCA |
| SNP163 | SNP163-R | 1057 | AAAAATTGTTTTTGATCTTGTCAAAGTCCG |
| SNP164 | SNP164-F | 1058 | GCAAGGGTACAATTCCTATCCCTGG |
| SNP164 | SNP164-R | 1059 | CCAAGGGCTCCTAGGTTCTCTTC |
| SNP165 | SNP165-F | 1060 | CTGAACGGCCAACCATTTCAG |
| SNP165 | SNP165-R | 1061 | CATGCTGGCAGAGGAAGTGAGAG |
| SNP166 | SNP166-F | 1062 | TAAGTTTCCGTTCGATGCCGG |
| SNP166 | SNP166-R | 1063 | TGATCAATTCTCCTCTTCCGGC |
| SNP167 | SNP167-F | 1064 | TGCAAGTTGCTGGTTAAGTGCC |
| SNP167 | SNP167-R | 1065 | TCACCCGAAAGAGTTAATTAAACATGC |
| SNP168 | SNP168-F | 1066 | TGATGGGTGTTCAATTATTGAGATGTG |
| SNP168 | SNP168-R | 1067 | GGCAGCTTTTCATCTTAAAGTGAGC |
| SNP169 | SNP169-F | 1068 | GCCATGTGGTGAGTGCTGC |
| SNP169 | SNP169-R | 1069 | TTGTTACAGTAACCCTTCTGCAGATCC |
| SNP170 | SNP170-F | 1070 | CTGGGGTGGGAAGTAGGGGAAT |
| SNP170 | SNP170-R | 1071 | TATGGAGCTCCAGTTCCTCCCG |
| SNP171 | SNP171-F | 1072 | AAATCTACAAAACGAAGCGGAACTG |
| SNP171 | SNP171-R | 1073 | CGTCAATTTCACTCCCTAAGAAGCC |
| SNP172 | SNP172-F | 1074 | CTGTTGCATTGTAGGTCTATCATCTGG |
| SNP172 | SNP172-R | 1075 | AGAAGCAATTATAGAAATAGCTGCAAGATG |
| SNP173 | SNP173-F | 1076 | CTGAGACTCATCAGAATCCGCC |
| SNP173 | SNP173-R | 1077 | CCGGCGAAGGTGATGGAGAC |
| SNP174 | SNP174-F | 1078 | CTCGTGGTGCAAAACTCGGAG |
| SNP174 | SNP174-R | 1079 | TCAAGGAGAATCTGTTGTACGATCTC |
| SNP175 | SNP175-F | 1080 | GCTGTGAACATTGAGACAGTTGAG |
| SNP175 | SNP175-R | 1081 | CTGAGAGCACCTGATAACTCACTTTATG |
| SNP176 | SNP176-F | 1082 | AAGCCAGAAATTGTGATTATTGATTGTGG |
| SNP176 | SNP176-R | 1083 | GGCCCTCCAATTATTCCCATCC |
| SNP177 | SNP177-F | 1084 | CGGCGACCGGAGAATCCTAC |
| SNP177 | SNP177-R | 1085 | TCGAGACACTCACGGTCGG |
| SNP178 | SNP178-F | 1086 | AGAAGCAGAAGAGACTATGACAGGAG |
| SNP178 | SNP178-R | 1087 | CGGCTGTGGCTTCTACTCCTAC |
| SNP179 | SNP179-F | 1088 | TTCCCTGCTCATGATCTGCAAG |
| SNP179 | SNP179-R | 1089 | AGGATCGTCTGAACATCCTTCCAC |
| SNP180 | SNP180-F | 1090 | CACCAGAAAATCGATCTGTTCTGTG |
| SNP180 | SNP180-R | 1091 | CGGGTGGGGGTAAGTTTATCC |
| SNP181 | SNP181-F | 1092 | CGTGCCTTCAGGAAATGCAGC |
| SNP181 | SNP181-R | 1093 | TGTGCAATGCCTGTTTCCCTTC |
| SNP182 | SNP182-F | 1094 | CCATCTTCATTCACTTTATTCTTCCCTTTG |
| SNP182 | SNP182-R | 1095 | TCAGGGTCCAAAACTGAATGACAC |
| SNP183 | SNP183-F | 1096 | TCTTAAAAGTTGTGAGCCATCAAATCTC |
| SNP183 | SNP183-R | 1097 | GTAGAGTCCGAAAACTACAACCCC |
| SNP184 | SNP184-F | 1098 | TACTTCCTGGACAACCATCAACC |
| SNP184 | SNP184-R | 1099 | GGTGAGGGCTACACAAAGGCAG |
| SNP185 | SNP185-F | 1100 | ACCACACAGATGCCGACGG |
| SNP185 | SNP185-R | 1101 | GCATGGACGATGAGAGCTTCAG |
| SNP186 | SNP186-F | 1102 | CAGCCAAACGATAAATATTCCTTCGAG |
| SNP186 | SNP186-R | 1103 | TAGGTTCAGATAGCCAGACGAGG |
| SNP187 | SNP187-F | 1104 | CGCACTGATGGTGGCAAATTC |
| SNP187 | SNP187-R | 1105 | TCCAGTGCCCACTTTGGATAGC |
| SNP188 | SNP188-F | 1106 | TTTATCTTTAAATTCCATGCCACCCC |
| SNP188 | SNP188-R | 1107 | GCTTCGTCAGAGAGGTACTTCTG |
| SNP189 | SNP189-F | 1108 | GCCATTTGCAATGTTTTAGTTGGTG |
| SNP189 | SNP189-R | 1109 | CTGAATTAGACAACGCATGCTTGC |
| SNP190 | SNP190-F | 1110 | GACATGGTCAATATCGATGCATCGC |
| SNP190 | SNP190-R | 1111 | AAATCATAATTGAACAATCACCAAGGGG |
| SNP191 | SNP191-F | 1112 | AGCAGACATCTTTATACAAGAAAATGTGGC |
| SNP191 | SNP191-R | 1113 | TTTTGTTGTGCCAATTTGTTATTTATTTGAC |
| SNP192 | SNP192-F | 1114 | GCCGCATCTACATAATGCCCAG |
| SNP192 | SNP192-R | 1115 | GGTTGTGGACTGACTTTTAGGTTTG |
| SNP193 | SNP193-F | 1116 | GCTTCCTGCTAGCATTATTGAGATGA |
| SNP193 | SNP193-R | 1117 | AGGTAAATAGAGTTGAAGAAAATACTATCGACAT |
| SNP194 | SNP194-F | 1118 | CTTGGAATGACTCCTCTTCATCTGG |
| SNP194 | SNP194-R | 1119 | AAGATTTCTCTTCGTGTTAAGTTCTCTTTAC |
| SNP195 | SNP195-F | 1120 | GGAGCTTTCGAGTGCTTCAATTGTTC |
| SNP195 | SNP195-R | 1121 | CAGATGGGAGCCAGCCAATAAG |
| SNP196 | SNP196-F | 1122 | TGCCACCTATGACAGTAAAGACATG |
| SNP196 | SNP196-R | 1123 | CTCTCCAACATCTCGAAGTTGCTTC |
| SNP197 | SNP197-F | 1124 | GAGTCAGGTATTAAGAAAGTGGCAAAG |
| SNP197 | SNP197-R | 1125 | ACAACTCTCAGCTTTACCAGGC |
| SNP198 | SNP198-F | 1126 | ACTGATGGTAAATTGAGCAAGAGAATCG |
| SNP198 | SNP198-R | 1127 | GCAATTAGCAGTCTCAATACAAATGGAG |
| SNP199 | SNP199-F | 1128 | GGGAGAAATAATATTGTGATATATGAAGAAGAGC |
| SNP199 | SNP199-R | 1129 | TTGGTTCATCTGTGACTTCCACC |
| SNP200 | SNP200-F | 1130 | GGAAACAATTGGAGTACTTTGAACAATATC |
| SNP200 | SNP200-R | 1131 | GTTTGTGCTGCTCCAATTAAACCAC |
| SNP201 | SNP201-F | 1132 | TGCATGTCTAAGCTTAAGCCTAATTGAC |
| SNP201 | SNP201-R | 1133 | ATATGCTGTTACCGGTGTCTGG |
| SNP202 | SNP202-F | 1134 | GCAGCATGTATTTAACAAACAAGGAAC |
| SNP202 | SNP202-R | 1135 | CAGTGACTTCATCTTGACTGACAGC |
| SNP203 | SNP203-F | 1136 | CAGATATTTTGAATTCGAGCTTTGTTCG |
| SNP203 | SNP203-R | 1137 | TCATTTCAAATATACATTAGCATAAAACGTTCCC |
| SNP204 | SNP204-F | 1138 | ACTAGCAGCAACAGAAGCAGC |
| SNP204 | SNP204-R | 1139 | CCTTCATTCTTATGGTATTTTCTCAGCC |
| SNP205 | SNP205-F | 1140 | GTCTCCTTGAGCACTAGTTCTAAGTATTC |
| SNP205 | SNP205-R | 1141 | ACTTGATAGAATGGTTGAGGAAGATGAC |
| SNP206 | SNP206-F | 1142 | TAACACTATACTAGTCTTTTTGCCGCC |
| SNP206 | SNP206-R | 1143 | ACCCTTTTCCCTTTTACCTGAATAAAC |
| SNP207 | SNP207-F | 1144 | CCCTTGATCGCCATTGTAGACC |
| SNP207 | SNP207-R | 1145 | CTCAGAAGTAAAACGTAAAGTGAGTGG |
| SNP208 | SNP208-F | 1146 | TTCAGAAGGTTTTGGAATTCACTGTAG |
| SNP208 | SNP208-R | 1147 | GAAGGGGACAGAGTGGGATCC |
| SNP209 | SNP209-F | 1148 | GTGTGCAGAGGAAGAGAAAATAGAGATG |
| SNP209 | SNP209-R | 1149 | GGTAAGTCTCTTTTGGCCTACAGG |
| SNP210 | SNP210-F | 1150 | GAAAAGAAGGTCCCTCCAACTGG |
| SNP210 | SNP210-R | 1151 | TGACATTATTATTGTCACCTGAGAAATCTCC |
| SNP211 | SNP211-F | 1152 | CCTTTACCACTATTATAAAGAAAAAGGACAACC |
| SNP211 | SNP211-R | 1153 | GACCATGCGTGAACATGTGATG |
| SNP212 | SNP212-F | 1154 | TGAAGTAGGAGCAATGTTGGTGATG |
| SNP212 | SNP212-R | 1155 | CATTTTTCTTAGGAGGAAGTAGGCTAAC |
| SNP213 | SNP213-F | 1156 | TGTTCATTTTAAGATGAATAAAGAATTAAGTCTGC |
| SNP213 | SNP213-R | 1157 | AATGAGTTCCAAGGGTAGTGGTTTG |
| SNP214 | SNP214-F | 1158 | AAGTACTATCTGCTGCAAGTTGTTTTTTC |
| SNP214 | SNP214-R | 1159 | TATGGTTTTTTGTGTTCTAAATAAACTTGATCTG |
| SNP215 | SNP215-F | 1160 | CGTGCAATCCTCTGGAGAAGC |
| SNP215 | SNP215-R | 1161 | GTTGGTCTGCTTTTTGTTGCGAC |
| SNP216 | SNP216-F | 1162 | TGGTGCCTTTATACTTGCTGTGTATG |
| SNP216 | SNP216-R | 1163 | AGTAACAAGAACTGTAACTAACGTAGGAC |
| SNP217 | SNP217-F | 1164 | TTGCTCTGAAAAACAAAATATGGAGTGATG |
| SNP217 | SNP217-R | 1165 | TTGGATGGGGTGGAGTTTACTTG |
| SNP218 | SNP218-F | 1166 | TTCGAGTGATTTTGGGCGTTCC |
| SNP218 | SNP218-R | 1167 | TGCATTGGTCAGATCAAACAGAGGA |
| SNP219 | SNP219-F | 1168 | TCCATTTCAGATAGTGTGCTGGC |
| SNP219 | SNP219-R | 1169 | ATGCACAAATCAAATTTTCAAGGCAG |
| SNP220 | SNP220-F | 1170 | TTCAGCTAAATCTCTGGCCAAAGTTG |
| SNP220 | SNP220-R | 1171 | GAGATCGGAAGGAGCAAAGGAG |
| SNP221 | SNP221-F | 1172 | TGATTTTTGGAGCTCAGAAGAAAGAAGA |
| SNP221 | SNP221-R | 1173 | TCTAGAAGGAAAAGGAATCGACCCT |
| SNP222 | SNP222-F | 1174 | GTTCTCTGATTTGAGCCATGATGAG |
| SNP222 | SNP222-R | 1175 | GCAACTCTTGATCAACTTTCAGTCC |
| SNP223 | SNP223-F | 1176 | CAAGCAGGTGCAGCTGCTTTTAG |
| SNP223 | SNP223-R | 1177 | TCACAACCAGTCAATGAAAGAGGGG |
| SNP224 | SNP224-F | 1178 | AGGATCGTACGTGTATGGTTCAAAGG |
| SNP224 | SNP224-R | 1179 | AGCCCGTCGTCCAGTTATTTTC |
| SNP225 | SNP225-F | 1180 | GGACGAAGAATGCTCAAAACAATAAGG |
| SNP225 | SNP225-R | 1181 | ACGCTTGGAACTGAAAAGATGC |
| SNP226 | SNP226-F | 1182 | ACGATAGAACAGAAAACATTGCACTG |
| SNP226 | SNP226-R | 1183 | GTTGCAGAGAAGATTGCTGATTTTGG |
| SNP227 | SNP227-F | 1184 | GTTGTGTGGGCAAAGGCAGAG |
| SNP227 | SNP227-R | 1185 | GAACTTGTTGTCTTGTACGCGTAG |
| SNP228 | SNP228-F | 1186 | CTCCTACACCGCCCTATCCTAC |
| SNP228 | SNP228-R | 1187 | AGTTGGAGACAGTGAGACTCAGATTG |
| SNP229 | SNP229-F | 1188 | TCCATAACCCATTGTGTTCAATTGGAC |
| SNP229 | SNP229-R | 1189 | TCTAATCGAAAGCTCTGGCGATTC |
| SNP230 | SNP230-F | 1190 | GAAGTTGAAGCAATATCTTTCGAGAATGATG |
| SNP230 | SNP230-R | 1191 | GATAAGTGATCCAACGACAGAACTTTC |
| SNP231 | SNP231-F | 1192 | AATCCTCATCGATCCGATCAAATCG |
| SNP231 | SNP231-R | 1193 | CCGCACGGAAATAGAGATGTTCG |
| SNP232 | SNP232-F | 1194 | GCTGAAGCAAGGAAAAAATCGAAAATTC |
| SNP232 | SNP232-R | 1195 | TTTCAAGAATGTAAGGTTCTTTGTTTCTCACC |
| SNP233 | SNP233-F | 1196 | AGTTTGCATATTTTCGTCCATGTTTTTAATC |
| SNP233 | SNP233-R | 1197 | AACCAATGATTGCATAGAAAGTCATATCAC |
| SNP234 | SNP234-F | 1198 | GTGGGTAGCTGAAGGGTTTGTACAAG |
| SNP234 | SNP234-R | 1199 | CAAGAAACCTTGTGCGGTATCTTC |
| SNP235 | SNP235-F | 1200 | GCCTATATTTCCTCTCACTACTGTGC |
| SNP235 | SNP235-R | 1201 | GGAATATCTAAGACAATGCCCGAGC |
| SNP236 | SNP236-F | 1202 | GAAAAGTCTACTTCCACTTCTCCTGTG |
| SNP236 | SNP236-R | 1203 | CATCCTCATCATCTGACGCATAGTTAG |
| SNP237 | SNP237-F | 1204 | TCCACCATAGAAGCCCCAAATCC |
| SNP237 | SNP237-R | 1205 | GGATTGTTAATGAAGAGAAGCATAGGGG |
| SNP238 | SNP238-F | 1206 | TCAGCATTTTCAAATCTGGTGGC |
| SNP238 | SNP238-R | 1207 | AGGTTAACCCCTTGGATGATCTCC |
| SNP239 | SNP239-F | 1208 | GCTTCCAAACTATGTCGTCTATCATG |
| SNP239 | SNP239-R | 1209 | CATCTCAACTGCACATACATTACGC |
| SNP240 | SNP240-F | 1210 | CCCTGGTAATACAGCAAGTACGAC |
| SNP240 | SNP240-R | 1211 | GGTAGCTATTACTTGCATCATGCCAG |
| SNP241 | SNP241-F | 1212 | TCAATCGATTCGGGAATATCTGCTTG |
| SNP241 | SNP241-R | 1213 | AAGCAGCGCAGAAAGAGAATACC |
| SNP242 | SNP242-F | 1214 | GAGACGAGTGAGATTTCAAATTACAAGTC |
| SNP242 | SNP242-R | 1215 | CATGTTGTTCGTTACTAGCCAACTTAG |
| SNP243 | SNP243-F | 1216 | TGGAGAAAACAAGAGGTGGTAAGG |
| SNP243 | SNP243-R | 1217 | GCTCCACCAGCCATTACTACACG |
| SNP244 | SNP244-F | 1218 | ATGCACTTCCTTCATCACATTGTTG |
| SNP244 | SNP244-R | 1219 | GGCTTTTCTTCGAAAATGTTGCATTAATCC |
| SNP245 | SNP245-F | 1220 | TTGTGGTCTCAGCAATCATGGATG |
| SNP245 | SNP245-R | 1221 | AGGCCTGCTTCGAACATGG |
| SNP246 | SNP246-F | 1222 | GAAAAGGGTTCATGAACTAGAAGCTG |
| SNP246 | SNP246-R | 1223 | ATCGAATATTTTGGACTCCGTTAATCG |
| SNP247 | SNP247-F | 1224 | TGGTCTTGTTTGTTTGGCCCAATAG |
| SNP247 | SNP247-R | 1225 | AGAAGAAGAAAAAACCCCGAATCTCC |
| SNP248 | SNP248-F | 1226 | GGAGTGCAATCTCATAATAGTGCTCCTG |
| SNP248 | SNP248-R | 1227 | AGCGACGGAATTGCCATAGG |
| SNP249 | SNP249-F | 1228 | TTTAACTGAAAAATGGGGAGATTTACCAC |
| SNP249 | SNP249-R | 1229 | TTTAATAGACCGTAAATTACCATATCTTCGG |
| SNP250 | SNP250-F | 1230 | TGTGTGTACAGTAAAACCTCTGGTC |
| SNP250 | SNP250-R | 1231 | GAGTCCATTTCAAAGCTTGTACTGC |
| SNP251 | SNP251-F | 1232 | TTCAGATGAAATTTATGATGCATGGGTG |
| SNP251 | SNP251-R | 1233 | GCAAATACTCGTAAACATACTAATCAATTCAAC |
| SNP252 | SNP252-F | 1234 | AAGTTGCCTTGACCTAATAATCTCCC |
| SNP252 | SNP252-R | 1235 | ATGGAGAAAAAAGGAAATGTACTGATGG |
| SNP253 | SNP253-F | 1236 | CTCAATAACAGGAAATCACACCTAATCC |
| SNP253 | SNP253-R | 1237 | TGTGGTTGCTATTATTCCGTAGATACATC |
| SNP254 | SNP254-F | 1238 | TCGCCATCTCCCTTAGCACATG |
| SNP254 | SNP254-R | 1239 | TTGAGCGGGTGTTCAGTAATTAATG |
| SNP255 | SNP255-F | 1240 | TACCGATGATGAAAACACTCTAGCTAG |
| SNP255 | SNP255-R | 1241 | AATCAACAATGGACATCAAATACCCC |
| SNP256 | SNP256-F | 1242 | GAGGCCTTTCCGTGCTCTC |
| SNP256 | SNP256-R | 1243 | ACGGTTCCCTGTAGTAGTTCTTAAGAG |
| SNP257 | SNP257-F | 1244 | TGTGAAAGGCATCATATGTTAGCTCTC |
| SNP257 | SNP257-R | 1245 | ACAAGTTCAAGGCGGTGAAATTATC |
| SNP258 | SNP258-F | 1246 | TCAGGGTCTGAGTAATTTGAGAGC |
| SNP258 | SNP258-R | 1247 | GAATTTCACCCATGAACTTATTTCTCCC |
| SNP259 | SNP259-F | 1248 | GGAACAAATTAAGTAAGATCATCTTCCTCAAG |
| SNP259 | SNP259-R | 1249 | AGGACAACACTACTTCTAAGAGTCAAGG |
| SNP260 | SNP260-F | 1250 | ATGCCCTTGTGAGATGGGTGTG |
| SNP260 | SNP260-R | 1251 | CAACCTATGCCCTTGAACATGTG |
| SNP261 | SNP261-F | 1252 | ACACAATTCAAAATCCCCTCCTCC |
| SNP261 | SNP261-R | 1253 | CGTACGGCTGCAGAGTTCAAAG |
| SNP262 | SNP262-F | 1254 | GCAGAAGACCTCCCACCAGAG |
| SNP262 | SNP262-R | 1255 | ATCCCTGAGAAGAAATGGTACCTAAG |
| SNP263 | SNP263-F | 1256 | TTGGGAGAAAATACCCCTGCTG |
| SNP263 | SNP263-R | 1257 | TGAATGTTTTCGCCATTTCTCTTGTAC |
| SNP264 | SNP264-F | 1258 | TGACCATCTGAGTGTGAGAAATATGC |
| SNP264 | SNP264-R | 1259 | AGCACTCTGATAGGCCTGCTG |
| SNP265 | SNP265-F | 1260 | GACAAGGTTTCATGTCTGTTGAGTTG |
| SNP265 | SNP265-R | 1261 | CTACCAAAAACATCATAATATTTGATCTCAGC |
| SNP266 | SNP266-F | 1262 | GCTATATTTTGAGGCCTGTTGGAC |
| SNP266 | SNP266-R | 1263 | AAACACAAATTCTTGAGGCAATACATGAC |
| SNP267 | SNP267-F | 1264 | TAAAATATATGGCGACTGGATTGATGAC |
| SNP267 | SNP267-R | 1265 | ACAGATCAGTAGCTAATTATGACAACTCC |
| SNP268 | SNP268-F | 1266 | GGGCAAGAATCGGTCGGAAATG |
| SNP268 | SNP268-R | 1267 | CCACTACCTTCTCATCAAGACGAC |
| SNP269 | SNP269-F | 1268 | CTAGGAAGAGAGTTGAAAAGACCTAGC |
| SNP269 | SNP269-R | 1269 | AAAAACTTATACCTTATTTATCATCTCCCCC |
| SNP270 | SNP270-F | 1270 | CATCTGCAGAAGAATTGTTGTCCAG |
| SNP270 | SNP270-R | 1271 | GGCCATAAAGATTCTGCCAACC |
| SNP271 | SNP271-F | 1272 | TGTCTCCTTTGTTTCAAGTGGTATCATG |
| SNP271 | SNP271-R | 1273 | TGTTTCGATTGATTTCTCCTCAACTTC |
| SNP272 | SNP272-F | 1274 | TAGTTGTCTGCCGACTTCCTGG |
| SNP272 | SNP272-R | 1275 | CTCAGGAACTTCTCTAAAGAACGTATCTTG |
| SNP273 | SNP273-F | 1276 | AGAAAGTGTGACTCCAACAAAGCG |
| SNP273 | SNP273-R | 1277 | TCTTTCACGTCCTTCTTAGAAGCTG |
| SNP274 | SNP274-F | 1278 | CGAATGCATATAAAGCTTTTGATCCAAG |
| SNP274 | SNP274-R | 1279 | TAGTGCGGGGAAGGACCAAG |
| SNP275 | SNP275-F | 1280 | CAGAAGGAATCAACTCGGGGGT |
| SNP275 | SNP275-R | 1281 | TAACAGCCTAGCTAACCACCAC |
| SNP276 | SNP276-F | 1282 | TTCAAGTGGTGAAGTTCACATAGTGTG |
| SNP276 | SNP276-R | 1283 | AAAATTAAGTAATGCATGTAGCATGAATAAAAGTG |
| SNP277 | SNP277-F | 1284 | ACAAAGTTCCCAGAACCTCCAC |
| SNP277 | SNP277-R | 1285 | TAGTTATTTCTTCAACTTCGACTTCTGTG |
| SNP278 | SNP278-F | 1286 | CTGCAACCGAAGCTATTGACTGTG |
| SNP278 | SNP278-R | 1287 | CGAAGAGGGAGGAGGTCATTGATC |
| SNP279 | SNP279-F | 1288 | GCTGTTTCGACTGATACTATTAATGGTGG |
| SNP279 | SNP279-R | 1289 | TGACATTAAGTCGAAGACTCTGATCGA |
| SNP280 | SNP280-F | 1290 | GAACAGGGAAGTGTCAAATCAATTGGG |
| SNP280 | SNP280-R | 1291 | TGCAGCCTGGGCAGTTG |
| SNP281 | SNP281-F | 1292 | TTCGAAGCGGTTAGTCACAGTAAG |
| SNP281 | SNP281-R | 1293 | AACCGCTTTACCCACCGC |
| SNP282 | SNP282-F | 1294 | GAAGGGTGGTGGTGATTTGGC |
| SNP282 | SNP282-R | 1295 | ACATCAAACGGAACATATCTATCTTTTCC |
| SNP283 | SNP283-F | 1296 | GTGGGTGTGACATTGGGGCC |
| SNP283 | SNP283-R | 1297 | CACCAAGAGCACGTAGACCATG |
| SNP284 | SNP284-F | 1298 | TTGGACCAACTTGCATTTTCGGG |
| SNP284 | SNP284-R | 1299 | CACCTAAGCAAGTTTCAGAAAAGGTTG |
| SNP285 | SNP285-F | 1300 | GTGATTTCTGGTGAACCTGATCCAG |
| SNP285 | SNP285-R | 1301 | TTTGTCTGACTTGATTGGTGCAAC |
| SNP286 | SNP286-F | 1302 | GAATCGGCGTGGTAGGGAGG |
| SNP286 | SNP286-R | 1303 | AACCTTCATCACTCAAAACGGTAAAC |
| SNP287 | SNP287-F | 1304 | TGAACGAAGTGCTTACAAGAGAGTTG |
| SNP287 | SNP287-R | 1305 | GAGTAACCCTAACTTCAACTCCCG |
| SNP288 | SNP288-F | 1306 | AGGAAGTGAAGTTGGAAGAAAGAGCAG |
| SNP288 | SNP288-R | 1307 | ACCAACCATACATCTTCCTTCTGGC |
| SNP289 | SNP289-F | 1308 | GATCTTTCTGGATTAAACGGGAAACTG |
| SNP289 | SNP289-R | 1309 | GCTGCATCTAAGCCTCTTGACATTAG |
| SNP290 | SNP290-F | 1310 | TCGTGTTAACATTTCCTTTAAGGTATGACG |
| SNP290 | SNP290-R | 1311 | TCCGGGCATGTAAATCGGATGC |
| SNP291 | SNP291-F | 1312 | AAGGGACACGATAAACTTGCTCC |
| SNP291 | SNP291-R | 1313 | ACCAACTTGGAACCACAACAGG |
| SNP292 | SNP292-F | 1314 | GAGGTTCTTCGGTAGGTATTGCTTG |
| SNP292 | SNP292-R | 1315 | GCGCCTGTAGCAATAAATTTTAATCCG |
| SNP293 | SNP293-F | 1316 | ATCCAAAAATTTCGTTGAATATTAGGTTACCTG |
| SNP293 | SNP293-R | 1317 | GCGAAGGGATTCAGATGGGTTG |
| SNP294 | SNP294-F | 1318 | ACGCTCCTTGCCTCAGTCAC |
| SNP294 | SNP294-R | 1319 | GCTTTGGTTAATCGATTTGCGGATC |
| SNP295 | SNP295-F | 1320 | GACACTGAATGAAGCTCCGAGTG |
| SNP295 | SNP295-R | 1321 | GATTTTCTCGAACCGGAAATGTCG |
| SNP296 | SNP296-F | 1322 | ACCCCAGATGACACCGAAGATG |
| SNP296 | SNP296-R | 1323 | AGACACAATAACGCCCAAGAGATG |
| SNP297 | SNP297-F | 1324 | TCATATACGGATGCTGCAGCTG |
| SNP297 | SNP297-R | 1325 | GGGCATGCAGAAGAAGACCAG |
| SNP298 | SNP298-F | 1326 | CAAAGGTTTGCTTTTCGGCTCC |
| SNP298 | SNP298-R | 1327 | TTTTCAGCCTATAATGTGAAGCACC |
| SNP299 | SNP299-F | 1328 | GCATGGGAAACAGAAAATTGAGTTTG |
| SNP299 | SNP299-R | 1329 | AAAGACGGTTCTGCTGATCCTTC |
| SNP300 | SNP300-F | 1330 | CATAAAGAGGCTGCGATGAGGAG |
| SNP300 | SNP300-R | 1331 | AGTGGAATATTGGGAGTAGTGTGTC |
| SNP301 | SNP301-F | 1332 | CAAATTCTGCAACCTTTCCACACT |
| SNP301 | SNP301-R | 1333 | CCAACCAAAGAAGACATCGCATC |
| SNP302 | SNP302-F | 1334 | TGGCTGCATTCGTAGATGTTGAATTTG |
| SNP302 | SNP302-R | 1335 | AGTTATGTCGTCTGTCATACAAAAGTTTG |
| SNP303 | SNP303-F | 1336 | TGTAGCATTGGAGCATGTTCCG |
| SNP303 | SNP303-R | 1337 | CGGGACTGGTACACTAGAAACATC |
| SNP304 | SNP304-F | 1338 | CAGCAACAAGCTTCTGAATGCCA |
| SNP304 | SNP304-R | 1339 | GGCAGTGTCTAAGTGAAAGGCGA |
| SNP305 | SNP305-F | 1340 | GCTGGTAGAGAATCATTGATTGGCTC |
| SNP305 | SNP305-R | 1341 | TGGTTACTTATCAATCTTTCAGTTCTTGC |
| SNP306 | SNP306-F | 1342 | TCTTTAGTGGATAGTAAAATGGTGGGTTC |
| SNP306 | SNP306-R | 1343 | CATCACGAACAGCGCACCTC |
| SNP307 | SNP307-F | 1344 | TGGTGACGTTTTGGTTGATTCTATG |
| SNP307 | SNP307-R | 1345 | CTAAACAGTTCAACGACTGCAGG |
| SNP308 | SNP308-F | 1346 | AACCCGACGAATGTCCAACTC |
| SNP308 | SNP308-R | 1347 | AGGTACCCTGGCATTCTCTTGC |
| SNP309 | SNP309-F | 1348 | TAAACCACACCCTACGCGTATAG |
| SNP309 | SNP309-R | 1349 | ACGCTTCAACAAATTGGATAATGGG |
| SNP310 | SNP310-F | 1350 | ATGTGCCATCTTTCCAATTTTCATCA |
| SNP310 | SNP310-R | 1351 | GGAAATCCAATTCCTGAGTCTCTAGTG |
| SNP311 | SNP311-F | 1352 | AGCAAAAATGGTGAAAGACAGAACC |
| SNP311 | SNP311-R | 1353 | CTGTTTTGCTGCTCTTTGAAAAATCTAC |
| SNP312 | SNP312-F | 1354 | CCACTTAAATAGTTTACGGGCAAGAC |
| SNP312 | SNP312-R | 1355 | TACTTGTGTCCCCACTGCGG |
| SNP313 | SNP313-F | 1356 | GTTTTCTTCAAACAACAAATGTCTCTTATTCC |
| SNP313 | SNP313-R | 1357 | CGAAGATATTGCTCCTCCGACCAC |
| SNP314 | SNP314-F | 1358 | GTTACTTCACTTGAACACCATTCCC |
| SNP314 | SNP314-R | 1359 | CAAGAGGAGCATGCACTACGG |
| SNP315 | SNP315-F | 1360 | ACACTTTTGAATCTGTCCATCCATGAC |
| SNP315 | SNP315-R | 1361 | TTGGAGTCTCCGCGACAAGC |
| SNP316 | SNP316-F | 1362 | CCCAGGATTACGCGATGCAG |
| SNP316 | SNP316-R | 1363 | GATGGAATTACACACAACCTCAGATG |
| SNP317 | SNP317-F | 1364 | CGGGTAAGGATGTTTAGGTGCGT |
| SNP317 | SNP317-R | 1365 | CCAGCATAAATTTAAGAATGGAGTAGAATCC |
| SNP318 | SNP318-F | 1366 | GACCTGGCATTGACATGTCCATG |
| SNP318 | SNP318-R | 1367 | CCTCGTAGCTTCCGGGAGAC |
| SNP319 | SNP319-F | 1368 | AACGAAGAGGATGATATGGAAAATGC |
| SNP319 | SNP319-R | 1369 | ATCGTCTTCTCTCCTCCTTCCC |
| SNP320 | SNP320-F | 1370 | AGTCGAGATATTGACCAAATTTGCTC |
| SNP320 | SNP320-R | 1371 | TGTACAACAGAAAGTTCAATTAGATGGAG |
| SNP321 | SNP321-F | 1372 | AATCAACACGTTCGTGCAATCG |
| SNP321 | SNP321-R | 1373 | GATGATCTGTTCTGGAGTTGTTGC |
| SNP322 | SNP322-F | 1374 | ACTAAAGATCTGAAGGCACAAGTGG |
| SNP322 | SNP322-R | 1375 | GGGAATTGCGATGGAGTGAATTTTAAGG |
| SNP323 | SNP323-F | 1376 | TCATTTTCAAGTTGCCGTCAGC |
| SNP323 | SNP323-R | 1377 | AACTTCATCATAAGACCTATTAATAATCTGAGTTC |
| SNP324 | SNP324-F | 1378 | CGGAGATATTCAGGAACCGTCAATTG |
| SNP324 | SNP324-R | 1379 | GCCAGGTGTTTGGGAATATGTTC |
| SNP325 | SNP325-F | 1380 | CAGCTGATTCGAGGGGTCTC |
| SNP325 | SNP325-R | 1381 | ACCAACTGAAGAACAAGTCTGTTAGAAC |
| SNP326 | SNP326-F | 1382 | TGCAAAAAAGGACTCTTCAACCAG |
| SNP326 | SNP326-R | 1383 | GGTCTCATTGGCCTTTGAGGATTG |
| SNP327 | SNP327-F | 1384 | CTAGATCTTAACTGTGATGTTCTGAGCTG |
| SNP327 | SNP327-R | 1385 | ACGTTGATCTGACTGATGATCGG |
| SNP328 | SNP328-F | 1386 | GGTGATACTAAAGTGGATTTAAAAGGCG |
| SNP328 | SNP328-R | 1387 | ACCAACAACTTCCCCTGCACC |
| SNP329 | SNP329-F | 1388 | ACCGTCATTGGTACAGTTGATCC |
| SNP329 | SNP329-R | 1389 | CCACCAAAACTTACGTAGCCTACTC |
| SNP330 | SNP330-F | 1390 | AAGTATCCTACGTCAACAAGCTTCG |
| SNP330 | SNP330-R | 1391 | TGAATCTACTTATGCTTCTTGGGGTG |
| SNP331 | SNP331-F | 1392 | TGTCTTGATCTTCTGAAGTCTCACTTAC |
| SNP331 | SNP331-R | 1393 | CCGAGCTGGGCAGTCTAGAG |
| SNP332 | SNP332-F | 1394 | TGGTGGCATTGTTCAGGTGATTG |
| SNP332 | SNP332-R | 1395 | AGAAATCGACATGATAAGTTGTTTAAAACATC |
| SNP333 | SNP333-F | 1396 | TGGGAAAATCTAATCGACGATTACAAC |
| SNP333 | SNP333-R | 1397 | GGCGGCGTAGTTGATGGAAG |
| SNP334 | SNP334-F | 1398 | GGGGATTTTGGTAAGAGATTGGGC |
| SNP334 | SNP334-R | 1399 | CACAGTGACTCAAGAATCCACCAG |
| SNP335 | SNP335-F | 1400 | TATCTGTTAGTCCACCAACATGACTATG |
| SNP335 | SNP335-R | 1401 | CAAGCTGGCAAAAGTATCTTCAGTTTC |
| SNP336 | SNP336-F | 1402 | TAACGAAATTTTAACCATCATAGAAATGACTTCC |
| SNP336 | SNP336-R | 1403 | TGGGTCCCACTATAAGAAATTGAATTCC |
| SNP337 | SNP337-F | 1404 | GGATTTTTATGTGGCAGTTGCTAGAC |
| SNP337 | SNP337-R | 1405 | ACATAAATGTAACGAAAGAAACTGCAAAAG |
| SNP338 | SNP338-F | 1406 | GTGCTTTGCTAGTAGTTTGAAGGAG |
| SNP338 | SNP338-R | 1407 | CAGCCCATTACAAAAAGAATCAAACCC |
| SNP339 | SNP339-F | 1408 | CAATGTAACAATGAGGTAATTCAACAGC |
| SNP339 | SNP339-R | 1409 | TCTTTATAAGAAATCGCTTTAATTTTTGTACAGG |
| SNP340 | SNP340-F | 1410 | TACCTTCTGGATCCTTAACGCTG |
| SNP340 | SNP340-R | 1411 | ACCTGATGAGTTGGCGCAGG |
| SNP341 | SNP341-F | 1412 | AACGTTTCATTCACCCGTACCC |
| SNP341 | SNP341-R | 1413 | ACGGCGGAGTTGATTCGGAG |
| SNP342 | SNP342-F | 1414 | ATTTTACTGTCTATATACACCATTCACTGC |
| SNP342 | SNP342-R | 1415 | GATCATGGAAGATGGTAACGCAGTC |
| SNP343 | SNP343-F | 1416 | TTGCAGATTTGGGATAGTTAGGGC |
| SNP343 | SNP343-R | 1417 | CATGATCATCCACCACCACCTCC |
| SNP344 | SNP344-F | 1418 | GCATCATACAGTTGATCGTGGGG |
| SNP344 | SNP344-R | 1419 | ATGACTGCATGCAAGATACAAAAGG |
| SNP345 | SNP345-F | 1420 | ATTGATAACGAGAACAACGATCTTTTCTC |
| SNP345 | SNP345-R | 1421 | GACACATCTCGTATATCAAGGCCTG |
| SNP346 | SNP346-F | 1422 | GAAAGAGAGGAAATTGTGGCTTGTG |
| SNP346 | SNP346-R | 1423 | CGGTTTTTAGCAGTTCTTTTTGGGG |
| SNP347 | SNP347-F | 1424 | TTGGGGCTTATTTACAGAGGAGC |
| SNP347 | SNP347-R | 1425 | TGGCTTCAATTCTACCGCAACTC |
| SNP348 | SNP348-F | 1426 | TCACCGGAAAGACCATCACTCTTG |
| SNP348 | SNP348-R | 1427 | CCTTGTCTTGAATCTTAGCTTTGACATTATC |
| SNP349 | SNP349-F | 1428 | AAACTCATGAATTTAAGCTTGTTCAAGC |
| SNP349 | SNP349-R | 1429 | ACAGGGACTCTAATTTCACGACC |
| SNP350 | SNP350-F | 1430 | TGTCAGTAGGATGGTACTTGTTAGGAC |
| SNP350 | SNP350-R | 1431 | TTGCTCTTATGATCATGAGACGCG |
| SNP351 | SNP351-F | 1432 | ATCAAGCAGCAAGAACAAGTGC |
| SNP351 | SNP351-R | 1433 | GTGACAGCCCAACCATTGTTACAG |
| SNP352 | SNP352-F | 1434 | CCATTCTAAGCAGCAACCACCTC |
| SNP352 | SNP352-R | 1435 | TGCAGCTACATCATGGTTGGAG |
| SNP353 | SNP353-F | 1436 | TAATTGTTTCTCTGTGTCACGGACTG |
| SNP353 | SNP353-R | 1437 | CAAGATGTGGACGAGCAATTTGAC |
| SNP354 | SNP354-F | 1438 | GGAGCAAAAATTGGGTTTAAACACC |
| SNP354 | SNP354-R | 1439 | TTGCTCTTCAGTTTCGGCCTTTTAAG |
| SNP355 | SNP355-F | 1440 | CCAACACAGTGGCATCAGCATG |
| SNP355 | SNP355-R | 1441 | GGGAGTAGCTTGTCCAGGGAATC |
| SNP356 | SNP356-F | 1442 | TGTCTCTGATGGCCTAAGGAAAC |
| SNP356 | SNP356-R | 1443 | GCTGGAACTTTTCTGGTCCATCAC |
| SNP357 | SNP357-F | 1444 | CACTCATTATAGTTATCATTCCACACATCAT |
| SNP357 | SNP357-R | 1445 | AGGTCAATTGAAAGAAAGCCCGAAG |
| SNP358 | SNP358-F | 1446 | TGTGATTTCACCTCTGGAATAATTTTCTTG |
| SNP358 | SNP358-R | 1447 | ATGACCCAGCGTTTTGTTGAATCTC |
| SNP359 | SNP359-F | 1448 | GTCGGCACATGTCCTATTACAGAGG |
| SNP359 | SNP359-R | 1449 | TCTGATTCAGAGAGTTGTTTCAATTTCTC |
| SNP360 | SNP360-F | 1450 | GCATCTGTATGTGTGTGGTACATG |
| SNP360 | SNP360-R | 1451 | TTGGAGTAGTCTCACTGTCATCTAAATTG |
| SNP361 | SNP361-F | 1452 | CCTATTGTCCAGGTGGCCAAC |
| SNP361 | SNP361-R | 1453 | ACGTAAAGGAGCTGATACCTAACTG |
| SNP362 | SNP362-F | 1454 | GGAGGACTTTACCCCTACCTTGG |
| SNP362 | SNP362-R | 1455 | CATCTTTTCAAGTTAGTTTTGAGCCGAGG |
| SNP363 | SNP363-F | 1456 | TGTCCTCCAAGTTTATGTGATAACCC |
| SNP363 | SNP363-R | 1457 | GTGGTCACAGTTTGCAACACTATGAAG |
| SNP364 | SNP364-F | 1458 | GCCTTTGCGAATACTGGAATTGAG |
| SNP364 | SNP364-R | 1459 | TTGAGAAAACGCCAACAAGTCTGAG |
| SNP365 | SNP365-F | 1460 | ATGGGCAAAGCTACGTTTGATTTTATATG |
| SNP365 | SNP365-R | 1461 | CATCGTGTCCTTTTTTGTCACTACTG |
| SNP366 | SNP366-F | 1462 | GTCGACCCATATTTCGTATTTATGGACAG |
| SNP366 | SNP366-R | 1463 | TGAGAACAAGCTTTTCGGAGACC |
| SNP367 | SNP367-F | 1464 | TCCACCTCCACCTCCACCTC |
| SNP367 | SNP367-R | 1465 | CGGTTAGAGAAAAATCTTCCTTCACATG |
| SNP368 | SNP368-F | 1466 | ACGAAGTTATGGCAAGCAAAACG |
| SNP368 | SNP368-R | 1467 | AGCTACTTTTTCGAACAATTCACCG |
| SNP369 | SNP369-F | 1468 | CTTGGTCCTTCATTCACTTGAGATG |
| SNP369 | SNP369-R | 1469 | AGCAGAAGTCGAGGAAGTGGAG |
| SNP370 | SNP370-F | 1470 | GGACGAGGTCAATAACAACGAGC |
| SNP370 | SNP370-R | 1471 | TTTTTCACTCTGATCCCAAACGTTAC |
| SNP371 | SNP371-F | 1472 | ACAAGCCTTATAAGACGCAACTCG |
| SNP371 | SNP371-R | 1473 | AACATTTTCGGATATGGCAATGGAAC |
| SNP372 | SNP372-F | 1474 | TCCTGGTTCCTTGCTGCTGTG |
| SNP372 | SNP372-R | 1475 | TTGCCAAACAAGAGTATAAGTTCCAC |
| SNP373 | SNP373-F | 1476 | GCTACAACTTTTCTTACTTTCCAGTATCCTC |
| SNP373 | SNP373-R | 1477 | CCGAATCAGACGTTAAATCTCGGG |
| SNP374 | SNP374-F | 1478 | TGATCAGCTGCTAAACACGCG |
| SNP374 | SNP374-R | 1479 | GTACTCGAAAGTGAATAATCTGAAATGTTACC |
| SNP375 | SNP375-F | 1480 | TGTTGAACAGATCATCACGGGTC |
| SNP375 | SNP375-R | 1481 | ATTATTCCAGCTGGTGTGCCAC |
| SNP376 | SNP376-F | 1482 | AGATCTGGAAGAAGCCTACTTGTG |
| SNP376 | SNP376-R | 1483 | CAGTCTCAGTATCTGTACCTGGTAAAG |
| SNP377 | SNP377-F | 1484 | TTGGTAGTGAAGTTGAGGCCTC |
| SNP377 | SNP377-R | 1485 | TGAGGTATGTTAAGAATCGGGTTTGATG |
| SNP378 | SNP378-F | 1486 | TGCATGGCTATTTTCCGTCATCTAAGG |
| SNP378 | SNP378-R | 1487 | GGTTGCACCGTGATCACTTGG |
| SNP379 | SNP379-F | 1488 | TCAAATCGACAAACAATACATATCTGGTTA |
| SNP379 | SNP379-R | 1489 | AACCCTCGTCTGTCAGTGGC |
| SNP380 | SNP380-F | 1490 | TGAAGCCAGTTGACACTACTGAAG |
| SNP380 | SNP380-R | 1491 | GAGAGACCGGCCAGCATTG |
| SNP381 | SNP381-F | 1492 | GCAGGACATCCAAGAAGAGCAAAG |
| SNP381 | SNP381-R | 1493 | TGTCAATAATGCACCAATTACAAGTGAAG |
| SNP382 | SNP382-F | 1494 | AAGGTAGTCCGAGTCAAACTGTATC |
| SNP382 | SNP382-R | 1495 | TCTGGGTTTGTAATGGAACTTCATATGG |
| SNP383 | SNP383-F | 1496 | GCATAACAGCCATGAACTTGATGAAAC |
| SNP383 | SNP383-R | 1497 | GACTTGGTGTTCGTTTGAGCC |
| SNP384 | SNP384-F | 1498 | CAGAAAATCAATTGTGTTCTCTCAATTCAG |
| SNP384 | SNP384-R | 1499 | GCTTTAAGCGGCTCTTCAAGTAGG |
| SNP385 | SNP385-F | 1500 | TTCAAAGCACGCCAAAGAATTAGTG |
| SNP385 | SNP385-R | 1501 | ACCTTCTCTATCAAAGTATGGATGTGC |
| SNP386 | SNP386-F | 1502 | CTGCCTGAATGTCATCAAACATGTG |
| SNP386 | SNP386-R | 1503 | ACAGATGTATACATATATATTTCATGCTAACAAGG |
| SNP387 | SNP387-F | 1504 | ATGGGCACTAAGCTGTCACATC |
| SNP387 | SNP387-R | 1505 | GCCAACACGTTGAACATTGGAC |
| SNP388 | SNP388-F | 1506 | AGTCCCATAGTTTATTCCGACACC |
| SNP388 | SNP388-R | 1507 | TCTTGTCTTATCCCTCTTCATTCTCCTC |
| SNP389 | SNP389-F | 1508 | TAGTGACTATTCTGACGGCTTAAACC |
| SNP389 | SNP389-R | 1509 | AAAGGAGCATCGAAGCAACAGTAAAG |
| SNP390 | SNP390-F | 1510 | GAACTGCTAGGAACATCTGACACTTC |
| SNP390 | SNP390-R | 1511 | TCATGACCAAAAAGAAAGATGTGGG |
| SNP391 | SNP391-F | 1512 | AACCATCTTCTTCGGTAACCCAAC |
| SNP391 | SNP391-R | 1513 | AAGCAGATGCACCGGCAATACT |
| SNP392 | SNP392-F | 1514 | GCAGAGTCCAATCCAGAGGATG |
| SNP392 | SNP392-R | 1515 | GTCATCTGATGTTGATTGTCTAACAAGTG |
| SNP393 | SNP393-F | 1516 | CAGCTCAGCAACAGTTCGTCC |
| SNP393 | SNP393-R | 1517 | AGCAGAAGACTGAAGAGCTGAGC |
| SNP394 | SNP394-F | 1518 | ACATCTTTCGAAAACTTCCACCTTGATC |
| SNP394 | SNP394-R | 1519 | GCATCAAGAGTTTTAACCACTTGAATCC |
| SNP395 | SNP395-F | 1520 | GGGCGAAGGTCCTGAATCAG |
| SNP395 | SNP395-R | 1521 | GCAGACCCTTTGCCTCCAGC |
| SNP396 | SNP396-F | 1522 | GTGTCTGCACTATTCATATTTTGATTCGA |
| SNP396 | SNP396-R | 1523 | GTCAAGAAATCCAACTGTATGGCTTG |
| SNP397 | SNP397-F | 1524 | TGCCCATACGATTTAAGGTTGCG |
| SNP397 | SNP397-R | 1525 | CAGGTGCAACCATTGTTACCGTG |
| SNP398 | SNP398-F | 1526 | CCCCAACCTAGGTGAGAGAACTC |
| SNP398 | SNP398-R | 1527 | TCCTTCACATCCTGCAAACGTCC |
| SNP399 | SNP399-F | 1528 | TTACGCACACGCCATGGGTTAG |
| SNP399 | SNP399-R | 1529 | CGTGACGTGGGCCTAAACC |

The primers from TABLE 10 were synthesized and tested using NGS. Anti-sense oligos were not added to the reaction mixture. TABLE 11 shows the average number of reads obtained on an NGS sequencer (AVG) using the primers from TABLE 10, as well as the standard deviation (STD) and coefficient of variance (% CV) for each target. All calculations were based on the average of 3 replicates for each SNP. The data in TABLE 11 is summarized in TABLE 12, demonstrating even multiplexing in one reaction.

TABLE 11
Average number of reads obtained on an NGS
sequencer (using the primers from TABLE 10)

| SNP | AVG | STD | CV % |
|---|---|---|---|
| 100 | 341 | 141 | 41.2% |
| 101 | 92 | 36 | 39.3% |
| 102 | 31 | 17 | 53.8% |
| 103 | 330 | 144 | 43.7% |
| 104 | 131 | 42 | 32.2% |
| 105 | 108 | 42 | 38.8% |
| 106 | 60 | 30 | 50.7% |
| 107 | 35 | 11 | 30.7% |
| 108 | 356 | 163 | 45.6% |
| 109 | 25 | 5 | 18.3% |
| 110 | 353 | 144 | 40.9% |
| 111 | 285 | 110 | 38.7% |
| 112 | 77 | 33 | 42.7% |
| 113 | 34 | 15 | 43.7% |
| 114 | 156 | 57 | 36.9% |
| 115 | 270 | 116 | 42.9% |
| 116 | 138 | 51 | 36.8% |
| 117 | 440 | 186 | 42.3% |
| 118 | 438 | 175 | 40.0% |
| 119 | 408 | 180 | 44.1% |
| 120 | 40 | 17 | 41.9% |
| 121 | 363 | 160 | 44.0% |
| 122 | 85 | 23 | 26.8% |
| 123 | 46 | 13 | 29.5% |
| 124 | 191 | 85 | 44.3% |
| 125 | 117 | 41 | 35.1% |
| 126 | 426 | 176 | 41.3% |
| 127 | 164 | 61 | 37.1% |
| 128 | 198 | 75 | 38.0% |
| 129 | 560 | 203 | 36.2% |
| 130 | 171 | 51 | 29.5% |
| 131 | 111 | 41 | 36.4% |
| 132 | 45 | 18 | 39.6% |
| 133 | 91 | 32 | 35.4% |
| 134 | 34 | 17 | 49.9% |
| 135 | 235 | 89 | 37.9% |
| 136 | 127 | 50 | 39.9% |
| 137 | 186 | 61 | 32.8% |
| 138 | 444 | 168 | 37.9% |
| 139 | 471 | 194 | 41.2% |
| 140 | 171 | 72 | 42.2% |
| 141 | 360 | 129 | 36.0% |
| 142 | 41 | 13 | 32.6% |
| 143 | 65 | 25 | 37.9% |
| 144 | 203 | 85 | 41.7% |
| 145 | 30 | 14 | 46.8% |
| 146 | 52 | 14 | 26.8% |
| 147 | 129 | 58 | 44.5% |
| 148 | 37 | 14 | 38.9% |
| 149 | 152 | 57 | 37.7% |
| 150 | 237 | 84 | 35.3% |
| 151 | 175 | 66 | 37.9% |
| 152 | 289 | 107 | 37.1% |
| 153 | 45 | 15 | 32.9% |
| 154 | 139 | 44 | 31.5% |
| 155 | 327 | 135 | 41.2% |
| 156 | 172 | 63 | 36.5% |
| 157 | 38 | 15 | 40.2% |
| 158 | 99 | 41 | 41.3% |
| 159 | 84 | 27 | 32.2% |
| 160 | 84 | 44 | 52.6% |
| 161 | 32 | 10 | 31.9% |
| 162 | 213 | 78 | 36.5% |
| 163 | 566 | 258 | 45.6% |
| 164 | 147 | 52 | 35.3% |
| 165 | 82 | 30 | 36.9% |
| 166 | 259 | 106 | 41.1% |
| 167 | 298 | 125 | 41.9% |
| 168 | 188 | 64 | 34.1% |
| 169 | 380 | 161 | 42.4% |
| 170 | 365 | 163 | 44.6% |
| 171 | 491 | 184 | 37.5% |
| 172 | 101 | 42 | 41.7% |
| 173 | 470 | 202 | 43.1% |
| 174 | 324 | 147 | 45.5% |
| 175 | 95 | 31 | 33.3% |
| 176 | 50 | 14 | 27.4% |
| 177 | 45 | 19 | 41.3% |
| 178 | 407 | 170 | 41.7% |
| 179 | 302 | 131 | 43.5% |
| 180 | 211 | 90 | 42.7% |
| 181 | 453 | 168 | 37.1% |
| 182 | 63 | 26 | 41.6% |
| 183 | 393 | 162 | 41.4% |
| 184 | 445 | 184 | 41.2% |
| 185 | 84 | 35 | 41.9% |
| 186 | 94 | 39 | 41.4% |
| 187 | 310 | 114 | 36.6% |
| 188 | 76 | 27 | 36.2% |
| 189 | 108 | 33 | 30.7% |
| 190 | 301 | 123 | 41.0% |
| 191 | 44 | 12 | 26.3% |
| 192 | 87 | 32 | 36.3% |
| 193 | 28 | 13 | 45.9% |
| 194 | 188 | 62 | 32.8% |
| 195 | 302 | 101 | 33.6% |
| 196 | 131 | 56 | 43.1% |
| 197 | 324 | 139 | 43.1% |
| 198 | 172 | 73 | 42.5% |
| 199 | 228 | 91 | 40.0% |
| 200 | 112 | 46 | 41.0% |
| 201 | 172 | 65 | 37.8% |
| 202 | 102 | 42 | 41.4% |
| 203 | 145 | 42 | 29.0% |
| 204 | 322 | 146 | 45.5% |
| 205 | 138 | 53 | 38.0% |
| 206 | 216 | 81 | 37.3% |
| 207 | 164 | 56 | 34.1% |
| 208 | 120 | 44 | 37.0% |
| 209 | 106 | 47 | 44.2% |
| 210 | 177 | 67 | 37.6% |
| 211 | 118 | 40 | 34.2% |
| 212 | 206 | 84 | 40.8% |
| 213 | 83 | 32 | 38.0% |
| 214 | 46 | 8 | 18.3% |
| 215 | 157 | 64 | 40.6% |
| 216 | 248 | 95 | 38.5% |
| 217 | 95 | 33 | 34.3% |
| 218 | 142 | 49 | 34.3% |
| 219 | 391 | 179 | 45.8% |
| 220 | 26 | 11 | 42.3% |
| 221 | 142 | 50 | 35.0% |
| 222 | 87 | 30 | 34.9% |
| 223 | 202 | 73 | 36.1% |
| 224 | 439 | 167 | 38.1% |
| 225 | 80 | 33 | 41.2% |
| 226 | 57 | 21 | 36.6% |
| 227 | 389 | 161 | 41.4% |
| 228 | 208 | 69 | 33.5% |
| 229 | 263 | 119 | 45.3% |
| 230 | 47 | 22 | 46.3% |
| 231 | 325 | 131 | 40.2% |
| 232 | 138 | 61 | 44.3% |
| 233 | 29 | 14 | 48.4% |
| 234 | 263 | 92 | 34.9% |
| 235 | 338 | 141 | 41.7% |
| 236 | 326 | 122 | 37.4% |
| 237 | 348 | 160 | 46.0% |
| 238 | 551 | 235 | 42.6% |
| 239 | 50 | 29 | 58.4% |
| 240 | 431 | 184 | 42.6% |
| 241 | 304 | 116 | 38.3% |
| 242 | 127 | 49 | 38.8% |
| 243 | 74 | 22 | 29.6% |
| 244 | 121 | 51 | 42.1% |
| 245 | 387 | 149 | 38.6% |
| 246 | 180 | 71 | 39.2% |
| 247 | 36 | 13 | 35.6% |
| 248 | 227 | 76 | 33.6% |
| 249 | 41 | 13 | 31.0% |
| 250 | 111 | 46 | 41.5% |
| 251 | 33 | 13 | 38.1% |
| 252 | 75 | 28 | 37.7% |
| 253 | 58 | 25 | 44.1% |
| 254 | 211 | 101 | 48.0% |
| 255 | 27 | 13 | 47.5% |
| 256 | 182 | 65 | 35.6% |
| 257 | 167 | 67 | 39.9% |
| 258 | 373 | 169 | 45.4% |
| 259 | 30 | 13 | 42.9% |
| 260 | 289 | 121 | 42.0% |
| 261 | 138 | 40 | 28.9% |
| 262 | 35 | 15 | 42.2% |
| 263 | 50 | 18 | 35.9% |
| 264 | 292 | 115 | 39.6% |
| 265 | 261 | 97 | 37.0% |
| 266 | 27 | 15 | 54.8% |
| 267 | 287 | 126 | 44.0% |
| 268 | 239 | 85 | 35.7% |
| 269 | 50 | 18 | 35.7% |
| 270 | 267 | 89 | 33.3% |
| 271 | 135 | 43 | 31.8% |
| 272 | 97 | 40 | 41.5% |
| 273 | 133 | 56 | 41.9% |
| 274 | 47 | 22 | 45.7% |
| 275 | 114 | 43 | 38.0% |
| 276 | 49 | 17 | 34.4% |
| 277 | 242 | 104 | 43.0% |
| 278 | 92 | 27 | 29.1% |
| 279 | 167 | 71 | 42.8% |
| 280 | 256 | 93 | 36.3% |
| 281 | 129 | 48 | 37.0% |
| 282 | 119 | 37 | 31.0% |
| 283 | 281 | 105 | 37.4% |
| 284 | 352 | 153 | 43.3% |
| 285 | 388 | 170 | 43.9% |
| 286 | 200 | 75 | 37.5% |
| 287 | 373 | 157 | 42.1% |
| 288 | 383 | 160 | 41.7% |
| 289 | 79 | 26 | 33.2% |
| 290 | 400 | 175 | 43.7% |
| 291 | 513 | 239 | 46.7% |
| 292 | 213 | 85 | 40.1% |
| 293 | 224 | 103 | 46.1% |
| 294 | 345 | 150 | 43.6% |
| 295 | 321 | 120 | 37.2% |
| 296 | 227 | 90 | 39.9% |
| 297 | 101 | 39 | 38.4% |
| 298 | 453 | 182 | 40.1% |
| 299 | 29 | 12 | 41.5% |
| 300 | 85 | 32 | 38.2% |
| 301 | 86 | 26 | 30.5% |
| 302 | 196 | 72 | 36.7% |
| 303 | 123 | 48 | 39.3% |
| 304 | 194 | 89 | 45.8% |
| 305 | 39 | 8 | 20.2% |
| 306 | 106 | 43 | 40.6% |
| 307 | 36 | 17 | 46.5% |
| 308 | 440 | 179 | 40.6% |
| 309 | 141 | 40 | 28.4% |
| 310 | 28 | 7 | 25.9% |
| 311 | 75 | 30 | 40.5% |
| 312 | 465 | 193 | 41.6% |
| 313 | 169 | 72 | 42.5% |
| 314 | 86 | 38 | 43.5% |
| 315 | 227 | 99 | 43.5% |
| 316 | 31 | 16 | 51.8% |
| 317 | 32 | 10 | 30.4% |
| 318 | 81 | 30 | 37.6% |
| 319 | 155 | 52 | 33.3% |
| 320 | 33 | 9 | 27.2% |
| 321 | 91 | 31 | 34.6% |
| 322 | 56 | 15 | 27.2% |
| 323 | 89 | 36 | 40.6% |
| 324 | 56 | 23 | 42.1% |
| 325 | 285 | 111 | 38.9% |
| 326 | 222 | 102 | 46.0% |
| 327 | 271 | 118 | 43.4% |
| 328 | 274 | 113 | 41.2% |
| 329 | 377 | 153 | 40.5% |
| 330 | 478 | 180 | 37.6% |
| 331 | 224 | 79 | 35.0% |
| 332 | 67 | 26 | 38.4% |
| 333 | 28 | 9 | 31.6% |
| 334 | 292 | 113 | 38.7% |
| 335 | 51 | 21 | 40.0% |
| 336 | 79 | 25 | 32.2% |
| 337 | 115 | 41 | 35.8% |
| 338 | 209 | 89 | 42.6% |
| 339 | 199 | 83 | 41.5% |
| 340 | 64 | 33 | 51.8% |
| 341 | 570 | 245 | 42.9% |
| 342 | 356 | 158 | 44.5% |
| 343 | 538 | 216 | 40.2% |
| 344 | 138 | 59 | 42.7% |
| 345 | 393 | 152 | 38.7% |
| 346 | 231 | 89 | 38.3% |
| 347 | 311 | 118 | 38.1% |
| 348 | 48 | 21 | 44.0% |
| 349 | 149 | 47 | 31.5% |
| 350 | 301 | 108 | 36.1% |
| 351 | 174 | 75 | 43.3% |
| 352 | 477 | 205 | 43.0% |
| 353 | 413 | 191 | 46.4% |
| 354 | 135 | 50 | 37.3% |
| 355 | 102 | 38 | 37.3% |
| 356 | 143 | 53 | 37.4% |
| 357 | 108 | 43 | 39.5% |
| 358 | 74 | 28 | 37.4% |
| 359 | 67 | 29 | 42.7% |
| 360 | 207 | 85 | 41.0% |
| 361 | 205 | 73 | 35.6% |
| 362 | 224 | 85 | 37.9% |
| 363 | 39 | 14 | 36.3% |
| 364 | 164 | 65 | 39.7% |
| 365 | 90 | 35 | 38.6% |
| 366 | 291 | 121 | 41.4% |
| 367 | 235 | 88 | 37.4% |
| 368 | 92 | 33 | 35.5% |
| 369 | 314 | 115 | 36.8% |
| 370 | 88 | 37 | 42.6% |
| 371 | 253 | 109 | 43.0% |
| 372 | 103 | 48 | 46.1% |
| 373 | 418 | 184 | 44.0% |
| 374 | 237 | 95 | 40.2% |
| 375 | 560 | 237 | 42.4% |
| 376 | 36 | 13 | 36.5% |
| 377 | 442 | 185 | 42.0% |
| 378 | 384 | 179 | 46.8% |
| 379 | 42 | 14 | 33.1% |
| 380 | 81 | 27 | 34.0% |
| 381 | 25 | 16 | 64.3% |
| 382 | 251 | 101 | 40.2% |
| 383 | 357 | 152 | 42.7% |
| 384 | 93 | 33 | 35.1% |
| 385 | 145 | 45 | 31.2% |
| 386 | 32 | 11 | 34.5% |
| 387 | 276 | 102 | 36.9% |
| 388 | 152 | 64 | 42.3% |
| 389 | 71 | 25 | 34.8% |
| 390 | 307 | 99 | 32.3% |
| 391 | 292 | 118 | 40.3% |
| 392 | 124 | 48 | 39.0% |
| 393 | 140 | 50 | 35.9% |
| 394 | 131 | 56 | 43.0% |
| 395 | 371 | 150 | 40.3% |
| 396 | 102 | 35 | 34.8% |
| 397 | 119 | 44 | 36.8% |
| 398 | 238 | 100 | 41.8% |
| 399 | 32 | 12 | 38.7% |

TABLE 12
Statistical Summary of TABLE 11

| Total Amplicons | 300 |
|---|---|
| Minimum Reads across | 25 |
| 300 SNPs | |
| Maximum Reads across | 570 |
| 300 SNPs | |
| Average Reads for all | 193 |
| 300 SNPs | |
| Standard Deviation of | 137 |
| Average Reads for all | |
| 300 SNPs | |
| CV % | 70.7% |
| # SNPS with > 10 reads | 300 |
| # SNPS with > 20 reads | 300 |
| # SNPS with > 50 reads | 254 |
| # SNPS with > 100 reads | 202 |
| # SNPS with > 200 reads | 123 |

1.-27. (canceled)

28. A method of preparing an optimized primer set for multiplex genotyping, the method comprising:
A) for a given set N of variable genomic target sequences of a genome to be genotyped in a sample, designing an initial set of forward and reverse amplification primers that will amplify a sequence comprising each variable genomic target sequence in a multiplex amplification reaction, wherein the designing includes the steps of:
1) identifying all possible primers of 17 to 35 nucleotides within 200 base pairs of each genomic target sequence variation in set N of variable genomic target sequences from a pool of primers;
2) for each member of set N, selecting a subset of primer pairs from the set of step (1) that satisfies the conditions of a primer selection algorithm;
3) evaluating specificity of primer pairs chosen in step (2) in the genome, keeping only those pairs predicted to be specific for their respective targets;
4) selecting a set of optimized primers for the amplification of target gene set N, where the optimal primers are selected to minimize primer-primer interactions with other primers in the set by iterative calculation of predicted ΔG for interactions between primers to generate a fitness score and use of a fitness score optimization method selected from one or a combination of the group consisting of:
a) a Monte Carlo random or pseudo-random selection method;
b) a golden section search;
c) gradient descent;
d) minima hopping;
e) genetic algorithm;
f) neural networks;
g) cluster analysis, in which substitution is picked to minimize score; and
h) cluster analysis to create bins; and
wherein the Fitness Score is generated according to the method:
a) determining G=the set of ΔG's for all possible interactions for members of the initial primer set; and
b) calculating the Fitness Score by:
i) determining the sum, S, of |ΔG|^{Q} for each ΔG value, wherein Q is a weighting factor constant exponent that makes large ΔG absolute values much larger than small values;
ii) determining S′=S/#of ΔG values in G;
iii) determining H=T/S′, wherein T is a constant that makes H small for large values of S′ and H large for small values of S′; and
iv) determining the Fitness Score=H^{R}, wherein R is a weighting factor constant exponent that makes large values of H larger, and small values of H smaller; and
B) synthesizing the optimized primer set selected in step (4).

29. The method of claim 28, wherein steps (2)-(4) comprise:
a) for the primers identified in step (1), randomly selecting a primer pair for each target in set N that satisfies the conditions of the primer selection algorithm;
b) evaluating specificity of primer pairs chosen in step (a) in the genome, keeping only those pairs predicted to be specific for their respective targets;
c) repeating step (a) on the primer pairs kept from step (b) to generate set P, a population of randomly selected primer sets for each target in set N;
d) generating the Fitness Score for each member of population P based upon ΔG for all possible interactions between the primers in each member of the population;
e) picking member(s) of the population P based on Fitness Score;
f) repeating steps (c)-(e) iteratively until a set of primer pairs for target genes identified in step (e) has the Fitness Score at a predetermined threshold.

30. The method of claim 28, wherein steps (2)-(4) comprise:
a) for the primers identified in step (1), randomly selecting a primer pair for each target in set N that satisfies the conditions of the primer selection algorithm and is predicted to be specific for its target in the genome, or providing a primer pair for each target in set N, that has been selected to reduce potential for primer:primer interactions with other primers in the set and is predicted to be specific for its target in the genome;
b) repeating step (a) to generate population Z, of size 2 or greater, of primer pair sets for each target in set N;
c) generating the Fitness Score for each member of population Z based upon ΔG for all possible interactions between the primers in each member of the population;
d) selecting the members of population Z with the lowest Fitness Scores as set W;
e) replacing a primer for a single target from W with another primer identified in step (a), and generating the Fitness Score for the resulting set; wherein if the change results in an improved Fitness Score relative to the Fitness Score generated in step (c), the resulting new set W′ replaces set W, and if the change results in a no change in Fitness Score or a decreased Fitness Score, keeping set W;
f) iteratively repeating steps (c)-(e) on the set W or W′ retained in each iteration of step (e) until a set of primer pairs for target genes in set N is identified that has the Fitness Score at a predetermined threshold, or, if a predetermined threshold is not reached by iteratively repeating steps (c)-(e), beginning again at step (a) and iteratively repeating steps (c)-(e) until a set of primer pairs for target genes in set N is identified that has the Fitness Score at the predetermined threshold.

31. The method of claim 30, wherein the step of providing a primer pair for each target in set N that has been selected to reduce potential for primer:primer interactions with other primers in the set provides primer sets selected using one or more of a Monte Carlo random or pseudo-random selection method, a golden section search, gradient descent, minima hopping, a genetic algorithm, neural networks, cluster analysis in which substitution is picked to minimize score, or cluster analysis to create bins.

32. The method of claim 28, wherein steps (2)-(4) comprise:
a) generating primer set Z, including a primer pair for each member of set N either by: (i) randomly selecting from the primers identified in step (1) a primer pair for each target in set N that satisfies the conditions of the primer selection algorithm and is predicted to be specific for its target in the genome; or (ii) providing a primer pair for each target in set N that is predicted to be specific for its target in the genome, and that has been selected to reduce potential for primer:primer interactions with other primers in the set;
b) generating the Fitness Score for primer set Z based upon ΔG for all possible interactions between the primers in each member of the population;
c) making a change to a primer for a single target from set Z to generate new set Z′, and generating the Fitness Score for set Z′, wherein if the change results in an improved Fitness Score relative to that generated in step (b), the resulting new set Z′ replaces set Z, and if the change results in no change in Fitness Score or a decreased Fitness Score, keeping set Z; and
d) repeating step (c) iteratively until further iterations do not improve fitness of set Z.

33. The method of claim 28, wherein steps (2)-(4) comprise:
a) providing a set of optimized primer pairs for the amplification of target gene set N, where the optimal primer pairs are predicted to be specific for their target genes in the genome, and are selected to minimize primer-primer interactions with other primers in the set by iterative calculation of predicted ΔG for all possible interactions between primers to generate the Fitness Score and use of the Fitness Score optimization method selected from one or a combination of the group consisting of:
i) a Monte Carlo random or pseudo-random selection method;
ii) a golden section search;
iii) gradient descent;
iv) minima hopping;
v) genetic algorithm;
vi) neural networks;
vii) cluster analysis, in which substitution is picked to minimize score; and
viii) cluster analysis to create bins;
b) adding the set of optimized primers of step a to set M;
c) while maintaining a degree of dissimilarity from primer sets included in set M, selecting a primer pair for each target in set N from step (1) and designating it set Z, wherein the primer pairs satisfy the conditions of the primer selection algorithm, and are predicted to be specific for their target genes in the genome;
d) optimizing primer pairs of set Z for the amplification of target gene set N, to minimize primer-primer interactions with other primers in the set by iterative calculation of predicted ΔG for all possible interactions between primers to generate the Fitness Score and use of the Fitness Score optimization method selected from one or a combination of methods (i)-(viii) of step (a); and
e) repeating steps (a)-(d) iteratively until a set of primer pairs for target gene set N identified in step (d) has the Fitness Score at a predetermined threshold.

34. The method of claim 28, wherein steps (2)-(4) comprise:
a) for a multi-layer neural network, for each primer identified in step (1) creating a node P_{nz} comprised by the neural network, such node connected to a node for a corresponding target (Tₙ), wherein:
(i) each node outputs its identifier (ID) and a numeric value;
(ii) each Tₙ produces the ID of one of the Pnz nodes connected to it;
(iii) each one of the Tₙ nodes is connected to all others; and
(iv) each node Tₙ is comprised by the multilayer neural network;
b) calculating the Fitness Score for output of the neural network, and on the basis of Fitness Score, the value produced by the network is compared to target, and neural network parameters for a plurality of the Tₙ are changed;
c) calculating Fitness Score again for output of the neural network with parameters changed in step (b);
d) determining if a change was beneficial or not to the fitness of the resulting set, wherein if the change was beneficial, the direction of change is maintained with smaller increments, and wherein if the change was not beneficial, either direction is reversed or the parameters revert to a previous state;
e) repeating steps (b)-(d) iteratively, wherein at a plurality of iterations random changes are made to the parameters of the network, and wherein when the rate of fitness improvement decreases, the frequency of such random changes is increased, until a set of primer pairs for target genes in set N is identified that has the fitness score at a predetermined threshold.

35. The method of claim 28, wherein steps (2)-(4) comprise:
a) picking the target at random, as well as a primer for such target, and placing it in set R;
b) picking an additional target, and calculating the Fitness Score evaluating all primers for this target in combination with primers already in set R on the basis of ΔG for all potential interactions, wherein the primer that results in the best Fitness Score is added to set R;
c) if fitness of set R is below a predetermined threshold T, removing one of the primers from R according to the following:
calculating the Fitness Score for set Ri, wherein the i^{th} target with its primer is removed from set R, and the set with the best Fitness Score determines the target with its primer to be removed from set R and placed back into the pool of primers of step (1); and
d) repeating steps (b) and (c) until all targets have optimized primers.

36. The method of claim 28, wherein steps (2)-(4) comprise:
a) picking the target at random, as well as a primer for such target, and placing it in set R;
b) picking an additional target, and calculating the Fitness Score evaluating all primers for this target in combination with primers already in set R on the basis of ΔG for all potential interactions, wherein the primer that results in the best Fitness Score is added to set R;
c) if fitness of set R is below a predetermined threshold T, removing one of the primers from R according to the following:
calculating the Fitness Score for set Ri, wherein the i^{th} target with its primer is removed from set R, and the set with the best Fitness Score determines the target with its primer to be removed from set R and placed back into the pool of primers of step (1);
d) repeating steps (b) and (c) until all targets have optimized primers;
e) once all targets have optimized primers, designating set R as R₁, and its fitness as F₁;
f) creating empty set R_{z+1}, where Z is the number of sets, with fitness F_{z+1};
g) for each set Rz, where z is an index from 1 to number of sets R, determining the element that is worst for the set's fitness, and removing this element, designated Target E;
h) recalculating Fz after removal of Target E;
i) for all Rz, determining where Target E can be added so as to maximize Fz and maximize the minimum of Fz; and
j) if the minimum of Fz is below the predetermined threshold, repeating steps (f)-(i) until the standard deviation of Fz is below the predetermined threshold, thereby designing the multiplex primer set.

37. The method of claim 36, wherein the step of determining the element in step (g) that is worst for fitness is performed in a method according to step (4).

38. A method of preparing an optimized primer set for multiplex genotyping, the method comprising:
A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of:
1) identifying all possible primers of 17 to 35 nucleotides within 200 base pairs of each genomic target sequence variation in set N of variable genomic target sequences;
2) for each member of set N, selecting from the set of primers in step (1) a subset of primer pairs that satisfies the conditions of a primer selection algorithm and is predicted to be specific for its target;
3) repeating step (2) to generate set P, a population of randomly selected primer sets for each target gene in set N;
4) calculating a Fitness Score for each member of the population P;
wherein the Fitness Score is generated according to the method:
a) determining G=the set of ΔG's for all possible interactions determined for members of the primer set; and
b) calculating the Fitness Score by:
i) determining the sum, S, of |ΔG|^{Q} for each ΔG value, wherein Q is a weighting factor constant exponent that makes large ΔG absolute values much larger than small values;
ii) determining S′=S/#of ΔG values in G;
iii) determining H=T/S′, wherein T is a constant that makes H small for large values of S′ and H large for small values of S′; and
iv) determining the Fitness Score=H^{R}, wherein R is a weighting factor constant exponent that makes large values of H larger, and small values of H smaller;
5) placing members of population P into a pool of candidate primer sets on the basis of Fitness Scores;
6) randomly selecting a plurality of “parent” sets of candidate primers from the pool of step (5), each parent set including a different pair of candidate primer sets, parent A and parent B;
7) for each parent set of candidate primers, creating a crossover set of candidate primers by replacing a subset of candidate primer pairs of parent A with the corresponding subset of primer pairs of parent B;
8) randomly replacing one primer pair in crossover set A with a different primer pair for the corresponding target sequence generated in step (2) to create a Generation 2 population of primer sets for each target gene in set N; and
9) repeating steps (4)-(8) iteratively until a set of primer pairs for target genes in set N is identified that has a Fitness Score at a predetermined threshold, and runs for an additional set amount of iterations with no measurable improvement in the fitness of the best member, whereby an optimized primer set is designed; and
B) synthesizing the optimized primer set designed in step (9).

39. A method of preparing a primer set for multiplex genotyping, the method comprising:
A) for a given set N of variable genomic target sequences to be genotyped in a sample, designing a set of forward and reverse amplification primers that will amplify a sequence comprising each variable genomic target sequence in a multiplex amplification reaction, wherein the designing includes the steps of:
1) identifying all possible primers of 17 to 35 nucleotides within 200 base pairs of each genomic target sequence variation in set N of variable genomic target sequences;
2) selecting a primer set for the multiplex amplification and genotyping of the members of set N comprising:
a) from the set of all possible primers for each genomic target sequence variation of step (1), randomly selecting set P, a population of sets of candidate primers, each individual set of candidate primers in population P including a primer pair for the amplification of each member of set N of variable genomic target sequences to be genotyped;
b) calculating a fitness score for each member of the population of set P by calculating ΔG for all possible interactions between candidate primers in each member of the population of set P, and assigning each member of set P a Fitness Score according to the rule:
i) G=the set of ΔG's for all possible interactions determined for a given member of set P;
ii) Number of top scorers to go into next generation=1 . . . N, Number of distinct populations sets=1 . . . N, and Population size=1 . . . N such that number of top scorers to go into next generation is greater or equal to population size;
wherein the fitness score is calculated by:
iii) for each member of set P, calculating the sum, S, of |ΔG|^{Q} for each ΔG value in that member, wherein Q is a weighting factor constant exponent that makes large ΔG absolute values much larger than small values;
iv) S′=S/#of ΔG values in G;
v) H=T/S′, wherein T is a constant that makes H small for large values of S′ and H large for small values of S′;
vi) Fitness Score=H^{R}, wherein R is a weighting factor constant exponent that makes large values of H larger, and small values of H smaller;
c) selecting a set of primers for the multiplex amplification and genotyping of members of set N by:
i) randomly selecting a plurality of sets of “parent” sets of candidate primers, each having parent set A and parent set B, from set P based upon Fitness Scores;
ii) for each member of the plurality of sets of parents, creating a crossover set of candidate primers by replacing a subset of candidate primers in parent set A with a corresponding subset of candidate primers in parent set B, resulting in two crossover sets, crossover set A and crossover set B; and
iii) randomly replacing one primer pair in crossover set A with a different primer pair for the corresponding variable genomic target sequence to create a next generation population of candidate sets of primers, Generation 2; and
d) iteratively repeating steps (a)-(c), whereby a primer set for the multiplex amplification and genotyping of set N of variable genomic target sequences is selected; and
B) synthesizing the primer set designed in step (A).

40. A method of multiplex amplification, sequencing, and/or genotyping comprising using an optimized primer set designed according to claim 28.

41. The method of claim 28, wherein the optimized primer set has decreased primer-primer interactions with other primers in the set, compared to the initial set of forward and reverse amplification primers.

42. The method of claim 28, wherein the Fitness Score of the optimized primer set is increased compared to the initial set of forward and reverse amplification primers.

43. The method of claim 28, wherein the Fitness Score of the optimized primer set is a threshold Fitness Score of at least 400.

44. The method of claim 28, wherein the fitness score optimization method is a method of genetic algorithm,
wherein steps (2) and (3) comprise:
i) for each member of set N, selecting from the set of primers in step (1) a subset of primer pairs that satisfies the conditions for a primer selection algorithm and is predicted to be specific for its target; and
ii) repeating step (i) to generate set P, a population of randomly selected primer sets for each target gene in set N; and
wherein step (4) comprises:
iii) calculating a Fitness Score for each member of the population P; and
iv) placing members of population P into a pool of candidate primer sets on the basis of Fitness Scores; and
v) randomly selecting a plurality of “parent” sets of candidate primers from the pool of step (iv), each parent set including a different pair of candidate primer sets, parent A and parent B; and
vi) for each parent set of candidate primers, creating a crossover set of candidate primers by replacing a subset of candidate primer pairs of parent A with the corresponding subset of primer pairs of parent B; and
vii) randomly replacing one primer pair in crossover set A with a different primer pair for the corresponding target sequence generated in step (i) to create a Generation 2 population of primer sets for each target gene in set N; and
viii) repeating steps (iii)-(vii) iteratively until a set of primer pairs for target genes in set N is identified that has a Fitness Score at a predetermined threshold, and runs for an additional set amount of iterations with no measurable improvement in the fitness of the best member, whereby an optimized primer set is designed.
